(19) United States
(12) Patent Application Publication
(10) Pub. No.: US 2025/0277786 A1
(43) Pub. Date: Sep. 4, 2025

(54) GENE ACTIVATION TARGETS FOR ENHANCED HUMAN T CELL FUNCTION

(71) Applicants: THE J. DAVID GLADSTONE INSTITUTES, A TESTAMENTARY TRUT UNDER THE WILL OF J. DAVID GLADSTONE, San Francisco, CA (US); THE REGEnS OF THE INVERSITY OF CALIFORNIA, usAakland, AA (US)

(72) Inventors: Alexander Marson, San Francisco, CA (US); Ralf Schmidt, Bernsdorf Saxany (DE); Zachary Steinhart, San Francisco, CA (US)

(21) Appl. No.: 18/261,954

(22) PCT Filed: Jan. 19, 2022
(86) PCT No.: PCT/US2022/012988

§ 371 Date: Jul. 18, 2023

Related U.S. Application Data
(60) Provisional application No. 63/138,841, filed on Jan. 19, 2021.

(51) Int. Cl. G01N 33/50; C12N 9/22; C12N 15/11; C12N 15/90

(52) U.S. Cl. CPC G01N 33/505; C12N 9/226; C12N 15/11; C12N 15/907; G01N 33/5023; C12N 2310/20

(57) ABSTRACT

Described herein are regulators of T cells as well as methods of modulating such T cell regulators, and methods of identifying new agents that modulate the T cell regulators. Modification of such T cell regulators in lymphoid and/or myeloid cells can provide lymphoid/myeloid cells that can be administered to subjects in need thereof, for example, subject suffering from immune disorders, cancer and other diseases and conditions.

### PRIORITY

This application claims the benefit of priority of U.S. Provisional Patent Application No. 63/138,841, filed on Jan. 19, 2021, the benefit of priority of which is claimed hereby, and which is incorporated by reference herein in its entirety.

### GOVERNMENT SUPPORT

This invention was made with government support under grant no. DK111914 awarded by The National Institutes of Health, The government has certain rights in the invention,

### INCORPORATION BY REFERENCE OF SEQUENCE LISTING

This application contains a Sequence Listing which has been submitted electronically in ST25 format and is hereby incorporated by reference in its entirety. Said ST25 file, created on Apr. 5, 2024, is named “3730183WO1.txt” and is 314,677 bytes in size.

### BACKGROUND

Examples of cellular therapeutic agents that can be useful as anticancer therapeutics include CD8+ T cells, CD4+ T cells, NK cells, macrophages, dendritic cells, and chimeric antigen receptor (CAR) T cells. Use of patient-derived immune cells can also be an effective cancer treatment that has little or no side effects. NK cells have cell-killing efficacy and have several side effects due to not having antigen specificity. Dendritic cells are therapeutic agents belonging to the vaccine concept in that they have no function of directly killing cells and are capable of delivering antigen specificity to T cells in the patient's body so that cancer cell specificity is imparted to T cells with high efficiency. In addition, CD4+ T cells play a role in promoting productive, antigen-dependent immune responses, and CD8+ T cells are known to have antigen specificity and cell-killing function.

However, most cell therapeutic agents, which have been used or developed to date, have major clinical limitations. For example, cancer cells, on their own, secrete substances that suppress immune responses in the human body, or do not present antigens necessary for production of antibodies against such cancer cells, thereby preventing an appropriate immune response from occurring.

### SUMMARY

Regulators of T cell function are described herein as well as methods of using such regulators. Genome-wide CRISPR activation (CRISPRa) and CRISPR interference (CRISPRi) screens were performed in primary human T cells to identify genetic regulators of therapeutically relevant T cell phenotypes. These screens identified 1074 genes exhibiting significant responses to those phenotypes. The screen identified known genes involved in T cell function, showing that the screens reliably identified genes that actually do affect T cell function. However, the screens also identified novel genes involved in T cell function.

Methods are described herein that involve ex vivo modification of any of the regulator genes listed in Tables 1-7 or FIGS. 1-4 within at least one lymphoid or myeloid cell, or a combination thereof to generate at least one modified lymphoid cell, at least one modified myeloid cell, or a mixture of modified lymphoid and modified myeloid cells. For example, the modification can be one or more deletions, substitutions or insertions into one or more endogenous genomic sites of any of the genes listed in Tables 1-7 or FIGS. 1-4. The modification can be reduction of expression or translation of any of the genes listed in Tables 1-7 or FIGS. 1-4. The reduction of expression or translation can be by an inhibitory nucleic acid (e.g., RNAi, shRNA, siRNA). The modification can be increased expression of any of the genes listed in Tables 1-7 or FIGS. 1-4. For example, the increased expression can be by modification of one or more promoters of any of the genes listed in Tables 1-7 or FIGS. 1-4. The modification can be one or more CRISPR-mediated modifications or activations of any of the genes listed in Tables 1-7 or FIGS. 1-4. The modification can involve transformation of at least one lymphoid or myeloid cell, or a combination thereof, with one or more expression cassettes comprising a promoter operably linked to a nucleic acid segment comprising a coding region of any of the genes listed in Tables 1-7 or FIGS. 1-4.

The methods can also include administering at least one of the modified lymphoid cells, at least one of the modified myeloid cells, or a mixture of modified lymphoid and modified myeloid cells to a subject.

In some cases, the method can include incubating the at least one modified lymphoid cell, at least one modified myeloid cell, or a mixture of modified lymphoid and modified myeloid cells to form a population of modified cells. Such a population of modified cells can be administered to a subject. In some cases, the subject can have a disease or condition. For example, the disease or condition is an immune condition or cancer.

Also described are methods that involve contacting at least one test agent with test cells to provide a test assay mixture, and measuring:
cellular proliferation of the test cells, cytokine release by the test cells, or a combination thereof:
activation of the test cells;
expression or activity of any of the regulators listed in Tables 1-7 or FIG. 1-4 in the cells; or
a combination thereof.

The methods can also include comparing the measured results to control results. The control results can be results of the test cells measured without any of the test agents.

For example, the test cells can include lymphoid and/or myeloid cells. Examples of the test cells can include cytotoxic T cells, helper T cells, regulatory T cells, naive T cells, activated T cells, CD4 T cells, CD8 T cells, gamma delta T cells, chimeric antigen receptor (CAR) cells, natural killer (NK) cells, induced pluripotent stem cell-derived immune (e.g., lymphoid and/or myeloid) cells, or a combination thereof.

The results so measured can be compared to results of a control cell mixture that includes the T cells and test cells measured without any of the test agents.

### DRAWINGS

FIGS. 1A-E. Genome-wide CRISPRa screens for cytokine production in stimulated primary human T cells. (A) Schematic of CRISPRa screens. (B) sgRNA iog₂-fold changes for genes of interest in IL-2 (left) and IFN-γ (right) screens. Bars represent the mean log₂-fold change for each sgRNA across two human blood donors. Density plots above represent the distribution of all sgRNAs. (C and D) Scatter plots of median sgRN-A log₂-fold change (high/low sorting bins) for each gene, comparing screens in two donors, for IL-2 (C) and IFN-γ screens (D). (E) Comparison of gene log₂-fold change (median sgRNA, mean of two donors) in IL-2 and IFN-γ screens.

FIGS. 2A-1H. Integrated CRLISPRi and CRISPRi screens map the genetic circuits underlying T cell cytokine response in high resolution. (A and B) Median sgRNA log₂-fold change (high/low sorting bins) for each gene, comparing CRISPRi screens in two donors, for IL-2 (A) and IFN-γ screens (B). (C) Distributions of gene mRNA expression for CRISPRa and CRISPRi cytokine screen hits in resting CD4-T cells (this study). (D) Comparison IL-2 CRISPRi and CRISPRa screens with genes belonging to the T cell receptor signaling pathway (KEGG pathways) indicated in colors other than gray. (E) Comparison IFN-γ CRISPRi and CRISPRa screens with manually selected NF-κB pathway regulators labeled. All other genes are shown in gray. (F) Map of NF-κB pathway regulators labeled in (D). (C) Map of screen hits with previous evidence of defined function in T cell stimulation and costimulation signal transduction pathways. Genes shown are significant hits in at least one screen and were selected based on review of literature and pathway databases (e.g., KEGG and Reactome). Tiles represent proteins encoded by indicated genes, with the caveat that due to space constraints, subcellular localization is inaccurate, as many of the components shown in the cytoplasm occur at the plasma membrane. Tiles are colored according to log₂-fold change Z-score as shown in the sub-panel, with examples of different hits. Large arrows at the top represent stimulation/costimulation sources. (H) Select screen hits with less well-described functions in T cells in the same format as (G), For (H), only significant hits from the top positive and negative ranked genes by log_-fold change for each screen were candidates for inclusion.

FIGS. 3A-1. Characterization of CRISPRa screen hits by arrayed profiling. (A) Schematic of arrayed experiments. (B) Comparison of IL-2 (in CD4 T cells) and IFN-γ (in CD8⁺ T cells) CRISPRa screens, with genes targeted by the arrayed sgRNA panel indicated, as well as their screen hit categorization. Paralogs of arrayed panel genes that were also highly ranked hits are additionally indicated. (C) Representative intracellular cytokine staining flow cytometry for indicated cytokines in control (NO-TARGET_1 sgRNA) or VAV1 (VAV1_1 sgRNA) CRISPRa T cells after 10 hours of stimulation. (D) Intracellular cytokine staining of full arrayed sgRNA panel, showing percent of cells gated positive for indicated cytokines in CD4+ or CD8V T cells. Points represent the mean value of four donors, with and without stimulation. Dashed vertical lines represent the mean no-target control sgRNA control value with stimulation. *q<0.05, **q<0.01, Mann-Whitney U test followed by q-value multiple comparison correction. Medium-stimulation dose is shown for IL-2 and IFN-γ and low-dose stimulation is shown for TNF-α. (E) Scatter plot comparison of log₂-fold changes in percent cytokine positive cells for arrayed panel sgRNAs versus the mean of no-target control sgRNAs in stimulated CD4⁺ and CD8⁺ cells, using the same data from (D). (F) Secreted cytokine staining arrayed panel grouped by indicated gene categories, with sgRNAs targeting IL2 and IFNG genes removed. Points represent a single gene and donor measurement. *P<0.05, **P<0.01, ***P<0.001, Mann-Whitney U test. (G) Principal component analysis of secreted cytokine measurements resulting from indicated CRISPRa sgRNAs. (H) Heatmap of selected secreted cytokine measurements grouped by indicated biological category. Values represent the median of four donors, followed by Z-score scaling for each cytokine.

FIGS. 4A-J. CRTSPRa perturb-seq captures diverse T cell states driven by genome-wide cytokine screen hits. (A) Schematic of CRISPRa Perturb-seq experiment. (B) Categorical breakdown of genes targeted by sgRNA library, with the library comprising hits from our primary genome wide CRISPRa cytokine screens as indicated. Genes with a summed log₂-fold change<0 across both screens (diagonal line) are categorized as negative regulators. (C) UMAP projection of post-quality control filtered restimulated T cells, colored by blood donor. (D) Distribution of CD4 and CD8 T cells across restimulated T cell UMAP projection. Each bin is colored by the average log₂(CD4/CD8) transcript levels of cells in that bin. (E) Restimulated T cell UMAP colored by average cell activation score in each bin. (F) Boxplots of restimulated T cells' activation scores grouped by sgRNA target genes. Dashed line represents the median activation score of no-target control cells. *P<0.05, **P<0.01, ***P<,0.001 Mann-Whitney U test with Bonferroni correction. (G) Restimulated T cell UMAP with cells colored by cluster. (H) Heatmap of differentially expressed marker genes in each cluster. The top 50 statistically significant (FDR<0.05) differentially upregulated genes for each cluster are shown, with genes that are upregulated in multiple clusters being given priority to the cluster with the higher log₂-fold change for the given gene. The top marker genes by log₂-fold change in each clusters' section are listed to the right. Top overrepresented sgRNAs in each cluster by odds ratio are listed to the next right. Top differentially upregulated cytokine genes in each cluster are listed to the next right. Mean cell log₂(CD4/CD8) cell transcript values in each cluster are shown on the far right. (I) Restimulated T cell UMAP with the expression of indicated genes shown. (J) Contour density plots of restimulated cells assigned to indicated sgRNA targets in UMAP space. The no-target control contour is shown in grayscale underneath. “Perturbed Cells” represents all cells assigned a single sgRNA other than no-target control sgRNAs.

FIG. 5 provides In vitro data using the identified hits for T cell cancer therapies.

### DETAILED DESCRIPTION

Methods and compositions are described herein for modulating T cell responses. The T cells can be modulated in vivo or ex vivo. T cells modulated ex vivo can be administered to a subject who may benefit from such administration. Methods are also described herein for evaluating test agents and identifying agents that are useful for modulating T cell functions.

Regulation of cytokine production in stimulated T cells can be disrupted in autoimmunity, immunodeficiencies, and cancer. Systematic discovery of stimulation-dependent cytokine regulators requires both loss-of-function and gain-of-function studies, which have been challenging in primary human cells. We now report genome wide CRISPR activation (CRISPRa) and interference (CRISPRi) screens in primary human T cells to identify gene networks controlling interleukin 2 and interferon gamma production. Arrayed CRISPRa confirmed key hits and enabled multiplexed secretome characterization, revealing reshaped cytokine responses. Coupling CRISPRa screening with single-cell RNA-seq enabled deep molecular characterization of screen hits, revealing how perturbations tuned T cell activation and promoted cell states characterized by distinct cytokine expression profiles. Together, these screens reveal genes that reprogram immune cell functions.

### Modulating T Cell Responses

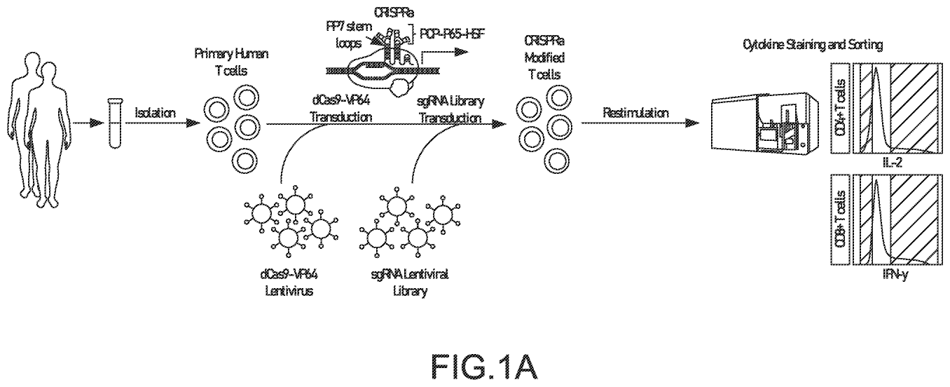

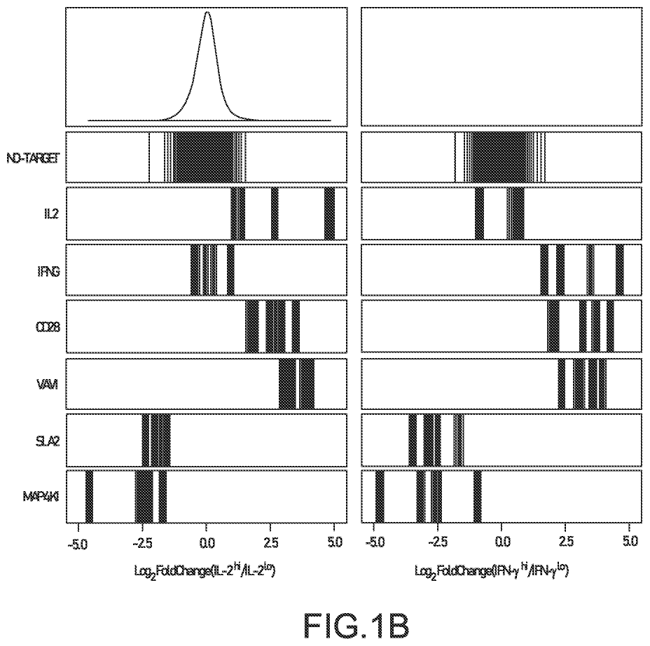

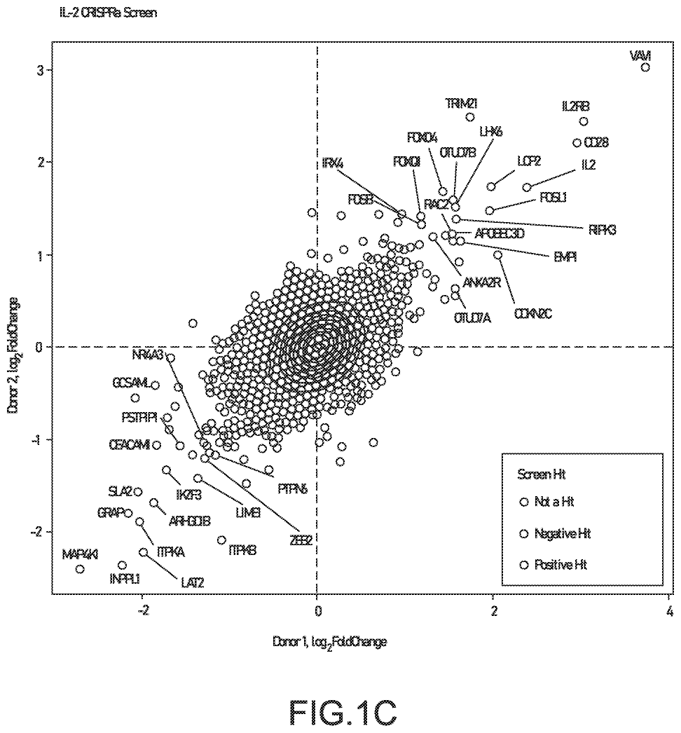

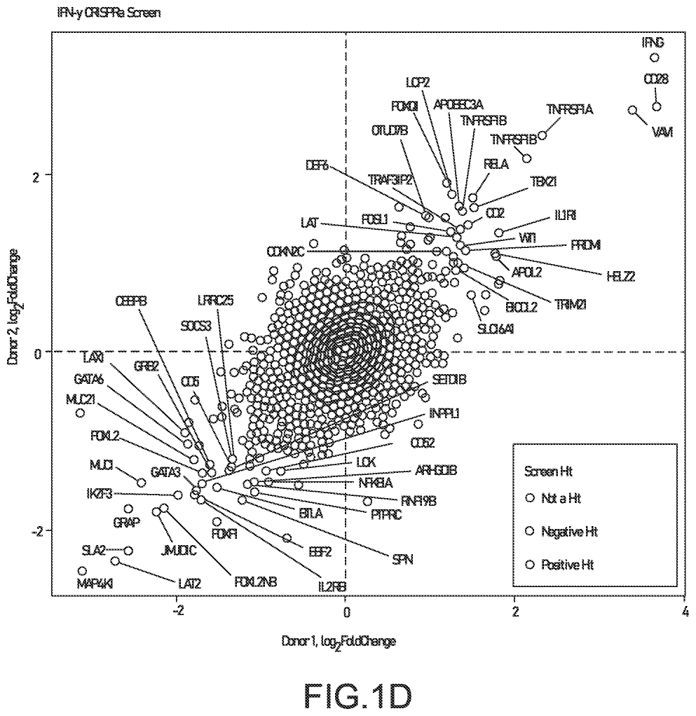

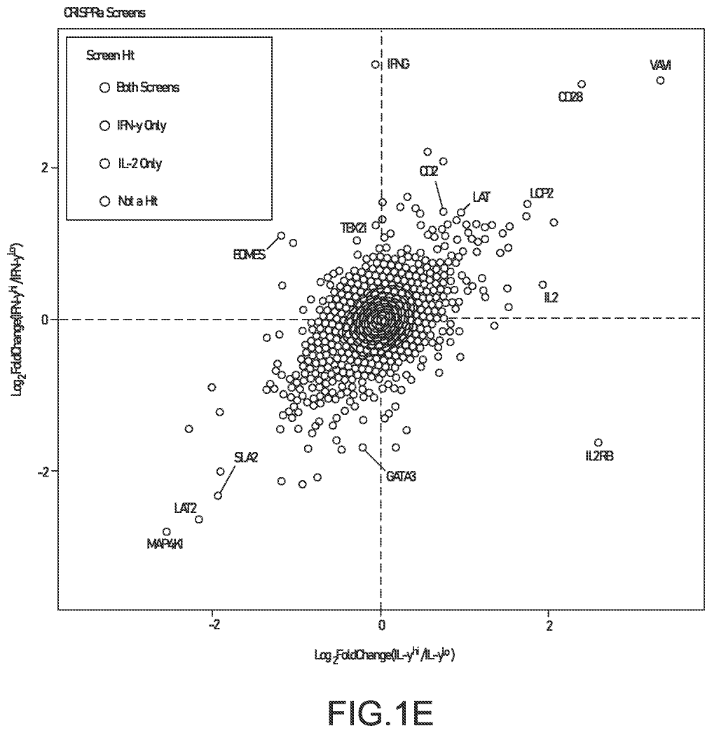

Lists of negative and positive regulators of T cells are provided in Tables 1-7 or FIGS. 1-4. Such regulators can modulate gamma interferon (IFN-γ) production, interleukin 2 (112) production, cellular proliferation of T cells, or a combination thereof Any of the regulators of T cells can be used in the methods and compositions described herein. Agents that modulate the listed regulators can also be used in the methods and compositions described herein. For example, to positively regulate T cells one or more expression cassettes encoding one or more positive T cell regulators, one or more agents that increase the expression or activity of such positive regulator, or agents that inhibit negative regulators of T cells can be used. To negatively regulate T cells, for example, antibodies, one or more expression cassettes encoding one or more negative T Cell regulators, one or more agents that increase the expression or activity of such negative 10 regulator, or agents that inhibit positive regulators of T cells can be used. Agents that can modulate the T cell regulators can include expression vectors, inhibitory nucleic acids, antibodies, small molecules, guide RNAs, nucleases (e.g., one or more cas nucleases), nuclease-dead cas variants (e.g., dCas9-VP64, dCas9-KRAB), or a combination thereof.

For example, T cells and other types of cells can be modified ex vivo to increase or decrease any of the T cell regulators listed in Tables 1-7 or FIGS. 1-4, and the modified cells can be administered to a subject that may benefit from such administration, In another example, the expression or activity of any of the T cell regulators listed in Tables 1-7 or FIGS. 1-4 can be modulated by in vivo administration of expression vectors, virus-like particles (VLP), CRISPR-related ribonucleoprotein (RNP) complexes, and combinations thereof that include or target any of the regulators listed in Tables 1-7 or FIGS. 1-4. The regulator nucleic acids, regulator protein, regulator guide RNAs and CRISPR nucleases can be introduced via one or more vehicles such as by one or more expression vectors (e.g., viral vectors), virus like particles, ribonucleoproteins (RNPs), nanoparticles, liposomes, or a combination thereof. The vehicles can include components or agents that can target particular cell types (e.g., antibodies that recognize cell-surface markers), facilitate cell penetration, reduce degradation, or a combination thereof.

In addition, new agents can be identified by screening methods described herein that include, for example, evaluating assay mixtures containing one or more test agents and a population of T cells after incubation of the assay mixtures for a time and under conditions sufficient for determining whether the test agent can modulate the expression or activities of any of the regulators described herein. In some cases, the assay mixtures can include T cells and other types of cells, for example, other immune cells such as those that can interact with T cells. Useful test agents identified by such methods can, for example, increase or decrease the expression or activities of any of the regulators listed in any of Tables 1-7 or FIGS. 1-4.

Hence, any of the regulators of T cells, as well as agents that can modulate those regulators (i.e., modulators), can be used in the methods and compositions described herein.

The T cell regulators were identified by detecting altered IL-2 cytokine production, 1IFN-γ production, and cell proliferation of T cell receptor (TCR) stimulated primary T cells isolated from two different donors that were subjected to CRISPR-meditated genetic modification. Both positive and negative regulators of T cells were identified.

The agents that can modulate T cells or the T cell regulators described herein can be expression systems encoding a regulator or modulating agent, antibodies, small molecules, inhibitory nucleic acids, peptides, polypeptides, guide RNAs, cas nucleases (e.g., a cas9 nuclease), nuclease-dead cas variants (e.g., dCas9-VP64, dCas9-KRAB), and combinations thereof Examples of such agents are described hereinbelow.

The regulators and/or the agents that modulate the regulators can be evaluated by various assay procedures. Such assay procedures can also be used to identify new T cell regulators. In some cases, the assay procedures can be used to evaluate the utility of a type (positive or negative effect), quantity, or extent of a. regulator or modulating agent activity on T cell activity or T cell numbers.

For example, the methods for evaluating Applicants' regulators/agents or new regulators/agents can involve contacting one or more T cells (or a T cell population) with a test agent to provide a test assay mixture, and evaluating the test assay mixture for at least one of:
Detecting and/or quantifying cytokine (e.g., interferon-γ, (IFN-γ, interleukin-2(IL-2)) production;
Quantifying the numbers of T cells within the test assay mixture;
Detecting proliferation via quantification of a dye that dilutes with cell divisions;
Detecting whether T cells in the test assay mixture express one or more of the positive or negative regulators described herein;
Quantifying the number of cells that express one or more of the positive or negative regulators expressed by a population of T cells; or
A combination thereof.

The T cells or T cell populations that are contacted with the test agent/test regulator can also include a variety of lymphoid and/or myeloid immune cells. For example, test agents can be introduced into an assay mixture that contains cytotoxic T cells, helper T cells, regulatory T cells, naive T cells, activated T cells, CD4 T cells, CD8 T cells, gamma delta T cells, chimeric antigen receptor (CAR) cells, natural killer (NK) cells, induced pluripotent stem cell-derived immune (e.g., lymphoid and/or myeloid) cells, or a combination thereof,

Test agents that exhibit in vitro activity for modulating the T cells or for modulating the amount or activity of any of the regulators described herein can be evaluated in animal disease models. Such animal disease models can include cancer disease animal models, immune system disease models, or combinations thereof.

### Positive T Cell Regulators

The following genes are positive regulators of T cells as detected by interferon-y production (see Table 1): APOBEC3C, APOBEC3D, APOL2, ASB12, BACE2, BCL9, BICDL2, C15orf52, C1 orf94, CD2, CD247, CD28, CNGB1, CTSK, DEAFI, DEF6, DEPDC7, DKK2, EMIP1, EOMES, EP300, FLT3, FOSL1, FOXQ1, GINS3, GLMN, GNA11, HELZ2, -RASLS5, IFNG, IL1IR, IL9R, KLHDC3, KLRC4, LAT, LCP2, LDB2, LTBR, MVB12A, NBPF6, NIT1, NLRC3, ORCI, OTUD7A, OTUD7B, PIK3AP1, PLCG2, PRDMI, PRKD2, PROCAI, RELA, RNF217, SAFB2, SLC16A1, SLC5AI0, SLC7A3, SPPL2B, TAGAP, TBX21, TMEM150B, TMILGD2, TNFRSF12A, TNFRSF14, TNFRSFIA, TNFRSFlB, TNFRSF8, TNIFRSF9, TORlA, TPGS2, TRADD, TRAF3TP2, TRIM21, VAV1, WT1, ZNF630, and ZNF717. Example 2 provides additional positive regulators T cells that were detected by interferon-y production.

Sequences and other information relating to these genes, and their encoded proteins, is available, for example from the NCBI and UniPROT databases, which are incorporated by reference.

A few examples of protein sequences encoded by some of the genes detected as positive regulators of T cells by interferon-y production are provided. For example, an amino acid sequence for the protein encoded by the human BICDL2 gene that is a positive regulator of T cells as detected by interferon-y production is available from the UniPROT database as accession no. A1 A5D9, shown below as SEQ ID NO:1.

```
                10 20 30 40 50
MSSPDGPSFP SGPLSGGASP SGDEGFFPFV LERRDSFLGG GPGPEEPEDL
                60 70 80 90 100
ALQLQQKEKD LLLAAELGKM LLERNEELRR QLETLSAQHL EREERLQQEN
              110 120 130 140 150
HELRRGLAAR GAEWEARAVE LEGDVEALRA QLGEQRSEQQ DSGRERARAL
              160 170 180 190 200
SELSEQNLRL SQQLAQASQT EQELQRELDA LRGQCQAQAL AGAELRTRLE
              210 220 230 240 250
SLQGENQMLQ SRRQDLEAQI RGLREEVEKG EGRLQTTHEE LLLLRRERRE
              260 270 280 290 300
HSLELERARS EAGEALSALR RLQRRVSELE EESRLQDADV SAASLQSELA
              310 320 330 340 350
HSLDDGDQGQ GADAPGDTPT TRSPKTRKAS SPQPSPPEEI LEPPKKRTSL
              360 370 380 390 400
SPAEILEEKE VEVAKLQDEI SLQQAELQSL REELQRQKEL RAQEDPGEAL
              410 420 430 440 450
HSALSDRDEA VNKALELSLQ LNRVSLERDS LSRELLRAIR QKVALTQELE
              460 470 480 490 500
AWQDDMQVVI GQQLRSQRQK ELSASASSST PRRAAPRFSL RLGPGPAGGF
LSNLFRRT
```

A cDNA and a chromosomal sequence encoding the BICDL2 protein is available from the NCBI database as accession no. AL833749 and ACI08134, respectively.

An amino acid sequence for the protein encoded by the human C1orf94 gene that is a positive regulator of T cells as detected by interferon-y production is available from the UniPROT database as accession no. Q6P1IW5, shown below as SEQ ID NO:2.

```
                10 20 30 40 50
MRGGGGCVLA LGGQRGFQKE RRRMASGNGL PSSSALVAKG PCALGPFPRY
                60 70 80 90 100
IWIHQDTPQD SLDKTCHEIW KRVQGLPEAS QPWTSMEQLS VPVVGTLRGN
              110 120 130 140 150
ELSFQEEALE LSSGKDEISL LVEQEFLSLT KEHSILVEES SGELEVPGSS
              160 170 180 190 200
PEGTRELAPC ILAPPLVAGS NERPRASIIV GDKLLKQKVA MPVISSRQDC
              210 220 230 240 250
DSATSTVTDI LCAAEVKSSK GTEDRGRILG DSNLQVSKLL SQFPLKSTET
              260 270 280 290 300
SKVPDNKNVL DKTRVTKDFL QDNLFSGPGP KEPTGLSPFL LLPPRPPPAR
              310 320 330 340 350
PDKLPELPAQ KRQLPVFAKI CSKPKADPAV ERHHLMEWSP GTKEPKKGQG
              360 370 380 390 400
SLFLSQWPQS QKDACGEEGC CDAVGTASLT LPPKKPTCPA EKNLLYEFLG
              410 420 430 440 450
ATKNPSGQPR LRNKVEVDGP ELKFNAPVTV ADKNNPKYTG NVFTPHEPTA
              460 470 480 490 500
MTSATLNQPL WLNLNYPPPP VFTNHSTFLQ YQGLYPQQAA RMPYQQALHP
              510 520 530 540 550
QLGCYSQQVM PYNPQQMGQQ IFRSSYTPLL SYIPFVQPNY PYPQRTPPKM
              560 570 580 590
SANPRDPPLM AGDGPQYLFP QGYGFGSTSG GPLMHSPYES SSGNGINF
```

A cDNA and a chromosomal sequence encoding the Q6P1W5 protein is available from the NCBI database as accession no. AK123355 and AC115286, respectively.

An amino acid sequence for the protein encoded by the human CNGB1 gene that is a positive regulator of T cells as detected by interferon-i production is available from the UniPROT database as accession no. Q14028, shown below as SEQ ID NO:³.

```
                10 20 30 40 50
MLGWVQRVLP QPPGTPRKTK MQEEEEVEPE PEMEAEVEPE PNPEEAETES
                60 70 80 90 100
ESMPPEESEK EEEVAVADPS PQETKEAALT STISLRAQGA EISEMNSPSR
              110 120 130 140 150
RVLTWLMKGV EKVIPQPVHS ITEDPAQILG HGSTGDTGCT DEPNEALEAQ
              160 170 180 190 200
DTRPGLRLLL WLEQNLERVL PQPPKSSEVW RDEPAVATGA ASDPAPPGRP
              210 220 230 240 250
QEMGPKLQAR ETPSLPTPIP LQPKEEPKEA PAPEPQPGSQ AQTSSLPPTR
              260 270 280 290 300
DPARLVAWVL HRLEMALPQP VLHGKIGEQE PDSPGICDVQ TISILPGGQV
              310 320 330 340 350
EPDLVLEEVE PPWEDAHQDV STSPQGTEVV PAYEEENKAV EKMPRELSRI
              360 370 380 390 400
EEEKEDEEEE EEEEEEEEEE EVTEVLLDSC VVSQVGVGQS EEDGTRPQST
              410 420 430 440 450
SDQKLWEEVG EEAKKEAEEK AKEEAEEVAE EEAEKEPQDW AETKEEPEAE
              460 470 480 490 500
AEAASSGVPA TKQHPEVQVE DTDADSCPLM AEENPPSTVL PPPSPAKSDT
              510 520 530 540 550
LIVPSSASGT HRKKLPSEDD EAEELKALSP AESPVVAWSD PTTPKDTDGQ
              560 570 580 590
DRAASTASTN SAIINDRLQE LVKLEKERTE KVKEKLIDPD VTSDEESPKP
              610 620 630 640 650
SPAKKAPEPA PDTKPAEAEP VEEEHYCDML CCKFKHRPWK KYQFPQSIDP
              660 670 680 690 700
LTNLMYVLWL FFVVMAWNWN CWLIPVRWAF PYQTPDNIHH WLLMDYLCDL
              710 720 730 740 750
IYFLDITVFQ TRLQFVRGGD IITDKKDMRN NYLKSRRFKM DLLSLLPLDF
              760 770 780 790 800
LYLKVGVNPL LRLPRCLKYM AFFEFNSRLE SILSKAYVYR VIRTTAYLLY
              810 820 830 840 850
SLHLNSCLYY WASAYQGLGS THWVYDGVGN SYIRCYYFAV KTLITIGGLP
              860 870 880 890 900
DPKTLFEIVF QLLNYFTGVF AFSVMIGQMR DVVGAATAGQ TYYRSCMDST
              910 920 930 940 950
VKYMNFYKIP KSVQNRVKTW YEYTWHSQGM LDESELMVQL PDKMRLDLAI
              960 970 980 990 1000
DVNYNIVSKV ALFQGCDRQM IFDMLKRLRS VVYLPNDYVC KKGEIGREMY
            1010 1020 1030 1040 1050
IIQAGQVQVL GGPDGKSVLV TLKAGSVFGE ISLLAVGGGN RRTANVVAHG
            1060 1070 1080 1090 1100
FTNLFILDKK DLNEILVHYP ESQKLLRKKA RRMLRSNNKP KEEKSVLILP
            1110 1120 1130 1140 1150
PRAGTPKLFN AALAMTGKMG GKGAKGGKLA HLRARLKELA ALEAAAKQQE
            1160 1170 1180 1190 1200
LVEQAKSSQD VKGEEGSAAP DQHTHPKEAA TDPPAPRTPP EPPGSPPSSP
            1210 1220 1230 1240 1250
PPASLGRPEG EEEGPAEPEE HSVRICMSPG PEPGEQILSV KMPEEREEKA
E
```

A cDNA and a chromosomal sequence encoding the Q14028 protein is available from the NCBI database as accession no. UI8945 and L 15296, respectively,

An amino acid sequence for the protein encoded by the human DEPDC7 gene that is a positive regulator of T cells as detected by interferon-y production is available from the UniPROT database as accession no. Q96QD5. shown below as SEQ ID NO. 4

```
                10 20 30 40 50
MATVQEKAAA LNLSALHSPA HRPPGFSVAQ KPFGATYVWS SIINTLQTQV
                60 70 80 90 100
EVKKRRHRLK RHNDCFVGSE AVDVIFSHLI QNKYFGDVDI PRAKVVRVCQ
              110 120 130 140 150
ALMDYKVFEA VPTKVFGKDK KPTFEDSSCS LYRFTTIPNQ DSQLGKENKL
              160 170 180 190 200
YSPARYADAL FKSSDIRSAS LEDLWENLSL KPANSPHVNI SATLSPQVIN
              210 220 230 240 250
EVWQEETIGR LLQLVDLPLL DSLLKQQEAV PKIPQPKRQS TMVNSSNYLD
              260 270 280 290 300
RGILKAYSDS QEDEWLSAAI DCLEYLPDQM VVEISRSFPE QPDRTDLVKE
              310 320 330 340 350
LLFDAIGRYY SSREPLLNHL SDVHNGIAEL LVNGKTEIAL EATQLLLKLL
              360 370 380 390 400
DFQNREEFRR LLYEMAVAAN PSEFKLQKES DNRMVVKRIF SKAIVDNKNL
              410 420 430 440 450
SKGKTDLLVL FLMDHQKDVF KIPGTLHKIV SVKLMAIQNG RDPNRDAGYI
              460 470 480 490 500
YCQRIDQRDY SNNTEKTTKD ELLNLLKTLD EDSKLSAKEK KKLLGQFYKC
              510
HPDIFIEHFG D
```

A cDNA and a chromosomal sequence encoding the Q96QD5 protein is available from the NCBI database as accession no. AJ245600 and AC107939, respectively.

An amino acid sequence for the protein encoded by the human HRASLS5 gene that is a positive regulator of T cells as detected by interferon-y production is available from the UniPROT database as accession no. Q96KN8, shown below as SEQ ID NO:5.

```
                10 20 30 40 50
MGLSPGAEGE YALRLPRIPP PLPKPASRTA STGPKDQPPA LRRSAVPHSG
                60 70 80 90 100
LNSISPLELE ESVGFAALVQ LPAKQPPPGT LEQGRSIQQG EKAVVSLETT
              110 120 130 140 150
PSQKADWSSI PKPENEGKLI KQAAEGKPRP RPGDLIEIFR IGYEHWAIYV
              160 170 180 190 200
EDDCVVHLAP PSEEFEVGSI TSIFSNRAVV KYSRLEDVLH GCSWKVNNKL
              210 220 230 240 250
DGTYLPLPVD KIIQRTKKMV NKIVQYSLIE GNCEHFVNGL RYGVPRSQQV
              260 270
EHALMEGAKA AGAVISAVVD SIKPKPITA
```

A cDNA and a chromosomal sequence encoding the HRASLS5 protein is available from the NCBI database as accession no. AB298804 and AP000484, respectively.

An amino acid sequence for the protein encoded by the human KLHDC3 gene that is a positive regulator of T cells as detected by interferon-y production is available from the UniPROT database as accession no. Q9BQ90, shown below as SEQ ID NO: 6.

```
                10 20 30 40 50
MLRWTVHLEG GPRRVNHAAV AVGHRVYSFG GYCSGEDYET LRQIDVHIFN
                60 70 80 90 100
AVSLRWTKLP PVKSAIRGQA PVVPYMRYGH STVLIDDTVL LWGGRNDTEG
              110 120 130 140 150
ACNVLYAFDV NTHKWFTPRV SGTVPGARDG HSACVLGKIM YIFGGYEQQA
              160 170 180 190 200
DCFSNDIHKL DTSTMTWTLI CTKGSPARWR DFHSATMLGS HMYVFGGRAD
              210 220 230 240 250
RFGPFHSNNE IYCNRIRVFD TRTEAWLDCP PTPVLPEGRR SHSAFGYNGE
              260 270 280 290 300
LYIEGGYNAR LNRHEHDLWK FNPVSFTWKK IEPKGKGPCP RRRQCCCIVG
              310 320 330 340 350
DKIVLFGGTS PSPEEGLGDE FDLIDHSDLH ILDFSPSLKT LCKLAVIQYN
              360 370 380
LDQSCLPHDI RWELNAMTIN SNISRPIVSS HG
```

A cDNA and a chromosomal sequence encoding the KLHDC3 protein is available from the NCBT database as accession no. AB055925 and AL136304, respectively,

An amino acid sequence for the protein encoded by the human NBPF6 gene that is a positive regulator of T cells as detected by interferon-y production is available from the UniPROT database as accession no. Q5VWK0, shown below as SEQ ID NO: 7.

```
                10 20 30 40 50
MVVSADPLSS ERAEMNILEI NQELRSQLAE SNQQFRDLKE KFLITQATAY
                60 70 80 90 100
SLANQLKKYK CEEYKDIIDS VLRDELQSME KLAEKLRQAE ELRQYKALVH
              110 120 130 140 150
SQAKELTQLR EKLREGRDAS RWLNKHIKTL LTPDDPDKSQ GQDLREQLAE
              160 170 180 190 200
GHRLAEHLVH KLSPENDEDE DEDEDDKDEE VEKVQESPAP REVQKTEEKE
              210 220 230 240 250
VPQDSLEECA VTCSNSHNPS NSNQPHRSTK ITFKEHEVDS ALVVESEHPH
              260 270 280 290 300
DEEEEALNIP PENQNDHEEE EGKAPVPPRH HDKSNSYRHR EVSFLALDEQ
              310 320 330 340 350
KVCSAQDVAR DYSNPKWDET SLGFLEKQSD LEEVKGQETV APRLSRGPLR
              360 370 380 390 400
VDKHEIPQES LDGCCLTPSI LPDLIPSYHP YWSTLYSFED KQVSLALVDK
              410 420 430 440 450
IKKDQEEIED QSPPCPRLSQ ELPEVKEQEV PEDSVNEVYL TPSVHHDVSD
              460 470 480 490 500
CHQPYSSTLS SLEDQLACSA LDVASPTEAA CPQGTWSGDL SHHRSEVQIS
              510 520 530 540 550
QAQLEPSTLV PSCLRLQLDQ GFHCGNGLAQ RGLSSTTCSF SANADSGNQW
              560 570 580 590 600
PFQELVLEPS LGMKNPPQLE DDALEGSASN TQGRQVTGRI RASLVLILKT
              610 620 630
IRRRLPFSKW RLAFRFAGPH AESAEIPNTA ERMQRMIG
```

A cDNA and a chromosomal sequence encoding the Q5VWK0 protein is available from the NCBT database as accession no. BC125161 and AL390038, respectively.

An amino acid sequence for the protein encoded by the human OTUTD7B gene that is a positive regulator of T cells as detected by interferon-y production is available from the UniPROT database as accession no. Q5VWK0, shown below as SEQ HD NO:8.

```
                10 20 30 40
MVVSADPLSS ERAEMNILEI NQELRSQLAE SNQQERDLKE
                50 60 70 80
KFLITQATAY SLANQLKKYK CEEYKDIIDS VERDELQSME
                90 100 110 120
KLAEKLRQAE ELRQYKALVH SQAKELTQLR EKLREGRDAS
              130 140 150 160
RWLNKHLKTL LTPDDPDKSQ GQDLREQLAE GHRLAEHLVH
              170 180 190 200
KLSPENDEDE DEDEDDKDEE VEKVQESPAP REVQKTEEKE
              210 220 230 240
VPQDSLEECA VTCSNSHNPS NSNQPHRSTK ITFKEHEVDS
              250 260 270 280
ALVVESEHPH DEEEEALNIP PENQNDHEEE EGKAPVPPRH
              290 300 310 320
HDKSNSYRHR EVSFLALDEQ KVCSAQDVAR DYSNPKWDET
              330 340 350 360
SLGFLEKQSD LEEVKGQETV APRLSRGPLR VDKHEIPQES
              370 380 390 400
LDGCCLIPSI LPDLIPSYHP YWSTLYSFED KQVSLALVDK
              410 420 430 440
IKKDQEEIED QSPPCPRLSQ ELPEVKEQEV PEDSVNEVYL
              450 460 470 480
TPSVHHDVSD CHQPYSSTLS SLEDQLACSA LDVASPTEAA
              490 500 510 520
CPQGTWSGDL SHHRSEVQIS QAQLEPSTLV PSCLRLQLDQ
              530 540 550 560
GFHCGNGLAQ RGLSSTICSE SANADSGNQW PFQELVLEPS
              570 580 590 600
LGMKNPPQLE DDALEGSASN TQGRQVTGRI RASLVLILKT
              610 620 630
IRRRLPESKW RLAFRFAGPH AESAEIPNTA ERMQRMIG
```

A cDNA and a chromosomal sequence encoding the Q5VWK0 protein is available from the NCBI database as accession no. BC125161 and AL390038, respectively.

An amino acid sequence for the protein encoded by the human TPGS2 gene that is a positive regulator of T cells as detected by interferon-x production is available from the UniPROT database as accession no. Q68CL5, shown below as SEQ ID NO:9.

```
                10 20 30 40
MEEEASSPGL GCSKPHLEKL TIGITRILES SPGVTEVTII
                50 60 70 80
EKPPAERHMI SSWEQKNNCV MPEDVKNFYL MINGFHMTWS
                90 100 110 120
VKLDEHIIPL GSMAINSISK LTQLTQSSMY SLPNAPTLAD
              130 140 150 160
LEDDTHEASD DQPEKPHEDS RSVIFELDSC NGSGKVCIVY
              170 180 190 200
KSGKPALAED TEIWELDRAL YWHFLIDTET AYYRLLITHL
              210 220 230 240
GLPQWQYAFT SYGISPQAKQ WESMYKPITY NTNLLTEETD
              250 260 270 280
SFVNKLDPSK VEKSKNKIVI PKKKGPVQPA GGQKGPSGPS
              290 300
GPSTSSTSKS SSGSGNPTRK
```

A cDNA and a chromosomal sequence encoding the TPGS2 protein is available from the NCBI database as accession no AK295817 and AC009854, respectively.

An amino acid sequence for the ZNF630 protein encoded by the human ZNF630 gene that is a positive regulator of T cells as detected by interferon-γ production is available from the UniPROT database as accession no. Q2M218, shown below as SEQ ID NO:10.

```
                10 20 30 40
MIESQEPVTF EDVAVDFTQE EWQQLNPAQK TLHRDVMLET
                50 60 70 80
YNHLVSVGCS GIKPDVIFKL EHGKDPWIIE SELSRWIYPD
                90 100 110 120
RVKGLESSQQ IISGELLFQR EILERAPKDN SLYSVLKIWH
              130 140 150 160
IDNQMDRYQG NQDRVLRQVT VISRETLTDE MGSKYSAFGK
              170 180 190 200
MENRCTDLAP LSQKFHKEDS CENSLKSNSD LLNYNRSYAR
              210 220 230 240
KNPTKRERCG RPPKYNASCS VPEKEGFIHT GMEPYGDSQC
              250 260 270 280
EKVISHKQAH VQYKKFQARE KPNVCSMCGK AFIKKSQLII
              290 300 310 320
HQRIHTGEKP YVCGDCRKAF SEKSHLIVHQ RIHTGEKPYE
              330 340 350 360
CTKYGRAFSR KSPFTVHQRV HTGEKPYECE ECPKAFSQKS
              370 380 390 400
HLIIHQRVHT REKPFECSEC RKAFCEMSHL FIHQITHTGK
              410 420 430 440
KPYECTECGK TFPRKTQLII HQRTHTGEKP YKCGECGKTF
              450 460 470 480
CQQSHLIGHQ RIHTGEKPYV CTDCGKAFSQ KSHLTGHQRL
              490 500 510 520
HTGEKPYMCT ECGKSFSQKS PLIIHQRIHT GEKPYQCGEC
              530 540 550 560
GKTFSQKSLL IIHLRVHTGE KPYECTECGR AFSLKSHLIL
              570 580 590 600
HQRGHTGEKP YECSECGKAF CGKSPLIIHQ KTHPREKTPE
              610 620 630 640
CAESGMTFEW KSQMITYQRR HTGEKPSRCS DCGKAFCQHV
              650
YFTGHQNPYR KDTLYIC
```

A cDNA and a chromosomal sequence encoding the Q2M218 protein is available from the NCBI database as accession no. BC112139 and Z98304, respectively.

An amino acid sequence for the ZNF717 protein encoded by the human ZNF717 gene that is a positive regulator of T cells as detected by interferon-y production is available from the UniPROT database as accession no. Q9BY31, shown below as SEQ ID NO:11.

```
                10 20 30 40
MLETYNSLVS LQELVSFEEV AVHFTWEEWQ DLDDAQRTLY
                50 60 70 80
RDVMLETYSS LVSLGHCITK PEMIFKLEQG AEPWIVEETP
                90 100 110 120
NLRLSAVQII DDLIERSHES HDRFEWQIVI TNSNTSTQER
              130 140 150 160
VELGKTENLN SNHVLNLIIN NGNSSGMKPG QENDCQNMLF
              170 180 190 200
PIKPGETQSG EKPHVCDITR RSHRHHEHLT QHHKIQTLLQ
              210 220 230 240
TFQCNEQGKT FNTEAMFFIH KRVHIVQTFG KYNEYEKACN
              250 260 270 280
NSAVIVQVIT QVGQPTCCRK SDFTKHQQTH TGEKPYECVE
              290 300 310 320
CEKPSISKSD LMLQCKMPTE EKPYACNWCE KLESYKSSLI
              330 340 350 360
IHQRIHTGEK PYGCNECGKT FRRKSFLTLH ERTHTGDKPY
              370 380 390 400
KCIECGKTFH CKSLLTLHHR THSGEKPYQC SECGKTESQK
              410 420 430 440
SYLTIHHRTH TGEKPYACDH CEEAFSHKSR LTVHQRTHTG
              450 460 470 480
EKPYECNECG KPFINKSNLR LHQRTHTGEK PYECNECGKT
              490 500 510 520
FHRKSFLTIH QWTHTGEKPY ECNECGKTER CKSELTVHQR
              530 540 550 560
THAGEKPYAC NECGKTYSHK SYLTVHHRTH TGEKPYECNE
              570 580 590 600
CGKSFHCKSF LTIHQRTHAG KKPYECNECE KTFINKLNLG
              610 620 630 640
IHKITHTGER PYECNECGKT FRQKSNISTH QGTHTGEKPY
              650 660 670 680
VCGKTFHRKS FLTIHQRTHT GKNRMDVMNV EKLFVRNHTL
              690 700 710 720
LYIRELTPGK SPMNVMNVEN PFIRRQIERS IKVFTRGRNP
              730 740 750 760
MNVANVEKPC QKSVLIVHHR THTGEKPYEC NECGKTFCHK
              770 780 790 800
SNLSTHQGTH SGEKPYECDE CRKTFYDKTV LTIHQRTHTG
              810 820 830 840
EKPFECKECR KTESQKSKLF VHHRTHTGEK PERCNECRKT
              850 860 870 880
FSQKSGLSIH QRTHTGEKPY ECKECGKTFC QKSHLSRHQQ
              890 900
THIGEKSDVA EAGYVFPQNH SFEP
```

A cDNA and a chromosomal sequence encoding the Q9BY31 protein is available from the NCBI database as accession no. AF226994 and AC 08724, respectively.

The following genes are positive regulators of T cells as detected by Interleukin-2 production (see Table 2): ABCBI0, ACSS2, ADAM19, ADAM23, ADAMTS5, ALKBH7, ALX4, ANXA2R, AP2AI, APOBEC3C, APOBEC3D, APOL2, ARNT, ART1, ASCL4, BEX4, BTG2, BTNL2, Cl1orf21, C12orf80 (also called LINC02874), CBX4, CBY1, CCDC183, CCDC71L. CD2, CD28, CD6, CDKNIB, CDKN2C, CHERP, CIPC, CLIP3, CNGBI, CNR2, CREB5, CUL3, DCTN5, DEF6, DEPDC7, DYNLL2, EAPP, EEPDI, ELFN2, EMB, EMP,I EMP3, EP300, ERCC3, ESRP1, F2, FBXL13, FBXO41, FNBPIL, FOSB, FOSLI, FOXO4, FOXQI, FUZ, GABRGI, GIGTLC2, GNPDA1, GPR18, GPR20, GPR21, GPR84, GRIN3A, GSDMD, GSTM1, HCST, HELZ2, HEPHL1, IL2, IL2RB, IRX4, ISM1, KLF7, KLRC4, KRT 8, LAT, LCP2, LHX6, LMNA, MAGEA9B, MAP3KI2, MERIK, MTMRI1, NDRG3, N1T1, NLRC3, NLRP2, NPLOC4, ORC1, OSBPL7, OTOP3, OTUD7A, OTUD7B, P2RY14, PAFAH11B2, PCP4, PDE3A, PHF8, PIK3API, PLA2G3, PLCG2, POLK, POU2F2, PPHL2, PRACI, PRKCB, PRKD2, RAB6A, RACI, RAC2, RIPK3, RRAS2, RYR1, SAFB2, SCN3A, SDCCA(uG8, SERPINF1, SGTA, SHOC2, SIGLECI, SIRT1, SLC16AI, SLC44A5, SLC5A5, SMC4, SPPL2B, SSU-12, SWAP70, TAF15, THEMIIS, TM4SF4, TMEM79, TNFRSF1(B, TNFSF11, TNRC6A, TPGS2, TRAF3IP2, TRIM21, TRMT5, TRPM4, TRPV5, TSPYL5, UBA52, UBL5, VAV1, WARS2, ZAP70, ZNF141, ZNF296, and ZNF701. Example 2 provides additional positive regulators T cells that were detected by Interleukin-2 production.

Sequences and other information relating to these genes, and their encoded proteins, is available, for example from the NCBI and UniPROT databases, which are incorporated by reference.

A few examples of protein sequences encoded by some of the genes detected as positive regulators of T cells by Interleukin-2 production are provided. For example, an amino acid sequence for the protein encoded by the human ADAMTS5 gene that is a positive regulator of T cells as detected by Interleukin-2 production is available from the UniPROT database as accession no. Q9UNA0, shown below as SEQ ID NO: 12.

```
                10 20 30 40
MLLGWASLLL CAFRLPLAAV GPAATPAQDK AGQPPTAAAA
                50 60 70 80
AQPRRRQGEE VQERAEPPGH PHPLAQRRRS KGLVQNIDQL
                90 100 110 120
YSGGGKVGYL VYAGGRRELL DLERDGSVGI AGFVPAGGGT
              130 140 150 160
SAPWRHRSHC FYRGTVDGSP RSLAVEDLCG GLDGFFAVKH
              170 180 190 200
ARYTLKPLLR GPWAEEEKGR VYGDGSARIL HVYTREGESE
              210 220 230 240
EALPPRASCE TPASTPEAHE HAPAHSNPSG RAALASQLLD
              250 260 270 280
QSALSPAGGS GPQTWWRRRR RSISRARQVE LLLVADASMA
              290 300 310 320
RLYGRGLQHY LLTLASIANR LYSHASIENH IRLAVVKVVV
              330 340 350 360
IGDKDKSLEV SKNAATTLKN FCKWQHQHNQ LGDDHEEHYD
              370 380 390 400
AAILFTREDL CGHHSCDTLG MADVGTICSP ERSCAVIEDD
              410 420 430 440
GLHAAFTVAH EIGHLIGLSH DDSKFCEETF GSTEDKRLMS
              450 460 470 480
SILTSIDASK PWSKCTSATI TEFLDDGHGN CLLDLPRKQI
              490 500 510 520
LGPEELPGQT YDATQQCNLT FGPEYSVCPG MDVCARLWCA
              530 540 550 560
VVRQGQMVCL TKKLPAVEGT PCGKGRICLQ GKCVDKTKKK
              570 580 590 600
YYSTSSHGNW GSWGSWGQCS RSCGGGVQFA YRHCNNPAPR
              610 620 630 640
NNGRYCTGKR AIYRSCSIMP CPPNGKSFRH EQCEAKNGYQ
              650 660 670 680
SDAKGVKTEV EWVPKYAGVL PADVCKLTCR AKGTGYYVVF
              690 700 710 720
SPKVTDGTEC RLYSNSVCVR GKCVRTGCDG IIGSKLQYDK
              730 740 750 760
CGVCGGDNSS CIKIVGTENK KSKGYTDVVR IPEGATHIKV
              770 780 790 800
RQFKAKDQTR FTAYLALKKK NGEYLINGKY MISTSETIID
              810 820 830 840
INGTVMNYSG WSHRDDELHG MGYSATKEIL IVQILATDPT
              850 860 870 880
KPLDVRYSFF VPKKSTPKVN SVTSHGSNKV GSHTSQPQWV
              890 900 910 920
TGPWLACSRT CDTGWHTRIV QCQDGNRKLA KGCPLSQRPS
              930
AFKQCLLKKC
```

A cDNA and a chromosomal sequence encoding the protein is available from the NCBI database as accession no. AF 42099 and AP001698, respectively.

A nucleotide sequence for human Cl2orf80 cDNA (also called LINC02874) that is a positive regulator of T cells as detected by Interleukin-2 production is available from the NCBI database as accession no. NR_164127.1, shown below as SEQ ID NO: 13.

| | |
|---|---|
| 1 | AGAGCGAGAA GATGATGCAT GTGAGCCCTG CCCTTGGGAA |
| 41 | GCTTCCAGGT TGGGAAATGA GGAATGAGCC TGACACCCAG |
| 81 | GGCCCAGAGA GACCCAGGAC AGAGGCAGGT CAGGAGGCAG |
| 121 | ACACGCGCTG CIGGGTAATG ACGACAGCAC CAGTAATCAC |
| 161 | GGCTACTCCT TGTTAAGTAC TTACTAAGTG CAAGACTCCA |
| 201 | AGCTAAGCAA TTAATAGACC TTTTCTTGTT TAATCCTTAC |
| 241 | CACAATTCCA TAGGGITGAG TAGGAAGTCC TTCTTGAAGT |
| 281 | CTAATCTCAA GAATTCATGC CATGGATTGG GCCAATGTTC |
| 321 | CACCTTTATT GGAGTCTGGG GACTTAGGTG GAAAAGGCAG |
| 361 | AACTGGCTGG TTGGAGGAGC CACCTCCCTG CAAGGGGCCA |
| 401 | GGAGGGAGAT TACGGAGGCG CCAAGCCCAG AGCTCCAACA |
| 441 | TGCACCTGCC ACAGCTCCAG GGGAGATCGG GGGCCTGCCA |
| 481 | ATTTACCCCG CCCCATGATC TCATGGCTGT GTGCGCCAGG |
| 521 | CACTGGCTCA GGGAGCAGCA TCTCACAGAG AAGATACTTG |
| 561 | GATGGGCCAC AGGCCAAAAC TGGGCCAAAA CCCTGGAGAG |
| 601 | GGGGACCTGG CTCAAGGCCA CACCACATTT CCATGTCATT |
| 641 | TTCCAGTGGC ACCAACTGAG CTGGACAGAT GTTCTCACAG |
| 681 | AGCTACACAT GCCACAGTCA TCACTAGTTG CGAGAGTCTC |
| 721 | CAGTGTTCAT TAAGCCTATT TTGCTGGAAG TTTAGTCCCT |
| 761 | GGAGGATTAG TCCCTGCCCT TAAGGAGTGC CAAAGAAGAC |
| 801 | TTTGGAATCT AAGTCACATC TCCCCTGCTG CAATTTTTTC |
| 841 | CTCTTGATAA TCAAGGAATC TCACTGATAA AACTCTCAAT |
| 881 | GAGGAGCAGC TCTCCCCATG AGCAAGTTCT TGCCTTCATT |
| 921 | GCCAGGGAGG CCCCAGCTAA GGTTATACTG GGAAAGGAAT |
| 961 | CTCTGGGGAG TACCTGGAAG AGGTCAGCTT CTCCTCAAAG |
| 1001 | GAAGCCTCTC CAGCTGGTGT ACTGCAAAGC CTTCCAAGAC |
| 1041 | CAAGTTCCCT CTTCCTGGCC TCTGGAACCA GCCTGTGTGC |
| 1081 | CAGCGCTGTG TCCCCAGTAA CACACATGAG TCTCTCCCTC |
| 1121 | AGAAGCCACA GAAGGATGCA GAGAGAGACC AACCCAGGAT |
| 1161 | TGATTCTCAG CACTTACTAA TTGTGTGGAC ATAACTGCTC |
| 1201 | TGGACCTCAG CTTTGCTATC TATAAAATGA GCACCCTTTT |
| 1241 | TATAGATATG AACACTTTAA GAGAGTCATA AAGATTGAAG |
| 1281 | TTGATTIGTG CTGCGCCTGG CACAGAGGTC TCCCTCAGTA |
| 1321 | AATGGCAGCC ACTGCTATTG GGGTGCCCCA ACACCCCTTG |
| 1361 | CCCCTGCCCA CCATTAGCTT TCACTTAGGC CCACACTGAG |
| 1401 | GGTGTGGCTG TTGTGTTGGG GGAAGGAAAA AAACCATGCC |
| 1441 | TGGGTTGCAG GGCCCCCACC ACCAACTGGT CTGCTTTTGT |
| 1481 | GGGAAGCATT TTCTGATGCC CTCGCCCTAC CCGTGGGCTG |
| 1521 | GTTGACTAAG TGTCCAGCAT CATGAGGAAA AGAGCAGGGT |
| 1561 | TCAGGGACAG CTTGGCTCAG CCCTGAAACT CATCTGGGCC |
| 1601 | TAGGTCCAGA TGAGAGGCAA GCCTGGGAGG CCTTCCGTTG |
| 1641 | TACCCTTGCC TATCCTGCAG CACCCTCTAG CCTGCAGGCC |
| 1681 | GCTCCTGGGT GGCATAGCAC CTTGGATGTC AGGTGTGGGC |
| 1721 | CTTCCCAGGC ACATGGCATG AGGGGGCACT TCTGTGGGTT |
| 1761 | GTTGGGGGCA GGAAGGGATG TGTCTCCAGC TGGACTTGGG |
| 1801 | CTCTCGCATT TTGGGGCCCA GCCATGCCAG GACAGCACAC |
| 1841 | ATGGGCGCTT AGTGCGAATC TCATGATGGA GCAGAGGAGG |
| 1881 | AGCAAAGCAA AACCAGGGAG TCCCCAGGCC CCTGCTTCTG |
| 1921 | CCCCGCCCAG AGACAGTGGA GCAGGTGTCT CCAGCTTCTT |
| 1961 | AACCTCAACG CACAGTAAGA AATACATTTT ACAGCAACCA |
| 2001 | ATAGACACAT ACATTTACAA AACGA |

An amino acid sequence for the protein encoded by the human CCDC183 gene that is a positive regulator of T cells as detected by Interleukin-2 production is available from the UniPROT database as accession no. Q5T5S1, shown below as SEQ ID NO: 14.

```
                10 20 30 40
MRRHSETDVE EQTQELKTIT QLQEQCRALQ IQGVKENMDQ
                50 60 70 80
NKATLALLRS NIRRGAQDWA LAKKYDQWTI SKACGKNLPL
                90 100 110 120
RLAHCRSTME VVREKLRKYV EDRVNMHNLL IHLVRRRGQK
              130 140 150 160
LESMQLELDS LRSQPDASKE ELRLLQIIRQ LENNIEKIMI
              170 180 190 200
KIITSQNIHL LYLDLLDYLK TVLAGYPIEL DKLQNLVVNY
              210 220 230 240
CSELSDMKIM SQDAMMITDE VKRNMRQREA SFIEERRARE
              250 260 270 280
NRLNQQKKLI DKIHTKETSE KYRRGQMDLD FPSNLMSTET
              290 300 310 320
LKLRRKETST AEMEYQSGVT AVVEKVKSAV RCSHVWDITS
              330 340 350 360
RELAQRNTEE NLELQMEDCE EWRVQLKALV KQLELEEAVL
              370 380 390 400
KFRQKPSSIS EKSVEKKMTD MLKEEEERLQ LAHSNMTKGQ
              410 420 430 440
ELLLTIQMGI DNLYVRLMGI NLPATQREVV LSNILDINSK
              450 460 470 480
LAYCEGKLTY LADRVQMVSR TEEGDTKVRD TLESSTLMEK
              490 500 510 520
YNTRISFENR EEDMIDTEQE PDMDHSYVPS RAEIKRQAQR
              530
LIEGKLKAAK KKKK
```

A cDNA and a chromosomal sequence encoding the protein is available from the NCBI database as accession no. AB075864 and AL355987, respectively.

An amino acid sequence for the protein encoded by the human CIPC gene that is a positive regulator of T cells as detected by Interleukin-2 production is available from the UniPROT database as accession no. Q9C0C6. shown below as SEQ ID NO: 15,

```
                10 20 30 40
MERKNPSRES PRRLSAKVGK GTEMKKVARQ LGMAAAESDK
                50 60 70 80
DSGESDGSSE CLSSAEQMES EDMLSALGWS REDRPRQNSK
                90 100 110 120
TAKNAFPTLS PMVVMKNVLV KQGSSSSQLQ SWIVQPSFEV
              130 140 150 160
ISAQPQLLFL HPPVPSPVSP CHTGEKKSDS RNYLPILNSY
              170 180 190 200
TKIAPHPGKR GLSLGPEEKG TSGVQKKICT ERLGPSLSSS
              210 220 230 240
EPTKAGAVPS SPSTPAPPSA KLAEDSALQG VPSLVAGGSP
              250 260 270 280
QTLQPVSSSH VAKAPSLIFA SPASPVCASD STLHGLESNS
              290 300 310 320
PLSPLSANYS SPLWAAEHLC RSPDIFSEQR QSKHRRFQNT
              330 340 350 360
LVVLHKSGLL EITLKTKELI RQNQATQVEL DQLKEQTQLF
              370 380 390
IEATKSRAPQ AWAKLQASLT PGSSNIGSDL EAFSDHPAI
```

A cDNA and a chromosomal sequence encoding the CIPC protein is available from the NCBI database as accession no. AB051524 and AC007686, respectively.

An amino acid sequence for the protein encoded by the human CUL3 gene that is a positive regulator of T cells as detected by Interleukin-2 production is available from the UniPROT database as accession no. Q13618, shown below as SEQ ID NO: 16.

```
                10 20 30 40
MSNLSKGTGS RKDTKMRIRA FPMTMDEKYV NSIWDLLKNA
                50 60 70 80
IQEIQRKNNS GLSFEELYRN AYTMVLHKHG EKLYTGLREV
                90 100 110 120
VTEHLINKVR EDVINSLNNN FLQTLNQAWN DHQTAMVMIR
              130 140 150 160
DILMYMDRVY VQQNNVENVY NEGLIIERDQ VVRYGCIRDH
              170 180 190 200
LRQTLLDMIA RERKGEVVDR GAIRNACQML MILGLEGRSV
              210 220 230 240
YEEDFEAPFL EMSAEFFQME SQKFLAENSA SVYIKKVEAR
              250 260 270 280
INEEIERVMH CLDKSTEEPI VKVVERELIS KHMKTIVEME
              290 300 310 320
NSGLVHMLKN GKTEDLGCMY KLFSRVPNGL KTMCECMSSY
              330 340 350 360
LREQGKALVS EEGEGKNPVD YIQGLLDLKS RFDRELLESF
              370 380 390 400
NNDRLFKQTI AGDFEYFLNL NSRSPEYLSL FIDDKLKKGV
              410 420 430 440
KGLTEQEVET ILDKAMVLER FMQEKDVFER YYKQHLARRI
              450 460 470 480
LINKSVSDDS EKNMISKLKT ECGCQFTSKL EGMERDMSIS
              490 500 510 520
NTIMDEFRQH LQATGVSLGG VDLTVRVLTT GYWPTQSATP
              530 540 550 560
KCNIPPAPRH AFEIFRRFYL AKHSGRQLTL QHHMGSADLN
              570 580 590 600
ATFYGPVKKE DGSEVGVGGA QVTGSNTRKH ILQVSTFQMT
              610 620 630 640
ILMLENNREK YTFEEIQQET DIPERELVRA LQSLACGKPT
              650 660 670 680
QRVLTKEPKS KEIENGHIFT VNDQFTSKLH RVKIQTVAAK
              690 700 710 720
QGESDPERKE TRQKVDDDRK HEIEAAIVRI MKSRKKMQHN
              730 740 750 760
VIVAEVTQQL KARFLPSPVV IKKRIEGLIE REYLARTPED
RKVYTYVA
```

A cDNA and a chromosomal sequence encoding the Q13618 protein is available from the NCBT database as accession no AF064087 and AC073052, respectively.

An amino acid sequence for the protein encoded by the human EMB (Embigin) gene that is a positive regulator of T cells as detected by Interleukin-2 production is available from the UniPROT database as accession no. Q6PCB8, shown below as SEQ ID NO: 17.

```
                10 20 30 40
MRALPGLLEA RARTPRLLLL QCLLAAARPS SADGSAPDSP
                50 60 70 80
FTSPPLREEI MANNFSLESH NISLTEHSSM PVEKNITLER
                90 100 110 120
PSNVNLICQF TISGDLNAVN VIWKKDGEQL ENNYLVSATG
              130 140 150 160
STLYTQYRFT IINSKQMGSY SCFFREEKEQ RGTFNFKVPE
              170 180 190 200
LHGKNKPLIS YVGDSTVLTC KCQNCEPLNW TWYSSNGSVK
              210 220 230 240
VPVGVQMNKY VINGTYANET KLKITQLLEE DGESYWCRAL
              250 260 270 280
FQLGESEEHI ELVVLSYLVP LKPFLVIVAE VILLVATILL
              290 300 310 320
CEKYTQKKKK HSDEGKEFEQ IEQLKSDDSN GIENNVPRHR
KNESLGQ
```

A cDNA and a chromosomal sequence encoding the protein is available from the NCBI database as accession no. AK300860 and AC035145, respectively.

An amino acid sequence for the protein encoded by the human ESRP1 gene that is a positive regulator of T cells as detected by Interleukin-2 production is available from the UniPROT database as accession no. Q6NXG1. shown below as SEQ ID NO: 18.

```
                10 20 30 40
MTASPDYLVV LFGITAGATG AKLGSDEKEL ILLEWKVVDL
                50 60 70 80
ANKKVGQLHE VIVRPDQLEL TEDCKEETKI DVESISSASQ
                90 100 110 120
LDQALRQENQ SVSNELNIGV GTSFCLCTDG QLHVRQILHP
              130 140 150 160
EASKKNVLLP ECFYSFFDLR KEFKKCCPGS PDIDKLDVAT
              170 180 190 200
MTEYLNFEKS SSVSRYGASQ VEDMGNIILA MISEPYNHRE
              210 220 230 240
SDPERVNYKF ESGTCSKMEL IDDNTVVRAR GLPWQSSDQD
              250 260 270 280
IARFFKGLNI AKGGAALCIN AQGRRNGEAL VRFVSEEHRD
              290 300 310 320
LALQRHKHHM GTRYIEVYKA TGEDELKIAG GTSNEVAQFL
              330 340 350 360
SKENQVIVRM RGLPFTATAE EVVAFFGQHC PITGGKEGIL
              370 380 390 400
FVTYPDGRPT GDAFVLFACE EYAQNALRKH KDLLGKRYIE
              410 420 430 440
LERSTAAEVQ QVLNRESSAP LIPLPTPPII PVLPQQFVPP
              450 460 470 480
INVRDCIRER GIPYAATIED ILDELGEFAT DIRTHGVHMV
              490 500 510 520
LNHQGRPSGD AFIQMKSADR AFMAAQKCHK KNMKDRYVEV
              530 540 550 560
FQCSAEEMNF VLMGGTLNRN GLSPPPCKLP CLSPPSYTEP
              570 580 590 600
APAAVIPTEA AIYQPSVILN PRALQPSTAY YPAGTQLEMN
              610 620 630 640
YTAYYPSPPG SPNSLGYEPT AANLSGVPPQ PGTVVRMQGL
              650 660 670 680
AYNTGVKEIL NFFQGYQYAT EDGLIHINDQ ARTLPKEWVC
I
```

A cDNA and a chromosomal sequence encoding the Q6NXG1 protein is available from the NCBI database as accession no. BC067098 and AP005660, respectively.

An amino acid sequence for the protein encoded by the human FBXL13 gene that is a positive regulator of T cells as detected by Interleukin-2 production is available from the UniPROT database as accession no. Q8NEE6, shown below as SEQ ID NO:19.

```
                10 20 30 40
MTPELMIKAC SFYTGHLVKT HFCTWRDIAR TNENVVLAEK
                50 60 70 80
MNRAVTCYNE RLQKSVFHHW HSYMEDQKEK LKNILLRIQQ
                90 100 110 120
IIYCHKLTII LTKWRNTARH KSKKKEDELI LKHELQLKKW
              130 140 150 160
KNRLILKRAA AEESNFPERS SSEVELVDET LKCDISLLPE
              170 180 190 200
RAILQIFFYL SLKDVIICGQ VNHAWMLMTQ INSLWNAIDE
              210 220 230 240
SSVENVIPDK YIVSTLQRWR INVIRLNERG CLLRPKTERS
              250 260 270 280
VSHCRNLQEL NVSDCPTFTD ESMRHISEGC PGVLCLNLSN
              290 300 310 320
TTITNRTMRL LPRHFHNLQN LSLAYCRRFT DKGLQYLNLG
              330 340 350 360
NGCHKLIYLD LSGCTQISVQ GERYIANSCT GIMHLTINDM
              370 380 390 400
PTLTDNCVKA LVEKCSRITS LVETGAPHIS DCTFRALSAC
              410 420 430 440
KLRKIRFEGN KRVIDASFKF IDKNYPNISH IYMADCKGIT
              450 460 470 480
DSSLRSLSPL KQLTVLNLAN CVRIGDMGLK QFLDGPASMR
              490 500 510 520
IRELNLSNCV RLSDASVMKL SERCPNLNYL SLRNCEHLTA
              530 540 550 560
QGIGYIVNIF SLVSIDLSGT DISNEGLNVL SRHKKLKELS
              570 580 590 600
VSECYRITDD GIQAFCKSSL ILEHLDVSYC SQLSDMIIKA
              610 620 630 640
LAIYCINITS LSIAGCPKIT DSAMEMLSAK CHYLHILDIS
              650 660 670 680
GCVLLTDQIL EDLQIGCKQL RILKMQYCIN ISKKAAQRMS
              690 700 710 720
SKVQQQEYNT NDPPRWEGYD REGNPVTELD NITSSKGALE
              730
LTVKKSTYSS EDQAA
```

A cDNA and a chromosomal sequence encoding the FBXL13 protein is available from the NCBI database as accession no. AY359238 and AC005250, respectively.

An amino acid sequence for the protein encoded by the human FBXO41 gene that is a positive regulator of T cells as detected by Interleukin-2 production is available from the UniPROT database as accession no. Q8TF61, shown below as SEQ ID NO:20.

```
                10 20 30 40
MASLDLPYRC PRCGEHKRER SLSSLRAHLE YSHTYETLYI
                50 60 70 80
LSKINSICDG AAAAAAAAAA ASGEPLAPEP AALLAVPGAR
                90 100 110 120
REVFESTSFQ GKEQAAGPSP AAPHLLHHHH HHAPLAHFPG
              130 140 150 160
DLVPASIPCE ELAEPGIVPA AAARYALREI EIPLGELFAR
              170 180 190 200
KSVASSACST PPPGPGPGPC PGPASASPAS PSPADVAYEE
              210 220 230 240
GLARLKIRAL EKLEVDRRLE RLSEEVEQKI AGQVGRLQAE
              250 260 270 280
LERKAAELET ARQESARLGR EKEELEERAS ELSRQVDVSV
              290 300 310 320
ELLASLKQDL VHKEQELSRK QQEVVQIDQF LKETAAREAS
              330 340 350 360
AKLRLQQFIE ELLERADRAE RQLQVISSSC GSTPSASIGR
              370 380 390 400
GGGGGGAGPN ARGPGRMREH HVGPAVPNTY AVSRHGSSPS
              410 420 430 440
TGASSRVPAA SQSSGCYDSD SLELPRPEEG APEDSGPGGL
              450 460 470 480
GTRAQAANGG SERSQPPRSS GLRRQAIQNW QRRPRRHSTE
              490 500 510 520
GEEGDVSDVG SRTTESEAEG PIDAPRPGPA MAGPLSSCRL
              530 540 550 560
SARPEGGSGR GRRAERVSPS RSNEVISPEI LKMRAALFCI
              570 580 590 600
FTYLDTRILL HAAEVCRDWR FVARHPAVWT RVLLENARVC
              610 620 630 640
SKFLAMLAQW CTQAHSLTLQ NLKPRQRGKK ESKEEYARST
              650 660 670 680
RGCLEAGLES LIKAAGGNLL ILRISHCPNI LTDRSLWLAS
              690 700 710 720
CYCRALQAVT YRSAIDPVGH EVIWALGAGC REIVSLQVAP
              730 740 750 760
LHPCQQPTRF SNRCLQMIGR CWPHLRALGV GGAGCGVQGL
              770 780 790 800
ASLARNCMRL QVLELDHVSE ITQEVAAEVC REGLKGLEML
              810 820 830 840
VLTATPVTPK ALLHENSICR NLKSIVVQIG IADYFKEPSS
              850 860 870
PEAQKLFEDM VTKLQALRRR PGESKILHIK VEGGC
```

A cDNA and a chromosomal sequence encoding the FBXO41 protein is available from the NCBI database as accession no. AB075820 and AC010913, respectively.

An amino acid sequence for the protein encoded by the human FOSL1 gene that is a positive regulator of T cells as detected by Interleukin-2 production is available from the UniPROT database as accession no. P15407, shown below as SEQ ID NO:21.

```
                10 20 30 40
MERDEGEPGP SSGNGGGYGG PAQPPAAAQA AQQKFHLVPS
                50 60 70 80
INTMSGSQEL QWMVQPHELG PSSYPRPITY PQYSPPQPRP
                90 100 110 120
GVIRALGPPP GVRRRPCEQI SPEEEERRRV RRERNKLAAA
              130 140 150 160
KCRNRRKELT DFLQAETDKL EDEKSGLQRE IEELQKQKER
              170 180 190 200
LELVLEAHRP ICKIPEGAKE GDTGSTSGTS SPPAPCRPVP
              210 220 230 240
CISLSPGPVL EPEALHTPTL MITPSLTPFT PSLVFTYPST
              250 260 270
PEPCASAHRK SSSSSGDPSS DPLGSPTLLA L
```

A cDNA and a chromosomal sequence encoding the FOSL1 protein is available from the NCBI database as accession no. X16707 and AP006287, respectively.

An amino acid sequence for the protein encoded by the human FOXO4 gene that is a positive regulator of T cells as detected by Interleukin-2 production is available from the UniPROT database as accession no. P98177, shown below as SEQ ID NO:22.

```
                10 20 30 40
MDPGNENSAT EAAAIIDLDP DEEPQSRPRS CTWPLPRPEI
                50 60 70 80
ANQPSEPPEV EPDLGEKVHT EGRSEPILLP SRLPEPAGGP
                90 100 110 120
QPGILGAVTG PRKGGSRRNA WGNQSYAELI SQAIESAPEK
              130 140 150 160
RLTLAQIYEW MVRTVPYEKD KGDSNSSAGW KNSIRHNISL
              170 180 190 200
HSKFIKVHNE ATGKSSWWML NPEGGKSGKA PRRRAASMDS
              210 220 230 240
SSKLLRGRSK APKKKPSVLP APPEGATPTS PVGHEAKWSG
              250 260 270 280
SPCSRNREEA DMWTTFRPRS SSNASSVSTR LSPLRPESEV
              290 300 310 320
LAEEIPASVS SYAGGVPPTL NEGLELLDGL NLTSSHSLLS
              330 340 350 360
RSGLSGESLQ HPGVTGPLHT YSSSLESPAE GPLSAGEGCF
              370 380 390 400
SSSQALEALL TSDTPPPPAD VLMTQVDPIL SQAPTLLLLG
              410 420 430 440
GIPSSSKLAT GVGLCPKPLE APGPSSLVPT LSMIAPPPVM
              450 460 470 480
ASAPIPKALG TPVLIPPTEA ASQDRMPQDL DLDMYMENLE
              490 500
CDMDNIISDL MDEGEGLDEN FEPDP
```

A cDNA and a chromosomal sequence encoding the FOXO4 protein is available from the NCBI database as accession no. X93996 and AL590764, respectively.

An amino acid sequence for the protein encoded by the human FUZ gene that is a positive regulator of T cells as detected by Interleukin-2 production is available from the UniPROT database as accession no. Q9BT04, shown below as SEQ ID NO:23.

```
                10 20 30 40
MGEEGTGGTV HLLCLAASSG VPLFCRSSRG GAPARQQLPF
                50 60 70 80
SVIGSLNGVH MFGQNLEVQL SSARTENTTV VWKSFHDSIT
                90 100 110 120
LIVLSSEVGI SELRLERLLQ MVFGAMVILV GLEELINIRN
              130 140 150 160
VERLKKDLRA SYCLIDSELG DSELIGDLTQ CVDCVIPPEG
              170 180 190 200
SLLQEALSGF AEAAGTTEVS LVVSGRVVAA TEGWWRLGTP
              210 220 230 240
EAVLLPWLVG SLPPQTARDY PVYLPHGSPT VPHRLLTLTL
              250 260 270 280
LPSLELCLIC GPSPPLSQLY PQLLERWWQP LLDPLRACLP
              290 300 310 320
LGPRALPSGF PLHTDILGLL LLHLELKRCL ETVEPLGDKE
              330 340 350 360
PSPEQRRRLL RNFYTIVIST HEPPEPGPPE KTEDEVYQAQ
              370 380 390 400
LPRACYLVLG TEEPGTGVRL VALQLGERRL LLLLSPQSPT
              410
HGLRSLATHT LHALTPLL
```

A cDNA and a chromosomal sequence encoding the FUZ protein is available from the NCBI database as accession no. AK026341 and AC006942, respectively.

An amino acid sequence for the protein encoded by the human IRX4 gene is available from the UniPROT database as accession no. P78413, shown below as SEQ ID NO:23.

```
                10 20 30 40
MSYPQFGYPY SSAPQELMAT NSISTCCESG GRTLADSGPA
                50 60 70 80
ASAQAPVYCP VYESRLLATA RHEINSAAAL GVYGGPYGGS
                90 100 110 120
QGYGNYVTYG SEASAFYSLN SEDSKDGSGS AHGGLAPAAA
              130 140 150 160
AYYPYEPALG QYPYDRYGTM DSGTRRKNAT RETTSTLKAW
              170 180 190 200
LQEHRKNPYP TKGEKIMLAI ITKMTLTQVS TWFANARRRI
              210 220 230 240
KKENKMTWPP RNKCADEKRP YAEGEEEEGG EEEAREEPLK
              250 260 270 280
SSKNAEPVGK EEKELELSDL DDEDPLEAEP PACELKPPFH
              290 300 310 320
SLDGGLERVP AAPDGPVKEA SGALRMSLAA GGGAALDEDL
              330 340 350 360
ERARSCLRSA AAGPEPLPGA EGGPQVCEAK LGFVPAGASA
              370 380 390 400
GLEAKPRIWS LAHTATAAAA AATSLSQTEF PSCMLKRQGP
              410 420 430 440
AAPAAVSSAP ATSPSVALPH SGALDRHQDS PVISLRNWVD
              450 460 470 480
GVFHDPILRH STlNQAWATA KGALLDPGPL GRSLGAGANV
              490 500 510
LTAPLARAFP PAVPQDAPAA GAARELLALP KAGGKPECA
```

A cDNA and a chromosomal sequence encoding the IRX4 protein is available from the NCBI database as accession no. AF124733 and AB690778, respectively.

An amino acid sequence for the protein encoded by the human ISM1 gene that is a positive regulator of T cells as detected by Interleukin-2 production is available from the UniPROT database as accession no. B1AKI, shown below as SEQ ID NO:24.

```
                10 20 30 40
MVRLAAELLL LLGLLLLTLH ITVLRGSGAA DGPDAAAGNA
                50 60 70 80
SQAQLQNNLN VGSDITSETS FSLSKEAPRE HLDHQAAHQP
                90 100 110 120
FPRPRERQET GHPSLQRDEP RSFLLDIPNF PDLSKADING
              130 140 150 160
QNPNIQVTIE VVDGPDSEAD KDQHPENKPS WSVPSPDWRA
              170 180 190 200
WWQRSLSLAR ANSGDQDYKY DSTSDDSNFL NPPRGWDHTA
              210 220 230 240
PGHRTFETKD QPEYDSTDGE GDWSLWSVCS VTCGNGNQKR
              250 260 270 280
TRSCGYACTA TESRTCDRPN CPGIEDTERT AATEVSLLAG
              290 300 310 320
SEEFNATKLF EVDTDSCERW MSCKSEFLKK YMHKVMNDLP
              330 340 350 360
SCPCSYPTEV AYSTADIEDR IKRKDERWKD ASGPKEKLEI
              370 380 390 400
YKPTARYCIR SMLSLESTIL AAQHCCYGDN MQLITRGKGA
              410 420 430 440
GTPNLISTEF SAELHYKVDV LPWIICKGDW SRYNEARPPN
              450 460
NGQKCTESPS DEDYIKQFQE AREY
```

A cDNA and a chromosomal sequence encoding the ISM1 protein is available from the NCBI database as accession no. BC017997 and AL050320, respectively.

An amino acid sequence for the protein encoded by the human MTMR-11 gene that is a positive regulator of T cells as detected by Interleukin-2 production is available from the UniPROT database as accession no. A4FU01, shown below as SEQ ID NO:25.

```
                10 20 30 40
MWWGGRGQSF NIAPQKEEPE MGSVQENRMP EPRSRQPSSC
                50 60 70 80
LASRCLPGEQ ILAWAPGVRK GLEPELSGTL ICTNERVTFQ
                90 100 110 120
PCGWQWNQDT PLNSEYDFAL VNIGRLEAVS GLSRVQLLRP
              130 140 150 160
GSLHKFIPEE ILIHGRDERL LRVGFEAGGL EPQAFQVIMA
              170 180 190 200
IVQARAQSNQ AQQYSGITLS KAGQGSGSRK PPIPLMETAE
              210 220 230 240
DWETERKKQA ARGWRVSTVN EREDVATSLP RYFWVPNRIL
              250 260 270 280
DSEVRRAFGH FHQGRGPRLS WHHPGGSDLL RCGGFYTASD
              290 300 310 320
PNKEDIRAVE LMLQAGHSDV VLVDTMDELP SLADVQLAHL
              330 340 350 360
RIRALCLPDS SVAEDKWLSA LEGTRWLDYV RACLRKASDI
              370 380 390 400
SVIVISRVRS VILQERGDRD LNGLLSSLVQ LISAPEARTL
              410 420 430 440
FGFQSLVQRE WVAAGHPELT RIGGTGASEE APVELLELDC
              450 460 470 480
VWQLLQQEPA DEEFSEFELL ALHDSVRVPD ILTFLRNTPW
              490 500 510 520
ERGKQSGQLN SYTQVYTPGY SQPPAGNSEN LQLSVWDWDL
              530 540 550 560
RYSNAQILQF QNPGYDPEHC PDSWLPRPQP SEMVPGPPSS
              570 580 590 600
VWLFSRGALT PLNQLCPWRD SPSLLAVSSR WLPRPAISSE
              610 620 630 640
SLADQEWGLP SHWGACPLPP GLLLPGYLGP QIRLWRRCYL
              650 660 670 680
RGRPEVQMGL SAPTISGLQD ELSHLQELLR KWTPRISPED
              690 700
HSKKRDPHTI LNPTEIAGIL KGRAEGDLG
```

A cDNA and a chromosomal sequence encoding the MTMR11 protein is available from the NCBI database as accession no. U78556 and AL590487, respectively.

An amino acid sequence for the protein encoded by the human NDRG3 gene that is a positive regulator of T cells as detected by Interleukin-2 production is available from the UniPROT database as accession no. Q9UGV2. shown below as SEQ ID NO:26.

```
                10 20 30 40
MDELQDVQLT EIKPLLNDKN GTRNFQDEDC QEHDIETTHG
                50 60 70 80
VVHVTIRGLP KGNRPVILTY HDIGLNHKSC FNAFENFEDM
                90 100 110 120
QEITQHFAVC HVDAPGQQEG APSFPTGYQY PTMDELAEML
              130 140 150 160
PPVLTHLSLK SIIGIGVGAG AYILSRFALN HPELVEGLVL
              170 180 190 200
INVDPCAKGW IDWAASKLSG LTINVVDIIL AHHFGQEELQ
              210 220 230 240
ANLDLIQTYR MHIAQDINQD NLQLFLNSYN GRRDLEIERP
              250 260 270 280
ILGQNDNKSK TLKCSTLLVV GDNSPAVEAV VECNSRLNPI
              290 300 310 320
NTILLKMADC GGLPQVVQPG KLTEAFKYFL QGMGYIPSAS
              330 340 350 360
MTRLARSRTH STSSSLGSGE SPESRSVTSN QSDGIQESCE
              370
SPDVLDRHQT MEVSC
```

A cDNA and a chromosomal sequence encoding the NDRG3 protein is available from the NCBI database as accession no. AB044943 and AL031662, respectively.

An amino acid sequence for the protein encoded by the human NPLOC4 gene that is a positive regulator of T cells as detected by Interleukin-2 production is available from the UniPROT database as accession no. Q8TAT6, shown below as SEQ ID NO:27.

```
                10 20 30 40
MAESIIIRVQ SPDGVKRITA TKRETAATFL KKVAKEFGFQ
                50 60 70 80
NNGESVYINR NKTGEITASS NKSLNLLKIK HGDLLFLFPS
                90 100 110 120
SLAGPSSEME TSVPPGEKVE GAPNVVEDEI DQYLSKQDGK
              130 140 150 160
IYRSRDPQLC RHGPLGKCVH CVPLEPEDED YLNHLEPPVK
              170 180 190 200
HMSFHAYIRK LIGGADKGKF VALENISCKI KSGCEGHLPW
              210 220 230 240
PNGICTKCQP SAITLNRQKY RHVDNIMFEN HTVADRELDF
              250 260 270 280
WRKTGNQHEG YLYGRYTEHK DIPIGIRAEV AAIYEPPQIG
              290 300 310 320
TQNSLELLED PKAEVVDEIA AKLGLRKVGW IFTDLVSEDT
              330 340 350 360
RKGIVRYSRN KDTYFLSSEE CITAGDEQNK HPNMCRLSPD
              370 380 390 400
GHFGSKEVTA VATGGPDNQV HFEGYQVSNQ CMALVRDECL
              410 420 430 440
LPCKDAPELG YAKESSSEQY VPDVFYKDVD KEGNEITQLA
              450 460 470 480
RPLPVEYLII DITTTEPKDP VYTFSISQNP FPIENRDVLG
              490 500 510 520
ETQDEHSLAT YLSQNTSSVE LDTISDEHLL LFLVINEVMP
              530 540 550 560
LQDSISILLE AVRIRNEELA QTWKRSEQWA TIEQLCSTVG
              570 580 590 600
GQLPGLHEYG AVGGSTHTAT AAMWACQHCT FMNQPGTGHC
EMCSLPRT
```

A cDNA encoding the NPLOC4 protein is available from the NCBI database as accession no. AB040932.

An amino acid sequence for the protein encoded by the human OTOP3 gene that is a positive regulator of T cells as detected by Interleukin-2 production is available from the UniPROT database as accession no. Q7RTS5. shown below as SEQ ID NO:28.

```
                10 20 30 40
MGRGARAAAA QSRWGRASRA SVSPGRTIRS APAVGEAQET
                50 60 70 80
EAAPEKENRV DVGAEERAAA TRPRQKSWLV RHESLLLRRD
                90 100 110 120
RQAQKAGQLF SGLLALNVVE LGGAFICSMI FNKVAVILGD
              130 140 150 160
VWILLATLKV LSLIWLLYYV ASTTRRPHAV LYQDPHAGPL
              170 180 190 200
WVRGSLVLFG SCTFCLNIFR VGYDVSHIRC KSQLDLVESV
              210 220 230 240
IEMVEIGVQT WVLWKHCKDC VRVQTNFTRC GLMLTLATNL
              250 260 270 280
LLWVLAVIND SMHREIEAEL GILMEKSTGN ETNTCLCLNA
              290 300 310 320
TACEAFRRGF LMLYPESTEY CLICCAVLEV MWKNVGRHVA
              330 340 350 360
PHMGAHPATA PFHLHGAIFG PLLGLLVLLA GVCVFVLFQI
              370 380 390 400
EASGPAIACQ YFTLYYAFYV AVLPTMSLAC LAGTAIHGLE
              410 420 430 440
ERELDTVKNP TRSLDVVLLM GAALGQMGIA YESIVAIVAK
              450 460 470 480
RPHELLNRLI LAYSLLLILQ HIAQNLFIIE GLHRRPLWET
              490 500 510 520
VPEGLAGKQE AEPPRRGSLL EIGQGLQRAS LAYIHSYSHL
              530 540 550 560
NWKRRALKEI SLFLILCNIT LWMMPAFGIH PEFENGLEKD
              570 580 590
FYGYQIWFAI VNFGLPLGVF YRMHSVGGLV EVYLGA
```

A cDNA and a chromosomal sequence encoding the OTOP3 protein is available from the NCBI database as accession no. BK000568 and AC087651, respectively.

An amino acid sequence for the protein encoded by the human OTUD7A gene that is a positive regulator of T cells as detected by Interleukin-2 production is available from the UniPROT database as accession no. Q8TE49, shown below as SEQ [D NO:29.

```
                10 20 30 40
MVSSVLPNPT SAECWAALLH DPMTLDMDAV LSDEVRSTGA
                50 60 70 80
EPGLARDLLE GKNWDLTAAL SDYEQLRQVH TANLPHVENE
                90 100 110 120
GRGPKQPERE PQPGHKVERP CLQRQDDIAQ EKRLSRGISH
              130 140 150 160
ASSAIVSLAR SHVASECNNE QFPLEMPIYT FQLPDLSVYS
              170 180 190 200
EDERSFIERD LIEQATMVAL EQAGRINWWS TVCTSCKRLL
              210 220 230 240
PLATTGDGNC LLHAASLGMW GFHDRDEVIR KALYTMMRTG
              250 260 270 280
AEREALKRRW RWQQTQQNKE EEWEREWTEL LKLASSEPRT
              290 300 310 320
HFSKNGGTGG GVDNSEDPVY ESLEEFHVEV LAHILRRPIV
              330 340 350 360
VVADTMLRDS GGEAFAPIPE GGIYLPLEVP PNRCHCSPLV
              370 380 390 400
LAYDQAHESA LVSMEQRDQQ REQAVIPLTD SEHKLLPLHE
              410 420 430 440
AVDPGKDWEW GKDDNDNARI AHLILSLEAK LNLLHSYMNV
              450 460 470 480
TWIRIPSETR APLAQPESPT ASAGEDVQSL ADSLDSDRDS
              490 500 510 520
VCSNSNSNNG KNGKDKEKEK QRKEKDKTRA DSVANKLGSF
              530 540 550 560
SKILGIKLKK NMGGLGGLVH GKMGRANSAN GKNGDSAERG
              570 580 590 600
KEKKAKSRKG SKEESGASAS TSPSEKTTPS PTDKAAGASP
              610 620 630 640
AEKGGGPRGD AWKYSTDVKL SLNILRAAMQ GERKFIFAGL
              650 660 670 680
LLTSHRHQFH EEMIGYYLTS AQERFSAEQE QRRRDAATAA
              690 700 710 720
AAAAAAAAAT AKRPPRRPET EGVPVPERAS PGPPTQLVLK
              730 740 750 760
LKERPSPGPA AGRAARAAAG GTASPGGGAR RASASGPVPG
              770 780 790 800
RSPPAPARQS VIHVQASGAR DEACAPAVGA LRPCATYPQQ
              810 820 830 840
NRSLSSQSYS PARAAALRTV NIVESLARAV PGALPGAAGT
              850 860 870 880
AGAAEHKSQT YINGEGALRD GLEFADADAP TARSNGECGR
              890 900 910 920
GGPGPVQRRC QRENCAFYGR AETEHYCSYC YREELRRRRE
ARGARP
```

A cDNA sequence encoding the OTUD7A protein is available from the NCBI database as accession no. AJ430383.

An amino acid sequence for the protein encoded by the human PDE3A gene that is a positive regulator of T cells as detected by Interleukin-2 production is available from the UniPROT database as accession no. Q14432, shown below as SEQ ID NO:30.

```
                10 20 30 40
MAVPGDAARV RDKPVHSGVS QAPTAGRDCH HRADPASPRD
                50 60 70 80
SGCRGCWGDL VLQPLRSSRK LSSALCAGSL SELLALLVRL
                90 100 110 120
VRGEVGCDLE QCKEAAAAEE EEAAPGAEGG VEPGPRGGAP
              130 140 150 160
GGGARLSPWL QPSALLESLL CAFEWMGLYL LRAGVRLPLA
              170 180 190 200
VALLAACCGG EALVQIGLGV GEDHLLSLPA AGVVLSCLAA
              210 220 230 240
ATWLVIRLRL GVLMIALISA VRTVSLISLE REKVAWRPYL
              250 260 270 280
AYLAGVLGIL LARYVEQILP QSAEAAPREH LGSQLIAGTK
              290 300 310 320
EDIPVFKRRR RSSSVVSAEM SGCSSKSHRR TSLPCIPREQ
              330 340 350 360
LMGHSEWDHK RGPRGSQSSG TSITVDIAVM GEAHGLITDL
              370 380 390 400
LADPSLPPNV CISLRAVSNL LSTQLTFQAI HKPRVNPVTS
              410 420 430 440
ISENYTCSDS EESSEKDKLA IPKRIRRSLP PGLLRRVSST
              450 460 470 480
WTTTTSATGE PTLEPAPVRR DRSTSIKLQE APSSSPDSWN
              490 500 510 520
NPVMMTLTKS RSFTSSYAIS AANHVKAKKQ SRPGALAKIS
              530 540 550 560
PLSSPCSSPL QGTPASSLVS KISAVQFPES ADTTAKQSLG
              570 580 590 600
SHRALTYTQS APDLSPQILT PPVICSSCGR PYSQGNPADE
              610 620 630 640
PLERSGVATR TPSRIDDTAQ VISDYEINNN SDSSDIVQNE
              650 660 670 680
DETECLREPL RKASACSTYA PETMMELDKP ILAPEPLVMD
              690 700 710 720
NLDSIMEQLN TWNFPIEDLV ENIGRKCGRI LSQVSYRLFE
              730 740 750 760
DMGLFEAFKI PIREEMNYFH ALEIGYRDIP YHNRIHATDV
              770 780 790 800
LHAVWYLTTQ PIPGLSTVIN DHGSTSDSDS DSGFTHGHMG
              810 820 830 840
YVESKTYNVT DDKYGCISGN IPALELMALY VAAAMHDYDH
              850 860 870 880
PGRTNAFLVA TSAPQAVLYN DRSVLENHHA AAAWNLEMSR
              890 900 910 920
PEYNFLINLD HVEFKHFREL VIEAILATDL KKHEDFVAKF
              930 940 950 960
NGKVNDDVGI DWTNENDRLL VCQMCIKLAD INGPAKCKEL
              970 980 990 1000
HLQWTDGIVN EFYEQGDEEA SLGLPISPFM DRSAPQLANL
            1010 1020 1030 1040
QESFISHIVG PLCNSYDSAG LMPGKWVEDS DESGDIDDPE
            1050 1060 1070 1080
EEEEEAPAPN EEETCENNES PKKKTEKRRK IYCQITQHLL
            1090 1100 1110 1120
QNHKMWKKVI EEEQRLAGIE NQSLDQTPQS HSSEQIQAIK
            1130 1140
EEEEEKGKPR GEEIPTQKPD Q
```

A cDNA sequence encoding the PDE3A protein is available from the NCBI database as accession no. M91667.

An amino acid sequence for the protein encoded by the human POLK gene that is a positive regulator of T cells as detected by Interleukin-2 production is available from the UniPROT database, shown below as SEQ ID NO:31.

```
                10 20 30 40
MDSTKEKCDS YKDDLLLRMG INDNKAGMEG LDKEKINKII
                50 60 70 80
MEATKGSRFY GNELKKEKQV NQRIENMMQQ KAQITSQQLR
                90 100 110 120
KAQLQVDRFA MELEQSRNLS NTIVHIDMDA FYAAVEMRDN
              130 140 150 160
PELKDKPIAV GSMSMLSTSN YHARREGVRA AMPGFIAKRL
              170 180 190 200
CPQLIIVPPN FDKYRAVSKE VKEILADYDP NEMAMSLDEA
              210 220 230 240
YLNITKHLEE RQNWPEDKRR YFIKMGSSVE NDNPGKEVNK
              250 260 270 280
LSEHERSISP LIFEESPSDV QPPGDPFQVN FEEQNNPQIL
              290 300 310 320
QNSVVFGTSA QEVVKEIRER IEQKTTLTAS AGIAPNTMLA
              330 340 350 360
KVCSDKNKPN GQYQILPNRQ AVMDFIKDLP IRKVSGIGKV
              370 380 390 400
TEKMLKALGI ITCTELYQQR ALLSLLESET SWHYFLHISL
              410 420 430 440
GLGSTHLIRD GERKSMSVER TESEINKAEE QYSICQELCS
              450 460 470 480
ELAQDLQKER LKGRTVTIKL KNVNFEVKTR ASTVSSVVST
              490 500 510 520
AEEIFAIAKE LLKTEIDADE PHPLRLRIMG VRISSFPNEE
              530 540 550 560
DRKHQQRSII GELQAGNQAL SATECTLEKT DKDKFVKPLE
              570 580 590 600
MSHKKSFEDK KRSERKWSHQ DTEKCEAVNK QSFQTSQPEQ
              610 620 630 640
VLKKKMNENL EISENSDDCQ ILTCPVCFRA QGCISLEALN
              650 660 670 680
KHVDECLDGP SISENEKMFS CSHVSATKVN KKENVPASSL
              690 700 710 720
CEKQDYEAHP KIKEISSVDC IALVDTIDNS SKAESIDALS
              730 740 750 760
NKHSKEECSS LPSKSFNIEH CHQNSSSTVS LENEDVGSFR
              770 780 790 800
QEYRQPYLCE VKTGQALVCP VCNVEQKTSD LTLENVHVDV
              810 820 830 840
CINKSFIQEL RKDKENPVNQ PKESSRSTGS SSGVQKAVIR
              850 860 870
TKRPGLMTKY STSKKIKPNN PKHTLDIFEK
```

A cDNA and a chromosomal sequence encoding the POLK protein is available from the NCBI database as accession no. AB027564 and AY273797, respectively.

An amino acid sequence for the protein encoded by the human PRACI gene that is a positive regulator of T cells as detected by Interleukin-2 production is available from the UniPROT database as accession no. Q96KF2, shown below as SEQ ID NO:32.

```
                10 20 30 40
MICAHFSDQG PAHLTTSKSA FLSNKKTSTL KHLLGETRSD
                50
GSACNSGISG GRGRKIP
```

A cDNA and a chromosomal sequence encoding the PRACI protein is available from the NCBI database as accession no. AF331 165 and CH471 109, respectively.

An amino acid sequence for the protein encoded by the human SERPINF1 gene that is a positive regulator of T cells as detected by Interleukin-2 production is available from the UniPROT database, shown below as SEQ ID NO:33.

```
                10 20 30 40
MQALVLLLCI GALLGHSSCQ NPASPPEEGS PDPDSTGALV
                50 60 70 80
EEEDPFFKVP VNKLAAAVSN FGYDLYRVRS STSPTINVLL
                90 100 110 120
SPLSVATALS ALSLGAEQRT ESIIHRALYY DLISSPDIHG
              130 140 150 160
TYKELLDTVT APQKNLKSAS RIVFEKKLRI KSSFVAPLEK
              170 180 190 200
SYGTRPRVLT GNPRLDLQEI NNWVQAQMKG KLARSTKEIP
              210 220 230 240
DEISILLLGV AHFKGQWVIK FDSRKTSLED FYLDEERTVR
              250 260 270 280
VPMMSDPKAV LRYGLDSDLS CKIAQLPLIG SMSIIFFLPL
              290 300 310 320
KVTQNLTLIE ESLTSEFIHD IDRELKTVQA VLTVPKLKLS
              330 340 350 360
YEGEVIKSLQ EMKLQSLEDS PDFSKITGKP IKLTQVEHRA
              370 380 390 400
GFEWNEDGAG TTPSPGLQPA HLTFPLDYHL NQPFIFVERD
              410
TDTGALLFIG KILDPRGP
```

A cDNA and a chromosomal sequence encoding the SERPINF1 protein is available from the NCBI database as accession no. M76979 and U29953, respectively.

An amino acid sequence for the protein encoded by the human SSUH-12 gene that is a positive regulator of T cells as detected by Interleukin-2 production is available from the UniPROT database as accession no. Q9Y2M2, shown below as SEQ ID NO:34.

```
                10 20 30 40
MPSPVGLIRA LPLPWPQFLA CTLRRLAGPR ESTGPSQKPP
                50 60 70 80
PLCSVPCRVP AMTEEVAREA LLSEVDSKCC YSSTVAGDLV
                90 100 110 120
IQELKRQTLC RYRLETESES RISEWTFQPF TNHSVDGPQR
              130 140 150 160
GASPRLWDIK VQGPPMFQED TRKFQVPHSS LVKECHKCHG
              170 180 190 200
RGRYKCSGCH GAGTVRCPSC CGAKRKAKQS RRCQLCAGSG
              210 220 230 240
RRRCSTCSGR GNKICATCKG EKKLLHFIQL VIMWKNSLEE
              250 260 270 280
EVSEHRLNCP RELLAKAKGE NLFKDENSVV YPIVDFPLRD
              290 300 310 320
ISLASQRGIA EHSAALASRA RVLQQRQTIE LIPLTEVHYW
              330 340 350
YQGKTYVYYI YGTDHQVYAV DYPERYCCGC TIV
```

A cDNA and a chromosomal sequence encoding the SSUH2 protein is available from the NCBI database as accession no. AB024705 and AC034187, respectively,

An amino acid sequence for the protein encoded by the human TMISF4 gene that is a positive regulator of T cells as detected by Interleukin-2 production is available from the UniPROT database as accession no, P48230, shown below as SEQ ID NO:35.

```
                10 20 30 40
MCTGGCARCL GGTLIPLAFF GFLANILLFF PGGKVIDDND
                50 60 70 80
HLSQEIWEEG GILGSGVLMI FPALVELGLK NNDCCGCCGN
                90 100 110 120
EGCGKRFAMF ISTIFAVVGE IGAGYSFIIS AISINKGPKC
              130 140 150 160
LMANSTWGYP FHDGDYLNDE ALWNKCREPL NVVPWNLILF
              170 180 190 200
SILLVVGGIQ MVLCAIQVVN GLLGTLCGDC QCCGCCGGDG
PV
```

A cDNA and a chromosomal sequence encoding the TM4SF4 protein is available from the NCBI database as accession no. U31449 and C1471052, respectively.

The following genes are positive regulators of T cells as detected by increased T cell proliferation (see Table 3): ABCB1, ASAP1, ATP10A, DEAF1, FOXK1, ITGAX, LCE6A, LCP2, LEFTY1, MYC, NAT8B, OLFM3, and PLD6. Table 7 provides additional positive regulators of T cells as detected by increased T cell proliferation.

An amino acid sequence for the protein encoded by the human ATP10A gene that is a positive regulator of T cells as detected by increased cell proliferation is available from the UniPROT database as accession no. 060312, shown below as SEQ ID NO:36.

```
                10 20 30 40
MEREPAGTEE PGPPGRRRRR EGRTRTVRSN LLPPPGAEDP
                50 60 70 80
AAGAAKGERR RRRGCAQHLA DNRLKTTKYT LLSFLPKNLF
                90 100 110 120
EQFHRPANVY FVFIALLNFV PAVNAFQPGL ALAPVLFILA
              130 140 150 160
ITAFRDLWED YSRHRSDHKI NHLGCLVESR EEKKYVNREW
              170 180 190 200
KEIHVGDEVR LRCNEIFPAD ILLLSSSDPD GLCHIETANL
              210 220 230 240
DGETNLKRRQ VVRGESELVS EENPLTFTSV IECEKPNNDL
              250 260 270 280
SRERGCIIHD NGKKAGLYKE NLLIRGCTLR NTDAVVGIVI
              290 300 310 320
YAGHETKALL NNSGPRYKRS KLERQMNCDV LWCVLLLVCM
              330 340 350 360
SLESAVGHGL WIWRYQEKKS LEYVPKSDGS SISPVTAAVY
              370 380 390 400
SELTMIIVIQ VLIPISLYVS IEIVKACQVY FINQDMQLYD
              410 420 430 440
EETDSQLQCR ALNITEDIGQ IQYIFSDKTG TLTENKMVER
              450 460 470 480
RCTVSGVEYS HDANAQRLAR YQEADSEEEE VVPRGGSVSQ
              490 500 510 520
RGSIGSHQSV RVVHRTQSTK SHRRIGSRAE AKRASMLSKH
              530 540 550 560
TAFSSPMEKD ITPDPKLLEK VSECDKSLAV ARHQEHLLAH
              570 580 590 600
LSPELSDVED FFIALTICNT VVVTSPDQPR TKVRVRFELK
              610 620 630 640
SPVKTIEDEL RRFTPSCLTS GCSSIGSLAA NKSSHKIGSS
              650 660 670 680
FPSTPSSDGM LIRLEERLGQ PTSAIASNGY SSQADNWASE
              690 700 710 720
LAQEQESERE LRYEAESPDE AALVYAARAY NCVIVERLHD
              730 740 750 760
QVSVELPHLG RLTFELLHTL GEDSVRKRMS VVIRHPLIDE
              770 780 790 800
INVYTKGADS VVMDLLQPCS SVDARGRHQK KIRSKTQNYL
              810 820 830 840
NVYAAEGLRT ICIAKRVISK EEYACWLQSH LEAESSLENS
              850 860 870 880
EELLFQSAIR LETNIHLIGA TGIEDRLQDG VPETISKLRQ
              890 900 910 920
AGLQIWVLTG DKQETAVNIA YACKLLDHDE EVITLNATSQ
              930 940 950 960
EACAALLDQC LCYVQSRGLQ RAPEKTKGKV SMRFSSLCPP
              970 980 990 1000
STSTASGRRP SLVIDGRSLA YALEKNLEDK FLFLAKQCRS
            1010 1020 1030 1040
VICCRSTPLQ KSMVVKLVRS KLKAMTLAIG DGANDVSMIQ
            1050 1060 1070 1080
VADVGVGISG QEGMQAVMAS DEAVPKERYL ERLLILHGHW
            1090 1100 1110 1120
CYSRLANMVL YFFYKNTMEV GLLFWFQFFC GFSASTMIDQ
            1130 1140 1150 1160
WYLIFENLLF SSLPPLVTGV LDRDVPANVL LINPQLYKSG
            1170 1180 1190 1200
QNMEEYRPRT FWENMADAAF QSLVCFSIPY LAYYDSNVDL
            1210 1220 1230 1240
FTWGTPIVTI ALLTFLLHLG IETKIWTWIN WITCGFSVLL
            1250 1260 1270 1280
FFTVALIYNA SCATCYPPSN PYWTMQALLG DPVFYLTCLM
            1290 1300 1310 1320
TPVAALLPRL FERSLQGRVE PTQLQLARQL TRKSPRRCSA
            1330 1340 1350 1360
PKETFAQGRL PKDSGTEHSS GRTVKTSVPL SQPSWHTQQP
            1370 1380 1390 1400
VCSLEASGEP STVDMSMPVR EHTLLEGLSA PAPMSSAPGE
            1410 1420 1430 1440
AVLRSPGGCP EESKVRAAST GRVTPLSSLF SLPTESLLNW
            1450 1460 1470 1480
ISSWSLVSRL GSVLQFSRTE QLADGQAGRG LPVQPHSGRS
            1490
GLQGPDHRLL IGASSRRSQ
```

An amino acid sequence for the protein encoded by the human LCE6A gene that is a positive regulator of T cells as detected by increased cell proliferation is available from the UniPROT database as accession no. A0A183, shown below as SEQ U) NO:37.

```
                10 20 30 40 50
MSQQKQQSWK PPNVPKCSPP QRSNPCLAPY STPCGAPHSE GCHSSSQRPE
                60 70 80
VQKPRRARQK LRCLSRGTTY HCKEEECEGD
```

A cDNA and a chromosomal sequence encoding the LCE6A protein is available from the NCBI database as accession no. DQ991251 and AL162596, respectively.

An amino acid sequence for the protein encoded by the human NAT8B gene that is a positive regulator of T cells as detected by increased cell proliferation is available from the UniPROT database as accession no. Q9UHF3, shown below as SEQ ID NO:38.

```
                10 20 30 40 50
MAPYHIRKYQ ESDRKSVVGL LSGGMAEHAP ATFRRLLKLP RTLILLLGGA
                60 70 80 90 100
LALLLVSGSW ILALVFSLSL LPALWFLAKK PWTRYVDIAL RTDMSDITKS
              110 120 130 140 150
YLSECGSCFW VAESEEKVVG TVGALPVDDP TLREKRLQLF HLSVDNEHRG
              160 170 180 190 200
QGIAKALVRT VLQFARDQGY SEVVLDTSNI QLSAMGLYQS LGFKKTGQSF
              210 220
FHVWARLVDL HTVHFIYHLP SAQAGRL
```

A cDNA sequence encoding the NAT8B protein is available from the NCBI database as accession no. AF185571

### Negative Regulators of T Cells

The following genes are negative regulators of T cells as detected by interferon-y production (see Table 4): ACER2, ADGRVI, AIF1L, ALPL, AMACR, AMZ1, ARHGAP30, ARHGDIB, ARHGEF11, ARLI1, ATP2A2, B3GNT5, BAC H2, BLM, BSG, BTBD2, BTLA, BTRC, CAll, CASTOR2, CBLB, CCNT2, CCSER1, CD37, CD44, CD5, CD52, CD55, CDK6, CEACAMI, CEBPA, CEBPB, CEP164, CKAP2L, CLCN2, CLDN25, COLQ, CST5, CTNNA1, CYP24A, DDIT4L, DENND3, DGKG, DGKK, DGKZ, DSCI, EBF2, ECEL1, EIF3K, EPB41, EPS8L1, FAM35A, FAM53B, FAM83A, FKRP, FOXA3, FOXF1, FOXF2, FOXI3, FOXJ1, FOXL2, FOXL2NB, GiABRQ, GATA3, GATA4, GATA6, GCM2, GCSAM, GCSAML, GMFG, GNL3L, GRAP, GRTB2, GRIA1, GTSF1IL, HRI-2, HiYLS1, IKZF1, IKZF3, IL2RB, INPPL1, JMJD1C, KCNVI, KRIT1, LAiIBI, LAPIM5, LAT2, LAXI, LCK, LENEP, LMO4, LRRC25, LRRC4B, LYN, MAB21L2, MAP4K1, MBIP, MBOA Ti, MIETITL23, MIPEP, MIPOLI, MMP2I. MSMB, MUCI, MUC21, MUC8, N4BPI, NAIF1, NDNF, NFFAIC1, NFKB2, NFKBIA, NKX2-1, NKX2-3, NMB, NR2F1, ODF4, OPRD1, ORC5, OTUD4, PASDI, PBK, PCBP2, PDLIM1, PDPN, PECAIM1, PIP5Kl A, PIP5K1B, PITPNA, POGZ, POLK, POU2AF1, PSTPI1, PTPN12, PTPRC, PVRIG, RAB14, RBP7, RETREGI, RFC2, RHCE, RN^{F}19B, RNF2, RUSC2, SELPLG, SETD1IB, SH3KBP1, SIGLEC6, SIPA1L1, SLA, SLA2, SLC26A4, SLC44A5, SLC45A1, SLC6A8, SLC6A9, SMAD9, SMLkGP, SOCS3, SOX13, SPATA31A1, SPN, SPOCK3, SPREDI, STAP1, STK35, SULLT6B1, SYTI5, TEC, TIAMI, TMEMl51A, TMEM87B, TMPRSSI IE, TNNT2, TRIB2, TRLM28, TSPAN UBASH3B, UBQLN4, UBXN7, UNC119, UPP1, VPS28, WLS, ZKSCAN4. ZNF445, and ZNF474. Table 7 provides additional negative regulators of T cells as detected by interferon-y production.

Sequences and other information relating to these genes, and their encoded proteins, is available, for example from the NCBI and UniPROT databases, which are incorporated by reference.

A few examples of protein sequences encoded by some of the genes detected as negative regulators of T cells by interferon-γ production are provided. For example, an amino acid sequence for the protein encoded by the human ATI 1L gene that is a negative regulator of T cells as detected by interferon-γ production is available from the UniPROT database as accession no. Q9BQI0, shown below as SEQ ID NO:39.

```
                10 20 30 40 50
MSGELSNRFQ GGKAFGLLKA RQERRLAEIN REFLCDQKYS DEENLPEKLT
                60 70 80 90 100
AFKEKYMEFD LNNEGEIDLM SLKRMMEKLG VPKTHLEMKK MISEVTGGVS
              110 120 130 140 150
DTISYRDFVN MMLGKRSAVL KLVMMFEGKA NESSPKPVGP PPERDIASLP
```

A cDNA and a chromosomal sequence encoding the AIF1L protein is available from the NCBI database as accession no. AL136566 and AL157938, respectively.

An amino acid sequence for the protein encoded by the human ARHGDIB gene that is a negative regulator of T cells as detected by interferon-γ production is available from the UniPROT database as accession no. P52566, shown below as SEQ ID NO:40.

```
                10 20 30 40 50
MTEKAPEPHV EEDDDDELDS KINYKPPPQK SLKELQEMDK DDESLIKYKK
                60 70 80 90 100
TLLGDGPVVT DPKAPNVVVT RLTLVCESAP GPITMDLTGD LEALKKETIV
              110 120 130 140 150
LKEGSEYRVK IHFKVNRDIV SGLKYVQHTY RTGVKVDKAT FMVGSYGPRP
              160 170 180 190 200
EEYEFLTPVE EAPKGMLARG TYHNKSFFTD DDKQDHLSWE WNLSIKKEWT
E
```

A cDNA and a chromosomal sequence encoding the ARHGDIB protein is available from the NCBI database as accession no. L20688 and CH471094, respectively.

An amino acid sequence for the protein encoded by the human BLM gene that is a negative regulator of T cells as detected by interferon-γ production is available from the UniPROT database as accession no. P54132, shown below as SEQ ID NO:41.

```
                10 20 30 40 50
MAAVPQNNLQ EQLERHSART LNNKLSLSKP KFSGFTFKKK TSSDNNVSVT
                60 70 80 90 100
NVSVAKTPVL RNKDVNVTED FSFSEPLPNT TNQQRVKDFF KNAPAGQETQ
              110 120 130 140 150
RGGSKSLLPD FLQTPKEVVC TTQNTPTVKK SRDTALKKLE FSSSPDSLST
              160 170 180 190 200
INDWDDMDDF DTSETSKSFV TPPQSHFVRV STAQKSKKGK RNFFKAQLYT
              210 220 230 240 250
TNTVKTDLPP PSSESEQIDL TEEQKDDSEW LSSDVICIDD GPIAEVHINE
              260 270 280 290 300
DAQESDSLKT HLEDERDNSE KKKNLEEAEL HSTEKVPCIE FDDDDYDTDF
              310 320 330 340 350
VPPSPEEIIS ASSSSSKCLS TLKDLDTSDR KEDVLSTSKD LLSKPEKMSM
              360 370 380 390 400
QELNPETSTD CDARQISLQQ QLIHVMEHIC KLIDTIPDDK LKLLDCGNEL
              410 420 430 440 450
LQQRNIRRKL LTEVDFNKSD ASLLGSLWRY RPDSLDGPME GDSCPTGNSM
              460 470 480 490 500
KELNFSHLPS NSVSPGDCLL TTTLGKTGFS ATRKNLFERP LFNTHLQKSF
              510 520 530 540 550
VSSNWAETPR LGKKNESSYF PGNVLTSTAV KDQNKHTASI NDLERETQPS
              560 570 580 590 600
YDIDNFDIDD FDDDDDWEDI MHNLAASKSS TAAYQPIKEG RPIKSVSERL
              610 620 630 640 650
SSAKTDCLPV SSTAQNINFS ESIQNYTDKS AQNLASRNLK HERFQSLSFP
              660 670 680 690 700
HTKEMMKIFH KKFGLHNFRT NQLEAINAAL LGEDCFILMP TGGGKSLCYQ
              710 720 730 740 750
LPACVSPGVT VVISPLRSLI VDQVQKLTSL DIPATYLTGD KTDSEATNIY
              760 770 780 790 800
LQLSKKDPII KLLYVTPEKI CASNRLISTL ENLYERKLLA RFVIDEAHCV
              810 820 830 840 850
SQWGHDFRQD YKRMNMLRQK FPSVPVMALT ATANPRVQKD ILTQLKILRP
              860 870 880 890 900
QVFSMSFNRH NLKYYVLPKK PKKVAFDCLE WIRKHHPYDS GIIYCLSRRE
              910 920 930 940 950
CDTMADTLQR DGLAALAYHA GLSDSARDEV QQKWINQDGC QVICATIAFG
              960 970 980 990 1000
MGIDKPDVRF VIHASLPKSV EGYYQESGRA GRDGEISHCL LFYTYHDVTR
            1010 1020 1030 1040 1050
LKRLIMMEKD GNHHTRETHF NNLYSMVHYC ENITECRRIQ LLAYFGENGF
            1060 1070 1080 1090 1100
NPDFCKKHPD VSCDNCCKTK DYKTRDVTDD VKSIVRFVQE HSSSQGMRNI
            1110 1120 1130 1140 1150
KHVGPSGRFT MNMLVDIFLG SKSAKIQSGI FGKGSAYSRH NAERLFKKLI
            1160 1170 1180 1190 1200
LDKILDEDLY INANDQAIAY VMLGNKAQTV LNGNLKVDFM ETENSSSVKK
            1210 1220 1230 1240 1250
QKALVAKVSQ REEMVKKCLG ELTEVCKSLG KVFGVHYFNI FNTVTLKKLA
            1260 1270 1280 1290 1300
ESLSSDPEVL LQIDGVTEDK LEKYGAEVIS VLQKYSEWTS PAEDSSPGIS
            1310 1320 1330 1340 1350
LSSSRGPGRS AAEELDEEIP VSSHYFASKT RNERKRKKMP ASQRSKRRKT
            1360 1370 1380 1390 1400
ASSGSKAKGG SATCRKISSK TKSSSIIGSS SASHTSQATS GANSKLGIMA
            1410
PPKPINRPFL KPSYAFS
```

A cDNA and a chromosomal sequence encoding the BLM protein is available from the NCBI database as accession no. U39817 and AY886902, respectively.

An amino acid sequence for the protein encoded by the human BSG gene that is a negative regulator of T cells as detected by interferon-γ production is available from the UniPROT database as accession no. Q7KTJ7, shown below as SEQ ID NO:42.

```
                10 20 30 40 50
MEAKFLASAL SFLSIFLAIY AQSLANDLSK ESTEFEESPT IYYGDPVVNL
                60 70 80 90 100
GQPFSITCII PITDQIHWLK NGEPITRHNL RHGRDDHAYV LSESAIEGEK
              110 120 130 140 150
HKIEAHLSVR HALKVHEGRY QCNRRRGSYI LHVRDPKGVG AGAGEPTESG
              160 170 180 190 200
YQTIDELTPN SADDFFTRAW LEQQQQQQQL PHQSHKLHKS HLGYGNASLS
              210 220 230 240 250
GSQPWHPSAG GGGIHRVYSA TPPDFPPPRL NLLEQTVAPP EPPTILYNPN
              260 270 280 290 300
PTHPTASATA TETSVLLTTA HHHAHHQQQL QQQSQHTLNA FQLPLPPRPN
              310 320 330 340 350
PGQNERYQTY APHYVPPVVV SGAGAGAGAD PGAGASGEQT TISAATSTRA
              360 370 380 390 400
MMGGGGGVAG AGFSAGASGP MLGAGGHMLM GGQGHQVHLQ HQTLLPVKMD
              410 420 430 440 450
KLVPNYDNAE HQMKFYDIRS PLVLSCNVKD GTPGGVLIWK KNGTAVTDVP
              460 470 480 490 500
SLRGRFKLIA DENKFIIDKT DTNDDGKYSC EFDGVSKEIE VIARVVVRVP
              510 520 530 540 550
SNTAVVEGEK MSVTCSVVGT KPELTWTFAN VTLTNATDRF ILKPDDNGVP
              560 570 580 590 600
NAILTLDNVT LDDRGEYKCI GRNAANVYGG NTTTPASDVT TVRVKGKFAA
              610 620 630 640 650
LWPFLGICAE VLILCIIILI YEKRRNKSEL EESDTDPQEQ KKKRRNYD
```

A cDNA and a chromosomal sequence encoding the BSG protein is available from the NCBI database as accession no, AE014134 and AAN10661.2, respectively.

An amino acid sequence for the protein encoded by the human BTBD2 gene that is a negative regulator of T cells as detected by interferon-v production is available from the UniPROT database as accession no. Q9BX70, shown below as SEQ ID NO: 43.

```
                10 20 30 40 50
MAAGGSGGRA SCPPGVGVGP GTGGSPGPSA NAAATPAPGN AAAAAAAAAA
                60 70 80 90 100
AAAAPGPTPP APPGPGTDAQ AAGAERAEEA AGPGAAALQR EAAYNWQASK
              110 120 130 140 150
PTVQERFAFL FNNEVLCDVH FLVGKGLSSQ RIPAHRFVLA VGSAVFDAMF
              160 170 180 190 200
NGGMATTSTE IELPDVEPAA FLALLKFLYS DEVQIGPETV MTTLYTAKKY
              210 220 230 240 250
AVPALEAHCV EFLKKNLRAD NAFMLLTQAR LFDEPQLASL CLENIDKNTA
              260 270 280 290 300
DAITAEGFTD IDLDTLVAVL ERDTLGIREV RLFNAVVRWS EAECQRQQLQ
              310 320 330 340 350
VTPENRRKVL GKALGLIRFP LMTIEEFAAG PAQSGILVDR EVVSLFLHFT
              360 370 380 390 400
VNPKPRVEFI DRPRCCLRGK ECSINRFQQV ESRWGYSGTS DRIRFSVNKR
              410 420 430 440 450
IFVVGFGLYG SIHGPTDYQV NIQIIHTDSN TVLGQNDTGF SCDGSASTFR
              460 470 480 490 500
VMFKEPVEVL PNVNYTACAT LKGPDSHYGT KGLRKVTHES PTTGAKTCFT
              510 520
FCYAAGNNNG TSVEDGQIPE VIFYT
```

A cDNA and a chromosomal sequence encoding the BTBD2 protein is available from the NCBI database as accession no. AF355797 and AC004678, respectively.

An amino acid sequence for the protein encoded by the human CASTOR2 gene that is a negative regulator of T cells as detected by interferon-γ production is available from the UniPROT database as accession no. A6NHX0, shown below as SEQ ID NO:44.

```
                10 20 30 40 50
MELHILEHRL QVASVAKESI PLFTYGLIKL AFLSSKTRCK FFSLTETPED
                60 70 80 90 100
YTIIVDEEGF LELPSSEHLS VADATWLALN VVSGGGSFSS SQPIGVTKIA
              110 120 130 140 150
KSVIAPLADQ NISVFMLSTY QTDFILVRER DLPFVTHTLS SEFTILRVVN
              160 170 180 190 200
GETVAAENLG ITNGFVKPKL VQRPVIHPLS SPSNRFCVTS LDPDTLPAVA
              210 220 230 240 250
TLLMDVMFYS NGVKDPMATG DDCGHIRFFS FSLIEGYISL VMDVQTQQRF
              260 270 280 290 300
PSNLLFTSAS GELWKMVRIG GQPLGFDECG IVAQISEPLA AADIPAYYIS
              310 320
TFKFDHALVP EENINGVISA LKVSQAEKH
```

A cDNA and a chromosomal sequence encoding the CASTOR2 protein is available from the NCBI database as accession no. BC147030 and AC245150, respectively.

An amino acid sequence for the protein encoded by the human CCSER1 gene that is a negative regulator of T cells as detected by interferon-γ production is available from the UniPROT database as accession no, Q9C013, shown below as SEQ ID NO:45.

```
                10 20 30 40 50
MGDSGSRRST LVSRLPIFRR SINRRHDSLP SSPSSSNTVG VHSSSPSSTN
                60 70 80 90 100
SSSGSTGKRR SIFRTPSISF HHKKGSEPKQ EPTNQNLSIS NGAQPGHSNM
              110 120 130 140 150
QKLSLEEHIK TRGRHSVGFS SSRNKKITRS LTEDFEREKE HSTNKNVFIN
              160 170 180 190 200
CLSSGKSEGD DSGFTEDQTR RSVKQSTRKL LPKSFESSHYK FSKPVLQSQS
              210 220 230 240 250
ISLVQQSEFS LEVTQYQERE PVLVRASPSC SVDVTERAGS SLQSPLLSAD
              260 270 280 290 300
LTTAQTPSEF LALTEDSVSE MDAFSKSGSM ASHCDNFGHN DSTSQMSLNS
              310 320 330 340 350
AAVIKTTTEL TGTVPCAIMS PGKYRLEGQC STESNSLPET SAANQKEVLL
              360 370 380 390 400
QIAELPATSV SHSESNLPAD SEREENIGLQ NGETMLGTNS PRKLGFYEQH
              410 420 430 440 450
KAIAEHVKGI HPISDSKIIP TSGDHHIFNK TSHGYEANPA KVLASSLSPE
              460 470 480 490 500
REGRFIERRL RSSSEGTAGS SRMILKPKDG NIEEVNSLRK QRAGSSSSKM
              510 520 530 540 550
NSLDVLNNLG SCELDEDDLM LDLEFLEEQS LHPSVCREDS YHSVVSCAAV
              560 570 580 590 600
VLTPMEPMIE MKKREEPEFP EPSKQNLSLK LTKDVDQEAR CSHISRMPNS
              610 620 630 640 650
PSADWPLQGV EENGGIDSLP FRLMLQDCTA VKTLLLKMKR VLQESADMSP
              660 670 680 690 700
ASSTTSLPVS PLTEEPVPFK DIMKDECSML KLQLKEKDEL ISQLQEELGK
              710 720 730 740 750
VRHLQKAFAS RVDKSTQTEL LCYDGLNLKR LETVQGGREA TYRNRIVSQN
              760 770 780 790 800
LSTRDRKAIH TPTEDRFRYS AADQTSPYKN KTCQLPSLCL SNFLKDKELA
              810 820 830 840 850
EVIKHSRGTY ETLTSDVTQN LRATVGQSSL KPTAKTEGLS TFLEKPKDQV
              860 870 880 890 900
ATARQHSTFT GRFGQPPRGP ISLHMYSRKN VFLHHNLHST ELQTLGQQDG
```

A cDNA and a chromosomal sequence encoding the CCSER1 protein is available from the NCBT database as accession no. AB051467 and AC093729, respectively.

An amino acid sequence for the protein encoded by the human CLCN2 gene that is a negative regulator of T cells as detected by interferon-γ production is available from the UniPROT database as accession no. P51788, shown below as SEQ ID NO:46,

```
                10 20 30 40 50
MAAAAAEEGM EPRALQYEQT LMYGRYTQDL GAFAKEEAAR IRLGGPEPWK
                60 70 80 90 100
GPPSSRAAPE LLEYGRSRCA RCRVCSVRCH KFLVSRVGED WIFLVLLGLL
              110 120 130 140 150
MALVSWVMDY AIAACLQAQQ WMSRGLNTSI LLQYLAWVTY PVVLITFSAG
              160 170 180 190 200
FTQILAPQAV GSGIPEMKTI LRGVVLKEYL TLKTFIAKVI GLTCALGSGM
              210 220 230 240 250
PLGKEGPFVH IASMCAALLS KFLSLFGGIY ENESRNTEML AAACAVGVGC
              260 270 280 290 300
CFAAPIGGVL FSIEVTSTFF AVRNYWRGFF AATFSAFIFR VLAVWNRDEE
              310 320 330 340 350
TITALFKTRF RLDFPFDLQE LPAFAVIGIA SGFGGALFVY LNRKIVQVMR
              360 370 380 390 400
KQKTINRFLM RKRLLFPALV TLLISTLTFP PGFGQFMAGQ LSQKETLVTL
              410 420 430 440 450
FDNRTWVRQG LVEELEPPST SQAWNPPRAN VFLTLVIFIL MKFWMSALAT
              460 470 480 490 500
TIPVPCGAFM PVEVIGAAFG RLVGESMAAW FPDGIHTDSS TYRIVPGGYA
              510 520 530 540 550
VVGAAALAGA VTHTVSTAVI VFELTGQIAH ILPVMIAVIL ANAVAQSLQP
              560 570 580 590 600
SLYDSIIRIK KLPYLPELGW GRHQQYRVRV EDIMVRDVPH VALSCTFRDL
              610 620 630 640 650
RLALHRTKGR MLALVESPES MILLGSIERS QVVALLGAQL SPARRRQHMQ
              660 670 680 690 700
ERRATQTSPL SDQEGPPTPE ASVCFQVNTE DSAFPAARGE THKPLKPALK
              710 720 730 740 750
RGPSVTRNLG ESPTGSAESA GIALRSLFCG SPPPEAASEK LESCEKRKLK
              760 770 780 790 800
RVRISLASDA DLEGEMSPEE ILEWEEQQLD EPVNFSDCKI DPAPFQLVER
              810 820 830 840 850
TSLHKTHTIF SLLGVDHAYV TSIGRLIGIV TLKELRKAIE GSVTAQGVKV
              860 870 880 890
RPPLASFRDS ATSSSDTETT EVHALWGPHS RHGLPREGSP SDSDDKCQ
```

A cDNA and a chromosomal sequence encoding the CLCN2 protein is available from the NCBI database as accession no. S77770 and AC078797, respectively.

An amino acid sequence for the protein encoded by the human EBF2 gene that is a negative regulator of T cells as detected by interferon-γ production is available from the UniPROT database as accession no. Q9 AK2, shown below as SEQ ID NO:47.

```
                10 20 30 40 50
MFGIQDTLGR GPTLKEKSLG AEMDSVRSWV RNVGVVDANV AAQSGVALSR
                60 70 80 90 100
AHFEKQPPSN LRKSNFFHFV LALYDRQGQP VEIERTAFVD EVENDKEQGN
              110 120 130 140 150
EKTNNGTHYK LQLLYSNGVR TEQDLYVRLI DSVTKQPIAY EGQNKNPEMC
              160 170 180 190 200
RVLLTHEVMC SRCCEKKSCG NRNETPSDPV IIDRFFLKFF LKCNQNCLKT
              210 220 230 240 250
AGNPRDMRRF QVVLSTTVNV DGHVLAVSDN MFVHNNSKHG RRARRLDPSE
              260 270 280 290 300
ATPCIKAISP SEGWTTGGAM VIIIGDNFFD GLQVVFGTML VWSELITPHA
              310 320 330 340 350
IRVQTPPRHI PGVVEVTLSY KSKQFCKGAP GRFIYTALNE PTIDYGFQRL
              360 370 380 390 400
QKVIPRHPGD PERLAKEMLL KRAADLVEAL YGTPHNNQDI ILKRAADIAE
              410 420 430 440 450
ALYSVPRNPS QLPALSSSPA HSGMMGINSY GSQLGVSISE STQGNNQGYI
              460 470 480 490 500
RNTSSISPRG YSSSSTPQQS NYSTSSNSMN GYSNVPMANL GVPGSPGFLN
              510 520 530 540 550
GSPTGSPYGI MSSSPTVGSS STSSILPFSS SVFPAVKQKS AFAPVIRPQG
              560 570
SPSPACSSGN GNGFRAMTGL VVPPM
```

A cDNA and a chromosomal sequence encoding the EBF2 (COE2) protein is available from the NCBI database as accession no. AY700779 and AC023566, respectively,

An amino acid sequence for the protein encoded by the human FAM83A gene that is a negative regulator of T cells as detected by interferon-7 production is available from the UniPROT database as accession no. Q86UY5, shown below as SEQ ID NO:48.

```
                10 20 30 40 50
MSRSRHLGKI RKRLEDVKSQ WVRPARADFS DNESARLATD ALLDGGSEAY
                60 70 80 90 100
WRVLSQEGEV DFLSSVEAQY IQAQAREPPC PPDTLGGAEA GPKGLDSSSL
              110 120 130 140 150
QSGTYFPVAS EGSEPALLHS WASAEKPYLK EKSSATVYFQ TVKHNNIRDL
              160 170 180 190 200
VRRCITRISQ VLVILMDVFT DVEIFCDILE AANKRGVFVC VILDQGGVKL
              210 220 230 240 250
FQEMCDKVQI SDSHLKNISI RSVEGEIYCA KSGRKFAGQI REKFIISDWR
              260 270 280 290 300
FVLSGSYSFT WLCGHVHRNI LSKFTGQAVE LFDEEFRHLY ASSKPVMGLK
              310 320 330 340 350
SPRLVAPVPP GAAPANGRLS SSSGSASDRT SSNPFSGRSA GSHPGTRSVS
              360 370 380 390 400
ASSGPCSPAA PHPPPPPRFQ PHQGPWGAPS PQAHLSPRPH DGPPAAVYSN
              410 420 430
LGAYRPTRLQ LEQLGLVPRL TPTWRPFLQA SPHF
```

A cDNA sequence encoding the FAM83A protein is available from the NCBI database as accession no. DQ280322.

An amino acid sequence for the protein encoded by the human FOXF1 gene that is a negative regulator of T cells as detected by interferon-γ, production is available from the UniPROT database, shown below as SEQ ID NO:49.

```
                10 20 30 40 50
MSSAPEKQQP PHGGGGGGGG GGGAAMDPAS SGPSKAKKTN AGIRRPEKPP
                60 70 80 90 100
YSYIALIVMA IQSSPTKRLT LSEIYQFLQS RFPFFRGSYQ GWKNSVRHNL
              110 120 130 140 150
SLNECFIKLP KGLGRPGKGH YWTIDPASEF MFEEGSFRRR PRGFRRKCQA
              160 170 180 190 200
LKPMYSMMNG LGFNHLPDTY GFQGSAGGLS CPPNSLALEG GLGMMNGHLP
              210 220 230 240 250
GNVDGMALPS HSVPHLPSNG GHSYMGGCGG AAAGEYPHHD SSVPASPLLP
              260 270 280 290 300
TGAGGVMEPH AVYSGSAAAW PPSASAALNS GASYIKQQPL SPCNPAANPL
              310 320 330 340 350
SGSLSTHSLE QPYLHQNSHN APAELQGIPR YHSQSPSMCD RKEFVFSFNA
              360 370
MASSSMHSAG GGSYYHQQVT YQDIKPCVM
```

A cDNA and a chromosomal sequence encoding the FOXF1 protein is available from the NCBI database as accession no. U13219 and AF085343, respectively.
An amino acid sequence for the protein encoded by the human FOXL3 gene that is a negative regulator of T cells as detected by interferon-production is available from the UniPROT database as accession no. A8MTJ6, shown below as SEQ ID NO:50.

```
                10 20 30 40 50
MALYCGDNFG VYSQPGLPPP AATAAAPGAP PAARAPYGLA DYAAPPAAAA
                60 70 80 90 100
NPYLWLNGPG VGGPPSAAAA AAAAYLGAPP PPPPPGAAAG PFLQPPPAAG
              110 120 130 140 150
TFGCSQRPFA QPAPAAPASP AAPAGPGELG WLSMASREDL MKMVRPPYSY
              160 170 180 190 200
SALIAMAIQS APERKLTLSH IYQFVADSFP FYQRSKAGWQ NSIRHNLSLN
              210 220 230 240 250
DCFKKVPRDE DDPGKGNYWT LDPNCEKMFD NGNFRRKRKR RSEASNGSTV
              260 270 280 290 300
AAGTSKSEEG LSSGLGSGVG GKPEEESPST LLRPSHSPEP PEGTKSTASS
              310 320 330 340 350
PGGPMLTSTP CLNTFFSSLS SLSVSSSVST QRALPGSRHL GIQGAQLPSS
              360 370 380 390 400
GVFSPTSISE ASADTLQLSN STSNSTGQRS SYYSPFPAST SGGQSSPFSS
              410 420
PFHNFSMVNS LIYPREGSEV
```

A cDNA and a chromosomal sequence encoding the FOXI3 protein is available from the NCBI database as accession no. BN001222 and AC012671, respectively.

An amino acid sequence for the protein encoded by the human FOXL2NB gene that is a negative regulator of T cells as detected by interferon-γ production is available from the UnIPROT database as accession no. Q6ZUIU3, shown below as SEQ ID NO:51.

```
                10 20 30 40 50
MTRTPVGSAR TRPKPRKLGP QRGKALQASS RLSESPALVK KRMPDACTLG
                60 70 80 90 100
RAGIGLPKMC LHMAVRHSKA QKTGPGILQQ RQKPPAPRAS GGPALLGKRR
              110 120 130 140 150
GCSEAGSASL EPLSSSRAAA GCLNQVPLSP FLAGPRNTRR LPAPERERIE
              160 170
LAATLCLEGW PLRCLASKGK LHCVY
```

A cDNA and a chromosomal sequence encoding the FOXL2NB protein is available from the N-CBI database as accession no. AK125319 and AC092947, respectively.

An amino acid sequence for the protein encoded by the human HYLS1 gene that is a negative regulator of T cells as detected by interferon-γ production is available from the UniPROT database as accession no. Q96M1 1, shown below as SEQ ID NO:52.

```
                10 20 30 40 50
MEELLPDGQI WANMDPEERM LAAATAFTHI CAGQGEGDVR REAQSIQYDP
                60 70 80 90 100
YSKASVAPGK RPALPVQLQY PHVESNVPSE TVSEASQRLR KPVMKRKVLR
              110 120 130 140 150
RKPDGEVLVT DESIISESES GTENDQDLWD LRQRLMNVQF QEDKESSFDV
              160 170 180 190 200
SQKFNLPHEY QGISQDQLIC SLQREGMGSP AYEQDLIVAS RPKSFILPKL
              210 220 230 240 250
DQLSRNRGKT DRVARYFEYK RDWDSIRLPG EDHRKELRWG VREQMLCRAE
              260 270 280 290
PQSKPQHIYV PNNYLVPTEK KRSALRWGVR CDLANGVIPR KLPFPLSPS
```

A cDNA and a chromosomal sequence encoding the HYLS1 protein is available from the NCBI database as accession no, AK057477 and AP000842, respectively.

An amino acid sequence for the protein encoded by the human LAMB1 gene that is a negative regulator of T cells as detected by interferon-γ production is available from the UniPROT database as accession no. P07942, shown below as SEQ ID NO:53,

```
                10 20 30 40 50
MGLLQLLAFS FLALCRARVR AQEPEFSYGC AEGSCYPATG DLLIGRAQKL
                60 70 80 90 100
SVTSTCGLHK PEPYCIVSHL QEDKKCFICN SQDPYHETLN PDSHLIENVV
              110 120 130 140 150
TTFAPNRLKI WWQSENGVEN VTIQLDLEAE FHFTHLIMTF KTFRPAAMLI
              160 170 180 190 200
ERSSDFGKTW GVYRYFAYDC EASFPGISTG PMKKVDDIIC DSRYSDIEPS
              210 220 230 240 250
TEGEVIFRAL DPAFKIEDPY SPRIQNLLKI TNLRIKFVKL HTLGDNLLDS
              260 270 280 290 300
RMEIREKYYY AVYDMVVRGN CFCYGHASEC APVDGFNEEV EGMVHGHCMC
              310 320 330 340 350
RHNTKGLNCE LCMDFYHDLP WRPAEGRNSN ACKKCNCNEH SISCHFDMAV
              360 370 380 390 400
YLATGNVSGG VCDDCQHNTM GRNCEQCKPF YYQHPERDIR DPNFCERCTC
              410 420 430 440 450
DPAGSQNEGI CDSYTDFSTG LIAGQCRCKL NVEGEHCDVC KEGFYDLSSE
              460 470 480 490 500
DPFGCKSCAC NPLGTIPGGN PCDSETGHCY CKRLVIGQHC DQCLPEHWGL
              510 520 530 540 550
SNDLDGCRPC DCDLGGALNN SCFAESGQCS CRPHMIGRQC NEVEPGYYFA
              560 570 580 590 600
TLDHYLYEAE EANIGPGVSI VERQYIQDRI PSWTGAGFVR VPEGAYLEFF
              610 620 630 640 650
IDNIPYSMEY DILIRYEPQL PDHWEKAVIT VQRPGRIPTS SRCGNTIPDD
              660 670 680 690 700
DNQVVSLSPG SRYVVLPRPV CFEKGTNYTV RLELPQYTSS DSDVESPYTL
              710 720 730 740 750
IDSLVLMPYC KSLDIFTVGG SGDGVVTNSA WETFQRYRCL ENSRSVVKTP
              760 770 780 790 800
MTDVCRNIIF SISALLHQTG LACECDPQGS LSSVCDPNGG QCQCRPNVVG
              810 820 830 840 850
RTCNRCAPGT FGFGPSGCKP CECHLQGSVN AFCNPVTGQC HCFQGVYARQ
              860 870 880 890 900
CDRCLPGHWG FPSCQPCQCN GHADDCDPVT GECLNCQDYT MGHNCERCLA
              910 920 930 940 950
GYYGDPIIGS GDHCRPCPCP DGPDSGRQFA RSCYQDPVTL QLACVCDPGY
              960 970 980 990 1000
IGSRCDDCAS GYFGNPSEVG GSCQPCQCHN NIDTTDPEAC DKETGRCLKC
            1010 1020 1030 1040 1050
LYHTEGEHCQ FCRFGYYGDA LQQDCRKCVC NYLGTVQEHC NGSDCQCDKA
            1060 1070 1080 1090 1100
TGQCLCLPNV IGQNCDRCAP NTWQLASGTG CDPCNCNAAH SFGPSCNEFT
            1110 1120 1130 1140 1150
GQCQCMPGFG GRTCSECQEL FWGDPDVECR ACDCDPRGIE TPQCDQSTGQ
            1160 1170 1180 1190 1200
CVCVEGVEGP RCDKCTRGYS GVFPDCTPCH QCFALWDVII AELTNRTHRF
            1210 1220 1230 1240 1250
LEKAKALKIS GVIGPYRETV DSVERKVSEI KDILAQSPAA EPLKNIGNLF
            1260 1270 1280 1290 1300
EEAEKLIKDV TEMMAQVEVK LSDTTSQSNS TAKELDSLQT EAESLDNTVK
            1310 1320 1330 1340 1350
ELAEQLEFIK NSDIRGALDS ITKYFQMSLE AEERVNASTT EPNSTVEQSA
            1360 1370 1380 1390 1400
LMRDRVEDVM MERESQFKEK QEEQARLLDE LAGKLQSLDL SAAAEMTCGT
            1410 1420 1430 1440 1450
PPGASCSETE CGGPNCRTDE GERKCGGPGC GGLVTVAHNA WQKAMDLDQD
            1460 1470 1480 1490 1500
VLSALAEVEQ LSKMVSEAKL RADEAKQSAE DILLKTNATK EKMDKSNEEL
            1510 1520 1530 1540 1550
RNLIKQIRNF LTQDSADLDS IEAVANEVLK MEMPSTPQQL QNLTEDIRER
            1560 1570 1580 1590 1600
VESLSQVEVI LQHSAADIAR AEMLLEEAKR ASKSATDVKV TADMVKEALE
            1610 1620 1630 1640 1650
EAEKAQVAAE KAIKQADEDI QGTQNLLTSI ESETAASEET LFNASQRISE
            1660 1670 1680 1690 1700
LERNVEELKR KAAQNSGEAE YIEKVVYTVK QSAEDVKKTL DGELDEKYKK
            1710 1720 1730 1740 1750
VENLIAKKTE ESADARRKAE MLQNEAKTLL AQANSKLQLL KDLERKYEDN
            1760 1770 1780
QRYLEDKAQE LARLEGEVRS LIKDISQKVA VYSTCL
```

A cDNA and a chromosomal sequence encoding the LAMB1 protein is available from the NCBI database as accession no. M61916 and M61950, respectively.

An amino acid sequence for the protein encoded by the human LENEP gene that is a negative regulator of T cells as detected by interferon-γ production is available from the UniPROT database as accession no. Q9Y5L5, shown below as SEQ ID N0:54.

```
                10 20 30 40 50
MQPRTQPLAQ TLPFFLGGAP RDTGLRVPVI KMGTGWEGFQ RTLKEVAYIL
                60
LCCWCIKELL D
```

A cDNA and a chromosomal sequence encoding the LENEP protein is available from the NCBI database as accession no. AF268478 and AF144412, respectively.

An amino acid sequence for the protein encoded by the human LRRC4B gene that is a negative regulator of T cells as detected by interferon-7 production is available from the UniPROT database as accession no. Q9NT99, shown below as SEQ ID NO:55.

```
                10 20 30 40 50
MARARGSPCP PLPPGRMSWP HGALLFLWLF SPPLGAGGGG VAVTSAAGGG
                60 70 80 90 100
SPPATSCPVA CSCSNQASRV ICTRRDLAEV PASIPVNTRY LNLQENGIQV
              110 120 130 140 150
IRTDTFKHLR HLEILQLSKN LVRKIEVGAF NGLPSLNTLE LFDNRLTTVP
              160 170 180 190 200
TQAFEYLSKL RELWLRNNPI ESIPSYAFNR VPSLRRLDLG ELKRLEYISE
              210 220 230 240 250
AAFEGLVNLR YLNLGMCNLK DIPNLTALVR LEELELSGNR LDLIRPGSFQ
              260 270 280 290 300
GLTSLRKLWL MHAQVATIER NAFDDLKSLE ELNLSHNNLM SLPHDLFTPL
              310 320 330 340 350
HRLERVHLNH NPWHCNCDVL WLSWWLKETV PSNTTCCARC HAPAGLKGRY
              360 370 380 390 400
IGELDQSHFT CYAPVIVEPP TDLNVTEGMA AELKCRTGTS MTSVNWLTPN
              410 420 430 440 450
GTLMTHGSYR VRISVLHDGT LNFTNVTVQD TGQYTCMVTN SAGNTTASAT
              460 470 480 490 500
LNVSAVDPVA AGGTGSGGGG PGGSGGVGGG SGGYTYFTTV TVETLETQPG
              510 520 530 540 550
EEALQPRGTE KEPPGPTTDG VWGGGRPGDA AGPASSSTTA PAPRSSRPTE
              560 570 580 590 600
KAFTVPITDV TENALKDLDD VMKTTKIIIG CFVAITFMAA VMLVAFYKLR
              610 620 630 640 650
KQHQLHKHHG PTRTVEIINV EDELPAASAV SVAAAAAVAS GGGVGGDSHL
              660 670 680 690 700
ALPALERDHL NHHHYVAAAF KAHYSSNPSG GGCGGKGPPG LNSIHEPLLF
              710
KSGSKENVQE TQI
```

A cDNA and a chromosomal sequence encoding the LRRC4B protein is available from the NCBI database as accession no. BC019687 and AC008743, respectively.

An amino acid sequence for the MAB21L2 protein encoded by the human gene that is a negative regulator of T cells as detected by interferon-γ production is available from the UniPROT database as accession no. Q9Y586, shown below as SEQ [D NO:56.

```
                10 20 30 40 50
MIAAQAKLVY QLNKYYTERC QARKAAIAKT IREVCKVVSD VLKEVEVQEP
                60 70 80 90 100
RFISSLSEID ARYEGLEVIS PTEFEVVLYL NQMGVFNFVD DGSLPGCAVL
              110 120 130 140 150
KLSDGRKRSM SLWVEFITAS GYLSARKIRS RFQTLVAQAV DKCSYRDVVK
              160 170 180 190 200
MIADTSEVKL RIRERYVVQI TPAFKCTGIW PRSAAQWPMP HIPWPGPNRV
              210 220 230 240 250
AEVKAEGFNL LSKECYSLTG KQSSAESDAW VLQFGEAENR LLMGGCRNKC
              260 270 280 290 300
LSVLKTLRDR HLELPGQPLN NYHMKTLLLY ECEKHPRETD WDESCLGDRL
              310 320 330 340 350
NGILLQLISC LQCRRCPHYF LPNLDLFQGK PHSALESAAK QTWRLAREIL
TNPKSLDKL
```

A cDNA and a chromosomal sequence encoding the MAB21L2 protein is available from the NCBI database as accession no. AF262032 and A-155219. respectively.

An amino acid sequence for the protein encoded by the human RETREGI gene that is a negative regulator of T cells as detected by interferon-γ production is available from the UniPROT database as accession no. Q9H6L5, shown below as SEQ ID NO:57.

```
                10 20 30 40 50
MASPAPPEHA EEGCPAPAAE EQAPPSPPPP QASPAERQQQ EEEAQEAGAA
                60 70 80 90 100
EGAGLQVEEA AGRAAAAVTW LLGEPVLWLG CRADELLSWK RPLRSLLGFV
              110 120 130 140 150
AANLLFWFLA LTPWRVYHLI SVMILGRVIM QIIKDMVLSR TRGAQLWRSL
              160 170 180 190 200
SESWEVINSK PDERPRLSHC IAESWMNFSI FLQEMSLFKQ QSPGKFCLLV
              210 220 230 240 250
CSVCTFFTIL GSYIPGVILS YLLLLCAFLC PLEKCNDIGQ KIYSKIKSVL
              260 270 280 290 300
LKLDFGIGEY INQKKRERSE ADKEKSHKDD SELDFSALCP KISLTVAAKE
              310 320 330 340 350
LSVSDTDVSE VSWTDNGTFN LSEGYTPQTD TSDDLDRPSE EVFSRDLSDF
              360 370 380 390 400
PSLENGMGTN DEDELSLGLP TELKRKKEQL DSGHRPSKET QSAAGLTLPL
              410 420 430 440 450
NSDQTFHLMS NLAGDVITAA VIAAIKDQLE GVQQALSQAA PIPEEDTDTE
              460 470 480 490
EGDDFELLDQ SELDQIESEL GLTQDQHAEA QQNKKSSGFL SNLLGGH
```

A cDNA sequence encoding the RETREG1 protein is available from the NCBI database as accession no. AK000159.

An amino acid sequence for the protein encoded by the human SMAD9 gene that is a negative regulator of T cells as detected by interferon-production is available from the UniPROT database as accession no. 015198, shown below as SEQ ID NO:58.

```
                10 20 30 40 50
MHSTTPISSL FSFTSPAVKR LLGWKQGDEE EKWAEKAVDS LVKKLKKKKG
                60 70 80 90 100
AMDELERALS CPGQPSKCVT IPRSLDGRLQ VSHRKGLPHV IYCRVWRWPD
              110 120 130 140 150
LQSHHELKPL ECCEFPFGSK QKEVCINPYH YRRVETPVLP PVLVPRHSEY
              160 170 180 190 200
NPQLSLLAKF RSASLHSEPL MPHNATYPDS FQQPPCSALP PSPSHAFSQS
              210 220 230 240 250
PCTASYPHSP GSPSEPESPY QHSVDTPPLP YHATEASETQ SGQPVDATAD
              260 270 280 290 300
RHVVLSIPNG DFRPVCYEEP QHWCSVAYYE LNNRVGETFQ ASSRSVLIDG
              310 320 330 340 350
FTDPSNNRNR FCLGLLSNVN RNSTIENTRR HIGKGVHLYY VGGEVYAECV
              360 370 380 390 400
SDSSIFVQSR NCNYQHGFHP ATVCKIPSGC SLKVFNNQLF AQLLAQSVHH
              410 420 430 440 450
GFEVVYELTK MCTIRMSFVK GWGAEYHRQD VTSTPCWIEI HLHGPLQWLD
              460
KVLTQMGSPH NPISSVS
```

A cDNA and a chromosomal sequence encoding the SMAD9 protein is available from the NCBI database as accession no. D83760 and AL138706, respectively.

An amino acid sequence for the protein encoded by the human SPATA31A1 gene that is a negative regulator of T cells as detected by interferon-γ production is available from the UniPROT database as accession no. Q5TZJ5, shown below as SEQ ID NO:59.

```
                10 20 30 40 50
MENLPFPLKL LSASSLNAPS STPWVLDIFL TLVFALGFFF LLLPYLSYFR
                60 70 80 90 100
CDDPPSPSPG KRKCPVGRRR RPRGRMKNHS LRAGRECPRG LQETSDLLSQ
              110 120 130 140 150
LQSLLGPHLD KGDFGQLSGP DPPGEVGERA PDGASQSSHE PMEDAAPILS
              160 170 180 190 200
PLASPDPQAK HPQDLASTPS PGPMTTSVSS LSASQPPEPS LPLEHPSPEP
              210 220 230 240 250
PALFPHPPHT PDPLACSPPP PKGFTAPPLR DSTLITPSHC DSVALPLGTV
              260 270 280 290 300
PQSLSPHEDL VASVPAISGL GGSNSHVSAS SRWQETARTS CAFNSSVQQD
              310 320 330 340 350
HLSRHPPETY QMEAGSLFLL SSDGQNAVGI QVTETAKVNI WEEKENVGSF
              360 370 380 390 400
TDRMTPEKHL NSLRNLAKSL DAEQDTTNPK PFWNMGENSK QLPGPQKLSD
              410 420 430 440 450
PRLWQESFWK NYSQLFWGLP SLHSESLVAN AWVTDRSYTL QSPPELFNEM
              460 470 480 490 500
SNVCPIQRET TMSPLLFQAQ PPSHLGPECQ PFISSTPQFR PTPMAQAEAQ
              510 520 530 540 550
AHLQSSFPVL SPAFPSLIKN TGVACPASQN KVQALSLPET QHPEWPLLRR
              560 570 580 590 600
QLEGRLALPS RVQKSQDVFS VSTPNLPQES LTSILPENFP VSPELRRQLE
              610 620 630 640 650
QHIKKWIIQH WGNLGRIQES LDLMQLRDES PGTSQAKGKP SPWQSSMSTG
              660 670 680 690 700
ESSKEAQKVK FQLERDPCPH LGQILGETPQ NLSRDMKSFP RKVLGVTSEE
              710 720 730 740 750
SERNLRKPLR SDSGSDLLRC TERTHIENIL KAHMGRNLGQ TNEGLIPVRV
              760 770 780 790 800
RRSWLAVNQA LPVSNTHVKT SNLAAPKSGK ACVNTAQVLS FLEPCTQQGL
              810 820 830 840 850
GAHIVRFWAK HRWGLPLRVL KPIQCFKLEK VSSLSLTQLA GPSSATCESG
              860 870 880 890 900
AGSEVEVDMF LRKPPMASLR KQVLTKASDH MPESLLASSP AWKQFQRAPR
              910 920 930 940 950
GIPSWNDHGP LKPPPAGQEG RWPSKPLTYS LTGSTQQSRS LGAQSSKAGE
              960 970 980 990 1000
TREAVPQCRV PLETCMLANL QATSEDMHGF EAPGTSKSSL HPRVSVSQDP
            1010 1020 1030 1040 1050
RKLCLMEEVV NEFEPGMATK SETQPQVCAA VVLLPDGQAS VVPHASENLV
            1060 1070 1080 1090 1100
SQVPQGHLQS MPAGNMRASQ ELHDLMAARR SKLVHEEPRN PNCQGSCKNQ
            1110 1120 1130 1140 1150
RPMFPPIHKS EKSRKPNLEK HEERLEGLRT PQLTPVRKTE DTHQDEGVQL
            1160 1170 1180 1190 1200
LPSKKQPPSV SHFGGNIKQF FQWIFSKKKS KPAPVTAESQ KTVKNRSCVY
            1210 1220 1230 1240 1250
SSSAEAQGLM TAVGQMLDEK MSLCHARHAS KVNQHKQKFQ APVCGFPCNH
            1260 1270 1280 1290 1300
RHLFYSEHGR ILSYAASSQQ ATLKSQGCPN RDRQIRNQQP LKSVRCNNEQ
            1310 1320 1330 1340
WGLRHPQILH PKKAVSPVSP LQHWPKTSGA SSHHHHCPRH CLLWEGI
```

A chromosomal sequence encoding the SPATA31A1 protein is available from the NCBI database as accession no. BX005214.

An amino acid sequence for the protein encoded by the human ZNF445 gene that is a negative regulator of T cells as detected by interferon-γ production is available from the UniPROT database as accession no. P59923, shown below as SEQ ID NO:60.

```
                10 20 30 40 50
MPPGRWHAAY PAQAQSSRER GRLQTVKKEE EDESYTPVQA ARPQTLNRPG
                60 70 80 90 100
QELFRQLFRQ LRYHESSGPL ETLSRLRELC RWWLRPDVLS KAQILELLVL
              110 120 130 140 150
EQFLSILPGE LRVWVQLHNP ESGEEAVALL EELQRDLDGT SWRDPGPAQS
              160 170 180 190 200
PDVHWMGTGA LRSAQIWSLA SPLRSSSALG DHLEPPYEIE ARDFLAGQSD
              210 220 230 240 250
TPAAQMPALF PREGCPGDQV TPTRSLTAQL QETMTFKDVE VTFSQDEWGW
              260 270 280 290 300
LDSAQRNLYR DVMLENYRNM ASLVGPFTKP ALISWLEARE PWGLNMQAAQ
              310 320 330 340 350
PKGNPVAAPT GDDLQSKTNK FILNQEPLEE AETLAVSSGC PATSVSEGIG
              360 370 380 390 400
LRESFQQKSR QKDQCENPIQ VRVKKEETNF SHRTGKDSEV SGSNSLDLKH
              410 420 430 440 450
VTYLRVSGRK ESLKHGCGKH FRMSSHHYDY KKYGKGLRHM IGGFSLHQRI
              460 470 480 490 500
HSGLKGNKKD VCGKDFSLSS HHQRGQSLHT VGVSFKCSDC GRTFSHSSHL
              510 520 530 540 550
AYHQRLHTQE KAFKCRVCGK AFRWSSNCAR HEKIHTGVKP YKCDLCEKAF
              560 570 580 590 600
RRLSAYRLHR ETHAKKKFLE LNQYRAALTY SSGFDHHLGD QSGEKLFDCS
              610 620 630 640 650
QCRKSFHCKS YVLEHQRIHT QEKPYKCTKC RKTFRWRSNF TRHMRLHEEE
              660 670 680 690 700
KFYKQDECRE GFRQSPDCSQ PQGAPAVEKT FLCQQCGKTF TRKKTLVDHQ
              710 720 730 740 750
RIHTGEKPYQ CSDCGKDFAY RSAFIVHKKK HAMKRKPEGG PSFSQDTVFQ
              760 770 780 790 800
VPQSSHSKEE PYKCSQCGKA FRNHSFLLIH QRVHTGEKPY KCRECGKAFR
              810 820 830 840 850
WSSNLYRHQR IHSLQKQYDC HESEKTPNVE PKILTGEKRF WCQECGKTFT
              860 870 880 890 900
RKRTLLDHKG IHSGEKRYKC NLCGKSYDRN YRLVNHQRIH STERPFKCQW
              910 920 930 940 950
CGKEFIGRHT LSSHQRKHTR AAQAERSPPA RSSSQDTKLR LQKLKPSEEM
              960 970 980 990 1000
PLEDCKEACS QSSRLTGLQD ISIGKKCHKC SICGKTFNKS SQLISHKRFH
            1010 1020 1030
TRERPFKCSK CGKTFRWSSN LARHMKNHIR D
```

A cDNA encoding the ZNF445 protein is available from the NCBI database as accession 45 no. AY262260.

The following genes are negative regulators of T cells as detected by Interleukin-2 production (see Table 5): ABI3BP, AEBP1, AHR, ANTXR2, ARHGAP15,ARHGAP27, ARHGDIB, ARID3A, ARL4D, B4GALNT3, BICD1, C10orf82, C17orf75, C19orf35, C1 RL, C2orf69, C6orf132, C9orf84, CABP1, CBLB, CCSER1, CD34, CD4, CD5, CD52, CEACAM11, CEACAM7, CEBPB, CES3, CGB3, COL11A1, COL4A3, COLQ, CPEB3, CRELD2, CST9L, DDX55, DLG4, DOK1, EBF3, EIF3K, EN2, EOMES, EPB41, ETSI, F5, FAM96A, FH1, FOXA3, FOXE1, FOX13, FOXL2NB, FUS, FUT4, GCSAM, GCSAML, GDAPIL1, GDPD2, (GMIP, GNL3L, GOLPH3, CRAP, GRB2, HAUS7, HERCI, HLA-DQB2, I-ISD17B11, IKZF1, IKZF3, INPPL1, INTSI1, ITIH2, ITPKA, 1T1 KB, ITPKC, JDP2, JKAMP, JMJDIC, KIAA1024, KIF15, KIF5A, KNTC1, LAT2, LAX1, LGR5, LLIME, LMBRD2, LOC401052, LONP2, LRCH3, LRRC23, LRRC25, LRRC52, LYN, LYPDI, MAATS1, MAB21L2, MAGEB17, MAP4K1, MEF2C, METTL9, MICU1, MRPL17, MUC1, NAIF1, NCF2, NDNF NDLUFB1, NH-P2, NKX2-6, NLGN4Y, NNL, NPIPB9, NR4AA, NR4A3, NRCAM,NRPI, NRSN2, NSUN7, OLFML1, OM1P, OPRD1, ORIK1, OR2BI1, OSBPL11, OTOG, OTUD4, PATL2, PAX5, PFKL, PHF2, PILBF1, P11P5KIA, PIP5KIB, P1TPNC1, PLCL1, PLEKHM2, PPARG, PP1C, PSRC1, PSTPIP1, PTPN12, PTPN22, PTPN6, PTPRC, PVRIG, RBP4, RPL13A, S100A2, SALL4, SAMD8, SENP6, SETDIB, SEZ6L, SFT2DI, SH3TC1, SIGIRR, SIT1, SLA, SLA2, SLC20A2, SLC39A2, SLC6A8, SMAGP, SNRNP48, SOCS2, SORBSI, SOX13, SPN, SPRED1, SPRFD2, SRPK1, STAP1, STK38L, SYPL1, TCF12, TEX35, TFCP2L1, TMEM14C, TME 1223, TMEIEM262, TNTNT2, TPRA1, TREM6-TRIM34, TSPANI, UBASH3B, UBE2W, UBR4, UBXN7, UCPI, UTMCl, ULLKI, U7PK3B, VPS28, VSTM5, XKR9, YLPM1, ZDHCC7, EB1, ZEB2, ZNF445, ZNF70, and ZN F831. Table 7 provides additional negative regulators of T cells as detected by interleukin-2 production

Sequences and other information relating to these genes, and their encoded proteins, is available, for example from the NCBI and UniPROT databases,

A few examples of protein sequences encoded by some of the genes detected as negative regulators of T cells by Interleukin-2 production are provided. For example, an amino acid sequence for the protein encoded by the human ABI3BP gene that is a negative regulator of T cells as detected by Interleukin-2 production is available from the UniPROT database as accession no. Q727G0, shown below as SEQ ID NO:61.

```
                10 20 30 40 50
MRGGKCNMLS SLGCLLLCGS ITLALGNAQK LPKGKRPNLK VHINTTSDSI
                60 70 80 90 100
LLKFLRPSPN VKLEGLLLGY GSNVSPNQYF PLPAEGKFTE AIVDAEPKYL
              110 120 130 140 150
IVVRPAPPPS QKKSCSGKTR SRKPLQLVVG TLTPSSVFLS WGFLINPHHD
              160 170 180 190 200
WTLPSHCPND RFYTIRYREK DKEKKWIFQI CPATETIVEN LKPNTVYEFG
              210 220 230 240 250
VKDNVEGGIW SKIFNHKTVV GSKKVNGKIQ STYDQDHTVP AYVPRKLIPI
              260 270 280 290 300
TIIKQVIQNV THKDSAKSPE KAPLGGVILV HLIIPGLNET TVKLPASLMF
              310 320 330 340 350
EISDALKTQL AKNETLALPA ESKTPEVEKI SARPTTVTPE TVPRSTKPTT
              360 370 380 390 400
SSALDVSETT LASSEKPWIV PTAKISEDSK VLQPQTATYD VFSSPTTSDE
              410 420 430 440 450
PEISDSYTAT SDRILDSIPP KTSRTLEQPR ATLAPSETPF VPQKLEIFTS
              460 470 480 490 500
PEMQPTTPAP QQTTSIPSTP KRRPRPKPPR TKPERTTSAG TITPKISKSP
              510 520 530 540 550
EPTWTTPAPG KTQFISLKPK IPLSPEVTHT KPAPKQTPRA PPKPKTSPRP
              560 570 580 590 600
RIPQTQPVPK VPQRVTAKPK TSPSPEVSYT TPAPKDVLLP HKPYPEVSQS
              610 620 630 640 650
EPAPLETRGI PFIPMISPSP SQEELQTTLE ETDQSTQEPF TTKIPRTTEL
              660 670 680 690 700
AKTTQAPHRF YTTVRPRTSD KPHIRPGVKQ APRPSGADRN VSVDSTHPTK
              710 720 730 740 750
KPGTRRPPLP PRPTHPRRKP LPPNNVTGKP GSAGIISSGP ITTPPLRSTP
              760 770 780 790 800
RPTGTPLERI ETDIKQPTVP ASGEELENIT DFSSSPTRET DPLGKPRFKG
              810 820 830 840 850
PHVRYIQKPD NSPCSITDSV KRFPKEEATE GNATSPPQNP PTNLTVVTVE
              860 870 880 890 900
GCPSFVILDW EKPLNDTVTE YEVISRENGS FSGKNKSIQM TNQTFSTVEN
              910 920 930 940 950
LKPNTSYEFQ VKPKNPLGEG PVSNTVAFST ESADPRVSEP VSAGRDAIWT
              960 970 980 990 1000
ERPFNSDSYS ECKGKQYVKR TWYKKFVGVQ LCNSLRYKIY LSDSLTGKFY
            1010 1020 1030 1040 1050
NIGDQRGHGE DHCQFVDSFL DGRTGQQLTS DQLPIKEGYF RAVRQEPVQF
            1060 1070
GEIGGHTQIN YVQWYECGTT IPGKW
```

A cDNA and a chromosomal sequence encoding the ABI3BP protein is available from the NCBT database as accession no. AB056106 and CH471052, respectively.

An amino acid sequence for the protein encoded by the human GCSAML gene that is a negative regulator of T cells as detected by Interleukin-2 production is available from the UniPROT database as accession no. 043741, shown below as SEQ ID NO:62.

```
                10 20 30 40 50
MGNTTSDRVS GERHGAKAAR SEGAGGHAPG KEHKIMVGST DDPSVFSLPD
                60 70 80 90 100
SKLPGDKEFV SWQQDLEDSV KPTQQARPTV IRWSEGGKEV FISGSFNNWS
              110 120 130 140 150
TKIPLIKSHN DFVAILDLPE GEHQYKFFVD GQWVHDPSEP VVTSQLGTIN
              160 170 180 190 200
NLIHVKKSDF EVFDALKLDS MESSETSCRD LSSSPPGPYG QEMYAFRSEE
              210 220 230 240 250
RFKSPPILPP HLLQVILNKD TNISCDPALL PEPNHVMLNH LYALSIKDSV
              260 270
MVLSATHRYK KKYVTTLLYK PI
```

A cDNA and a chromosomal sequence encoding the GCSAML protein is available from the NCBI database as accession no. AJ224538 and AL356378, respectively. PGP-83,DNA The following genes are negative regulators of T cells as detected by reduced cellular proliferation (see Table 6): ABCB1, ASAP1, ATP10A, DEAF1, FOXK1, ITGAX, LCE6A, LCP2, LEFTY1, MYC, NAT8B, OLFM3, PLD6, PREP, SULT1A1, SULT1A4, A-INAK, AR-IODLB, B3GNT5, CASZ1, CD27, CEBPB, CRIBP, FL11, FOSL2, HLX, MAP4K1, MUC21, MXI, NDRG1, NEUROD2, SLC2A1, SLC43A3, SMAGP, SOX13, SP140, TP11, and TTC39C. Table 7 provides additional negative regulators of T cells as detected by reduced cellular proliferation.

An amino acid sequence for the protein encoded by the human SULT1A4 gene that is a negative regulator of T cells as detected by reduced cellular proliferation is available from the UniPROT database as accession no. PODMIN0, shown below as SEQ ID NO:63.

```
                10 20 30 40 50
MELIQDTSRP PLEYVKGVPL IKYFAEALGP LQSFQARPDD LLINTYPKSG
                60 70 80 90 100
TTWVSQILDM IYQGGDLEKC NRAPIYVRVP FLEVNDPGEP SGLETLKDTP
              110 120 130 140 150
PPRLIKSHLP LALLPQTLLD QKVKVVYVAR NPKDVAVSYY HFHRMEKAHP
              160 170 180 190 200
EPGTWDSFLE KFMAGEVSYG SWYQHVQEWW ELSRTHPVLY LFYEDMKENP
              210 220 230 240 250
KREIQKILEF VGRSLPEETM DFMVQHTSFK EMKKNPMTNY TTVPQELMDH
              260 270 280 290
SISPFMRKGM AGDWKTTFTV AQNERFDADY AEKMAGCSLS FRSEL
```

A chromosomal sequence encoding the SULT1A4 protein is available from the NCBI database as accession no. AC106782.

An amino acid sequence for the protein encoded by the human SLC43A3 gene that is a negative regulator of T cells as detected by reduced cellular proliferation is available from the UniPROT database as accession no. Q8NB15, shown below as SEQ ID NO:64.

```
                10 20 30 40 50
MAGQGLPLHV ATLLTGLLEC LGFAGVLFGW PSLVFVFKNE DYFKDLCGPD
                60 70 80 90 100
AGPIGNATGQ ADCKAQDERF SLIFTLGSFM NNFMTFPTGY IFDRFKTTVA
              110 120 130 140 150
RLIAIFFYTT ATLIIAFTSA GSAVLLFLAM PMLTIGGILF LITNLQIGNL
              160 170 180 190 200
FGQHRSTIIT LYNGAFDSSS AVFLIIKLLY EKGISLRASF IFISVCSTWH
              210 220 230 240 250
VARTFLLMPR GHIPYPLPPN YSYGLCPGNG TTKEEKETAE HENRELQSKE
              260 270 280 290 300
FLSAKEETPG AGQKQELRSF WSYAFSRRFA WHLVWLSVIQ LWHYLFIGTL
              310 320 330 340 350
NSLLTNMAGG DMARVSTYTN AFAFTQFGVL CAPWNGLLMD RLKQKYQKEA
              360 370 380 390 400
RKTGSSTLAV ALCSTVPSLA LTSLLCLGFA LCASVPILPL QYLTFILQVI
              410 420 430 440 450
SRSFLYGSNA AFLTLAFPSE HFGKLFGLVM ALSAVVSLLQ FPIFTLIKGS
              460 470 480 490 500
LQNDPFYVNV MFMLAILLTF FHPFLVYREC RTWKESPSAI A
```

A cDNA and a chromosomal sequence encoding the SLC43A3 protein is available from the NCBI database as accession no. AB028927 and AP000781 Any of these genes or the proteins encoded by these genes that are described herein can regulate T cells.

The sequences provided herein are exemplary. Isoforms and variants of these sequences and of any of regulators listed in Tables 1-7 or FIGS. 1-4 can also be used in the methods and compositions described herein.

For example, isoforms and variants of the proteins and nucleic acids can be used in the methods and compositions described herein when they are substantially identical to the genes or the encoded proteins listed in Tables 1-7 or FIGS. 1-4. The terms “substantially identity” indicates that a polypeptide or nucleic acid comprises a sequence with between 55-100% sequence identity to a reference sequence, for example with at least 55% sequence identity, preferably 60%, preferably 70%, preferably 80%, preferably at least 90%, preferably at least 95%, preferably at least 96%, preferably at least 97% sequence, preferably at least 98%, preferably at least 99% identity to a reference sequence over a specified comparison window. Optimal alignment may be ascertained or conducted using the homology alignment algorithm of Needleman and Wunsch, J. Mol. Biol. 48:443-53 (1970).

An indication that two polypeptide sequences are substantially identical is that both polypeptides have the same function acting as a regulator of T cells or T cell activity. The polypeptide that is substantially identical to a regulator sequence and may not have exactly the same level of activity as the regulator. Instead, the substantially 10 identical polypeptide may exhibit greater or lesser levels of regulator activity than the those listed in Tables 1-7 or FIG. 1-4, or any of the sequences recited herein. For example, the substantially identical polypeptide or nucleic acid may have at least about 40%, or at least about 50%, or at least about 60%, or at least about 70%, or at least about 80%, or at least about 90%, or at least about 95%, or at least about 97%, or at least about 98%, or at least about 100%, or at least about 105%, or at least about 110%, or at least about 120%, or at least about 130%, or at least about 140%, or at least about 150%, or at least about 200% of the activity of a regulator described herein a when measured by similar assay procedures.

Alternatively, substantial identity is present when second polypeptide is immunologically reactive with antibodies raised against the first polypeptide (e.g., a polypeptide with encoded by any of the genes listed in Tables 1-7 or FIGS. 1-4). Thus, a polypeptide is substantially identical to a first polypeptide, for example, where the two polypeptides differ only by a conservative substitution. In addition, a polypeptide can be substantially identical to a first polypeptide when they differ by a non-conservative change if the epitope that the antibody recognizes is substantially identical. Polypeptides that are “substantially similar” share sequences as noted above except that sore residue positions, which are not identical, may differ by conservative amino acid changes.

### Expression Systems

Nucleic acid segments encoding one or more regulator proteins, or nucleic acid segments that are inhibitory nucleic acids or such regulators, can be inserted into or employed with any suitable expression system. Nucleic acids segments encoding one or more agents that can modulate a regulator protein expression or activity can be inserted into or employed with any suitable expression system. A therapeutically effective quantity of one or more regulator proteins or modulators of such regulator proteins can be generated from such expression systems. A therapeutically effective of one or more inhibitory nucleic acids can also be generated from such expression systems.

Recombinant expression of nucleic acids (or inhibitory nuclei acids) is usefully accomplished using a vector, such as a plasmid. The vector can include a promoter operably linked to nucleic acid segment encoding one or more regulator/modulator proteins. In another example, a vector can include a promoter operably linked to nucleic acid segment that encodes a regulator/modulator inhibitory nucleic acid.

The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying, encoding and/or expressing regulator/modulator. A variety of prokaryotic and eukaryotic expression vectors suitable for carrying, encoding and/or expressing regulator/modulator inhibitory nucleic acids can be employed. Such expression vectors include, for example, pET, pET3d, pCR2.1, pBAD, pUC, and yeast vectors. The vectors can be used, for example, in a variety of in vivo and in vitro situations.

The expression cassette, expression vector, and sequences in the cassette or vector can be heterologous. As used herein, the term “heterologous” when used in reference to an expression cassette, expression vector, regulatory sequence, promoter, or nucleic acid refers to an expression cassette, expression vector, regulatory sequence, or nucleic acid that has been manipulated in some way. For example, a heterologous promoter can be a promoter that is not naturally linked to a nucleic acid of interest, or that has been introduced into cells by cell transformation procedures. A heterologous nucleic acid or promoter also includes a nucleic acid or promoter that is native to an organism but that has been altered in some way (e.g., placed in a different chromosomal location, mutated, added in multiple copies, linked to a non-native promoter or enhancer sequence, etc.). Heterologous nucleic acids may comprise sequences that comprise cDNA forms; the cDNA sequences may be expressed in either a sense (to produce mRNA) or anti-sense orientation (to produce an anti-sense RNA transcript that is complementary to the mRNA transcript). Heterologous coding regions can be distinguished from endogenous coding regions, for example, when the heterologous coding regions are joined to nucleotide sequences comprising regulatory elements such as promoters that are not found naturally associated with the coding region, or when the heterologous coding regions are associated with portions of a chromosome not found in nature (e.g., genes expressed in loci where the protein encoded by the coding region is not normally expressed). Similarly, heterologous promoters can be promoters that at linked to a coding region to which they are not linked in nature.

Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in by Verma, T. M., Retroviral vectors for gene transfer. In Microbiology-1985, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

A variety of regulatory elements can be included in the expression cassettes and/or expression vectors, including promoters, enhancers, translational initiation sequences, transcription termination sequences and other elements. A “promoter” is generally a sequence or sequences of DNA that function when in a relatively fixed location in regard to the transcription start site. For example, the promoter can be upstream of the nucleic acid segment encoding a regulator protein. In another example, the promoter can be upstream of an inhibitory nucleic acid segment of a modulating agent for one or more regulators.

A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences for the termination of transcription, which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The expression of regulator/modulator proteins or inhibitory nucleic acid molecules therefor from an expression cassette or expression vector can be controlled by any promoter capable of expression in prokaryotic cells or eukarvotic cells. Examples of prokaryotic promoters that can be used include, but are not limited to, SP6, T7, T5, tac, bla, frp, gal, lac, or maltose promoters. Examples of eukaryotic promoters that can be used include, but are not limited to, constitutive promoters, e.g., viral promoters such as CM V, SV40 and RSV promoters, as well as regulatable promoters, e.g., an inducible or repressible promoter such as the tet promoter, the hsp70 promoter and a synthetic promoter regulated by CRE. Vectors for bacterial expression include pGEX-5X-3, and for eukaryotic expression include pCIneo-CMV.

The expression cassette or vector can include nucleic acid sequence encoding a marker product. This marker product is used to determine if the gene has been delivered to the cell and once delivered is being expressed. Marker genes can include the E coli lacZ gene which encodes P-galactosidase, and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern P. and Berg, P, J. Molec. Appl. Genet, 1: 327 (1982)), mycophenolic acid, (Mulligan., R. C. and Berg, P. Science 209: 1422 (1980)) or hygromycin, (Sugden, B. et al., Mol. Cell Biol. 5: 410-413 (1985)).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomnes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Rain et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, . A. Nature, 352, 815-818, (1991).

For example, the nucleic acid molecules, expression cassette and/or vectors encoding regulator/modulator proteins or encoding inhibitory nucleic acid molecules therefor can be introduced to a cell by any method including, but not limited to, calcium-mediated transformation, electroporation, microinjection, lipofection, particle bombardment and the like. The cells can be expanded in culture and then administered to a subject, e.g., a mammal such as a human. The amount or number of cells administered can vary but amounts in the range of about 10⁶ to about 10⁹ cells can be used. The cells are generally delivered in a physiological solution such as saline or buffered saline. The cells can also be delivered in a vehicle such as a population of liposomes, exosomes or microvesicles.

In some cases, the transgenic cell can produce exosomes or microvesicles that contain nucleic acid molecules, expression cassettes and/or vectors encoding one or more regulator/modulator. In some cases, the transgenic cell can produce exosomes or microvesicles that contain inhibitory nucleic acid molecules that can target regulator/modulator nucleic acids, one or more nucleic acids for regulator, or a combination thereof. Microvesicles can mediate the secretion of a wide variety of proteins, lipids, mRNAs, and micro RNAs, interact with neighboring cells, and can thereby transmit signals, proteins, lipids, and nucleic acids from cell to cell (see, e.g., Shen et al., J Biol Chem, 286(16): 14383-14395 (2011); Hu et al., Frontiers in Genetics 3 (April 2012); Pegtel et al., Proc. Nat'l Acad Sci 107(14): 6328-6333 (2010); WO/201 3/084000; each of which is incorporated herein by reference in its entirety. Cells producing such microvesicles can be used to express the one or more regulator/modulator protein and/or inhibitory nucleic acids for one or more regulator/modulators, or a combination thereof Transgenic vectors or cells with a heterologous expression cassette or expression vector can express one or more regulator, can optionally also express one or more regulator inhibitory nucleic acids, or a combination thereof. Any of these vectors or cells can be administered to a subject. Exosomes produced by transgenic cells can be used to administer regulator/modulator proteins, regulator/modulator nucleic acids, regulator/modulator inhibitory nucleic acids, or a combination thereof to a. subject or to tumor and cancer cells in the subject.

Methods and compositions that include inhibitors of one or regulators such as inhibitory nucleic acids, antibodies, or any combination thereof.

### CRISPR Modifications

In some cases, clustered regularly interspaced short palindromic repeats (CRISPR)/CRISPR-associated (Cas) systems can be used to create one or more modifications in genomic regulator genes. Such CRISPR modifications can reduce or activate the expression or functioning of the regulator gene products. CRISPR/Cas systems are useful, for example, for RNA-programmable genome editing (see e.g., Marraffini and Sontheimer. Nature Reviews Genetics 11: 181-190 (2010); Sorek et al. Nature Reviews Microbiology 2008 6: 181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12: 177-186; all of which are incorporated by reference herein in their entireties).

A CRISPR guide RNA can be used that can target a Cas enzyme to the desired location in the genome, where it can cleave the genomic DNA for generation of a genomic modification. This technique is described, for example, by Mali et al. Science 2013 339:823-6; which is incorporated by reference herein in its entirety. Kits for the design and use of CRISPR-mediated genome editing are commercially available, e.g. the PRECISION X CAS9 SMART NUCLEASE™ System (Cat No. CAS900A-1) from System Biosciences, Mountain View, CA.

In some cases, transcriptional activators can be linked to defective Cas9 or to one or more guide RNAs to target the transcriptional activator. Such transcriptional activators include protein domains or whole proteins that assist in the recruitment of co-factors and RNA Polymerase to increase transcription of one or more of the regulator gene(s) listed in Tables 1-7 or FIGS. 1-4.

In some cases, a cre-lox recombination system of bacteriophage P1, described by Abremski et al. 1983. Cell 32:1301 (1983), Sternberg et al., Cold Spring Harbor Symposia on Quantitative Biology, Vol. XLV 297 (1981) and others, can be used to promote recombination and alteration of the regulator genomic site(s). The cre-lox system utilizes the cre recombinase isolated from bacteriophage P1 in conjunction with the DNA sequences that the recombinase recognizes (termed lox sites). This recombination system has been effective for achieving recombination in plant cells (see, e.g., U.S. Pat. No. 5,658,772), animal cells (U.S. Pat. Nos. 4,959,317; 5,801,030), and in viral vectors (Hardy et al., .J. Virology 71:1842 (1997).

The genomic mutations so incorporated can alter one or more amino acids in the encoded regulator gene products. For example, genomic sites modified so that the encoded regulator protein is more prone to degradation, is less stable so that the half-life of such protein(s) is reduced, or so that the regulator has improved expression or functioning. In another example, genomic sites can be modified so that at least one amino acid of a regulator polypeptide is deleted or mutated to alter its activity. For example, a conserved amino acid or a conserved domain can be modified to improve or reduce of the activity of the regulator polypeptide. For example, a conserved amino acid or several amino acids in a conserved domain of the regulator polypeptide can be replaced with one or more amino acids having physical and/or chemical properties that are different from the conserved amino acid(s). For example, to change the physical and/or chemical properties of the conserved amino acid(s), the conserved amino acid(s) can be deleted or replaced by amino acid(s) of another class, where the classes are identified in the following table.

| Classification | Genetically Encoded |
|---|---|
| Hydrophobic | A, G, F, I, L, M, P, V, W |
| Aromatic | F, Y, W |
| Apolar | M, G, P |
| Aliphatic | A, V, L, I |
| Hydrophilic | C, D, E, H, K, N, Q, R, S, T, Y |
| Acidic | D, E |
| Basic | H, K, R |
| Polar | Q, N, S, T, Y |
| Cysteine-Like | C |

The guide RNAs and nuclease can be introduced via one or more vehicles such as by one or more expression vectors (e.g., viral vectors), virus like particles, ribonucleoproteins (RNPs), via nanoparticles, liposomes, or a combination thereof. The vehicles can include components or agents that can target particular cell types (e.g., antibodies that recognize cell-surface markers), facilitate cell penetration, reduce degradation, or a combination thereof.

### Inhibitory Nucleic Acids

The expression of one or more regulators/modulators can be inhibited, for example by use of an inhibitory nucleic acid that specifically recognizes a nucleic acid that encodes the regulator or modulator.

An inhibitory nucleic acid can have at least one segment that will hybridize to a regulator nucleic acid or modulator under intracellular or stringent conditions. The inhibitory nucleic acid can reduce expression of a regulator/modulator nucleic acid. A nucleic acid may hybridize to a genomic DNA, a messenger RNA, or a combination thereof. An inhibitory nucleic acid may be incorporated into a plasmid vector or viral DNA. It may be single stranded or double stranded, circular or linear.

An inhibitory nucleic acid is a polymer of ribose nucleotides or deoxyribose nucleotides having more than 13 nucleotides in length. An inhibitory nucleic acid may include naturally occurring nucleotides; synthetic, modified, or pseudo-nucleotides such as phosphorothiolates; as well as nucleotides having a detectable label such as P³², biotin or digoxigenin. An inhibitory nucleic acid can reduce the expression and/or activity of a regulator/modulator nucleic acid. Such an inhibitory nucleic acid may be completely complementary to a segment of an endogenous regulator/modulator nucleic acid (e.g, an RNA). Alternatively, some variability is permitted in the inhibitory nucleic acid sequences relative to regulator/modulator sequences. An inhibitory nucleic acid can hybridize to a regulator/modulator nucleic acid under intracellular conditions or under stringent hybridization conditions and is sufficiently complementary to inhibit expression of the endogenous regulator/modulator nucleic acid. Intracellular conditions refer to conditions such as temperature, pH and salt concentrations typically found inside a cell, e.g. an animal or mammalian cell. One example of such an animal or mammalian cell is a myeloid progenitor cell, Another example of such an animal or mammalian cell is a more differentiated cell derived from a myeloid progenitor cell. Generally, stringent hybridization conditions are selected to be about 5° C. lower than the thermal melting point (Tₘ) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory oligonucleotides that comprise, for example, 2, 3, 4, or 5 or more stretches of contiguous nucleotides that are precisely complementary to a regulator/modulator coding sequence, each separated by a stretch of contiguous nucleotides that are not complementary to adjacent coding sequences, can inhibit the function of one or more nucleic acids for any of the regulators or modulators described herein. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a short hairpin RNA, a small interfering RNA, a ribozyme or an antisense nucleic acid molecule.

The inhibitory nucleic acid molecule may be single or double stranded (e.g. a small interfering RNA (siRNA)) and may function in an enzyme-dependent manner or by steric blocking. Inhibitory nucleic acid molecules that function in an enzyme-dependent manner include forms dependent on RNase H activity to degrade target mRNA. These include single-stranded DNA, RNA, and phosphorothioate molecules, as well as the double-stranded RNAi/siRNA system that involves target mRNA recognition through sense-antisense strand pairing followed by degradation of the target mRNA by the RNA-induced silencing complex. Steric blocking inhibitory nucleic acids, which are RNase-H independent, interfere with gene expression or other mRNA-dependent cellular processes by binding to a target mRNA and getting in the way of other processes. Steric blocking inhibitory nucleic acids include 2′-0 alkyl (usually in chimeras with RNase-H dependent antisense), peptide nucleic acid (PNA), locked nucleic acid (LNA) and morpholino antisense.

Small interfering RNAs, for example, may be used to specifically reduce translation of regulator/Modulator such that translation of the encoded regulator/modulator polypeptide is reduced. SiRNAs mediate post-transcriptional gene silencing in a sequence-specific manner. See, for example, website at invitrogen.con/lsite/us/en/home/Products-and-Services/Applications/rnai.html. Once incorporated into an RNA-Induced silencing complex, siRNA mediate cleavage of the homologous endogenous mRNA transcript by guiding the complex to the homologous mRNA transcript, which is then cleaved by the complex. The siRNA may be homologous and/or complementary to any region of the regulator/modulator transcript and/or any of the transcripts of the regulators/modulators. The region of homology may be 30 nucleotides or less in length, preferable less than 25 nucleotides, and more preferably about 21 to 23 nucleotides in length. SiRNA is typically double stranded and may have two-nucleotide 3′ overhangs, for example, 3′ overhanging UU dinucleotides. Methods for designing siRNAs are known to those skilled in the art See, for example, Elbashir et al. Nature 411: 494-498 (2001): Harborth et al. Antisense Nucleic Acid Drug Dev. 13: 83-106 (2003).

The pSuppressorNeo vector for expressing hairpin siRNA, commercially available from IMGENEX (San Diego, California), can be used to generate siRNA for inhibiting expression of regulators/modulators. The construction of the siRNA expression plasmid involves the selection of the target region of the mRNA, which can be a trial-and-error process. However, Elbashir et al. have provided guidelines that appear to work ˜80% of the time. Elbashir, S. M., et al., Analysis of gene function in somatic mammalian cells using small interfering RNAs. Methods, 2002. 26(2): p. 199-213. Accordingly, for synthesis of synthetic siRNA, a target region may be selected preferably 50 to 100 nucleotides downstream of the start codon. The 5′ and 3′ untranslated regions and regions close to the start codon should be avoided as these may be richer in regulatory protein binding sites. As siRNA can begin with AA, have 3′ UU overhangs for both the sense and antisense siRNA strands, and have an approximate 50% G/C content. An example of a sequence for a synthetic siRNA is 5′-A A(N 9)UU, where N is any nucleotide in the mnRNA sequence and should be approximately 50% G-C content. The selected sequence(s) can be compared to others in the human genome database to minimize homology to other known coding sequences (e.g., by Blast search, for example, through the NCBI website).

SiRNAs may be chemically synthesized, created by in vitro transcription, or expressed from an siRNA expression vector or a PCR expression cassette. See, e.g., website at invitrogen.com/site/us/en/home/Products-and-Services/Applications/rnai.html. When an siRNA is expressed from an expression vector or a PCR expression cassette, the insert encoding the siRNA may be expressed as an RNA transcript that folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, preferably, 3 to 23 nucleotides in length, and may be of various nucleotide sequences including, AUG, CCC, U,UCG, CCACC, CTCGAG, AAGCUU, CCACACC and UUCAAGAGA (SEQ ID NO:61). SiRNAs also may be produced in vivo by cleavage of double-stranded RNA introduced directly or via a transgene or virus. Amplification by an RNA-dependent RNA polymerase may occur in some organisms.

An inhibitory nucleic acid such as a short hairpin RNA siRNA or an antisense oligonucleotide may be prepared using methods such as by expression from an expression vector or expression cassette that includes the sequence of the inhibitory nucleic acid. Alternatively, it may be prepared by chemical synthesis using naturally occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the inhibitory nucleic acid and the target regulators/modulators nucleic acids.

An inhibitory nucleic acid may be prepared using available methods, for example, by expression from an expression vector encoding a complementarity sequence of the regulator/modulator nucleic acids described herein. Alternatively, it may be prepared by chemical synthesis using naturally occurring nucleotides, modified nucleotides or any mixture of combination thereof. In some embodiments, the nucleic acids of the regulators/modulators described herein are made from modified nucleotides or non-phosphodiester bonds, for example, that are designed to increase biological stability of the nucleic acids or to increase intracellular stability of the duplex formed between the inhibitory nucleic acids and other (e.g., endogenous) nucleic acids.

For example, the regulator/modulator nucleic acids can be peptide nucleic acids that have peptide bonds rather than phosphodiester bonds.

Naturally occurring nucleotides that can be employed in the regulator/modulator nucleic acids include the ribose or deoxyribose nucleotides adenosine, guanine, cytosine, thymine and uracil. Examples of modified nucleotides that can be employed in the regulator/modulator nucleic acids include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methyiguanine, 1-methylinosine, 2,2-dimethylguanine, 2-methyladenine, 2-methylguanine, 3-methylcytosine, 5-methylcytosine, N6-adenine, 7-methylguanine, 5-methiylaminomethiyluracil, 5-methoxyaminomethyl-2-thiouracil, beta-D-mannosylqueosine, 5′-methoxycarboxymethyluracil, 5-methoxyuracil, 2-methythio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine.

Thus, inhibitory nucleic acids of the regulators/modulators described herein may include modified nucleotides, as well as natural nucleotides such as combinations of ribose and deoxyribose nucleotides. The inhibitory nucleic acids and may be of same length as wild type regulators/modulators described herein. The inhibitory nucleic acids of the regulators described herein can also be longer and include other useful sequences. In some embodiments, the inhibitory nucleic acids of the regulators/modulators described herein are somewhat shorter. For example, inhibitory nucleic acids of the regulators/modulators described herein can include a segment that has a nucleic acid sequence that can be missing up to 5 nucleotides, or missing up to 10 nucleotides, or missing up to 20 nucleotides, or missing up to 30 nucleotides, or missing up to 50 nucleotides, or missing up to 100 nucleotides from the 5′ or 3′ end.

### Antibodies

Antibodies can be used as inhibitors or activators of any of the regulators/modulators described herein. For example, in some cases, antibody preparations can target one or more of the regulators or modulators described herein to block interactions by the regulators/modulators described herein or to reduce the activities or the regulators/modulators. In other cases, for example, antibodies can activate one or more of the regulator or modulators described herein that are cell surface receptors. One example of such activation is Varlilumab (a CD27 activating antibody) currently in clinical trial and that has been shown to increase anti-tumor T cell function Ansell et al. (2020) Blood Adv. 4(9): 1917-1926.

Antibodies can be raised against various epitopes of the regulators/modulator described herein. Some antibodies for regulators/modulators described herein may also be available commercially. However, the antibodies contemplated for treatment pursuant to the methods and compositions described herein are preferably human or humanized antibodies and are highly specific for their targets.

In one aspect, the present disclosure relates to use of isolated antibodies that bind specifically to regulators/modulators described herein. Such antibodies may be monoclonal antibodies. Such antibodies may also be humanized or fully human monoclonal antibodies. The antibodies can exhibit one or more desirable functional properties, such as high affinity binding to one or more regulators/modulators described herein, or the ability to inhibit functioning of any of the regulators/modulators described herein.

Methods and compositions described herein can include antibodies that bind any of the regulators/modulators described herein, or a combination of antibodies where each antibody type can separately bind one of the regulators/modulators described herein.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., a peptide or domain of an of the regulators/modulators described herein). It has been shown that the antigen-binding function of an antibody can be performed by fragments of a full-length antibody.

Examples of binding fragments encompassed within the term “antigen-binding portion” of an antibody include (i) a Fab fragment, a monovalent fragment consisting of the VT, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L}, and V_{H}, are coded for by separate genes, they can be joined, using recombinant methods, by a synthetic linker that enables them to be made as a single protein chain in which the V_{L}, and V_{H}, regions pair to from monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds any of the regulators/modulators described herein is substantially free of antibodies that specifically bind antigens other than any of he regulators/modulators described herein). An isolated antibody that specifically binds regulators/modulators described herein may, however, have cross-reactivity to other antigens, such as isoforms or related forms of the regulators modulators proteins from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a. light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, 10 expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{H} regions of the recombinant antibodies are sequences that, while derived from and related to human germline V_{L} and V_{H} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to a human regulator/modulator protein described herein” is intended to refer to an antibody that binds to the human regulator/modulator protein described herein with a K_{D} of 1×10⁻⁷ M or less, more preferably 5×10⁻⁸ M or less, more preferably 1×10⁻⁸ M or less, more preferably 5×10⁻⁹ M or less, even more preferably between 1×10⁻⁸ M and 1×10⁻¹⁰ M or less.

The term “Kₐₛₛₒₑ” or “Kₐ,” as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D},” as used herein, is intended to refer to the dissociation constant, which is obtained from the ratio of K_{d} to Kₐ (i.e., K_{d}/Kₐ) and is expressed as a molar concentration (M) K_{D} values for antibodies can be determined using methods well established in the art. A preferred method for determining the K_{D} of an antibody is by using surface plasmon resonance, preferably using a biosensor system such as a Biacore™ system.

The antibodies of the invention are characterized by particular functional features or properties of the antibodies. For example, the antibodies bind specifically to a human regulator/modulator described herein. Preferably, an antibody of the invention binds to a regulator/modulator described herein with high affinity, for example with a K_{D} of 1×10⁻⁷ M or less. The antibodies can exhibit one or more of the following characteristics:
(a) binds to a human regulators/modulators described herein with a Kr) of 1×10⁻⁷ M or less;
(b) inhibits the function or activity of a human regulators/modulators described herein;
(c) inhibits cancer (e.g., metastatic cancer); or
(d) a combination thereof.

Assays to evaluate the binding ability of the antibodies toward a human regulators/modulators described herein can be used, including for example, ELISAs, Western blots and RIAs. The binding kinetics (e.g., binding affinity) of the antibodies also can be assessed by standard assays known in the art, such as by Biacore™. analysis.

Given that each of the subject antibodies can bind to a human regulator/modulator described herein, the V_{L} and V_{H} sequences can be “mixed and matched” to create other binding molecules that bind to a human regulators/modulators described herein. The binding properties of such “mixed and matched” antibodies can be tested using the binding assays described above and assessed in assays described in the examples. When V_{L} and V_{H} chains are mixed and matched, a V_{H} sequence from a particular V_{H}/V_{L} pairing can be replaced with a structurally similar V_{H} sequence. Likewise, preferably a V_{L} sequence from a particular V_{H}/V_{L} pairing is replaced with a structurally similar V_{L} sequence.

Accordingly, in one aspect, the invention provides an isolated monoclonal antibody, or antigen binding portion thereof comprising:
(a) a heavy chain variable region comprising an amino acid sequence; and
(b) a light chain variable region comprising an amino acid sequence; wherein the antibody specifically binds a human regulators/modulators described herein.

In some cases, the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example, Klimka et al., British 1 of Cancer 83(2):252-260 (2000) (describing the production of a humanized anti-CD30 antibody using only the heavy chain variable domain CDR3 of murine anti-CD30 antibody Ki-4), Beiboer et al., J. Mol. Biol. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad. Sci. U.S.A. 95:8910-8915 (1998) (describing a panel of humanized anti-integrin alphavbetaw antibodies using a heavy and light chain variable CDR3 domain. Hence, in some cases a mixed and matched antibody or a humanized antibody contains a CDR3 antigen binding domain that is specific for regulators/modulators described herein.

### Assays for Drug Development

Methods are also described herein for evaluating whether test agents can modulate the expression or activity of any of the regulators/modulators described herein. T cells, cancer cells, and combinations thereof can be evaluated for susceptibility to treatment with candidate compounds.

specifically, the methods can include assay steps for identifying a candidate test agent that selectively modulates the proliferation, functioning, or visibility of T cells or cancer cells, or for increasing or decreasing the levels or functioning of regulators described herein. For example, if the proliferation, cytokine production, activity, or viability of T cells is increased or decreased in the presence of one or more of the regulators described herein but the proliferation, cytokine production, activity, or the proliferation, activity, or viability of the T cells in the T cell-regulator assay mixture changes in the presence of a test agent then that test agent has utility for modulating the regulator of the T cells. Such a test agent is referred to as a modulator.

An assay can include determining whether a test agent can specifically cause decreased or increased numbers of T cells or whether a compound can specifically cause decreased or increased functioning of T cells. If the test agent does cause altered T cell numbers or T cell functioning, then the test agent can be selected/identified for further study, such as for its suitability as a therapeutic agent to treat a cancer or an immune condition or disease. For example, the test agent identified by the selection methods featured in the invention can be further examined for their ability to target a tumor, target an immune cell, or to treat cancer by, for example, administering the test agent (modulator) to an animal model.

The cells that are evaluated can include cytoxin T cells, helper T cells, regulatory T cells, naive T cells, activated T cells, CD4 cells, CD8 T cells, metastatic cells, benign cell samples, cell lines (including as cancer cell lines), or a combination thereof. The cells that are evaluated can also include cells from a patient with cancer (including a patient with metastatic cancer), or cells from a known cancer type or cancer cell line, or cells exhibiting an overproduction of any of the regulators described herein. A test agent that can modulate the production or activity of any of these cell types can be adminstered to an animal, including a patient.

For example one method can include (a) obtaining a cell sample from a patient; (b) measuring the amount or concentration of T cells/regulators/modulators in a known number of weight of cells from the sample to generate a reference value; (c) mixing the known number or weight of cells from the sample with a test agent to generate a test assay; to generate a test assay T cell/regulator/modulator value; (e) optionally repeating steps (c) and (d) with separate samples; and (f) selecting a test agent with a lower or a test assay T cell/regulator/modulator value than the reference value. The method can further include administering a test agent to an animal model, for example, to further evaluate the toxicity and/or efficacy of the test agent. in some cases, the method can further include administering the test agent to the patient from whom the cell or tissue sample as obtained.

Test agent or modulators (e.g., top hits identified by any method described herein) can be used in a cell-based assay using T cells or cells that express any of the regulators described herein as a readout of the efficacy of the test agents or modulators.

Assay methods are also described herein for identifying and assessing the potency of agents that may modulate T cells any of the regulators listed in tables 1-7 of FIGS. 1-4.

For example, T cells can release cytokines, such as Interferon γ or Interleukin-2, T cells or T cells expressing any of the modulators described herein can be contacted with a test agent and the release of cytokines by the T-cells can be measured, Such a test agent-related level of cytokines can be compared to the level observed for T cells not contacted with a test agent..

Useful test regulators, modulators, and test agents can be administered to a. test animal or a patient.

“Treatment” or “treating” refers to both therapeutic treatment and to prophylactic or preventative measures. Those in need of treatment include those already with the disorder as well as those prone to have the disorder, or those in whom the disorder is to be prevented.

“Subject” for purposes of administration of a regulator, modulator, test agent or composition described herein refers to administration to any animal classified as a mammal or bird, including humans, domestic animals, farm animals, zoo animals, experimental animals, pet animals, such as dogs, horses, cats, cows, etc. The experimental animals can include mice, rats, guinea pigs, goats, dogs, monkeys, or a combination thereof. In some cases, the subject is human.

As used herein, the term “cancer” includes solid animal tumors as well as hematological malignancies. The terms “tumor cell(s)” and “cancer cell(s)” are used interchangeably herein.

“Solid animal tumors” include cancers of the head and neck, lung, mesothelioma, mediastinum, lung, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone; and melanoma of cutaneous and intraocular origin. In addition, a metastatic cancer at any stage of progression can be treated, such as micrometastatic tumors, megametastatic tumors, and recurrent cancers.

In some cases, a hematological cancer or hematological malignancy can be treated. The term “hematological malignancies” includes adult or childhood leukemia and lymphomas, Hodgkin's disease, lymphomas of lymphocytic and cutaneous origin, acute and chronic leukemia, plasma cell neoplasm, and cancers associated with AIDS.

The inventive methods and compositions can also be used to treat leukemias, lymph nodes, thymus tissues, tonsils, spleen, cancer of the breast, cancer of the lung, cancer of the adrenal cortex, cancer of the cervix, cancer of the endometrium, cancer of the esophagus, cancer of the head and neck, cancer of the liver, cancer of the pancreas, cancer of the prostate, cancer of the thyrnus, carcinoid tumors, chronic lymphocytic leukemia, Ewing's sarcoma, gestational trophoblastic tumors, hepatoblastoma, multiple myeloma, non-small cell lung cancer, retinoblastoma, or tumors in the ovaries. A cancer at any stage of progression can be treated or detected, such as primary, metastatic, and recurrent cancers. In some cases, metastatic cancers are treated but primary cancers are not treated. Information regarding numerous types of cancer can be found, e.g., from the American Cancer Society (cancer.org), or from, e.g., Wilson et al. (1991) Harrison's Principles of Internal Medicine, 12th Edition, McGraw-Hill, Inc.

In some embodiments, the cancer and/or tumors to be treated are hematological malignancies, or those of lymphoid origin such as cancers or tumors of lymph nodes, thyrnus tissues, tonsils, spleen, and cells related thereto. In some embodiments, the cancer and/or tumors to be treated are those that have been resistant to T cell therapies.

Treatment of, or treating, metastatic cancer can include the reduction in cancer cell migration or the reduction in establishment of at least one metastatic tumor. The treatment also includes alleviation or diminishment of more than one symptom of metastatic cancer such as coughing, shortness of breath, hemoptysis, lymphadenopathy, enlarged liver, nausea, jaundice, bone pain, bone fractures, headaches, seizures, systemic pain and combinations thereof. The treatment may cure the cancer, e.g., it may prevent metastatic cancer, it may substantially eliminate metastatic tumor formation and growth, and/or it may arrest or inhibit the migration of metastatic cancer cells.

Anti-cancer activity can reduce the progression of a variety of cancers (e.g., breast, lung, pancreatic, or prostate cancer) using methods available to one of skill in the art. Anti-cancer activity, for example, can determined by identifying the lethal dose (LD₁₀₀) or the 50% effective dose (ED50) or the dose that inhibits growth at 50% (GI₅₀) of an agent of the present invention that prevents the migration of cancer cells. In one aspect, anti-cancer activity is the amount of the agent that reduces 50%., 60%, 70%, 80%, 90%, 95%, 97%, 98%, 99% or 100% of cancer cell migration, for example, when measured by detecting expression of a cancer cell marker at sites proximal or distal from a primary tumor site, or when assessed using available methods for detecting metastases.

In another example, agents that increase or decrease regulator/modulator expression or function can be administered to sensitize tumor cells to immune therapies. Hence, by administering an agent that increase or regulators/modulator expression or function, tumor cells can become more sensitive to the immune system and to various immune therapies.

### Compositions

The invention also relates to compositions containing one or more active agents such as any of the regulators described herein, modulators described herein, or combinations thereof. Such active agents can be a polypeptide, a nucleic acid encoding a polypeptide (e.g., within an expression cassette or expression vector), a modified cell, an inhibitory nucleic acid, a small molecule, a compound identified by a method described herein, or a combination thereof. The compositions can be pharmaceutical compositions.

In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof.

The composition can be formulated in any convenient form. In some embodiments, the compositions can include a protein or polypeptide encoded by any of the genes listed in Tables 1-7 or FIGS. 1-4. In other embodiments, the compositions can include at least one nucleic acid or expression cassette encoding a polypeptide listed in Tables 1-7 or FIGS. 1-4. In other embodiments, the compositions can include at least one nucleic acid or expression cassette that includes a nucleic acid segment complementarity to gene listed in Table 1-2 (e.g., an inhibitory nucleic acid). In other embodiments, the compositions can include at least one nucleic acid or expression cassette that includes a nucleic acid segment encoding a cas nuclease and at least one guide RNA that can target a regulator or modulator described herein. In other embodiments, the compositions can include at least one antibody that binds at least one protein encoded by at least one gene listed in Tables 1-7 or FIGS. 1-4 In other embodiments, the compositions can include at least one small molecule that binds, that activates, or that inhibits at least one gene listed in Tables 1-7 or FIG. 1-4, or at least one small molecule that binds, that activates, or that inhibits at least one protein encoded by at least one gene listed in Tables 1-7 or FIGS. 1-4. In other embodiments, the compositions can include cells with at least one modified genomic regulator or modulator genetic site, cells that express one or more of the regulators described herein, cells that express a cas nuclease and at least one guide RNA that can target at least one regulator or modulator gene, cells that express one or more inhibitory nucleic acids, or a combination thereof. The cells can be immune cells. In some cases, the cells can be one or more types of lymphoid cells, myeloid cells, cytotoxic T cells, helper T cells, regulatory T cells, naive T cells, activated T cells, CD4 T cells, CD8 T cells, gamma delta T cells, chimeric antigen receptor (CAR) cells, natural killer (NK) cells, induced pluripotent stem cell-derived immune (e.g., lymphoid and/or myeloid) cells, or a combination thereof.

The amount or number of cells administered can vary but amounts in the range of about 10⁶ to about 10⁹ cells can be used. The cells are generally delivered in a physiological solution such as saline or buffered saline. The cells can also be delivered in a vehicle such as within a population of liposomes, exosomes or microvesicles.

In some embodiments, the active agents of the invention (e.g., polypeptide, a nucleic acid encoding a polypeptide (e.g., within an expression cassette or expression vector), an antibody, an inhibitory nucleic acid, a small molecule, a compound identified by a method described herein, modified cells, or a combination thereof), are administered in a “therapeutically effective amount.” Such a therapeutically effective amount is an amount sufficient to obtain the desired physiological effect, such a reduction of at least one symptom of disease.

The disease can be cancer or an immune disease or condition. For example, active agents can reduce the symptoms of disease by 5%, or 10%, or 15%, or 20%, or 25%, or 30%, or 35%, or 40%, or 45%, or 50%, or 55%, or 60%, or 65%, or %70, or 80% or 90%, 095%, or 97%, or 99%, or any numerical percentage between 5% and 100%. For example, symptoms of cancer can also include tumor cachexia, tumor-induced pain conditions, tumor-induced fatigue, tumor growth, and metastatic spread. Hence, the active agents may also reduce tumor cachexia, tumor-induced pain conditions, tumor-induced fatigue, tumor growth, or a combination thereof by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%.

To achieve the desired effect(s), the active agents may be administered as single or divided dosages. For example, active agents can be administered in dosages of at least about 0.01 mg/kg to about 500 to 750 mg/kg, of at least about 0.01 mg/kg to about 300 to 500 mg/kg, at least about 0. 1 mg/kg to about 100 to 300 mg/kg or at least about 1 mg/kg to about 50 to 100 mg/kg of body weight, although other dosages may provide beneficial results. The amount administered will vary depending on various factors including, but not limited to, the type of small molecules, compounds, peptides, or nucleic acid chosen for administration, the disease, the weight, the physical condition, the health, and the age of the mammal. Such factors can be readily determined by the clinician employing animal models or other test systems that are available in the art.

Administration of the active agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the active agents and compositions of the invention may be essentially continuous over a preselected period of time or may be in a series of spaced doses. Both local and systemic administration is contemplated.

To prepare the composition, small molecules, compounds, polypeptides, nucleic acids, expression cassettes, ribonucleoprotein complexes, and other agents are synthesized or otherwise obtained, purified as necessary or desired. These small molecules, compounds, polypeptides, nucleic acids, expression cassettes, ribonucleoprotein complexes, and other agents can be suspended in a pharmaceutically acceptable carrier and/or lyophilized or otherwise stabilized. The small molecules, compounds, polypeptides, nucleic acids, expression cassettes, ribonucleoprotein complexes, other agents, and combinations thereof can be adjusted to an appropriate concentration, and optionally combined with other agents. The absolute weight of a given small molecule, compound, polypeptide, nucleic acid, ribonucleoprotein complex, and/or other agents included in a unit dose can vary widely. For example, about 0.01 to about 2 g, or about 0.1 to about 500 mg, of at least one molecule, compound, polypeptide, nucleic acid, ribonucleoprotein complexes, and/or other agent, or a plurality of molecules, compounds, polypeptides, nucleic acids, ribonucleoprotein complexes, and/or other agents can be administered, Alternatively, the unit dosage can vary from about 0.01 g to about 50 g, from about 0.01 g to about 35 g, from about, I g to about 25 g, from about 0.5 g to about 12 g, from about 0.5 g to about 8 g, from about 0.5 g to about 4 g, or from about 0.5 g to about 2 g.

Daily doses of the active agents of the invention can vary as well. Such daily doses can range, for example, from about 0. 1 g/day to about 50 g/day, from about 0, 1 g/day to about 25 g/day, from about 0 1 g/day to about 12 g/day, from about 0.5 g/day to about 8 g/day, from about 0.5 g/day to about 4 g/day, and from about 0.5 g/day to about 2 g/day.

It will be appreciated that the amount of active agent for use in treatment will vary not only with the particular carrier selected but also with the route of administration, the nature of the cancer condition being treated and the age and condition of the patient. Ultimately the attendant health care provider can determine proper dosage. In addition, a pharmaceutical composition can be formulated as a single unit dosage form.

Thus, one or more suitable unit dosage forms comprising the active agent(s) can be administered by a variety of routes including parenteral (including subcutaneous, intravenous, intramuscular and intraperitoneal), oral, rectal, dermal, transdermal, intrathoracic, intrapulmonary and intranasal (respiratory) routes. The active agent(s) may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the active agent with liquid carriers, solid matrices, semi-solid carriers, finely divided solid carriers or combinations thereof, and then, if necessary, introducing or shaping the product into the desired delivery system. For example, the active agent(s) can be linked to a convenient carrier such as a nanoparticle, albumin, polyalkylene glycol, or be supplied in prodrug form. The active agent(s), and combinations thereof can be combined with a carrier and/or encapsulated in a vesicle such as a liposome.

The compositions of the invention may be prepared in many forms that include aqueous solutions, suspensions, tablets, hard or soft gelatin capsules, and liposomes and other slow-release formulations, such as shaped polymeric gels. Administration of inhibitors can also involve parenteral or local administration of the in an aqueous solution or sustained release vehicle.

Thus, while the active agent(s) and/or other agents can sometimes be administered in an oral dosage form, that oral dosage form. can be formulated so as to protect the small molecules, compounds, polypeptides, nucleic acids, expression cassettes, ribonucleoprotein complexes, and combinations thereof from degradation or breakdown before the small molecules, compounds, polypeptides, nucleic acids encoding such polypeptides, expression cassettes, ribonucleoprotein complexes, and combinations thereof provide therapeutic utility. For example, in some cases the small molecules, compounds, polypeptides, nucleic acids encoding such polypeptide, expression cassettes, ribonucleoprotein complexes, and/or other agents can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g., liposomes), and other materials. The active agent(s) and/or other agents can be formulated in dry form (e.g., in freeze-dried form), in the presence or absence of a carrier.

If a carrier is desired, the carrier can be included in the pharmaceutical formulation, or can be separately packaged in a separate container, for addition to the inhibitor that is packaged in dry form, in suspension or in soluble concentrated form in a convenient liquid.

An active agent(s) and/or other agents can be formulated for parenteral administration (e.g., by injection, for example, bolus injection or continuous infusion) and may be presented in unit dosage form in ampoules, prefilled syringes, small volume infusion containers or multi-dose containers with an added preservative.

The compositions can also contain other ingredients such as active agents, anti-viral agents, antibacterial agents, antimicrobial agents and/or preservatives. Examples of additional therapeutic agents that may be used include, but are not limited to: alkylating agents, such as nitrogen mustards, alkyl sulfonates, nitrosoureas, ethyleniinnes, and triazenes; antimetabolites, such as folate antagonists, purine analogues, and pyriridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing horinone-releasing hormone anatagonists, octreotide acetate; microtubule-disruptor agents, such as ecteinascidins or their analogs and derivatives; microtubule-stabilizing agents such as paelitaxel (Taxol®), docetaxel (Taxotere®>), and epothilones A-F or their analogs or derivatives; plant-derived products, such as vinca alkaloids, epipodophyllotoxins, taxanes; and topoisomerase inhibitors; prenyl-protein transferase inhibitors, and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators, and monoclonal antibodies. The compositions can also be used in conjunction with radiation therapy.

The present description is further illustrated by the following examples, which should not be construed as limiting in any way. The contents of all cited references (including literature references, issued patents, published patent applications as cited throughout this application) are hereby expressly incorporated by reference.

### Example 1: CRISPRa Screening Primary Human T cells to Identify Genetic Regulators

This Example describes use of CRISPRa for screening of primary human T cells to identify genetic regulators of therapeutically relevant T cell phenotypes.

T cells were isolated from two separate donors. The two populations of T cells were transduced with a dCas9-VP64-expressing lentivirus (CRISPRa), or KRAB-dCas9-expressing lentivirus (CRISPRi) and T cells that stably expressed dCas9 were selected with mCherry. The dCas9-VP64 or KRAB-dCas9 expressing T cell populations were then transfected with two genorne-wide sgRNA libraries each to initiate CRISPR activation or interference of the T cells' genomes. For CRISPR activation Calabrese Sets A & B were used (see website at addgene.org/pooled-library/broadgpp-hurnan-crispra-calabrese-p65hsf/). For CRISPR interference, Dolcetto Sets A & B were used (see, addgene.org/pooled-library/broadgpp-hurnan-crispri-dolcetto/).

The T cell populations were stimulated with Immunocult0′ CD3/CD28/CD2 T cell activator (Sterncell Technologies, Vancouver, Canada), and the stimulated CRISPRa/i edited T cells from the two donors were sorted using fluorescent activated cell sorting (FACS) for the following markers: 1L-2 cytokine production, IFN-γ production, and CellTraceM Violet for cell proliferation. Sorted cells were subjected to genomic DNA extraction, and sgRNAs were PCR amplified, followed by next-generation-sequencing, to determine sgRNA frequencies in each population. Data was analyzed using MaGeck version 0.5.9.2 Li et al. Genome Biol 15:544 (2014).

These screens identified 1074 unique genes with significant responses to those phenotypes (FDR<−0.01), including both known and novel genes in T cell function.

Table 1 below lists positive regulators of T cell functions as detected by IFN-γ production.

TABLE 1
Positive Regulators of T Cell Functions
As detected by Interferon-γ Production

| Positive | Positive |
|---|---|
| Regulator | Interferon-γ |
| Gene | Production |
| APOBEC3C | IFNG |
| APOBEC3D | IFNG |
| APOL2 | IFNG |
| ASB12 | IFNG |
| BACE2 | IFNG |
| BCL9 | IFNG |
| BICDL2 | IFNG |
| C15orf52 | IFNG |
| C1orf94 | IFNG |
| CD2 | IFNG |
| CD247 | IFNG |
| CD28 | IFNG |
| CNGB1 | IFNG |
| CTSK | IFNG |
| DEAF1 | IFNG |
| DEF6 | IFNG |
| DEPDC7 | IFNG |
| DKK2 | IFNG |
| EMP1 | IFNG |
| EOMES | IFNG |
| EP300 | IFNG |
| FLT3 | IFNG |
| FOSL1 | IFNG |
| FOXQ1 | IFNG |
| GINS3 | IFNG |
| GLMN | IFNG |
| GNA11 | IFNG |
| HELZ2 | IFNG |
| HRASLS5 | IFNG |
| IFNG | IFNG |
| IL1R1 | IFNG |
| IL9R | IFNG |
| KLHDC3 | IFNG |
| KLRC4 | IFNG |
| LAT | IFNG |
| LCP2 | IFNG |
| LDB2 | IFNG |
| LTBR | IFNG |
| MVB12A | IFNG |
| NBPF6 | IFNG |
| NIT1 | IFNG |
| NLRC3 | IFNG |
| ORC1 | IFNG |
| OTUD7A | IFNG |
| OTUD7B | IFNG |
| PIK3AP1 | IFNG |
| PLCG2 | IFNG |
| PRDM1 | IFNG |
| PRKD2 | IFNG |
| PROCA1 | IFNG |
| RELA | IFNG |
| RNF217 | IFNG |
| SAFB2 | IFNG |
| SLC16A1 | IFNG |
| SLC5A10 | IFNG |
| SLC7A3 | IFNG |
| SPPL2B | IFNG |
| TAGAP | IFNG |
| TBX21 | IFNG |
| TMEM150B | IFNG |
| TMIGD2 | IFNG |
| TNFRSF12A | IFNG |
| TNFRSF14 | IFNG |
| TNFRSF1A | IFNG |
| TNFRSF1B | IFNG |
| TNFRSF8 | IFNG |
| TNFRSF9 | IFNG |
| TOR1A | IFNG |
| TPGS2 | IFNG |
| TRADD | IFNG |
| TRAF3IP2 | IFNG |
| TRIM21 | IFNG |
| VAV1 | IFNG |
| WT1 | IFNG |
| ZNF630 | IFNG |
| ZNF717 | IFNG |

Table 2 below lists positive regulators of T cell functions as detected by Interleukin-2 production.

TABLE 2
Positive Regulators of T Cells as Detected
by Interleukin-2 Production

| Positive | |
|---|---|
| Regulator | Positive IL2 |
| Gene | Production |
| ABCB10 | IL2 |
| ACSS2 | IL2 |
| ADAM19 | IL2 |
| ADAM23 | IL2 |
| ADAMTS5 | IL2 |
| ALKBH7 | IL2 |
| ALX4 | IL2 |
| ANXA2R | IL2 |
| AP2A1 | IL2 |
| APOBEC3C | IL2 |
| APOBEC3D | IL2 |
| APOL2 | IL2 |
| ARNT | IL2 |
| ART1 | IL2 |
| ASCL4 | IL2 |
| BEX4 | IL2 |
| BTG2 | IL2 |
| BTNL2 | IL2 |
| C11orf21 | IL2 |
| C12orf80 | IL2 |
| CBX4 | IL2 |
| CBY1 | IL2 |
| CCDC183 | IL2 |
| CCDC71L | IL2 |
| CD2 | IL2 |
| CD28 | IL2 |
| CD6 | IL2 |
| CDKN1B | IL2 |
| CDKN2C | IL2 |
| CHERP | IL2 |
| CIPC | IL2 |
| CLIP3 | IL2 |
| CNGB1 | IL2 |
| CNR2 | IL2 |
| CREB5 | IL2 |
| CUL3 | IL2 |
| DCTN5 | IL2 |
| DEF6 | IL2 |
| DEPDC7 | IL2 |
| DYNLL2 | IL2 |
| EAPP | IL2 |
| EEPD1 | IL2 |
| ELFN2 | IL2 |
| EMB | IL2 |
| EMP1 | IL2 |
| EMP3 | IL2 |
| EP300 | IL2 |
| ERCC3 | IL2 |
| ESRP1 | IL2 |
| F2 | IL2 |
| FBXL13 | IL2 |
| FBXO41 | IL2 |
| FNBP1L | IL2 |
| FOSB | IL2 |
| FOSL1 | IL2 |
| FOXO4 | IL2 |
| FOXQ1 | IL2 |
| FUZ | IL2 |
| GABRG1 | IL2 |
| GGTLC2 | IL2 |
| GNPDA1 | IL2 |
| GPR18 | IL2 |
| GPR20 | IL2 |
| GPR21 | IL2 |
| GPR84 | IL2 |
| GRIN3A | IL2 |
| GSDMD | IL2 |
| GSTM1 | IL2 |
| HCST | IL2 |
| HELZ2 | IL2 |
| HEPHL1 | IL2 |
| IL2 | IL2 |
| IL2RB | IL2 |
| IRX4 | IL2 |
| ISM1 | IL2 |
| KLF7 | IL2 |
| KLRC4 | IL2 |
| KRT18 | IL2 |
| LAT | IL2 |
| LCP2 | IL2 |
| LHX6 | IL2 |
| LMNA | IL2 |
| MAGEA9B | IL2 |
| MAP3K12 | IL2 |
| MERTK | IL2 |
| MTMR11 | IL2 |
| NDRG3 | IL2 |
| NIT1 | IL2 |
| NLRC3 | IL2 |
| NLRP2 | IL2 |
| NPLOC4 | IL2 |
| ORC1 | IL2 |
| OSBPL7 | IL2 |
| OTOP3 | IL2 |
| OTUD7A | IL2 |
| OTUD7B | IL2 |
| P2RY14 | IL2 |
| PAFAH1B2 | IL2 |
| PCP4 | IL2 |
| PDE3A | IL2 |
| PHF8 | IL2 |
| PIK3AP1 | IL2 |
| PLA2G3 | IL2 |
| PLCG2 | IL2 |
| POLK | IL2 |
| POU2F2 | IL2 |
| PPIL2 | IL2 |
| PRAC1 | IL2 |
| PRKCB | IL2 |
| PRKD2 | IL2 |
| RAB6A | IL2 |
| RAC1 | IL2 |
| RAC2 | IL2 |
| RIPK3 | IL2 |
| RRAS2 | IL2 |
| RYR1 | IL2 |
| SAFB2 | IL2 |
| SCN3A | IL2 |
| SDCCAG8 | IL2 |
| SERPINF1 | IL2 |
| SGTA | IL2 |
| SHOC2 | IL2 |
| SIGLEC1 | IL2 |
| SIRT1 | IL2 |
| SLC16A1 | IL2 |
| SLC44A5 | IL2 |
| SLC5A5 | IL2 |
| SMC4 | IL2 |
| SPPL2B | IL2 |
| SSUH2 | IL2 |
| SWAP70 | IL2 |
| TAF15 | IL2 |
| THEMIS | IL2 |
| TM4SF4 | IL2 |
| TMEM79 | IL2 |
| TNFRSF10B | IL2 |
| TNFSF11 | IL2 |
| TNRC6A | IL2 |
| TPGS2 | IL2 |
| TRAF3IP2 | IL2 |
| TRIM21 | IL2 |
| TRMT5 | IL2 |
| TRPM4 | IL2 |
| TRPV5 | IL2 |
| TSPYL5 | IL2 |
| UBA52 | IL2 |
| UBL5 | IL2 |
| VAV1 | IL2 |
| WARS2 | IL2 |
| ZAP70 | IL2 |
| ZNF141 | IL2 |
| ZNF296 | IL2 |
| ZNF701 | IL2 |

Table 3 below lists positive regulators of T cell functions as detected by T cell proliferation.

TABLE 3
Positive Regulators of T Cells as Detected Cell Proliferation

| Positive | |
|---|---|
| Regulator | Increased Cell |
| Gene | Proliferation |
| ABCB1 | Proliferation |
| ASAP1 | Proliferation |
| ATP10A | Proliferation |
| DEAF1 | Proliferation |
| FOXK1 | Proliferation |
| ITGAX | Proliferation |
| LCE6A | Proliferation |
| LCP2 | Proliferation |
| LEFTY1 | Proliferation |
| MYC | Proliferation |
| NAT8B | Proliferation |
| OLFM3 | Proliferation |
| PLD6 | Proliferation |

Table 4 below lists negative regulators of T cell functions as detected by reduced IFN-γ production.

TABLE 4
Negative Regulators of T Cell Functions As
detected by Less Interferon-γ Production

| Negative | Negative |
|---|---|
| Regulator | Interferon-γ |
| Genes | Production |
| ACER2 | IFNG |
| ADGRV1 | IFNG |
| AIF1L | IFNG |
| ALPL | IFNG |
| AMACR | IFNG |
| AMZ1 | IFNG |
| ARHGAP30 | IFNG |
| ARHGDIB | IFNG |
| ARHGEF11 | IFNG |
| ARL11 | IFNG |
| ATP2A2 | IFNG |
| B3GNT5 | IFNG |
| BACH2 | IFNG |
| BLM | IFNG |
| BSG | IFNG |
| BTBD2 | IFNG |
| BTLA | IFNG |
| BTRC | IFNG |
| CA11 | IFNG |
| CASTOR2 | IFNG |
| CBLB | IFNG |
| CCNT2 | IFNG |
| CCSER1 | IFNG |
| CD37 | IFNG |
| CD44 | IFNG |
| CD5 | IFNG |
| CD52 | IFNG |
| CD55 | IFNG |
| CDK6 | IFNG |
| CEACAM1 | IFNG |
| CEBPA | IFNG |
| CEBPB | IFNG |
| CEP164 | IFNG |
| CKAP2L | IFNG |
| CLCN2 | IFNG |
| CLDN25 | IFNG |
| COLQ | IFNG |
| CST5 | IFNG |
| CTNNA1 | IFNG |
| CYP24A1 | IFNG |
| DDIT4L | IFNG |
| DENND3 | IFNG |
| DGKG | IFNG |
| DGKK | IFNG |
| DGKZ | IFNG |
| DSC1 | IFNG |
| EBF2 | IFNG |
| ECEL1 | IFNG |
| EIF3K | IFNG |
| EPB41 | IFNG |
| EPS8L1 | IFNG |
| FAM35A | IFNG |
| FAM53B | IFNG |
| FAM83A | IFNG |
| FKRP | IFNG |
| FOXA3 | IFNG |
| FOXF1 | IFNG |
| FOXF2 | IFNG |
| FOXI3 | IFNG |
| FOXJ1 | IFNG |
| FOXL2 | IFNG |
| FOXL2NB | IFNG |
| GABRQ | IFNG |
| GATA3 | IFNG |
| GATA4 | IFNG |
| GATA6 | IFNG |
| GCM2 | IFNG |
| GCSAM | IFNG |
| GCSAML | IFNG |
| GMFG | IFNG |
| GNL3L | IFNG |
| GRAP | IFNG |
| GRB2 | IFNG |
| GRIA1 | IFNG |
| GTSF1L | IFNG |
| HRH2 | IFNG |
| HYLS1 | IFNG |
| IKZF1 | IFNG |
| IKZF3 | IFNG |
| IL2RB | IFNG |
| INPPL1 | IFNG |
| JMJD1C | IFNG |
| KCNV1 | IFNG |
| KRIT1 | IFNG |
| LAMB1 | IFNG |
| LAPTM5 | IFNG |
| LAT2 | IFNG |
| LAX1 | IFNG |
| LCK | IFNG |
| LENEP | IFNG |
| LMO4 | IFNG |
| LRRC25 | IFNG |
| LRRC4B | IFNG |
| LYN | IFNG |
| MAB21L2 | IFNG |
| MAP4K1 | IFNG |
| MBIP | IFNG |
| MBOAT1 | IFNG |
| METTL23 | IFNG |
| MIPEP | IFNG |
| MIPOL1 | IFNG |
| MMP21 | IFNG |
| MSMB | IFNG |
| MUC1 | IFNG |
| MUC21 | IFNG |
| MUC8 | IFNG |
| N4BP1 | IFNG |
| NAIF1 | IFNG |
| NDNF | IFNG |
| NFATC1 | IFNG |
| NFKB2 | IFNG |
| NFKBIA | IFNG |
| NKX2-1 | IFNG |
| NKX2-3 | IFNG |
| NMB | IFNG |
| NR2F1 | IFNG |
| ODF4 | IFNG |
| OPRD1 | IFNG |
| ORC5 | IFNG |
| OTUD4 | IFNG |
| PASD1 | IFNG |
| PBK | IFNG |
| PCBP2 | IFNG |
| PDLIM1 | IFNG |
| PDPN | IFNG |
| PECAM1 | IFNG |
| PIP5K1A | IFNG |
| PIP5K1B | IFNG |
| PITPNA | IFNG |
| POGZ | IFNG |
| POLK | IFNG |
| POU2AF1 | IFNG |
| PSTPIP1 | IFNG |
| PTPN12 | IFNG |
| PTPRC | IFNG |
| PVRIG | IFNG |
| RAB14 | IFNG |
| RBP7 | IFNG |
| RETREG1 | IFNG |
| RFC2 | IFNG |
| RHCE | IFNG |
| RNF19B | IFNG |
| RNF2 | IFNG |
| RUSC2 | IFNG |
| SELPLG | IFNG |
| SETD1B | IFNG |
| SH3KBP1 | IFNG |
| SIGLEC6 | IFNG |
| SIPA1L1 | IFNG |
| SLA | IFNG |
| SLA2 | IFNG |
| SLC26A4 | IFNG |
| SLC44A5 | IFNG |
| SLC45A1 | IFNG |
| SLC6A8 | IFNG |
| SLC6A9 | IFNG |
| SMAD9 | IFNG |
| SMAGP | IFNG |
| SOCS3 | IFNG |
| SOX13 | IFNG |
| SPATA31A1 | IFNG |
| SPN | IFNG |
| SPOCK3 | IFNG |
| SPRED1 | IFNG |
| STAP1 | IFNG |
| STK35 | IFNG |
| SULT6B1 | IFNG |
| SYT15 | IFNG |
| TEC | IFNG |
| TIAM1 | IFNG |
| TMEM151A | IFNG |
| TMEM87B | IFNG |
| TMPRSS11E | IFNG |
| TNNT2 | IFNG |
| TRIB2 | IFNG |
| TRIM28 | IFNG |
| TSPAN1 | IFNG |
| UBASH3B | IFNG |
| UBQLN4 | IFNG |
| UBXN7 | IFNG |
| UNC119 | IFNG |
| UPP1 | IFNG |
| VPS28 | IFNG |
| WLS | IFNG |
| ZKSCAN4 | IFNG |
| ZNF445 | IFNG |
| ZNF474 | IFNG |

Table 5 below lists negative regulators of T cell functions as detected by reduced Interleukin-2 production.

TABLE 5
Negative Regulators of T Cell Functions As
detected by Less Interleukin-2 Production

| Negative | |
|---|---|
| Regulator | Negative IL2 |
| Gene | Production |
| ABI3BP | IL2 |
| AEBP1 | IL2 |
| AHR | IL2 |
| ANTXR2 | IL2 |
| ARHGAP15 | IL2 |
| ARHGAP27 | IL2 |
| ARHGDIB | IL2 |
| ARID3A | IL2 |
| ARL4D | IL2 |
| B4GALNT3 | IL2 |
| BICD1 | IL2 |
| C10orf82 | IL2 |
| C17orf75 | IL2 |
| C19orf35 | IL2 |
| C1RL | IL2 |
| C2orf69 | IL2 |
| C6orf132 | IL2 |
| C9orf84 | IL2 |
| CABP1 | IL2 |
| CBLB | IL2 |
| CCSER1 | IL2 |
| CD34 | IL2 |
| CD4 | IL2 |
| CD5 | IL2 |
| CD52 | IL2 |
| CEACAM1 | IL2 |
| CEACAM7 | IL2 |
| CEBPB | IL2 |
| CES3 | IL2 |
| CGB3 | IL2 |
| COL11A1 | IL2 |
| COL4A3 | IL2 |
| COLQ | IL2 |
| CPEB3 | IL2 |
| CRELD2 | IL2 |
| CST9L | IL2 |
| DDX55 | IL2 |
| DLG4 | IL2 |
| DOK1 | IL2 |
| EBF3 | IL2 |
| EIF3K | IL2 |
| EN2 | IL2 |
| EOMES | IL2 |
| EPB41 | IL2 |
| ETS1 | IL2 |
| F5 | IL2 |
| FAM96A | IL2 |
| FHL1 | IL2 |
| FOXA3 | IL2 |
| FOXE1 | IL2 |
| FOXI3 | IL2 |
| FOXL2NB | IL2 |
| FUS | IL2 |
| FUT4 | IL2 |
| GCSAM | IL2 |
| GCSAML | IL2 |
| GDAP1L1 | IL2 |
| GDPD2 | IL2 |
| GMIP | IL2 |
| GNL3L | IL2 |
| GOLPH3 | IL2 |
| GRAP | IL2 |
| GRB2 | IL2 |
| HAUS7 | IL2 |
| HERC1 | IL2 |
| HLA-DQB2 | IL2 |
| HSD17B11 | IL2 |
| IKZF1 | IL2 |
| IKZF3 | IL2 |
| INPPL1 | IL2 |
| INTS10 | IL2 |
| ITIH2 | IL2 |
| ITPKA | IL2 |
| ITPKB | IL2 |
| ITPKC | IL2 |
| JDP2 | IL2 |
| JKAMP | IL2 |
| JMJD1C | IL2 |
| KIAA1024 | IL2 |
| KIF15 | IL2 |
| KIF5A | IL2 |
| KNTC1 | IL2 |
| LAT2 | IL2 |
| LAX1 | IL2 |
| LGR5 | IL2 |
| LIME1 | IL2 |
| LMBRD2 | IL2 |
| LOC401052 | IL2 |
| LONP2 | IL2 |
| LRCH3 | IL2 |
| LRRC23 | IL2 |
| LRRC25 | IL2 |
| LRRC52 | IL2 |
| LYN | IL2 |
| LYPD1 | IL2 |
| MAATS1 | IL2 |
| MAB21L2 | IL2 |
| MAGEB17 | IL2 |
| MAP4K1 | IL2 |
| MEF2C | IL2 |
| METTL9 | IL2 |
| MICU1 | IL2 |
| MRPL17 | IL2 |
| MUC1 | IL2 |
| NAIF1 | IL2 |
| NCF2 | IL2 |
| NDNF | IL2 |
| NDUFB1 | IL2 |
| NHP2 | IL2 |
| NKX2-6 | IL2 |
| NLGN4Y | IL2 |
| NNT | IL2 |
| NPIPB9 | IL2 |
| NR4A1 | IL2 |
| NR4A3 | IL2 |
| NRCAM | IL2 |
| NRP1 | IL2 |
| NRSN2 | IL2 |
| NSUN7 | IL2 |
| OLFML1 | IL2 |
| OMP | IL2 |
| OPRD1 | IL2 |
| OR1K1 | IL2 |
| OR2B11 | IL2 |
| OSBPL11 | IL2 |
| OTOG | IL2 |
| OTUD4 | IL2 |
| PATL2 | IL2 |
| PAX5 | IL2 |
| PFKL | IL2 |
| PHF2 | IL2 |
| PIBF1 | IL2 |
| PIP5K1A | IL2 |
| PIP5K1B | IL2 |
| PITPNC1 | IL2 |
| PLCL1 | IL2 |
| PLEKHM2 | IL2 |
| PPARG | IL2 |
| PPIC | IL2 |
| PSRC1 | IL2 |
| PSTPIP1 | IL2 |
| PTPN12 | IL2 |
| PTPN22 | IL2 |
| PTPN6 | IL2 |
| PTPRC | IL2 |
| PVRIG | IL2 |
| RBP4 | IL2 |
| RPL13A | IL2 |
| S100A2 | IL2 |
| SALL4 | IL2 |
| SAMD8 | IL2 |
| SENP6 | IL2 |
| SETD1B | IL2 |
| SEZ6L | IL2 |
| SFT2D1 | IL2 |
| SH3TC1 | IL2 |
| SIGIRR | IL2 |
| SIT1 | IL2 |
| SLA | IL2 |
| SLA2 | IL2 |
| SLC20A2 | IL2 |
| SLC39A2 | IL2 |
| SLC6A8 | IL2 |
| SMAGP | IL2 |
| SNRNP48 | IL2 |
| SOCS2 | IL2 |
| SORBS1 | IL2 |
| SOX13 | IL2 |
| SPN | IL2 |
| SPRED1 | IL2 |
| SPRED2 | IL2 |
| SRPK1 | IL2 |
| STAP1 | IL2 |
| STK38L | IL2 |
| SYPL1 | IL2 |
| TCF12 | IL2 |
| TEX35 | IL2 |
| TFCP2L1 | IL2 |
| TMEM14C | IL2 |
| TMEM223 | IL2 |
| TMEM262 | IL2 |
| TNNT2 | IL2 |
| TPRA1 | IL2 |
| TRIM6- | IL2 |
| TRIM34 | |
| TSPAN1 | IL2 |
| UBASH3B | IL2 |
| UBE2W | IL2 |
| UBR4 | IL2 |
| UBXN7 | IL2 |
| UCP1 | IL2 |
| UIMC1 | IL2 |
| ULK1 | IL2 |
| UPK3B | IL2 |
| VPS28 | IL2 |
| VSTM5 | IL2 |
| XKR9 | IL2 |
| YLPM1 | IL2 |
| ZDHHC7 | IL2 |
| ZEB1 | IL2 |
| ZEB2 | IL2 |
| ZNF445 | IL2 |
| ZNF70 | IL2 |
| ZNF831 | IL2 |

Table 6 below lists negative regulators of T cell functions as detected by reduced cell proliferation.

TABLE 6
Negative Regulators of T Cell Functions
As detected by Less Cell Proliferation

| Negative | Decreased |
|---|---|
| Regulator | Cell |
| Gene | Proliferation |
| ABCB1 | Proliferation |
| ASAP1 | Proliferation |
| ATP10A | Proliferation |
| DEAF1 | Proliferation |
| FOXK1 | Proliferation |
| ITGAX | Proliferation |
| LCE6A | Proliferation |
| LCP2 | Proliferation |
| LEFTY1 | Proliferation |
| MYC | Proliferation |
| NAT8B | Proliferation |
| OLFM3 | Proliferation |
| PLD6 | Proliferation |
| PREP | Proliferation |
| SULT1A1 | Proliferation |
| SULT1A4 | Proliferation |
| AHNAK | Proliferation |
| ARHGDIB | Proliferation |
| B3GNT5 | Proliferation |
| CASZ1 | Proliferation |
| CD27 | Proliferation |
| CEBPB | Proliferation |
| CRHBP | Proliferation |
| FLI1 | Proliferation |
| FOSL2 | Proliferation |
| HLX | Proliferation |
| MAP4K1 | Proliferation |
| MUC21 | Proliferation |
| MXI1 | Proliferation |
| NDRG1 | Proliferation |
| NEUROD2 | Proliferation |
| SLC2A1 | Proliferation |
| SLC43A3 | Proliferation |
| SMAGP | Proliferation |
| SOX13 | Proliferation |
| SP140 | Proliferation |
| TPI1 | Proliferation |
| TTC39C | Proliferation |

These regulators and agents that modulate these regulators can be used as T cell related immunotherapies for cancer or autoimmune diseases

### Example 2: CRISPRi Identification of Genes that Regulate T Cells

This Example describes use of CRISPRi for screening of primary human T cells to identify genetic regulators of therapeutically relevant I cell phenotypes.

The two populations of T cells were transduced with a KRAB-dCas9-expressing lentivirus (CRISPRi) and T cells that stably expressed dCas9 were selected with mCherry. The KRAB-dCas9 expressing T cell populations were then transfected with two genome-wide sgRNA libraries each to initiate CRISPR interference of the T cells' genomes. For CRISPR interference, Dolcetto Sets A & B were used (see, addgene.org!pooled-library/broadgpp-human-crispri-dolcetto/).

The T cell populations were stimulated with Immunocult™ CD3/CD28/CD2 T cell activator (Stemncell Technologies, Vancouver, Canada), and the stimulated CRISPRi edited T cells from the two donors were sorted using fluorescent activated cell sorting (FACS) for the following markers: IL-2 cytokine production, IFN-γ production, and CellTrace™ Violet for cell proliferation. Sorted cells were subjected to genomic DNA extraction, and sgRNAs were PCR amplified, followed by next-generation-sequencing, to determine sgRNA frequencies in each population, Data was analyzed using MaIGeck version 0.5.9.2 Li et al. Genome Biol 15:544 (2014). Table 7 lists the genes that modulated T cell functions.

TABLE 7
Genes from CRISPRi Screen that Modulate T Cells

| Gene | Screen | Positive or Negative Regulator |
|---|---|---|
| ANKRD17 | IFNG | positive |
| AQP3 | IFNG | positive |
| ARID4B | IFNG | positive |
| ATP6V1C1 | IFNG | positive |
| ATPAF1 | IFNG | positive |
| ATXN7 | IFNG | positive |
| BCAT2 | IFNG | positive |
| BCL10 | IFNG | positive |
| CASD1 | IFNG | positive |
| CBFB | IFNG | positive |
| CD2 | IFNG | positive |
| CD247 | IFNG | positive |
| CD28 | IFNG | positive |
| CD3D | IFNG | positive |
| CD3E | IFNG | positive |
| CD3G | IFNG | positive |
| CD4 | IFNG | positive |
| CHUK | IFNG | positive |
| CNP | IFNG | positive |
| COG3 | IFNG | positive |
| CREBBP | IFNG | positive |
| CUL1 | IFNG | positive |
| DDA1 | IFNG | positive |
| DDX60L | IFNG | positive |
| DEF6 | IFNG | positive |
| DHDDS | IFNG | positive |
| DHX29 | IFNG | positive |
| DPP9-AS1 | IFNG | positive |
| ELOF1 | IFNG | positive |
| ERC1 | IFNG | positive |
| ETNK1 | IFNG | positive |
| EXOC4 | IFNG | positive |
| FAM133B | IFNG | positive |
| FGFR1OP | IFNG | positive |
| FITM2 | IFNG | positive |
| FLVCR2 | IFNG | positive |
| FNDC4 | IFNG | positive |
| GOSR1 | IFNG | positive |
| GPX7 | IFNG | positive |
| GRAP2 | IFNG | positive |
| HARS | IFNG | positive |
| HNRNPL | IFNG | positive |
| HOXD13 | IFNG | positive |
| IFNG | IFNG | positive |
| IFNGR1 | IFNG | positive |
| IFNGR2 | IFNG | positive |
| IKBKB | IFNG | positive |
| IKBKG | IFNG | positive |
| IL21R | IFNG | positive |
| INPP1 | IFNG | positive |
| ITK | IFNG | positive |
| JAK1 | IFNG | positive |
| JUN | IFNG | positive |
| KAT7 | IFNG | positive |
| KCNIP3 | IFNG | positive |
| KIAA1109 | IFNG | positive |
| KIDINS220 | IFNG | positive |
| LAT | IFNG | positive |
| LCK | IFNG | positive |
| LCP2 | IFNG | positive |
| LIMS2 | IFNG | positive |
| LOC101927322 | IFNG | positive |
| LRIG1 | IFNG | positive |
| MALT1 | IFNG | positive |
| MAP3K7 | IFNG | positive |
| MBD2 | IFNG | positive |
| MEAF6 | IFNG | positive |
| MEN1 | IFNG | positive |
| MMP24 | IFNG | positive |
| MOB4 | IFNG | positive |
| MYLIP | IFNG | positive |
| NDFIP2 | IFNG | positive |
| NSD2 | IFNG | positive |
| NSFL1C | IFNG | positive |
| NYNRIN | IFNG | positive |
| OSBP | IFNG | positive |
| PCYT2 | IFNG | positive |
| PGBD5 | IFNG | positive |
| PI4KB | IFNG | positive |
| PLCG1 | IFNG | positive |
| PRDM1 | IFNG | positive |
| PRKAR1A | IFNG | positive |
| PRKD2 | IFNG | positive |
| PRRC2B | IFNG | positive |
| PTPRC | IFNG | positive |
| RAC2 | IFNG | positive |
| RAET1L | IFNG | positive |
| RBCK1 | IFNG | positive |
| RDX | IFNG | positive |
| RHOA | IFNG | positive |
| RHOG | IFNG | positive |
| ROPN1B | IFNG | positive |
| RRAS2 | IFNG | positive |
| RTP2 | IFNG | positive |
| SAE1 | IFNG | positive |
| SCRIB | IFNG | positive |
| SEC61A1 | IFNG | positive |
| SEC62 | IFNG | positive |
| SEH1L | IFNG | positive |
| SEL1L | IFNG | positive |
| SH2D1A | IFNG | positive |
| SHOC2 | IFNG | positive |
| SLC38A6 | IFNG | positive |
| SLC3A2 | IFNG | positive |
| SPCS2 | IFNG | positive |
| SPTLC2 | IFNG | positive |
| SPTSSA | IFNG | positive |
| SRD5A2 | IFNG | positive |
| SRP19 | IFNG | positive |
| SRP68 | IFNG | positive |
| SRP72 | IFNG | positive |
| SRPRB | IFNG | positive |
| SSB | IFNG | positive |
| STAT3 | IFNG | positive |
| SUGT1 | IFNG | positive |
| SULT2B1 | IFNG | positive |
| SUPT5H | IFNG | positive |
| SYT15 | IFNG | positive |
| TADA1 | IFNG | positive |
| TADA2B | IFNG | positive |
| TAF11 | IFNG | positive |
| TAF13 | IFNG | positive |
| TAF2 | IFNG | positive |
| TAF6L | IFNG | positive |
| TARS | IFNG | positive |
| TBX21 | IFNG | positive |
| TLN1 | IFNG | positive |
| TMX1 | IFNG | positive |
| TNFRSF1A | IFNG | positive |
| TRAF6 | IFNG | positive |
| TRIM21 | IFNG | positive |
| TXK | IFNG | positive |
| UBA2 | IFNG | positive |
| VAV1 | IFNG | positive |
| VPS29 | IFNG | positive |
| VPS35 | IFNG | positive |
| VPS37C | IFNG | positive |
| VPS41 | IFNG | positive |
| WAS | IFNG | positive |
| XPO6 | IFNG | positive |
| ZAP70 | IFNG | positive |
| ARHGAP15 | IFNG | negative |
| BRD9 | IFNG | negative |
| BRIP1 | IFNG | negative |
| CAD | IFNG | negative |
| CBLB | IFNG | negative |
| CBLL1 | IFNG | negative |
| CD5 | IFNG | negative |
| CDK12 | IFNG | negative |
| CHERP | IFNG | negative |
| CPSF2 | IFNG | negative |
| CPSF6 | IFNG | negative |
| CSTF3 | IFNG | negative |
| CTDSPL2 | IFNG | negative |
| DGKZ | IFNG | negative |
| E2F1 | IFNG | negative |
| EIF3B | IFNG | negative |
| EIF3D | IFNG | negative |
| EIF3K | IFNG | negative |
| EIF4E2 | IFNG | negative |
| GCN1 | IFNG | negative |
| GIGYF2 | IFNG | negative |
| GNAI2 | IFNG | negative |
| HIF1AN | IFNG | negative |
| IKBKE | IFNG | negative |
| LARGE2 | IFNG | negative |
| LAT2 | IFNG | negative |
| MAP4K1 | IFNG | negative |
| MCM2 | IFNG | negative |
| METAP2 | IFNG | negative |
| METTL3 | IFNG | negative |
| MTF1 | IFNG | negative |
| MYB | IFNG | negative |
| NFKB2 | IFNG | negative |
| NIT1 | IFNG | negative |
| NMT1 | IFNG | negative |
| NRF1 | IFNG | negative |
| NUDC | IFNG | negative |
| PCBP2 | IFNG | negative |
| PDGFRA | IFNG | negative |
| PITPNB | IFNG | negative |
| PNISR | IFNG | negative |
| PPP1R8 | IFNG | negative |
| PRMT1 | IFNG | negative |
| PSMD13 | IFNG | negative |
| PSMD4 | IFNG | negative |
| PTMA | IFNG | negative |
| RAB4A | IFNG | negative |
| RBPJ | IFNG | negative |
| RIOK2 | IFNG | negative |
| RNF20 | IFNG | negative |
| RNF40 | IFNG | negative |
| RPL19 | IFNG | negative |
| RPL26 | IFNG | negative |
| RPL35A | IFNG | negative |
| RPL38 | IFNG | negative |
| RPL6 | IFNG | negative |
| RPS13 | IFNG | negative |
| RPS17 | IFNG | negative |
| RPS8 | IFNG | negative |
| SCRN3 | IFNG | negative |
| SF3A1 | IFNG | negative |
| SLA2 | IFNG | negative |
| SLAMF6 | IFNG | negative |
| SMC3 | IFNG | negative |
| SP1 | IFNG | negative |
| SPN | IFNG | negative |
| SYMPK | IFNG | negative |
| THOC3 | IFNG | negative |
| TONSL | IFNG | negative |
| TSC1 | IFNG | negative |
| U2AF2 | IFNG | negative |
| UBASH3A | IFNG | negative |
| UNCX | IFNG | negative |
| USP5 | IFNG | negative |
| ZC3H18 | IFNG | negative |
| BCL10 | IL2 | positive |
| CASD1 | IL2 | positive |
| CD2 | IL2 | positive |
| CD247 | IL2 | positive |
| CD28 | IL2 | positive |
| CD3D | IL2 | positive |
| CD3E | IL2 | positive |
| CD3G | IL2 | positive |
| CHD7 | IL2 | positive |
| DEF6 | IL2 | positive |
| DNTTIP1 | IL2 | positive |
| ELOF1 | IL2 | positive |
| GRAP2 | IL2 | positive |
| IFNGR2 | IL2 | positive |
| IL2 | IL2 | positive |
| ITK | IL2 | positive |
| KIDINS220 | IL2 | positive |
| LAT | IL2 | positive |
| LCP2 | IL2 | positive |
| NDFIP2 | IL2 | positive |
| NDUFB1 | IL2 | positive |
| NYNRIN | IL2 | positive |
| PGBD5 | IL2 | positive |
| PLCG1 | IL2 | positive |
| PRKD2 | IL2 | positive |
| RAC2 | IL2 | positive |
| RHOG | IL2 | positive |
| RPN2 | IL2 | positive |
| SCRIB | IL2 | positive |
| SHOC2 | IL2 | positive |
| SIN3B | IL2 | positive |
| SPRYD3 | IL2 | positive |
| SRP19 | IL2 | positive |
| SRP68 | IL2 | positive |
| SRP72 | IL2 | positive |
| SULT2B1 | IL2 | positive |
| TAF11 | IL2 | positive |
| TAF13 | IL2 | positive |
| TAF2 | IL2 | positive |
| TAF8 | IL2 | positive |
| TLN1 | IL2 | positive |
| TRIM21 | IL2 | positive |
| VAV1 | IL2 | positive |
| VPS29 | IL2 | positive |
| VPS35 | IL2 | positive |
| WAS | IL2 | positive |
| ZAP70 | IL2 | positive |
| ACTL6A | IL2 | negative |
| ADSS | IL2 | negative |
| ANLN | IL2 | negative |
| ARHGAP15 | IL2 | negative |
| ARID2 | IL2 | negative |
| ATP1A1 | IL2 | negative |
| AUNIP | IL2 | negative |
| BECN1 | IL2 | negative |
| BMS1 | IL2 | negative |
| BOP1 | IL2 | negative |
| C21orf62 | IL2 | negative |
| CAD | IL2 | negative |
| CBFB | IL2 | negative |
| CBLB | IL2 | negative |
| CD5 | IL2 | negative |
| CDC23 | IL2 | negative |
| CDK12 | IL2 | negative |
| CENPE | IL2 | negative |
| CENPI | IL2 | negative |
| CEP192 | IL2 | negative |
| CHAF1B | IL2 | negative |
| CHERP | IL2 | negative |
| CHMP3 | IL2 | negative |
| CHMP5 | IL2 | negative |
| CHMP6 | IL2 | negative |
| CNOT1 | IL2 | negative |
| CPSF4 | IL2 | negative |
| CPSF6 | IL2 | negative |
| CTDSPL2 | IL2 | negative |
| CTPS1 | IL2 | negative |
| DDX47 | IL2 | negative |
| DGKZ | IL2 | negative |
| DHODH | IL2 | negative |
| DLST | IL2 | negative |
| DNTTIP2 | IL2 | negative |
| DPY19L3 | IL2 | negative |
| E2F1 | IL2 | negative |
| EDC4 | IL2 | negative |
| EFTUD2 | IL2 | negative |
| EIF3B | IL2 | negative |
| EIF3D | IL2 | negative |
| EIF3E | IL2 | negative |
| EIF3K | IL2 | negative |
| EIFSA | IL2 | negative |
| EP400 | IL2 | negative |
| ESF1 | IL2 | negative |
| FADD | IL2 | negative |
| FAM49B | IL2 | negative |
| FAM60A | IL2 | negative |
| FAU | IL2 | negative |
| GCN1 | IL2 | negative |
| GIGYF2 | IL2 | negative |
| GINS3 | IL2 | negative |
| HGS | IL2 | negative |
| IL2RA | IL2 | negative |
| IL2RB | IL2 | negative |
| IL2RG | IL2 | negative |
| ILF2 | IL2 | negative |
| INTS3 | IL2 | negative |
| JPH1 | IL2 | negative |
| KANSL3 | IL2 | negative |
| KAT5 | IL2 | negative |
| KLF2 | IL2 | negative |
| LAMTOR2 | IL2 | negative |
| LOC401052 | IL2 | negative |
| MAD2L1BP | IL2 | negative |
| MAK16 | IL2 | negative |
| MAP4K1 | IL2 | negative |
| MAU2 | IL2 | negative |
| MCM2 | IL2 | negative |
| MCM3AP | IL2 | negative |
| MEMO1 | IL2 | negative |
| METAP2 | IL2 | negative |
| MMP16 | IL2 | negative |
| MRPL22 | IL2 | negative |
| MST1L | IL2 | negative |
| MYCBP2 | IL2 | negative |
| NARFL | IL2 | negative |
| NEPRO | IL2 | negative |
| NFKB2 | IL2 | negative |
| NMT1 | IL2 | negative |
| NRF1 | IL2 | negative |
| NUDC | IL2 | negative |
| OTUB1 | IL2 | negative |
| PCBP1 | IL2 | negative |
| PCBP2 | IL2 | negative |
| PDGFRA | IL2 | negative |
| PFAS | IL2 | negative |
| PITPNB | IL2 | negative |
| PNISR | IL2 | negative |
| POLE | IL2 | negative |
| POLR1B | IL2 | negative |
| PPAN | IL2 | negative |
| PPIH | IL2 | negative |
| PPP1R8 | IL2 | negative |
| PRMT1 | IL2 | negative |
| PRPF4B | IL2 | negative |
| PRR12 | IL2 | negative |
| PSMD2 | IL2 | negative |
| PTPN23 | IL2 | negative |
| PUM1 | IL2 | negative |
| RAB4A | IL2 | negative |
| RASA2 | IL2 | negative |
| RBM14 | IL2 | negative |
| RBM25 | IL2 | negative |
| RBM42 | IL2 | negative |
| RBSN | IL2 | negative |
| RCL1 | IL2 | negative |
| RMND5A | IL2 | negative |
| RNF20 | IL2 | negative |
| RNF40 | IL2 | negative |
| RPL10 | IL2 | negative |
| RPL10A | IL2 | negative |
| RPL13 | IL2 | negative |
| RPL14 | IL2 | negative |
| RPL15 | IL2 | negative |
| RPL18 | IL2 | negative |
| RPL19 | IL2 | negative |
| RPL23A | IL2 | negative |
| RPL24 | IL2 | negative |
| RPL26 | IL2 | negative |
| RPL27 | IL2 | negative |
| RPL34 | IL2 | negative |
| RPL35 | IL2 | negative |
| RPL36 | IL2 | negative |
| RPL37A | IL2 | negative |
| RPL38 | IL2 | negative |
| RPL6 | IL2 | negative |
| RPL7A | IL2 | negative |
| RPL8 | IL2 | negative |
| RPL9 | IL2 | negative |
| RPLP1 | IL2 | negative |
| RPS11 | IL2 | negative |
| RPS13 | IL2 | negative |
| RPS16 | IL2 | negative |
| RPS17 | IL2 | negative |
| RPS20 | IL2 | negative |
| RPS23 | IL2 | negative |
| RPS24 | IL2 | negative |
| RPS25 | IL2 | negative |
| RPS3 | IL2 | negative |
| RPS3A | IL2 | negative |
| RPS4X | IL2 | negative |
| RPS5 | IL2 | negative |
| RPS7 | IL2 | negative |
| RPS8 | IL2 | negative |
| RUVBL2 | IL2 | negative |
| SART1 | IL2 | negative |
| SETD1A | IL2 | negative |
| SLAMF6 | IL2 | negative |
| SLBP | IL2 | negative |
| SMARCE1 | IL2 | negative |
| SMC1A | IL2 | negative |
| SMC3 | IL2 | negative |
| SNRNP27 | IL2 | negative |
| SNRNP70 | IL2 | negative |
| SNRPC | IL2 | negative |
| SNRPF | IL2 | negative |
| SP1 | IL2 | negative |
| SRFBP1 | IL2 | negative |
| SRSF1 | IL2 | negative |
| STAT5B | IL2 | negative |
| SURF6 | IL2 | negative |
| SYMPK | IL2 | negative |
| TBL1X | IL2 | negative |
| THOC3 | IL2 | negative |
| TNPO3 | IL2 | negative |
| TRAF2 | IL2 | negative |
| TRAIP | IL2 | negative |
| TSC1 | IL2 | negative |
| TSG101 | IL2 | negative |
| TUBGCP5 | IL2 | negative |
| TYMS | IL2 | negative |
| U2AF1 | IL2 | negative |
| U2AF2 | IL2 | negative |
| UBASH3A | IL2 | negative |
| UBASH3B | IL2 | negative |
| UPF1 | IL2 | negative |
| UTP14A | IL2 | negative |
| UTP15 | IL2 | negative |
| VPS28 | IL2 | negative |
| WDR45 | IL2 | negative |
| WDR5 | IL2 | negative |
| YEATS4 | IL2 | negative |
| ZMAT2 | IL2 | negative |
| AAMP | Proliferation | positive |
| AARS | Proliferation | positive |
| AATF | Proliferation | positive |
| AK2 | Proliferation | positive |
| ALDH18A1 | Proliferation | positive |
| AP2M1 | Proliferation | positive |
| ATIC | Proliferation | positive |
| ATP1A1 | Proliferation | positive |
| ATP5O | Proliferation | positive |
| ATP6V1B2 | Proliferation | positive |
| ATP6V1F | Proliferation | positive |
| ATXN10 | Proliferation | positive |
| BMS1 | Proliferation | positive |
| BOP1 | Proliferation | positive |
| BUD23 | Proliferation | positive |
| C12orf60 | Proliferation | positive |
| CAD | Proliferation | positive |
| CARS | Proliferation | positive |
| CCDC86 | Proliferation | positive |
| CCT6A | Proliferation | positive |
| CD247 | Proliferation | positive |
| CD3D | Proliferation | positive |
| CD3E | Proliferation | positive |
| CD3EAP | Proliferation | positive |
| CD3G | Proliferation | positive |
| CINP | Proliferation | positive |
| CLNS1A | Proliferation | positive |
| CPSF4 | Proliferation | positive |
| CRCP | Proliferation | positive |
| CTPS1 | Proliferation | positive |
| DAD1 | Proliferation | positive |
| DDX27 | Proliferation | positive |
| DDX52 | Proliferation | positive |
| DGCR8 | Proliferation | positive |
| DHODH | Proliferation | positive |
| DHX29 | Proliferation | positive |
| DHX37 | Proliferation | positive |
| DICER1 | Proliferation | positive |
| DNAJA3 | Proliferation | positive |
| DNM2 | Proliferation | positive |
| DNTTIP2 | Proliferation | positive |
| DPH6 | Proliferation | positive |
| DROSHA | Proliferation | positive |
| EIF2B2 | Proliferation | positive |
| EIF2B3 | Proliferation | positive |
| EIF2B4 | Proliferation | positive |
| EIF5A | Proliferation | positive |
| ELP4 | Proliferation | positive |
| ESF1 | Proliferation | positive |
| EXOSC4 | Proliferation | positive |
| EXOSC5 | Proliferation | positive |
| EXOSC7 | Proliferation | positive |
| EXOSC9 | Proliferation | positive |
| FAM149B1 | Proliferation | positive |
| FARSB | Proliferation | positive |
| FBL | Proliferation | positive |
| FCF1 | Proliferation | positive |
| FH | Proliferation | positive |
| FLVCR1 | Proliferation | positive |
| FTSJ3 | Proliferation | positive |
| GFER | Proliferation | positive |
| GMPS | Proliferation | positive |
| GNL2 | Proliferation | positive |
| GNL3L | Proliferation | positive |
| GNPAT | Proliferation | positive |
| GTF3C1 | Proliferation | positive |
| HARS | Proliferation | positive |
| HAUS4 | Proliferation | positive |
| HCCS | Proliferation | positive |
| HEATR3 | Proliferation | positive |
| HSD17B10 | Proliferation | positive |
| HSD17B12 | Proliferation | positive |
| HSPA9 | Proliferation | positive |
| IL2RG | Proliferation | positive |
| IMPDH2 | Proliferation | positive |
| ISG20L2 | Proliferation | positive |
| KARS | Proliferation | positive |
| LAGE3 | Proliferation | positive |
| LAT | Proliferation | positive |
| LCP2 | Proliferation | positive |
| LETM1 | Proliferation | positive |
| LONP1 | Proliferation | positive |
| MARS2 | Proliferation | positive |
| MDN1 | Proliferation | positive |
| METTL16 | Proliferation | positive |
| MMACHC | Proliferation | positive |
| MRPL16 | Proliferation | positive |
| MRPL22 | Proliferation | positive |
| MRPL35 | Proliferation | positive |
| MRPL36 | Proliferation | positive |
| MRPL37 | Proliferation | positive |
| MRPL41 | Proliferation | positive |
| MRPL42 | Proliferation | positive |
| MRPL45 | Proliferation | positive |
| MRPL54 | Proliferation | positive |
| MRPS11 | Proliferation | positive |
| MRPS14 | Proliferation | positive |
| MRPS17 | Proliferation | positive |
| MRPS18A | Proliferation | positive |
| MRPS2 | Proliferation | positive |
| MRPS23 | Proliferation | positive |
| MRPS33 | Proliferation | positive |
| MRPS5 | Proliferation | positive |
| MRPS9 | Proliferation | positive |
| MTHED1L | Proliferation | positive |
| MTOR | Proliferation | positive |
| MYBBP1A | Proliferation | positive |
| NAT10 | Proliferation | positive |
| NCL | Proliferation | positive |
| NEPRO | Proliferation | positive |
| NIFK | Proliferation | positive |
| NOC2L | Proliferation | positive |
| NOL10 | Proliferation | positive |
| NOL6 | Proliferation | positive |
| NOL8 | Proliferation | positive |
| NOP14 | Proliferation | positive |
| NOP2 | Proliferation | positive |
| NOP56 | Proliferation | positive |
| NOP58 | Proliferation | positive |
| NUBP1 | Proliferation | positive |
| NUFIP1 | Proliferation | positive |
| ORAOV1 | Proliferation | positive |
| PAM16 | Proliferation | positive |
| PCYT2 | Proliferation | positive |
| PDCD11 | Proliferation | positive |
| PDGFRA | Proliferation | positive |
| PDHA1 | Proliferation | positive |
| PDSS2 | Proliferation | positive |
| PELP1 | Proliferation | positive |
| PGD | Proliferation | positive |
| PGM3 | Proliferation | positive |
| PHB | Proliferation | positive |
| PHB2 | Proliferation | positive |
| PISD | Proliferation | positive |
| PITRM1 | Proliferation | positive |
| PMPCA | Proliferation | positive |
| PNO1 | Proliferation | positive |
| PNPT1 | Proliferation | positive |
| POLG2 | Proliferation | positive |
| POLR1A | Proliferation | positive |
| POLR1C | Proliferation | positive |
| POLR1D | Proliferation | positive |
| POLR2E | Proliferation | positive |
| POLR3B | Proliferation | positive |
| POLRMT | Proliferation | positive |
| POP1 | Proliferation | positive |
| POP4 | Proliferation | positive |
| POP5 | Proliferation | positive |
| POT1 | Proliferation | positive |
| PPAN | Proliferation | positive |
| PPAT | Proliferation | positive |
| PSMG1 | Proliferation | positive |
| QARS | Proliferation | positive |
| RAC2 | Proliferation | positive |
| RBM19 | Proliferation | positive |
| RCL1 | Proliferation | positive |
| RIOK1 | Proliferation | positive |
| RIOK2 | Proliferation | positive |
| ROMO1 | Proliferation | positive |
| RPF1 | Proliferation | positive |
| RPF2 | Proliferation | positive |
| RPL28 | Proliferation | positive |
| RPL30 | Proliferation | positive |
| RPL39 | Proliferation | positive |
| RPLP2 | Proliferation | positive |
| RPN2 | Proliferation | positive |
| RPP21 | Proliferation | positive |
| RPP30 | Proliferation | positive |
| RPS11 | Proliferation | positive |
| RPS12 | Proliferation | positive |
| RPS15 | Proliferation | positive |
| RPS17 | Proliferation | positive |
| RPS19BP1 | Proliferation | positive |
| RPS27 | Proliferation | positive |
| RPS4X | Proliferation | positive |
| RPS6 | Proliferation | positive |
| RPSA | Proliferation | positive |
| RRP12 | Proliferation | positive |
| RRP36 | Proliferation | positive |
| RRP7A | Proliferation | positive |
| RRP9 | Proliferation | positive |
| RSL24D1 | Proliferation | positive |
| SAMM50 | Proliferation | positive |
| SARS | Proliferation | positive |
| SDHC | Proliferation | positive |
| SEH1L | Proliferation | positive |
| SLC35B1 | Proliferation | positive |
| SLC38A6 | Proliferation | positive |
| SLC7A11 | Proliferation | positive |
| SPOUT1 | Proliferation | positive |
| SRFBP1 | Proliferation | positive |
| SSB | Proliferation | positive |
| SURF6 | Proliferation | positive |
| TAF1A | Proliferation | positive |
| TAF1C | Proliferation | positive |
| TAF1D | Proliferation | positive |
| TAF8 | Proliferation | positive |
| TAMM41 | Proliferation | positive |
| TARS | Proliferation | positive |
| TEX10 | Proliferation | positive |
| TIMM44 | Proliferation | positive |
| TNKS1BP1 | Proliferation | positive |
| TOMM20 | Proliferation | positive |
| TOMM40 | Proliferation | positive |
| TP53I13 | Proliferation | positive |
| TRMT112 | Proliferation | positive |
| TRMT5 | Proliferation | positive |
| TRNT1 | Proliferation | positive |
| TSEN2 | Proliferation | positive |
| TSEN54 | Proliferation | positive |
| TSR1 | Proliferation | positive |
| TTI1 | Proliferation | positive |
| TWISTNB | Proliferation | positive |
| TWNK | Proliferation | positive |
| UMPS | Proliferation | positive |
| UQCR10 | Proliferation | positive |
| UQCRB | Proliferation | positive |
| UQCRC1 | Proliferation | positive |
| UTP11 | Proliferation | positive |
| UTP14A | Proliferation | positive |
| UTP3 | Proliferation | positive |
| UTP6 | Proliferation | positive |
| VAV1 | Proliferation | positive |
| VPS29 | Proliferation | positive |
| VPS72 | Proliferation | positive |
| WAS | Proliferation | positive |
| WDR12 | Proliferation | positive |
| WDR3 | Proliferation | positive |
| WDR36 | Proliferation | positive |
| WDR55 | Proliferation | positive |
| XRCC5 | Proliferation | positive |
| XRCC6 | Proliferation | positive |
| YAE1D1 | Proliferation | positive |
| ZAP70 | Proliferation | positive |
| ZCCHC9 | Proliferation | positive |
| ZNHIT3 | Proliferation | positive |
| ZNHIT6 | Proliferation | positive |
| ZNRD1 | Proliferation | positive |

This screen identified quite a few of the same genes as were identified in the screen described in Example 1. The following genes were new genes identified by this CRISPRi screen: HNRNPL, IHOXD13, IFNGR1, IFNGR2, IKBKB, IKBKG, IL21R, INPPi, ITKJAKI,JUN, KA T7, KCNIP3, KIAAI A109, KID[NS220, LIMS2, LOC101927322, LRIGI, MALTI, MAP3K7, IViBD2, MEAF6, MENI, MMP4, MOB4, MYLIP, NDFIP2, NSD2, NSFL1C, NYNRIN, OSBP˜, PCY T2, PGBD5, PI4 KB, PLCG1, PRKAR1A, PRRC2B, RAETI L, RBCK1, RDX, RHOA, RIOGi, ROPNIB, RTP2, SAE1, SCRIB, SEC61AI, SEC62, SEH1L, SEL1L, SI-2D1IA, SLC38A6, SLC3A2, SPCS2, SPTLC2, SPTSSA, SRD5A2, SRP19, SRP68, SRP72, SRPRB, SSB, STAT3, SUGTI, SULT2B1, SUPTIH1, TADA1, TADA2B, TAF 1, TAF13, TAF2, TAF6L, TARS, TLN1, TMNIX1, TRAF6, TXK, UBA2, VPS29, VPS35, VPS37C, VPS41, WAS, XPO6, BRD9, BIRIPI, CAD, CBLLI, CDKI2, CPSF2, CPSF6, CSTF3, CTDSPL2, E2F1, EIF3B, EJF3D, EIF4E2, GCN1, GIGYF2, iNA12, HIFIAN, IKBKE, LARGE2, MCCM2, METAP2, METTL3, M-TF1, MYB, NMT1, NRFI, NUDC, PDGFRA, PITPNB, PNISR, PPP1R8, PRMTI, PSMD13, PSMD4, PTMA, RAB4A, RBPJ, RIOK2, RNF20, RNF40, RPL19, RPL26, RPL35A, RPL38, RPL6, RPS13, RPS17, RPS8, SCRN3, SF3AI, SLAMF6, SMC3, SPI, SYMHPK, THOC3, TONSL, TSCI, U2AF2, UBASH3A, 10 UNCX, USP5, ZC3HI8, BCLI0, CASDI, CD3D, CD3E, CD3G, CHD7, DNTTIP1, ELOFI, GRAP2, IFNGR2, ITK, KIDINS220, NDFIP2, NYNRIN, PGBD5, PLCG1, RHOG, RPN2, SCRIB, SIN3B, SPRYD3, SRP19, SRP68, SRP72, SULT2B1, TAFI1, TAF13, TAF2, TAF8, TLN1, VPS29, VPS35, WAS, ACTL6A, ADSS, ANLN, ARID2, ATP1A1, AUNIP, BECNI, BMS1, BOP1, C2Iorf62, CAD, CBFB, CDC23, CDK12, CEN^{P}E, CENPI, CEPi192, CHAFIB, CIHMfP3, CHMP5, CHMP6, CNOTI, CPSF4, CPSF6, CTDSPL2, CTPS1, DDX4.7, DHODH, DLST, DNTTIP2, DPY19L3, E2FI, EDCi4, EFTUID2, EIF3B, EIF3D, EIF3E, EF5A, EP400, ESFI, FADD, FAM-149B, FAM60A, FAU, GCNI, GIGYF2, HGS, IL2RA, IL2EG, ILF2, INTS3, JPH1, KANSL3, KAT5, KLF2, LAMTOR2, MAD21IBP, MAK16, MAU2, MC 12, MCM3AP, MEMO1, T MT P2, MMIvP16, MRPL22. MSTIL, MYCBP2, NAREL, NEPRO, IMT, N^{R}FI, NUDC, OTUBI, PCBPI, PDGFRA, PEAS, PITPN3, PNISR, POLE, POLRIB, PPAN, PPI-H, PPPlR8, PRMT1, PRPF4B, PRRI2, PSMD2, PTPN23, PUM1, RAB4A, RASA2, RBM14, RBM25, RBM42, RBSN, RCLI, RMND5A, RNF20, RNF40, RPLI0, RPLI 0A, RPL13, RPL14, RPL15, RPLI8, RPL19, RPL23A, RPL24, RPL26, RPL27, RPL34, RPL35, RPL36, RPL37A, RPL38, RPL6, RPL7A, RPL8, RPL9, RPLP1, RPS11, RPS13, RPS16, RPS17, RPS20, RPS23, RPS24, RPS25, RPS3, RPS3A, RPS4X, RPS5, RPS7, RPS8, RUVBL2, SARTI, SETDIA, SLAMF6, SLBP, SMARCEI, SMCIA, SMC3, SNRNP27, SNRNP70, SNRPC, SNRPF, SPI, SRFBP1, SRSFI, STAT5B, SURF6, SYMPK, TBLIX, THOC3, TNPO3, TRAF2, TRAIP, TSCI, TSGII01, 30⁺ T1CP51³5, TYMS, U2AF1, U2AF2, UBASL3A, UPF1, UTPI4A, UJTP15, WDR45, WDR5, YEATS4, ZMAT2, AAMP, AARS, AATF, AK2, ALDHI8A1, AP2M1, A TIC, ATPIA1, ATP5O, ATP6VIB2, ATP6V1F, ATXN10, BMS1, BOPI, BUD23, C12orf60, CAD, CARS, CCDC86, CCT6A, CD3D, CD3E, CD3EAP, CD3G, CINP, CLNSIA, CPSF4, CRCP, CTPS1, DAD1, DDX27, DDX52, DGCR8, DHODI-1, D-1X29, DUX37, DICERI, DNAJA3, DNM2, DNTT^{I}P2, DPH6, DROSHA, EIF2B2, EIF2B3, EIF2B4, EF5A, ELP4, ESFI, EXOSC4, EXOSC5, EXOSC7, EXOSC9, FAM149B1, FARSB, FBL, FCFI, FH, FLNVCR-1, FTSJ3, GFER, GMPS, GNL2, GNPAT, GTF3C1, H ARS, HAUS4, HCCS, HEATR3, USD17B1 0, HSDI7IB12, HSPA9, IL2RG, IMPD2, ISG20L2, KARS, LAGE3, LETMI, LONPI1 MARS2, MDN1 MIETTLi6, MMACHC, MRPI16, MRPL22, MRPL5, MIRPL36, MRPL37, MRPL41 MIRPL42, MRPL45, MRPL54, MRPS11, MRPS14, MRPSI7, MRPS18A, MRPS2, MRPS23, MRPS33, MRPS5, MRPS9, MTHFD1L, MOR, MYBBPIA, NAT0I NCL, NEPRO, NIFK, NOC2L, NOLI, NOL6, NOL8, NOP14, NOP2, NOP56, NOP58, NUBPI, NLUFIPI, ORAOV1, PAM16, PCYT2, PDCD11, PDGFRA, PDHAI, PDSS2, PELPI, PGD, PGI3, PHB, PHB2, PISD, PVTRMI, PMPCA, PNOI, PNPTI, POLG2, POLRI A, POLRIC, POLRID, POLR2E, POLR3B, POLRMT, POP1, POP4, POP5, POTI, PPAN, PPAT, PSMGI, QARS, RBMI19, RCCLI, RIOKI, RJOK2, ROMOI, RPF1, RPF2, RPL28, RPL30, RPL39, RPLP2, RPN2, RPP21, RPP30, RPSI1, RPS12, RPS15, RPSI7, RPS19BPI, RPS27, RPS4X, RPS6, RPSA, RRP12, RRP36, RRP7A, RRP9, RSL24-DI, SAMM50, SARS, SDHC, SEH1L, SLC35BI, SLC38A6, SLC7A1I, SPOUT1, SRFBPI, SSB, SURF6, TAFIA, TAF1C. TAFID, TAF8, TAMM41, TARS., TEX10, TIMM44, TNKS1BPI, TOMM20, TOMM40, TP531I3, TRMT 12, TRNTI, TSEN2, TSEN54, TSRI, TT 1, TWJSTNB, TWNK, UMPS, UQCR10, UQCRB, UQCRC1, UTP1 1, UTP14A, UTP3, UTP6, VPS29, VPS72, WAS, WDRI2, W^{T}DR3, WDR36, WDR55, XRCC5, XRCC6, YAEID1, ZCCHC9, ZNHIT3, ZNHIT6, and ZNRD1.

These regulators and agents that modulate these regulators can be used as T cell related immunotherapies for cancer or autoimmune diseases.

### Example 3: CRISPRa Screening Primary fHunan T cells to Identify Genetic Regulators

### INTRODUCTION

Regulated T cell cytokine production in response to stimulation plays a role in balanced immune responses. Cytokine dysregulation can lead to autoimmunity, immunodeficiency, and immune evasion in cancer (1-4). Interleukin 2 (IL-2), secreted predominantly by CD4⁺ cells, drives T cell expansion (5) and is therapeutically applied in autoimmunity and cancer at different doses (6). Interferon gamma (IFN-γ) is a cytokine secreted by both CD4⁺ and CD8⁺ cells that promotes a type I immune response against intracellular pathogens including viruses (4) and correlates with positive cancer immunotherapy responses (7-9). Much of our current understanding of the pathways leading to cytokine production in humans originates from studies in transformed T cell lines, which often are not representative of primary human cell biology (10-12). Comprehensive understanding of pathways that control cytokine production in primary human T cells would facilitate the development of next-generation immunotherapies.

Unbiased forward genetic approaches can uncover the components of regulatory networks systematically but challenges with efficient Cas9 delivery have limited their application in primary cells. Genome-wide CRISPR knockout screens have been completed using primary mouse immune cells from Cas9-expressing transgenic mice (13-15), including a screen for regulators of innate cytokine production in dendritic cells (13). Genome-scale CRISPR studies in human primary cells have recently been accomplished using transient Cas9 electroporation to introduce gene knockouts (16, 17), However, comprehensive discovery of regulators requires both gain-of-function and loss-of-function studies. For example, CRISPRa gain-of-function screens can discover genes that may not normally be active in the tested conditions, but which can promote phenotypes of interest (18, 19). In contrast to a CRISPR knockout, CRISPRa or CRISPRi require the sustained expression of an activator-linked endonuclease-dead Cas9 (dCas9) and due to poor lentiviral delivery has been limited to small scale experiments in primary cells (20, 21). Here we developed a CRISPRa and CRISPRi screening platform in primary human T cells, which allowed for the systematic discovery of genes and pathways that can be perturbed to tune stimulation-dependent cytokine responses.

### Materials and Methods

Isolation and Culture of Human IT cells

Human T cells were sourced from PBMC-enriched leukapheresis products (Leukopaks, Stemcell Technologies cat 70500.2) from healthy donors, following IRB approved informed written consent (Stemcell Technologies). Bulk T cells were isolated from Leukopaks using EasySep magnetic selection following manufacturers' recommended protocol (Stemcell Technologies cat 17951). Unless stated otherwise, bulk T cells were frozen in Bambanker Cell Freezing Medium at 5×10⁷ cells/ml (Bulldog Bio cat BB01) and stored at −80° C. for short-term or in liquid nitrogen for long-term storage immediately after isolation. Unless otherwise noted, thawed T cells were cultured in X-VIVO 15 (Lonza Bioscience cat 04-418Q) supplemented with 5% FCS, 55 mM 2-mercaptoethanol, 4 mM N-acetyl L-cysteine, and 500 IU/ml of recombinant human 1L-2 (Amerisource Bergen cat 10101641). Primary T cells were activated using anti-humanCD3/CD28 CTS dynabeads (Fisher Scientific cat 40203D) at a 1:1 cell-to-bead ratio at 10⁶ cells/ml.

Cell line maintenance

Lenti-X HEK293T cells (Takara Bio cat 632180) were maintained in DMEM high glucose with GlutaMAIX™ (Fisher Scientific cat 10566024), supplemented with 10% FCS, 100 U/ml of PenStrep (Fisher Scientific cat 15140122), 1 mM sodium pyruvate (Fisher Scientific cat 11360070), 1X MEM non-essential amino acids (Fisher Scientific cat 11140050), and 10 mM HEPES solution (Sigma cat H0887-1 00ML) Cells were passaged every 2 days using Tryple Express (Fisher Scientific cat 12604013) for dissociation and maintained at <60% confluency. NALM6 cells were engineered to express NY-ESO-1 peptide in an HLA-A0201 background, recognizable with the 1G4 TCR by the Fyquem lab at UCSF and provided for TCR stimulation coculture experiments. For sinplicity, these cells are referred to as NALM6. NALM6 cells were cultured in RPML (Gibco cat 21870092) supplemented with 10% FCS, 100 U/ml PenStrep (Fisher Scientific cat 15140122), 1 mM sodium pyruvate (Fisher Scientific cat 11360070), and 1X MEM non-essential amino acids (Fisher Scientific cat 11140050), 10 mM HEPES solution (Sigma cat 140887-100ML), and 2 mM L-glutarnine (Lonza Bioscience cat 17-605E).

### Plasmids

dCas9-VP64 originated from lentiSAMv2 (addgene 75112) and cloned into the lentiCRfSPRv2-dCas9 backbone (addgene 112233) with Gibson Assembly. The promoter was switched to SFFV and mCherry was introduced upstream of dCas9-VP64, separated by a P2A sequence resulting in the pZRI 12 plasmid. The LTR-LTR range was minimized to enhance lentiviral titer. For CRISPRi, BFP in pHR-SFFV-dCas9-BFP-KRAB (addgene 46911) was switched to mCherry with Gibson Assembly resulting in pZR0 71.

Single sgRNAs for arrayed experiments have been introduced by Golden Gate Cloning as described before (22). Briefly, DNA oligomers with Golden Gate overhangs were annealed and subsequently cloned into the non-digested target plasmid using the NEB®Golden Gate Assembly Kit (BsmBI-v2, New England Biolabs cat E1602L). sgRNAs have been cloned into pXPR_502 (addgene 96923) for CRISPRa and into CROPseq-Guide-Puro (43) (addgene 86708) for CRISPRi. All single sgRNAs used in this study are found in Table 8.

| | | Target-Guide- | CRISPR | |
|---|---|---|---|---|
| # | Target | Number | system | Original Guide Sequence |
| guideRS002 | EGFR | 1 | CRISPRa | CCACCGCTGTCCACCGCCTC |
| guideRS003 | EGFR | 2 | CRISPRa | GACCCAAGGCCAGCGGCCGC |
| guideRS004 | EGFR | 3 | CRISPRa | GGAGGGAGGAGAACCAGCAG |
| guideRS015 | GARP | 1 | CRISPRa | AAATTGCAGCCGGAGCGCGG |
| guideRS017 | GARP | 2 | CRISPRa | TCCGGATAAACCGAGGCACG |
| guideRS019 | GARP | 3 | CRISPRa | GCGAAGCATCTTCACCACCC |
| guideRS005 | IL1R2 | 1 | CRISPRa | GACCCAGCACTGCAGCCTGG |
| guideRS006 | IL1R2 | 2 | CRISPRa | AAACTTATGCGGCGTTTCCT |
| guideRS007 | IL1R2 | 3 | CRISPRa | ATCACTTTAAAACCACCTCT |
| guideRS001 | NT-CTRL | 1 | CRISPRa | CTGAAAAAGGAAGGAGTTGA |
| guideRS022 | B2M | 1 | CRISPRi | CGCGAGCACAGCTAAGGCCA |
| guideRS023 | B2M | 2 | CRISPRi | GAGTAGCGCGAGCACAGCTA |
| guideRS024 | B2M | 3 | CRISPRi | GGCCGAGATGTCTCGCTCCG |
| guideRS025 | CD4 | 1 | CRISPRi | AACAAAGCACCCTCCCCACT |
| guideRS026 | CD4 | 2 | CRISPRi | CAAACAGGCGTATCTGTGTG |
| guideRS027 | CD4 | 3 | CRISPRi | CTCTGCAACCAGGAGCCCAG |
| guideRS028 | CD45 | 1 | CRISPRi | CACTGTTGTCTTATCAGACG |
| guideRS029 | CD45 | 2 | CRISPRi | CTCGTCTGATAAGACAACAG |
| guideRS030 | CD45 | 3 | CRISPRi | GTTTGTTCTTAGGGTAACAG |
| guideRS021 | NT-Ctr | 1 | CRISPRi | ACTCAGCCATTTTATTAGAA |
| pZR073 | APOBEC3C | 1 | CRISPRa | GAGCAGCCTGTCTTTATCGG |
| pZR074 | APOBEC3C | 2 | CRISPRa | GTTCTCCGGGCCCCTCCTAC |
| pZR077 | FOXQ1 | 1 | CRISPRa | CGCCTGGTGCGCGCCCGTTG |
| pZR078 | FOXQ1 | 2 | CRISPRa | GAGGCCACACTGCAGCGCGG |
| pZR079 | IFNG | 1 | CRISPRa | TGGGTCTGTCTCATCGTCAA |
| pZR080 | IFNG | 2 | CRISPRa | GTGGCACAGGTGGGCATAAT |
| pZR081 | IL1R1 | 1 | CRISPRa | GGGTGGAGAGTTGGGACACC |
| pZR082 | IL1R1 | 2 | CRISPRa | GCTCGGCTGGGCCAGTCCGC |
| pZR083 | IL2 | 1 | CRISPRa | TCCATTCAGTCAGTCTTTGG |
| pZR084 | IL2 | 2 | CRISPRa | GAGAGCTATCACCTAAGTGT |
| pZR085 | IL2RB | 1 | CRISPRa | TATCTGGCCCTGGGTGCTTG |
| pZR086 | IL2RB | 2 | CRISPRa | GGTGCCGCCCCCAGCGTAGG |
| pZR087 | LAT2 | 1 | CRISPRa | GACAGGCTCAGCTATGAAGA |
| pZR088 | LAT2 | 2 | CRISPRa | GCGGCAGTGCGGCGGATGTA |
| pZR089 | LHX6 | 1 | CRISPRa | TCCCCCTCCAGCTGCAACGG |
| pZR090 | LHX6 | 2 | CRISPRa | GGAGGACTACCAAGAGGGGG |
| pZR091 | MAP4K1 | 1 | CRISPRa | ACAGTCGTGCAGTGCAGCTG |
| pZR092 | MAP4K1 | 2 | CRISPRa | GGGGCTCTGAGAGCCTCTGA |
| pZR093 | NT-CTRL | 1 | CRISPRa | GAGTCAACGGGGAATACCAT |
| pZR094 | NT-CTRL | 2 | CRISPRa | GAACCATTAGATCAATGCGA |
| pZR095 | OTUD7B | 1 | CRISPRa | GGGGAGCGGCGCTAAAGGCG |
| pZR096 | OTUD7B | 2 | CRISPRa | GAAAACACGGGGTCACGCGC |
| pZR097 | PIK3AP1 | 1 | CRISPRa | ACCTGCACCCGCGGCCGTTG |
| pZR098 | PIK3AP1 | 2 | CRISPRa | GCCGAGTCCCGCAGGCGGGG |
| pZR099 | TNFRSF1A | 1 | CRISPRa | TTGGGAGTGGTCGGATTGGT |
| pZR100 | TNFRSF1A | 2 | CRISPRa | GGCACAAGGCAGCCAGATCT |
| pZR101 | TRIM21 | 1 | CRISPRa | AAAGGGTGTGTGGAGAAATG |
| pZR102 | TRIM21 | 2 | CRISPRa | GAGCGCGCAACCAGGACCAC |
| pZR103 | VAV1 | 1 | CRISPRa | CCAGGCCTGTGTCGAGTGGG |
| pZR104 | VAV1 | 2 | CRISPRa | GAGGAGGAGCCATGGGGCGG |

The genome wide CRISPRa (Calabrese A, cat 92379 and Calabrese B, cat 92380) and CRISPRi libraries (Dolcetto A, cat 92385 and Dolcetto B, cat 92386) (22) were obtained from addgene. Forty nanograms of each library were transformed into Endura™ ElectroCompetent Cells (Lucigen cat 60242-2) following the manufacturer's instructions. After transformation, Endura cells were grown in a shaking incubator for 16 hours at 30° C. in the presence of ampicillin. Library plasmid has been isolated using the Qiagen Plasmid Plus MaxiKit (Qiagen 12963) and sequenced for sgRNA representation as described under “Genome-wide CRISPRa and CRISPRi screens”.

For cDNA mediated target overexpression, the lentiCRISPRv2 (addgene 75112) backbone was rebuilt to a lentiviral cDNA cloning plasmid with an SFFV promoter followed by BsmBT restriction sites and P2A-Puro, Transgene cDNAs were purchased from Genscript, choosing the canonical (longest) isoforn for each gene, and BsmBI restriction sites were introduced by PCR. The final lentiviral transfer plasmids were assembled using the NEB®Golden Gate Assembly Kit (BsmBI-v2, New England Biolabs cat E1602L).

To clone direct-capture compatible CRISPRa-SAM plasmids for Perturb-seg, different sgRNA designs were synthesized as G-Blocks (integrated DNA technologies) and cloned into pXPR_502 (addgene 96923) by Gibson assembly, replacing its sgRNA cassette.

Lentivirus production

Unless otherwise stated, HEK293T cells were seeded in Opti-MEM™ Reduced Serum Medium (OPTI-MEM) with GlutaMAvX™ Supplement (Gibco cat 31985088) supplemented with 5% FCS, 1 mM Sodium Pyruvate (Fisher Scientific) and IX MEM non-essential amino acids (Fisher Scientific) (cOPTI-MEM) at 3.6×107 cells per T225 flask in 45 ml of medium overnight to achieve confluency between 85% and 95% at the time point of transfection. The following morning, HEK293 Ts cells were transfected with second generation lentiviral packaging plasmids and transfer plasmid using Lipofectamine 3000 transfection reagent (Fisher Scientific cat 1.3000075). Briefly, 165 μl of Lipofectamine 3000 reagent was added to 5 ml of room temperature OPTI-M-TEM without supplements. Forty-two micrograms of Cas9 transfer plasmid, 30 μg of psPAX2 (addgene 12260), 13 μg of pMD2 G (addgene 12259), and 145 μl of p3000 reagent were added to 5 ml of room temperature OPTI-MEM without supplements and mixed by gentle inversion. The plasmid and Lipofectamine 3000 mixes were combined, mixed by gentle inversion, and incubated for 15 min at room temperature. Following incubation, 20 ml of medium was removed from the T225 flask and the 10 ml transfection mixture was carefully added without detaching HFEK293T cells. After 6 hours, the transfection medium was replaced with 45 ml of cOPTI-MEM supplemented with IX ViralBoost (Alstern Bio cat VB100). Lentiviral supernatant was harvested 24 hours after transfection (first harvest) and replaced with 45 ml of fresh cOPTI-MEM. A second harvest was performed 48 hours after transfection. Immediately after collection, the media was centrifuged at 500g, 5 min, and 4° C. to clear cellular debris. Unless otherwise noted, Lenti-X-Concentrator (Takara Bio 631232) was added to the collected supernatant and lentivirus was concentrated following the manufacturer's instructions and resuspended in OPTI-MEM in 1% of the original culture volume without supplements. Lentiviral particles were subsequently aliquoted and frozen at −80C.

Flow cytometry

Aria 2, Aria 3 and Aria Fusion cell sorters (BD Biosciences) at the UCSF Parnassus Flow Core and the Gladstone Institute Flow Core were used for sorting. The Attune NxT flow cytometer (Thermo Fisher) and LSRFortessa X-20 (BD Biosciences) was used for flow cytometry. Antibodies used for flow eytonetric analyses and sorting are summarized in Table 9.

| Antigen Name | Target Species | Fluorochrome | Clone | Vendor |
|---|---|---|---|---|
| EGFR | Human | BV421 | EGFR.1 | BD |
| IL1R2 | Human | APC | 34141 | Thermo |
| IL1R2 | Human | FITC | 34141 | Thermo |
| GARP | Human | APC | 7B11 | Biolegend |
| IFN-gamma | Human | FITC | 4S.B3 | Biolegend |
| TNF-alpha | Human | APC | MAb11 | Biolegend |
| | | | MQ1- | |
| IL-2 | Human | Pacific Blue | 17H12 | Biolegend |
| IFN-gamma | Human | Pacific Blue | B27 | Biolegend |
| CD45 | Human | PE | H130 | Biolegend |
| B2M | Human | APC | 2M2 | Biolegend |
| CD45 | Human | AF488 | | Biolegend |
| CD4 | Human | PE | RPA-T4 | Biolegend |
| CD4 | Human | BV421 | 38261 | Biolegend |
| GARP | Human | PE | 7B11 | Biolegend |
| CD45 | Human | APC | H130 | Biolegend |
| IFN-gamma | Human | BV421 | B27 | Biolegend |
| | | | MQ1- | |
| IL-2 | Human | APC | 17H12 | Biolegend |
| CD4 | Human | FITC | 38261 | Biolegend |
| IFN-gamma | Human | BV605 | B27 | Biolegend |
| TNF-alpha | Human | BV421 | Mab11 | Biolegend |
| TNF-alpha | Human | BV711 | Mab11 | Biolegend |
| EGFR | Human | PE | AY13 | Biolegend |
| CD4 | Human | PE-Cy7 | 38261 | Biolegend |
| CD22 | Human | PerCp-Cy5.5 | HIB22 | Biolegend |
| CD45RA | Human | BV711 | HI100 | Biolegend |
| CD62L | Human | FITC | DREG-56 | Biolegend |
| CD4 | Human | BUV395 | SK3 | BD |
| CD8a | Human | BUV496 | SK1 | BD |

### Intracellular Cytokine Staining

Unless indicated otherwise, T cells were stimulated with ImmunoCult™ Human CD3/CD28/CD2 T Cell Activator (Stemeell Technologies cat 10990) with 6.25 μl per milliliter of culture media at 2×10⁶ cells/mi. One hour after restimulation, Golgi Plug protein transport inhibitor (BD Biosciences, cat 555029) was added at a 1/1000 dilution. Nine hours after addition of Golgi Plug, T cells were stained for surface antigens prior to fixation and subsequently processed for intracellular cytokine staining following BD Cytofix/Cytopernm™ kit (BD Biosciences cat 554714) instructions.

Genome-wide CRISPRa and CRISPRi screens

One day after activation, T cells from two human blood donors were infected with 2% v/v concentrated dCas9-VP64 lentivirus. Two days after activation, T cells were split into two populations and infected with 1% v/v (MOI-0.5) Calabrese Set A (addgene 92379) or 0.8% v/v (MOI ˜0.5) Calabrese Set B (addgene 92380) lentivirus. These two sets were independently cultured and processed in parallel until analysis. Three days following activation, fresh media with IL-2 (final concentration 500 TU/ml) and puromycin (final concentration 2 μg/ml) was added to bring cells to 3x 105 cells/mi. Cells were split two days later and fresh media with IL-2 was added to bring cells to 3×105 cells/mi-Two days later, fresh media without IL-2 was added to bring the concentration to 10⁶/ml. Eight days after initial activation, cells were harvested, centrifuged at 500g for 5 min and resuspended at 2x 06 cells/mi X-VIVO 15 without supplements. The following day, cells were restimulated and stained for FACS as described under “intracellular cytokine staining”, Over subsequent 2 days, cells were sorted at the Parnassus Flow Cytometry Core Facility (PFCC) into [L-21 and IL-2′ CD4+ T cell and IFN-1.l and IFN-thi CD4 T cell populations. Sorted cells were stored in EasySep Buffer (PBS with 2% FCS and 1 mM EDTA) overnight until genomic DNA isolation.

The same experimental procedure using T cells from the same donors was followed for the CRISPRi screens. T cells were infected with dCas9-mCherry-KRAB at 2% v/v and Dolcetto A (addgene 92385) and B (addgene 92386) sgRNA libraries at 10% v/v or 25% v/v unconcentrated virus, respectively (-0.5 M1 O).

Genornic DNA was extracted from fixed cells as described previously (44). Integrated sgRNA sequences were amplified as previously described (22), and sequencing libraries were subsequently agarose gel purified using NucleoSpin Gel and PCR Clean-up Mini kit (Machery Nagel cat 740609.50). Libraries were sequenced on a NextSeq500 instrument to a targeted depth of 100-fold coverage.

For the supplementary CD4′ T cell set of genome wide CRISPRa screens, CD4‘T cells were isolated from Leukopaks using magnetic negative selection (Stemcell Technologies, cat 17952) and subsequently stimulated as described under “Isolation and culture of human “T cells”. I cells were then cultured and infected with lentivirus as described for the primary CRISPRa screens above. For library lentivirus production, Calabrese Set A and Set B plasmid were mixed at equimolar ratios before transfection and the pooled lentiviral particles from both sets was used for transduction. CD4 flow cytometry staining on day 7 after T cell activation confirmed >98% purity. T cells were further processed and restimulated as described above. T cells were separately stained for IL-2, IFN-γ, or TNF-r for FACS. After our initial analysis, it appeared the IFN-γ screen was potentially under-sampled due to lower hit resolution than the other screens. To address this, additional fixed cells from the same experiment were stained and sorted as an additional technical replicate and then computationally merged (described below).

CRISPR screen analysis

Reads were aligned to the appropriate reference library using MAGeCK version 0.5.9.2 (45) using-trim-5 22,23,24,25,26,28,29,30 argument to remove the staggered 5′ adapter. Next, raw read counts across both library sets were normalized to the total read count in each sample and each of the matching samples across two sets were merged to generate a single normalized read count table. Normalized read counts in high versus low bins were compared using mageck test with-norm-method none, paired, and control-sgrna options, pairing samples by donor and using non-targeting sgRNAs as controls, respectively. Gene hits were classified as having a median absolute iog₂-fold change value greater than 0.5 and an FDR <0.05. For supplemental CD4′ screens, reads were aligned to the full Calabrese A and B library in a single reference file. For the supplemental CD4^{T} IFN-γ screen, which was sorted and sequenced as two technical replicates, normalized counts were averaged across technical replicates before analyzing with mageck test.

Gene set-enrichment analysis (GSEA)

Gene set-enrichment analysis was completed with the fgsea Bioconductor R package using default settings (46). KEGG pathways v7.4 were obtained from GSEA mSigDB http://www.gsea-msigdb.org!gsea/downloads.jsp. The KEGG NF-xB signaling pathway (entry hsa04064) was missing from this dataset and added manually from https://www.genome.jp/entry/pathway+hsa04064.

s-LDSC analysis

GWAS summary statistics were downloaded from the Price lab website (https://alkesgroup.broadinstitute.org/sumstats formatted/and https://alkesgroup.broadinstitute.org/UKBB/). LD scores were created for each screen (corresponding to a set of SNPs within 100 kb of genes identified as significant hits in each screen or their corresponding matched background sets) using the 1000G Phase 3 population reference. Each annotation's heritability enrichment for a given trait was computed by adding the annotation to the baselineLD model and regressing against trait chi-squared statistics using HapMap3 SNPs with the stratified LiD score regression package (47). Heritability enrichments were then meta-analyzed across immune or non-immune traits using inverse variance weighting. The sets of background genes were sampled from the set of all genes that were expressed in the control sgRNA, stimulated bulk RNA-Seq data. For each screen, the background genes were sampled to match the significant screen hits in number and based on deciles of gene expression. Immune traits used for analysis were: “Eosinophil Counf”, “Lrymphocyte Count”, “Monocyte Count”, “White Count”, “Autoimmune Disease All”, “Allergy Eczema Diagnosed”. “Asthma Diagnosed”, “Celiac”, “Crohn's Disease”, “Inflaimatory Bowel Disease”, “Lupus”, “Multiple Sclerosis”, “Primary Biliary Cirrhosis”, “Rheumatoid Arthritis”, “Type I Diabetes”, “Ulcerative Colitis”. Non-Immune traits used were: “Heel Tscore”, “Baldingl”, “Balding4”, “Bmi”, “Height”, “Type 2 Diabetes”, “Neuroticism”, “Anorexia”, “Autism”, “Bipolar Disorder”, “Depressive Symptoms”, “Fasting Glucose”, “ldl”, “Ldl”, “Triglycerides”, “Fasting Glucose”

Arrayed CRISPRa experiments For each gene chosen to target in follow up experiments, one sgRNA was chosen from the Calabrese library used in screens. The first sgRNAs (“_1”) were manually chosen for consistent log₂ fold-change observed in both donors. The second sgRNA (“2”) was picked from the hCRISPRa-v2 genome-wide library (48), choosing the top ranked sgRNA not present in Calabrese libraries for each gene. sgRNAs were cloned into the pXPR 502 vector as described in the plasmid section.

Primary human T cells were transduced with 2% v/v inCherry-2A-dCas9-VP64 lentivirus (pZR112) 1-day post-activation. The following day (day 2), the dCas9-VP64 transduced cells were split into 96-well flat-bottom plates, avoiding edge wells, and transduced with a different sgRNA lentivirus in each well (5% v/V). One day after sgRNA transduction, fresh medium was added with IL-2 (500 IU/ml) and 2 μg/ml puromycin (final culture concentrations). Cells were passaged 2 days later, adding fresh medium with 500 IU/ml of IL-2 and maintaining a concentration of 3×10⁵ to 1 ×10⁶ cells/ml with 96-well plates copied as needed to maintain this concentration. On day 8, cells from copied plates were pooled and samples were counted. Cells were pelleted and resuspended at a concentration of 2×10⁶ cells/ml in fresh X-VIVO-15 without additives. On day 9, cells were restimulated with anti-CD3/CD28/CD2 ImmunoCult T Cell Activator (as described in “Intracellular cytokine staining”) or left resting.

### RT-gcR

T cells were prepared as described under Arrayed CRISPRa experiments. Seven days post sgRNA transduction 100,000 T cells per well were pelleted at 500g, 5 mins, and 4° C. Cells were lysed and RNA was extracted using Quick-RNA 96 kit (Zymo Research), following manufacturer's protocol, skipping the option of in-well DNase treatment. DNase treatment and cDNA synthesis were subsequently completed with Maxima First Strand cDNA Synthesis Kit for RT-qPCR, with dsDNase (Thermofisher Scientific). qPCR was performed with PrimeTime PCR Master Mix (Integrated DNA technologies) and PrimeTime qPCR probe assays (Integrated DNA Technologies, list of probes used in Table 10) on an Applied Biosystemns Quantstudio 5 real-time PCR system. Data was analyzed using the deltaDeltaCt method. The mean Ct values of two housekeeping genes, PPIA and (GUSB, to calculate the deltaCt, and the mean deltaCt of non-targeting controls to calculate deltaDeltaCt.

TABLE 10

| Primer 1 | Primer 2 |
|---|---|
| GGA AGT AGA ATG TGC CTG GAT | GTA AGC AGG AAG AGA AGC CA |
| GAG ACC ACA GTT AGA GAA CCA C | TCT TGC TAT TGA CCG ATG CTT |
| CGA CAG TTC AGC CAT CAC TT | GCA ACA AAA AGA AAC GAG ATG AC |
| CAC TGT TTT TCC AAG ACC TCA | TTC CTG CTA TGA TTT TCT CCC A |
| CTC CAG AGG TTT GAG TTC TTC T | AAA CTC ACC AGG ATG CTC AC |
| GCG AAG AGA GCC ACT TCT G | GTG TAC TTG CTG ATC AAC TGC |
| CTG GTG TTG CCT CTT GTG AT | AGT GTC AGT GGT GTT GGC |
| CAG CTT GGA CAC TGG ATC TC | CCT GCA CGG CTA CAT TGA G |
| AAG CAG ACG GAA AGT GAG G | GTG GCT ATG GTT GGA GGT C |
| GAT CCT CAA GTA CTT TCA GCC A | CAC TGC CGA GGA ATG AAG AG |
| TGA TCT CCA AGT CTG TCT GC | GAG TCC TTT CGT TTC CAG CA |
| ACA ACT TCG TGC ACT CCA | CAG CCT CTG CCT CAA TGG |
| TCT GCG TGA ATC CTA GAT TTC TG | GCT GAG AAG TTG GAA GTG GAA |
| GCC GAA CTT CTC ACA GCA | CTT AAC AAC CTG CTA CCC CAT |
| GTT TTT GAT CCA GAC CCA GAT G | GCC CAT TAT TCA GAG CGA GTA |
| CAA GAC TGA GAT GCA CAA GTG | GTG GCG GAT TTG ATC ATT TGG |

Probe
/56-FAM/CCA CAG ATC/ZEN/AGA AAC CCG ATG AAG GC/3IABKFQ/
/56-FAM/TAA AGC TGT/ZEN/AGC CCG TTG CCT GC/3IABKFQ/
/56-FAM/TCG GTA ACT/ZEN/GAC TTG AAT GTC CAA CGC/3IABKFQ/
/56-FAM/TCT ACC TCT/ZEN/GAC TGT GAT ATT TTT GTG TTT AAA GTC T/3IABKFQ/
/56-FAM/TTA CAT GCC/ZEN/CAA GAA GGC CAC AGA/3IABKFQ/
/56-FAM/TGT AAC ACC/ZEN/CCA GAC CCC TCG AA/3IABKFQ/
/56-FAM/AGG TGT GCG/ZEN/GGC TCA GGA T/3IABKFQ/
/56-FAM/CAC TTC CGC/ZEN/ATC TGC CCG TG/3IABKFQ/
/56-FAM/TGT AGC AGC/ZEN/TGA TCC GAG CCT AGA/3IABKFQ/
/56-FAM/CGA GGG TGG/ZEN/AGG CCT GAA TTT TGA/3IABKFQ/
/56-FAM/TGC TTC TCT/ZEN/CTC TGT CTT CGG GTG A/3IABKFQ/
/56-FAM/ACG GTG TTC/ZEN/TGT TTC TCC TGG CA/3IABKFQ/
/56-FAM/AGA GAG CAG/ZEN/ACT GGA AGA AAA CAG TGG/3IABKFQ/
/56-FAM/CAG ATG TCC/ZEN/CAG TTC CTG TGC CTT/3IABKFQ/
/5Cy5/TGC AGG GTT TCA CCA GGA TCC AC/3IAbRQSp/
/5Cy5/AAT TCA CGC AGA AGG AAC CAG ACA GT/3IAbRQSp/

cDNA experiments

One day after activation, T cells were transduced with the 1G4 TCR lentivirus recognizing the NY-ESO-1 antigen or non-transduced for immunocult assay. One day later, cells were transduced with the transgenes in cDNA format. Three days after initial activation, puromycin was added to obtain a final concentration of 2 pig/ml along with fresh X-VIVO 15 media with 500 IU/ml of HL-2 and further cultured and expanded analogous to the genome wide CRISPR screens, Nine days after initial activation, T cells were centrifuged and resuspended at 2×10⁶ cells/ml in X-Vivo 15 without supplements. On the same day, 1G4 TCR expression was assessed by flow cytometry following dextrainer staining (Immudex cat WB3247-PE) to ensure even expression across different cDNA constructs. The following day, T cells were restimulated with either 6.25 μl per milliliter of Immunocult or NALM6 cells at an effector-target ratio of 1:2 for 1G4 TCR-transduced cells. Cells were further processed as described under “intracellular cytokine staining”. CD22 was used as a marker for NALM6 cells to discriminate them from T cells in the coculture. Overexpression of OTUD7B cDNA together with the IG4 TCR (but not alone) caused toxicity and was therefore excluded from analyses. Two donors were excluded from the 1G4 TCR assay due to poor TCR transduction.

Cytokine Luminex assay

T cells were prepared as explained under “Arrayed CRISPRa experiments.” On day 9 after activation, T cells at a concentration of 2×10⁶ cells/ml were restimulated with ImmunoCult™ Human CD3/CD28/CD2 (Stemcell Technologies cat 10970) at 6.25 μl per milliliter. Twenty-four hours after restimulation, supernatant was collected and frozen at −20° C. Following a serial pilot titration, cytokine analyses were performed at a 1/200 dilution by Eve Technologies with the Luminex xMAP technology on the Luminex 200 system (Luminex). To remove very-low-expressed cytokines for downstream analysis, any group where three of four donors had undetectable cytokines, the cytokine was removed. Additionally, the sgfLIRl-1-Donor 4 measurement for IL-I a was removed manually, as this was an extremely high outlier.

Bulk RNA-seq sample preparation

FOXQI and non-targeting sgRNA control primary human T cells from four donors were transduced and expanded as described in “Arrayed CRISPRa experiments” section. On day 8, mCherry+CD4+populations were sorted and resuspended in X-VIVO-15 without additives at 2×10⁶ cells/mil. On day 9, cells were restimulated with 6.25 μl per milliliter of anti CD3/CD28/CD2 ImmunoCult™ or left unperturbed for resting (non-stimulated) condition. Twenty-four hours later, cells were lysed for RNA.

RNA was purified using Quick-RNA Microprep kit (Zymo Research) without the optional in-well DNase treatment step. Purified RNA was treated with TURBO DNase (Thermofisher Scientific) to remove potential contaminating DNA. RNA was subsequently purified using RNA Clean & Concentrator-5 kit (Zymo Research). RNA quality control was performed using an RNA ScreenTape assay (Agilent), with all samples having an RNA integrity number >7. RNA-seq libraries were prepared using the Illumina Stranded mRNA Prep kit, with 100 ng of input RNA. Libraries were sequenced using paired-end 72-bp reads on a NextSeq500 instrument to an average depth of 3.2×10⁷ clusters per sample.

Bulk RNA-seg data analysis

Adapters were trimmed from fastq files using cutadapt version 2.10 (49) with default settings keeping a minimum read length of 20 bp. Reads were mapped to the human genome GRCh38 keeping only uniquely mapping reads using STAR version 2.7.5b (50) with the following settings “-outFilterMultimapNmax 1”. Reads overlapping genes were then counted using featureCounts version 2.0.1 (51) with the following settings “-s 2” and using the Gencode version 35 basic transcriptome annotation. The count matrix was imported into R. Only genes with at least I count per million (CPM) across at least four samples were kept. TMM normalized counts were used for heatmaps. Differentially expressed genes between FOXQI overexpression and control samples were then identified using limna version 3.44.3 (52) while controlling for any differences between donors. Significant differentially expressed genes were defined as having an FDR-adjusted P-value <0.05.

### Perturb-seq Library Design and Cloning

The CRISPRa Perturb-seq target genes were selected from the primary IL-2 and IFN-γ CRISPRa screen results. First, genes that had a significant fitness defect removed from the gene list. Next, genes were ranked by median sgRNA log₂-fold change and the top ranked, not previously selected gene, was picked in the following order: (1) IL-2-positive hit, (2) IFN-γ positive hit, (3) IL-2-positive hit, (4) IFN-γ-positive hit, and (5) IL-2- or IFN-γ-positive hit (alternating each round), such that positive hits outnumbered negative hits at a 4:1 ratio. Only hits that were significant (FDR<0.05) were selected in each round. The one exception was TCF7, which was added manually as we considered it worthwhile to analyze due to its known effects on T cell function. To select sgRNAs, the top two enriched sgRNAs by log₂ fold-change in the screen for which the gene was selected were used. The library was ordered as pooled single stranded oligos, PCR amplified, and cloned into the CRISPRa-SAM direct-capture design I cloning vector (pZR158).

### Perturb-seq Sample Preparation and Sequencing

Bulk CD3⁺ primary human T cells from two donors were transduced and cultured as described in the “⁴Genome-wide CRISPRa and CRISPRi screens” section, except library transduction was completed at lower MOI, of 0.3. Cells in the stimulated condition were stimulated with 6.25 μl per milliliter of anti-CD3/CD28/CD2 immunocult. Twenty-four hours later, cells from both the stimulated and non-stimulated condition were sorted for mCherry™ (marking dCas9-VP64). Sorted cells were processed to single-cell RNA-seq and sgRNA sequencing libraries by the Institute for Human Genetics (IHG) Genomics Core using Chromium Next GEM Single Cell 3′ Reagent Kit v3.1 with Feature Barcoding technology for CRISPR screening, following manufacturer's protocol, Before loading the Chromium chip., sorted cells from two blood donors were normalized to 1000 cells/μl and mixed at a 1:1 ratio, for each condition. Twenty microliters of cell suspension was loaded into four replicate wells per condition, for a total 80,000 cells loaded per condition. Final sgRNA sequencing libraries were further purified for the correct size fragment by 4% agarose F-Gel EX Gels (ThermoFisher Scientific) and gel extracted. Libraries were sequenced over two NovaSeq S4 lanes (2 stimulated wells, two non-stimulated wells per lane), at a 2:1 molar ratio of the gene expression libraries to sgRNA libraries.

Perturb-seq Analysis Alignments and count aggregation of gene expression and sgRNA reads were completed with Cell Ranger version 6.1.1. Gene expression and sgRNA reads were aligned using cellranger count, with default settings. Gene expression reads were aligned to the “refdata-gex-GRCh38-2020-A” human transcriptome reference downloaded from 1Ox Genomics. sgRN A reads were aligned to the Perturb-seq library using the pattern (BC)GTTTAAGAGCTATG. Counts were aggregated with cellranger aggr with default arguments. To assign sgRNAs to cells, cellranger count output files “protospacer_calls per_cell.csv” were used, filtering out droplets with >1 sgRNA called, returning a median of 133 sgRNA UMIs in sgRNA singlets. For increased stringency, only droplets with >5 sgRNA UMIs were used in further analysis. Cell donors were genetically demultiplexed using Souporcell (53) (https:%/github.corn/wheaton5/souporcell). The input for each run was the bam file and barcodes.tsv file from the cellranger count output, and the reference fasta. Donor calls across wells were harmonized using the vcf file outputs from Souporcell using a publically available python script (https://github.cornihyunminkang/apigenome/blob/master/scripts/vcf-match sample-ids).

Gene expression data were imported and analyzed in R with Seurat version 4.0.3 ReadlOX function (54). Cells were initially quality filtered for percent mitochondrial reads <25%, number of detected RNA features >400 and <6000, removing 4% of cells.

After filtering, we recovered a median of 401 cells per sgRNA target gene per condition (median of 127 sgRNA unique molecular indices (UMIs) per singlet), and ˜2000 cells with no-target control guides per condition. Four sgRA targets (HELZ2, TCF7, PRDM1, and IRX4) were removed from downstream analysis due to low cell counts (<100).

Gene-expression counts were normalized and transform-ed using the Seurat SCTransform function (55), with the following variables regressed: percent mitochondrial reads, S-phase score, and G2M-phase score, performing the regression as described on the Satija Lab website (https://satijalab.org/seurat/articles/cell_cycle_vignette.html). Normalized and transformed counts were used for all downstream analysis. To call CD4′ and CD8′ T cells, a CD4/CD8 score for each cell using following formula was used: log₂(CD4/mean(CD8A, CD8B)), with a score <−0.9 called as a CD3′ cell, and >1.4 called a CD4 cell.

For both restimulated and resting conditions, UMAP reduction was performed with dimensions 1-20, and otherwise default settings of the RunUMAP Seurat function. For clustering, FindClusters was run using algorithm 3, and resolution 0.4 for restimulated, and 0.5 for resting condition. Two clusters in the restimulated condition were manually merged to form “Cluster 2: Negative Regulators”. The merged clusters showed highly similar gene expression patterns, with one cluster containing the bulk of cells containing negative regulator sgRNAs, and the other cluster containing sgRNAs targeting the negative regulator, MUC1. Cluster trees shown were generated using the Seurat BuiidClusterTree function with default arguments. For pseudobulk differential expression analyses the Seurat FindM/larkers function was used with the default method, Wilcoxon Rank Sur test.

To generate the T cell activation score, pseudobulk differential expression analysis was first performed on restimulated versus resting no-target control sgRNAs and log 2-fold change outputs were used as gene weights. Only genes with an absolute log-fold change >0.25 and which were detected in 10% of restimulated or resting cells were used for gene weights. For a given cell, the activation score is calculated as sum(GE x GW/GM), where GE is a gene's normalized/transformed expression count, GW is the gene's weight, and GM is the gene's mean expression in no-target control cells (to correct for differential levels of baseline expression).

Statistical analysis

All statistical analyses were performed in R version 4 0.2, unless otherwise noted. In order to deal with ties in non-parametric tests, Mann-Whitney U tests were performed using the wilcox_test function of the Coin R package (version 1.4-1), with default arguments. For q-value based rnultiple comparison correction, the R qvalue package (version 2 20.0) was used with defatilt arguments.

### Results

Genome-wide CRISPRa screens identify regulators of IL-2 and IFN-γ production in T cells

To enable scalable CRISPRa in primary human T cells, we developed an optimized high-titer lentiviral production protocol with a minimal dCas9-VP64 vector (pZRI12), allowing for transduction efficiencies up to 80%. A second-generation CRISPRa synergistic activation mediator (SAM) system (22, 23) induced robust increases in target expression of established surface markers. Next, we scaled up our platform to perform pooled genome wide CRISPRa screens targeting >18,800 protein coding genes with >112,000 sgRNAs (22). We used fluorescence-activated cell sorting (FACS) to separate IL-2-producing CD4-T cells and IFN-,-producing CD8-T cells into high and low bins (FIG. 1A). Subsequent sgRNA quantification confirmed sgRNAs targeting LL-2 (IL2) and IFN-γ (IFNG) were strongly enriched in the respective cytokine high populations and non-targeting control sgRNAs were not enriched in either bin (FIG. 1B). Both CRISPRa screens were highly reproducible in two different human blood donors (FIGS. 1, C and D). Gene level statistical analysis of the IL-2 and IFN-, CRISPRa screens revealed 444 and 471 hits, respectively, including 171 shared hits (FIG. 1E). Thus, CRISPRa screens provide a robust platform to discover gain-of-function regulators of stimulation-dependent responses in primary cells.

CRISPRa hits included components of the TCR signaling pathway and T cell transcription factors. Activation of TBX2I (encoding T-bet), which promotes both memory CDS⁺ T cell and CD4+ T helper I (Th1) cell differentiation (24-26), selectively enhanced the signature type I cytokine IFN-γ (FIG. 1E). By contrast, sgRNAs activating GATA3, which promotes type II differentiation by antagonizing T-bet (25, 27), had opposite effects (FIG. 1E). Overexpression of members of the proximal TCR signaling complex, such as VAV1, CD28, LCP2 (encoding SLP 76), and LAT (28, 29) reinforced T cell activation and were enriched in both cytokine-high bins. Conversely, negative TCR signaling regulators MAP4KI and SLA2 were depleted in these bins (FIGS. 1, B and E) (30, 31). Thus, CRISPRa identifies critical “bottlenecks” in signals leading to cytokine production.

Complementary CRISPRa and CRISPRi screens comprehensively reveal circuits of cytokine production in T cells

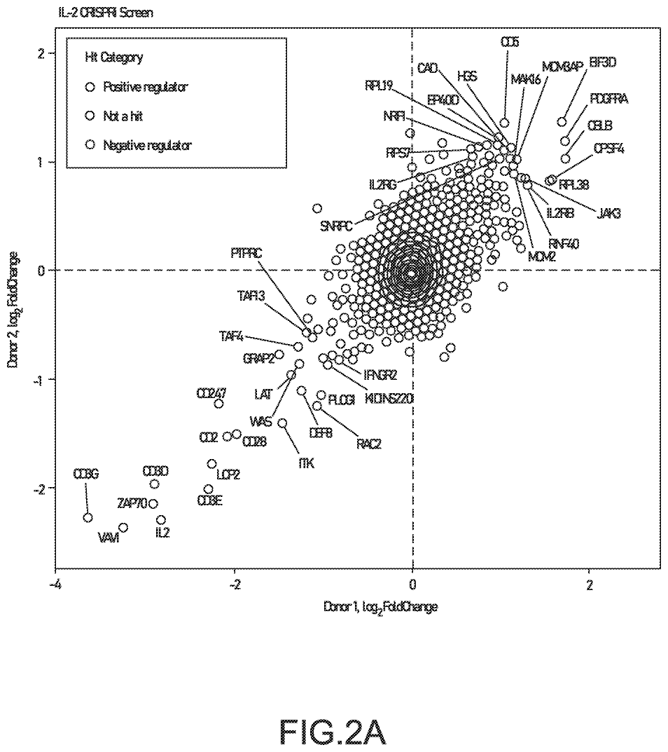

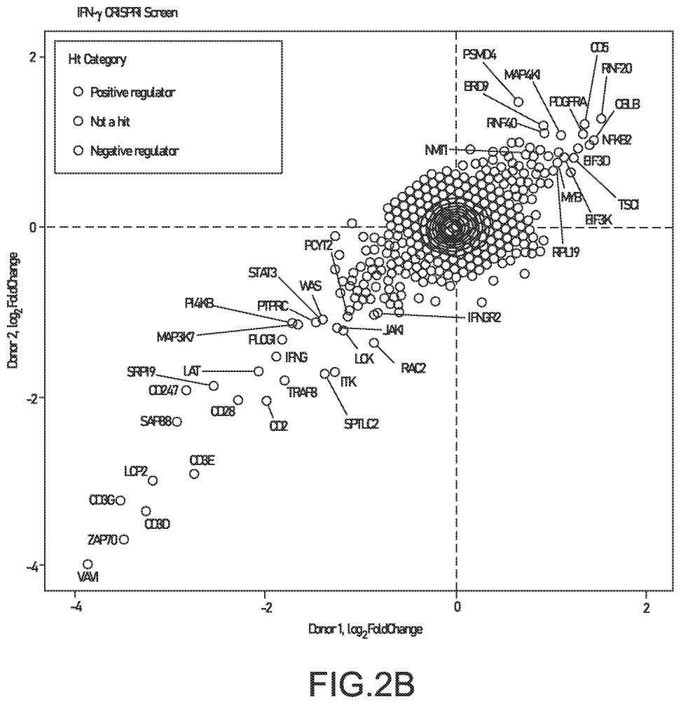

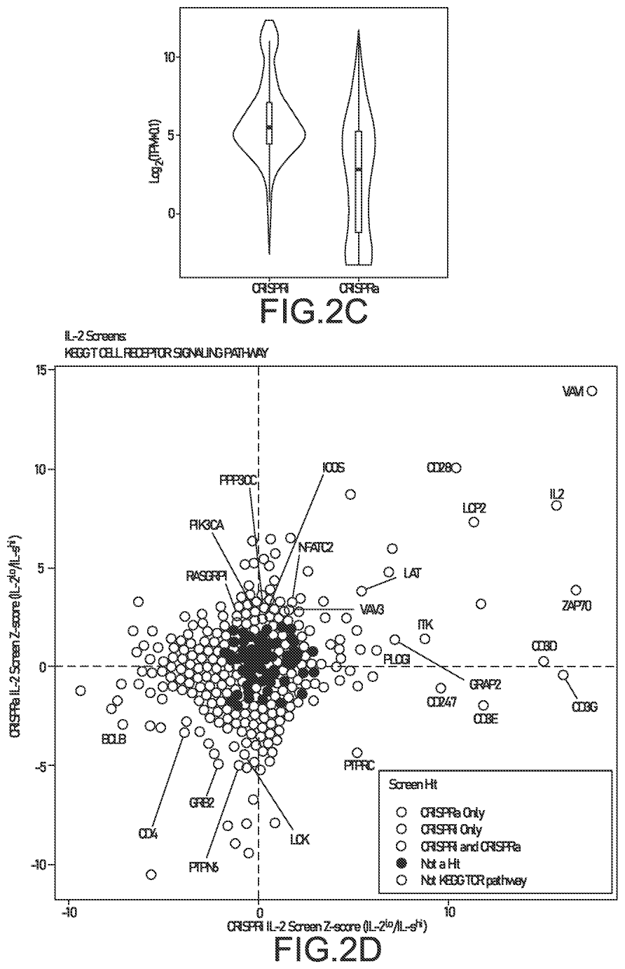

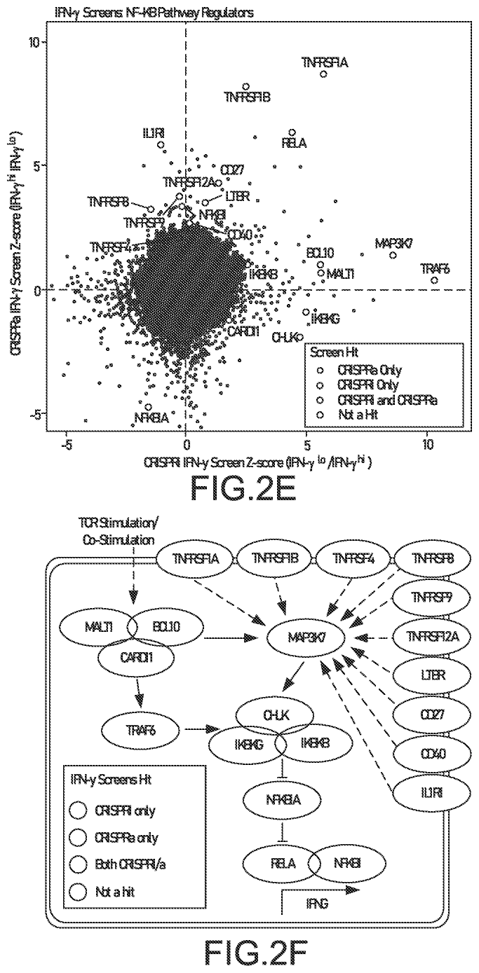

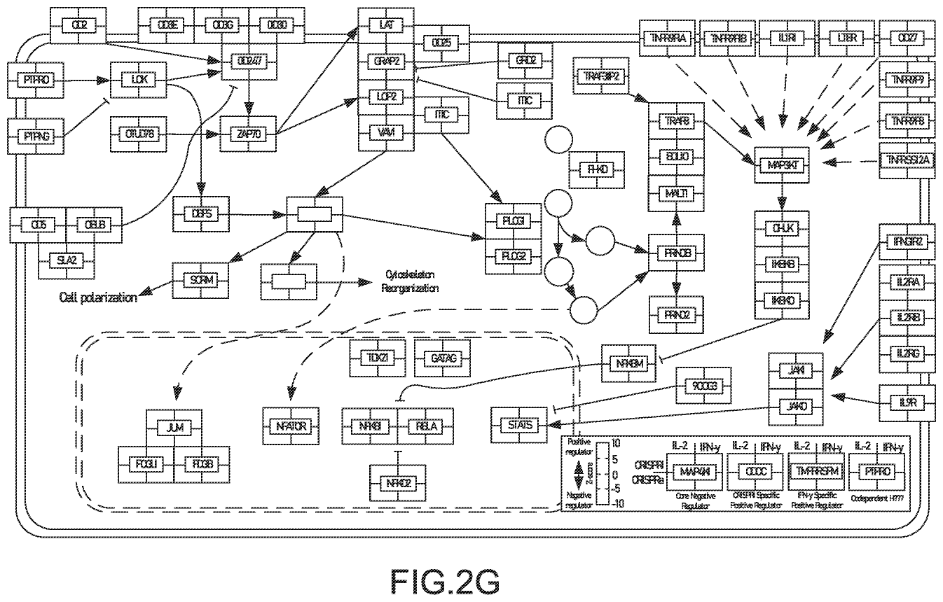

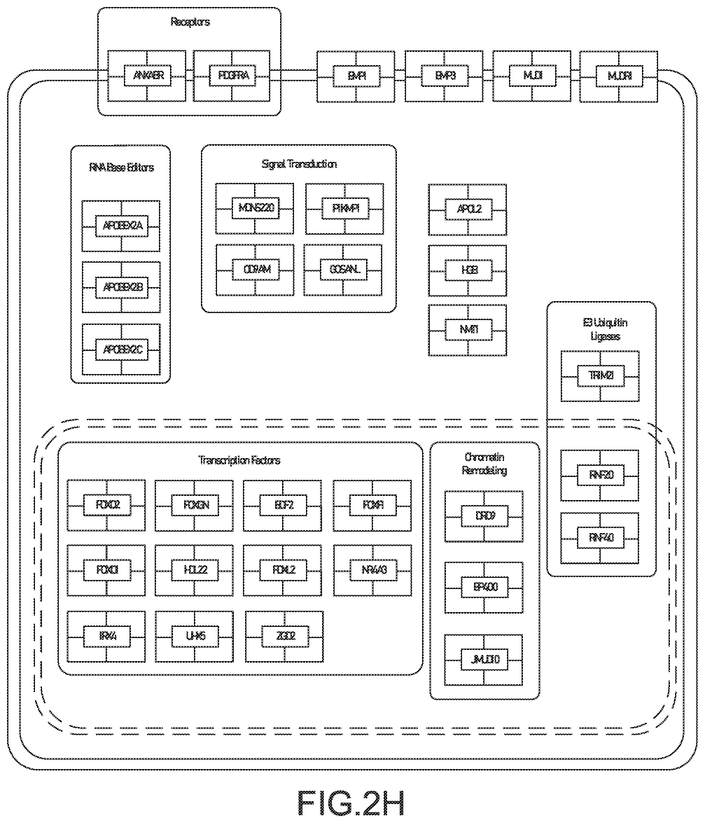

CRISPRa screens were effective in identifying limiting factors in cytokine production, but they could miss necessary components that would only be identified through loss-of-function studies. We therefore performed reciprocal geriome wide CRISPRi screens, adapting our optimized lentiviral protocols (FIGS. 2, A and B) Drop out of gold-standard essential genes (32) and reproducibility across two human donors confirmed the screen quality. The CRIfSPRi fL-2 and IFN-γ screens identified 226 and 203 gene hits, respectively, including 92 shared hits (FIGS. 2, A and B). As expected, the CRISPRi hits were biased towards genes with high mRNA expression including members of the CD³ complex, whereas CRISPRa additionally identified regulators that are expressed either at low levels or not at all in T cells under the screened conditions. (FIGS. 2, C and D). For example, PIK3AP1 and ILlRI were expressed at low levels under the screened conditions (FIG. S7A). They are potentially inducible in some T cell contexts however were detected as hits by CRISPRa but not CRISPRi.

The power of coupling activation and interference screening was exemplified further by the identification of two IFN-γ-regulating circuits. CRISPRi screens identified components of the NF-κB pathway that are required for IFN-γproduction (and to a lesser extent IL-2 production). CRISPRi detected a circuit of T cell stimulation signaling through MALTI, BCLI0, TRAF6, and TAKI (encoded by MAP3K⁷) to the inhibitor of NF-κB complex (IcB complex, encoded by CHUK, IIKBKIB, and IKBKG) that promotes IFN-γ production (FIGS. 2, E and F). By contrast, CR1SPRa revealed a set of positive IFN-γ regulators that included members of the tumor necrosis factor receptor superfamily (TNFRSF) and ILiR1. These regulators also signal through NF-κB, even though they are not individually required and therefore not detected by CRISPRi (FIGS. 2, E and F). Thus, CRISPRa and CRISPRi complement each other for the comprehensive discovery of functional cytokine regulators. To gain insights into functional pathways enriched across CRISPRi and CRISPRa screens, we completed gene set enrichment analysis (GSEA) of KEGG pathways, identifying multiple immune-related pathways as enriched across screens. Furthermore, we analyzed data from numerous genome-wide association studies (GWAS) to ask if the heritability of complex immune traits was enriched in genomic regions harboring our screen hits by stratified linkage disequilibrium score (s-LDSC) regression. Both CRISPRi and CRISPRa regulators of IFN-γ and CRISPRa regulators of IL.-2 were in regions enriched for immune trait heritability compared to non-immune traits or an expression matched background set. Thus, these forward genetic screens may serve as a resource to help prioritize candidate functional genes in genomic regions associated with complex immune diseases.

We next completed integrative analyses of gene hits across CRISPRa and CRISPRi screens for both cytokines. We found that a handful of genes were identified across all screens (e.g., ZAP70 as a positive regulator and CBLB as a negative regulator), representing core regulators of stimulation-responsive cytokine production in T cells. The majority of hits however were either cytokine-(IL-2 in CD4+ T cells or IFN-γ in CD8′ T 30 cells) or perturbation-(activation or interference) specific. For a few target genes including PTPRC (CD45), CRISRPa and CRISPRi both influenced cytokine production in the same direction, suggesting that for some genes activation and interference both impair optimal levels. The striking overlap in regulators between IL-2 in CD4⁺ T cells and IFN-γ in CD8-T cells led us to perform additional genome-wide CRISPRa screens for IL-2, IFN-γ, and TNF-α in CD4′ T cells, allowing for direct comparisons of type 1 cytokine regulators in CD4^{T} T cells. Many of the strongest positive (e.g., VA V1, CD28, and LCP2) and negative hits (e.g., MAP4KI, LAT2, and GRAP) overlapped across all CRISPRa screens, likely representing core regulators of type I cytokine production in response to stimulation/costimulation. Additionally, these screens identified hits that could potentially increase or decrease individual cytokines selectively. Thus, CRISPRi and CRISPRa hits reveal both core and context-specific regulators of cytokine production.

We used our integrated dataset combined with literature review to build a high-resolution map of tunable regulators of signal transduction pathways leading to cytokine production (FIG. 2G). This included calcium pathway signaling genes (e.g., PLCGI, PLCG2, PRKCB, PRKD2, and NFATC2), and cytokine signaling genes (e.g., STAT3, JAKI, JAK3, and SOCS3), the latter suggesting feedback circuits among cytokine signals. In particular, CRISPRa identified regulators absent from previous literature (e.g., APOBEC3A/D/C, FOXQI, and EMIPI) (FIG. 2H), underscoring the need for gain-of-function screens for comprehensive discovery. Thus, CRISPRa and CRISPRi screens complement one another to map the tunable genetic circuits controlling T cell stimulation-responsive cytokine production.

Arrayed characterization of selected CRISPRa screen hits

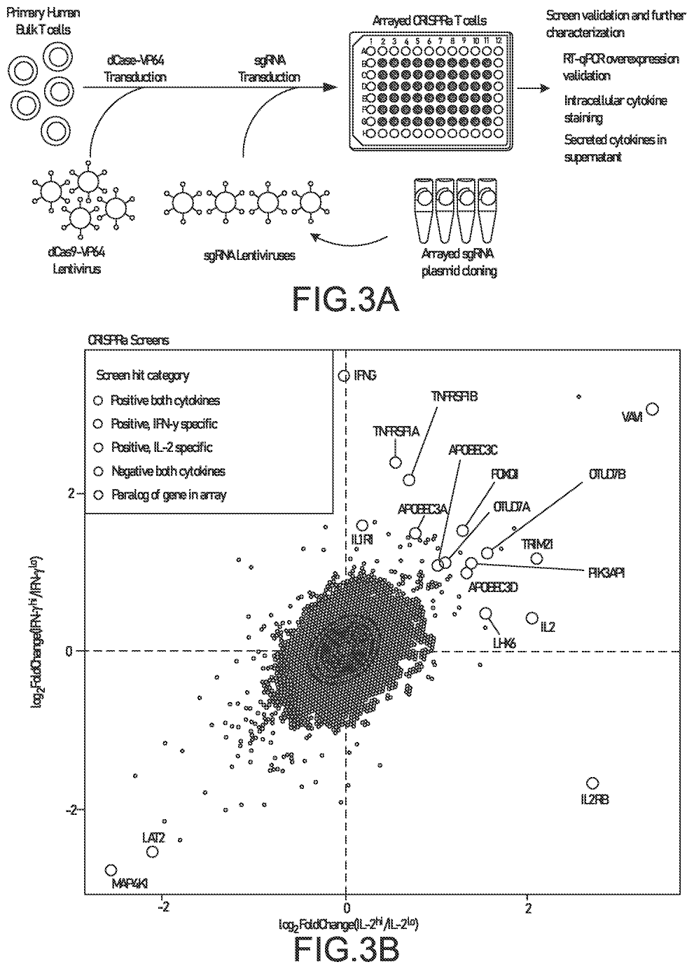

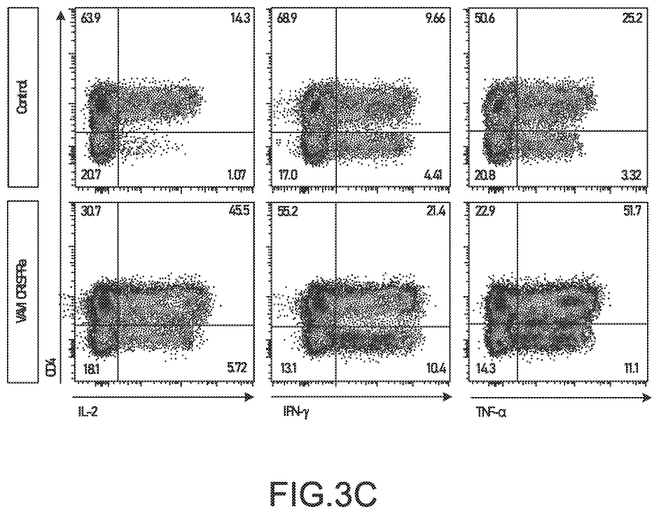

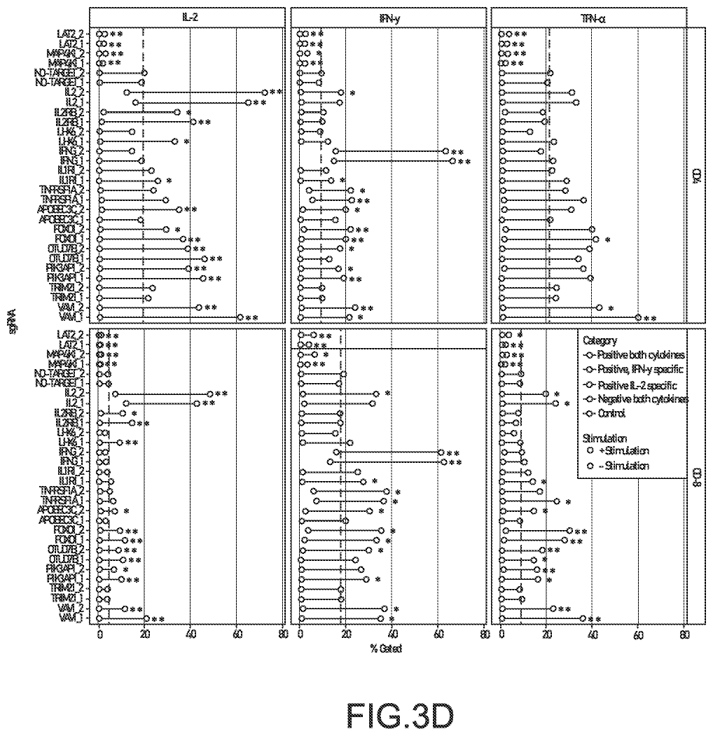

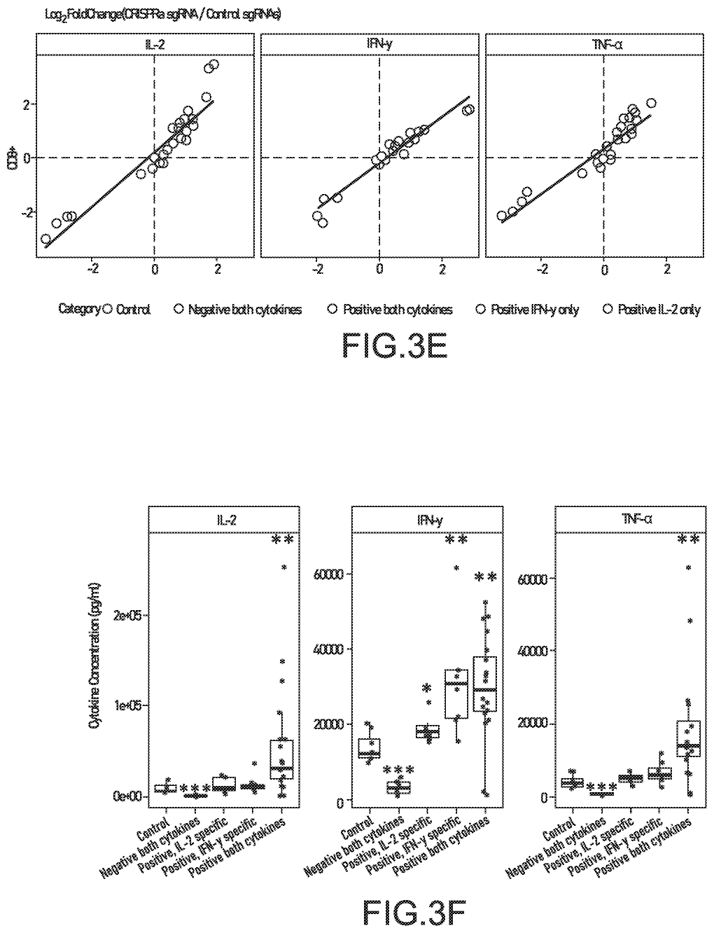

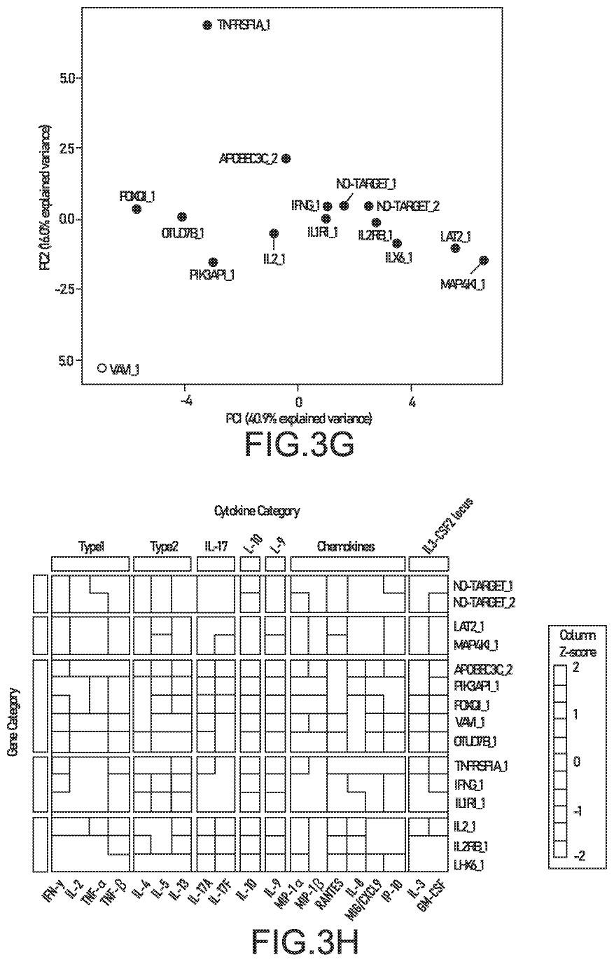

We next performed arrayed CRISPRa experiments for deeper phenotypic characterization of screen hits (FIG. 3A). We selected 14 screen hits (from different screen categories) (FIG. 3B) including the established regulators VAV1, MAP4K1, and positive controls IL2 and IFNG. Notably, we included genes with relatively low expression in T cells under our experimental conditions, FOXQ1, IL1 R1, LHX6, and PIK3API. First, we validated that selected sgRNAs increased the expression of target gene mRNA. Next, we assessed IL-2, IFN y, and TNF-α by intracellular staining in both CD4- and CD8′ T cells. Thirteen of 14 target genes caused significant changes in the proportion of cells positive for the relevant cytokine(s), with at least one sgRNA (FIG. 3, C and D). Furthermore, we observed effects on both IL-2 and IFN-γ double- and single-positive populations. With the exception of TNFRSF1 A (and I1L2 or IFNG), positive regulators did not cause spontaneous cytokine production without stimulation (FIG. 3D).

Although IL-2 was screened in CD4+ T cells and IFN-γ in CD8⁺ T cells, CRISPRa sgRNA effects were highly correlated across both lineages (FIG. 3F). We also assessed T cell differentiation and observed that FOXQ1 and TNFRSFIA significantly decreased the percentage of CD62L-cells, indicating a shift towards effector T cell states as a potential mechanism. Thus, these studies validate the pooled CRISPRa screens and begin to characterize cytokine production and cell differentiation states promoted by activation of key target genes.

We next tested if genes identified by CRISPRa could also regulate cytokines when overexpressed as cDNA transgenes, because continuous expression of CRISPRa would present challenges in cell therapies due to Cas9 immunogenicity (33). cDNA transgene overexpression of CRISPRa hits affected cytokine production in T cells stimulated with antibodies or antigen-positive cancer cells. Thus, this strategy could potentially be used to implement CRISPRa discoveries in engineered T cell therapies.

We next assessed how individual CRISPRa perturbations reprogram cytokine production by measuring a broad panel of 48 secreted cytokines and chemokines, 32 of which were detected in control samples. After confirming that the effects on IL-2, IFN-γ, and TNT a measurements were consistent with intracellular staining (FIG. 3F), we performed principal component analysis (PCA) and hierarchical clustering on all cytokines. We observed sgRNA categorical grouping consistent with that observed in the screens, with sgRNAs targeting genes identified as regulators of both cytokines causing broad increases or decreases in cytokine concentration (FIG. 3G). Notably, there were distinct patterns in the classes of cytokines increased by different regulators (FIG. 3H). VAV1 and FOXQI (a transcription factor that has not been well characterized in T cells) led to preferential increases in type 1 signature cytokines and dampened type 2 cytokines. Surprisingly, OTUDB, a positive regulator of proximal TCR signaling (34), had a distinct effect and increased type 2 cytokines. We next asked if modulations in the secretome correlated with transcriptional control of the corresponding genes. Taking FOXQ1 as an example, we performed bulk RNA-seq on FOXQI and control sgR^{N}A CD4-T cells and found it correlated highly with the secretorne effects. Thus, the identified regulators may not only modulate TCR stimulation and signaling, but also tune the T cell secretome towards specific signatures.

CRISPRa Perturb-seq characterizes the molecular phenotypes of cytokine regulators

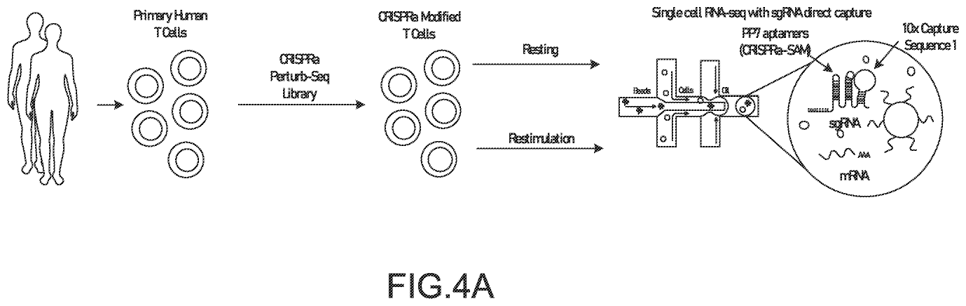

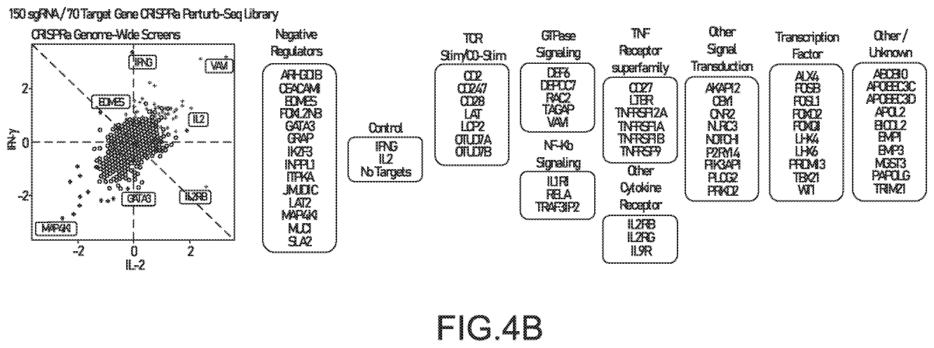

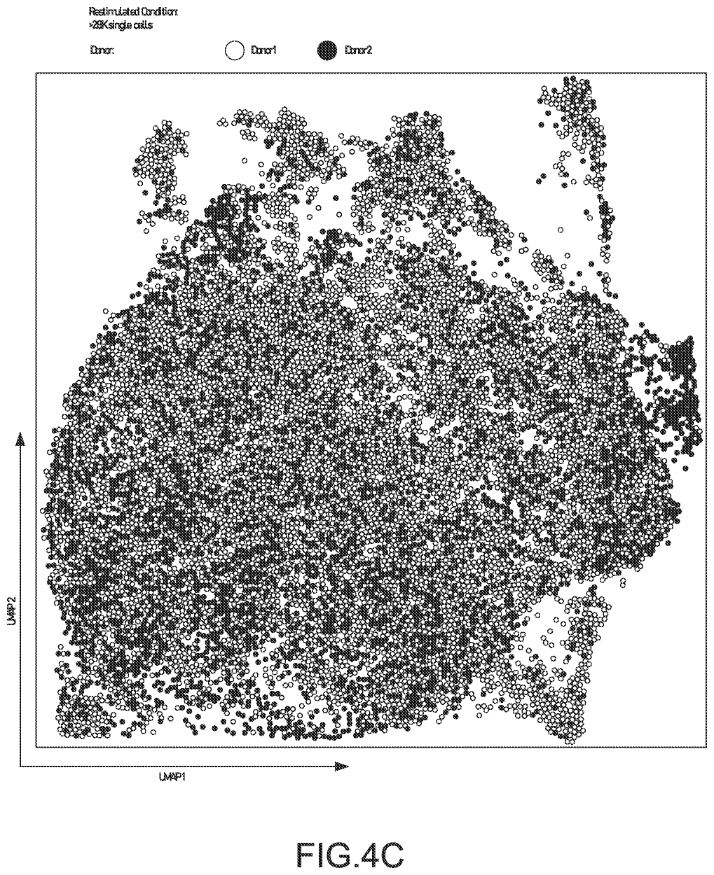

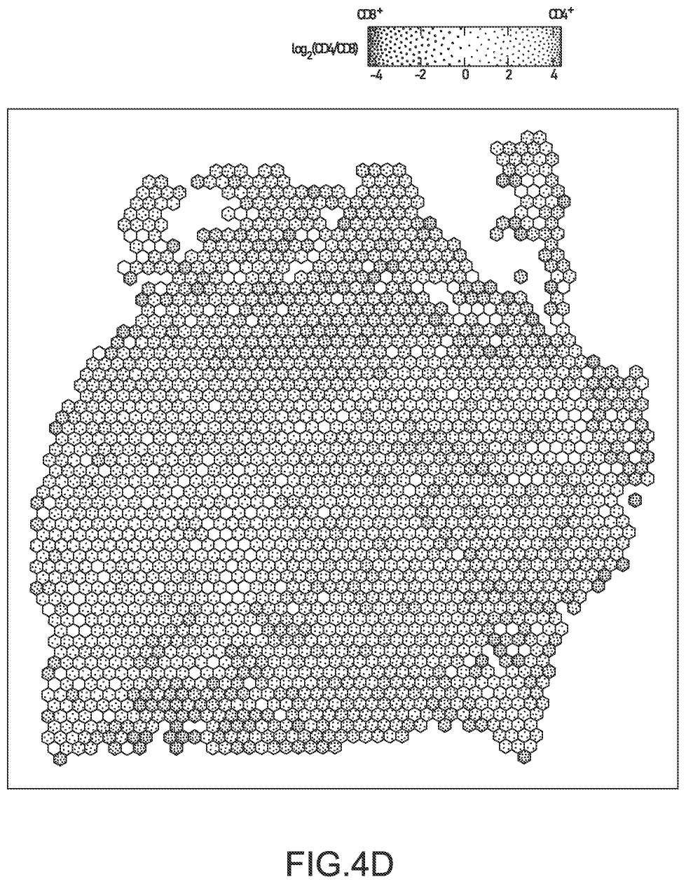

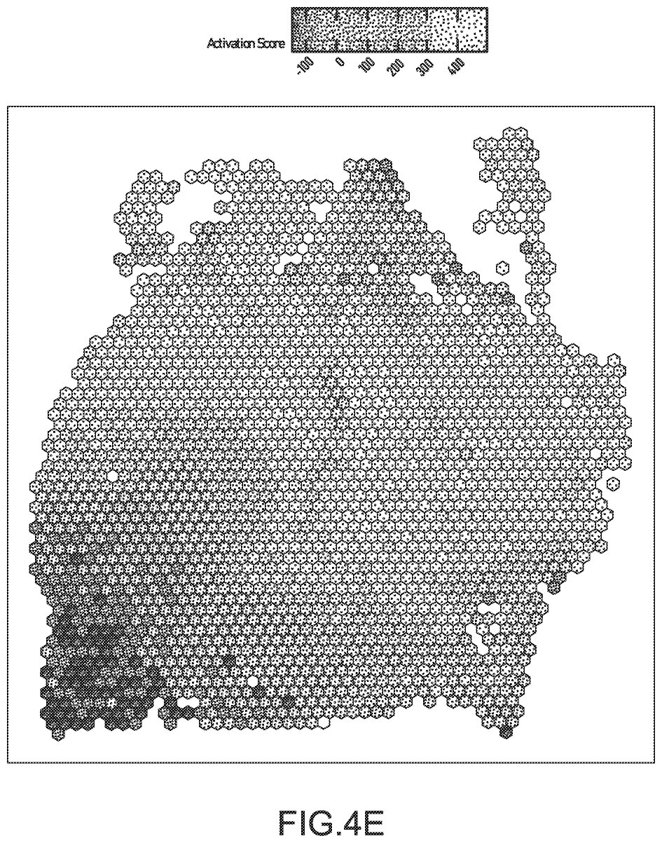

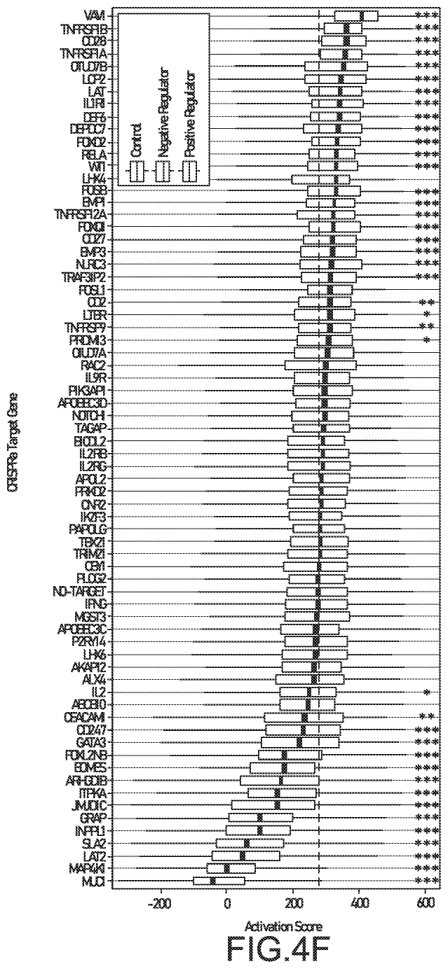

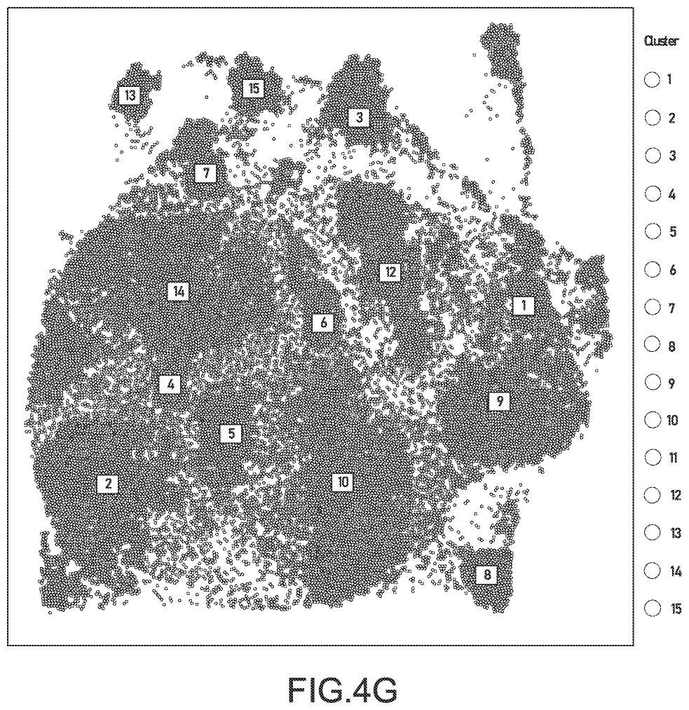

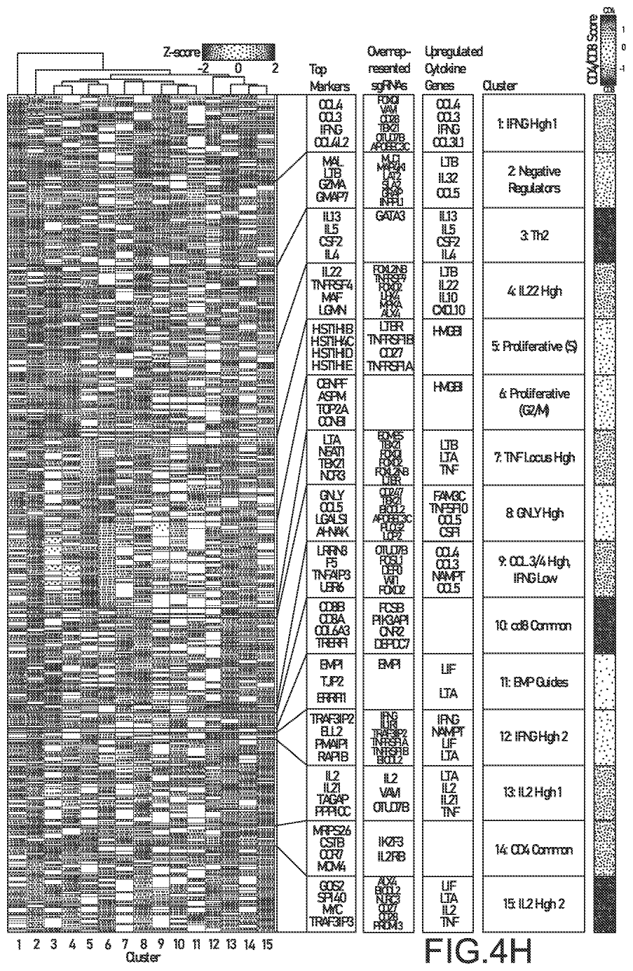

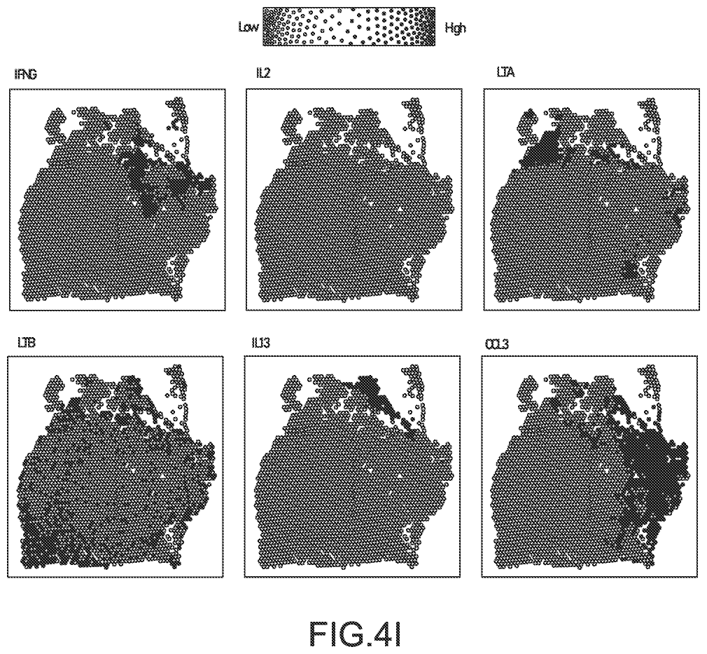

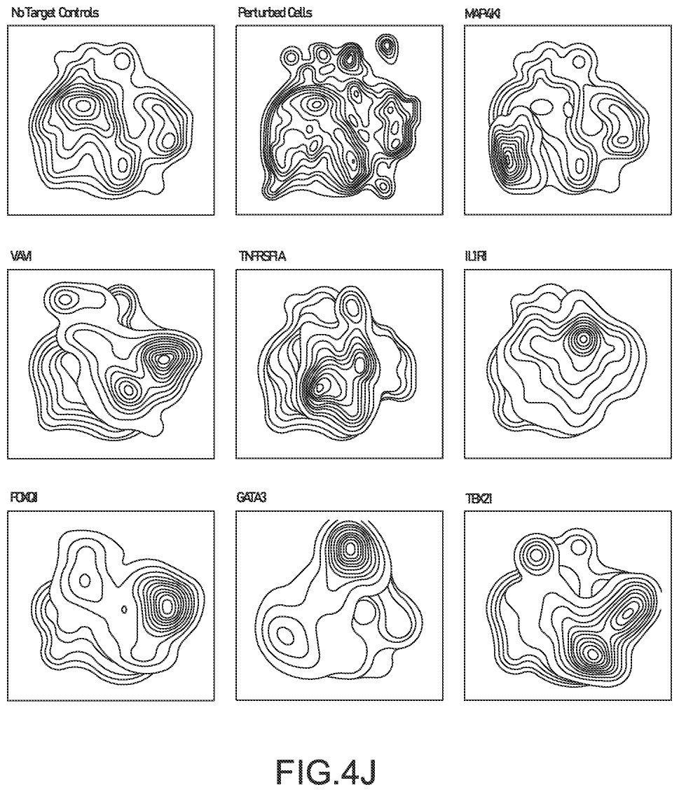

To assess the global molecular signatures resulting from each CRISPRa gene induction we developed a platform to couple pooled CRISPRa perturbations with barcoded single-cell RNA sequencing (scRNA-seq) read-outs (CRISPRa Perturb-seq) (FIG. 4A). As similar CRISPRa Perturb-seq approaches have been powerful in cell lines and animal models (35-37), we incorporated a direct-capture sequence into the CRISPRa-SAM modified sgRNA scaffold to enable compatibility with droplet-based scRNA-seq methods.

We performed CRISPRa Perturb-seq characterization of regulators of stimulation responses in 56,000 primary human T cells targeting 70 hits and controls from our genome wide CRISPRa cytokine screens (FIGS. 4, A and B). First, we confirmed that sgRNAs led to significant increases in the expression of their target genes. Next, uniform manifold approximation and projection (UMAP) dimensionality reduction revealed discrete separation of the resting and restimulated cells and showed relatively even distribution of cells from two donors (FIG. 4C. Gene signatures allowed us to resolve most T cells as either CD4 or CD8′ (FIG. 4D). Thus, we generated a high-quality CRISPRa Perturb-seq dataset.

Cytokine production can be tuned by reinforced TCR signaling. To identify CRISPRa gene perturbations that tune the general strength of stimulation-responsive genes, we calculated a scRNA-seq “activation” score based on a gene signature we derived from comparing resting and restimulated cells within the non-targeting control sgRNA group. Projecting activation scores on the stimulated cell UMAP revealed discrete regions of higher and lower activation scores among the restimulated cells (FIG. 4E). We next examined activation scores across CRISPRa perturbations (FIG. 4F). Strikingly, negative regulators except IKZF3 (encoding the transcription factor Aiolos) decreased activation scores, suggesting they act to broadly dampen stimulation strength. By contrast, IKZF3 reduced IFNG expression without reducing the overall activation score (FIG. 4F), indicative of a possible distinct mechanism of cytokine gene regulation. Many of the positive regulators significantly increased activation score, with VAV1 causing the strongest activation potentiation (FIG. 4F). Thus, many, but not all, hits act by tuning overall T cell activation to varying degrees.

We next asked how different perturbations affected the expression of cytokine and other effector genes in stimulated cells. We analyzed pseudobulk differential gene expression under restimulated conditions for each sgRNA target cell group, compared with no-target control cells. JFNG was differentially expressed in 29 different sgRNA targets, with only sgRNAs targeting negative regulators causing decreased expression. 1L2, however, was barely detectable by scRNA-seq. Only IL2 and VAV1 sgRNAs caused its increased expression, consistent with our observations that VAV1 activation caused the greatest level of IL 2 release (FIG. 3H). Many of the negative regulators drove a stereotyped pattern of differential cytokine gene expression, whereas positive regulators generally promoted more diverse cytokine expression patterns than negative regulators. Notably, TBX21 (T-bet) modulated the expression of most detectable cytokine genes. Furthermore, unlike most perturbations, it altered cytokine expression independently of stimulation.

We next used clustering analysis to characterize CRISPRa-driven cell states in restimulated and resting T cells (FIG. 4G). For each cluster, we identified the top upregulated gene expression markers and cytokine genes, contributions of CD4′/CD8 T cells, and overrepresented sgRNAs revealing a diverse landscape of T cell states promoted by CRISPRa (FIG. 4, H to J). Negative cytokine regulators (e.g., MAP4KI) were highly enriched in cluster 2, marked by LTB expression and low activation score. Notably, only GATA3 promoted a Th2 phenotype (cluster 3) suggesting that altered T helper differentiation was not a common mechanism among negative LFNG regulators. Thus, Perturb seq reveals cell states promoted by the overexpression of different key regulators.

We identified two 112-expressing clusters, despite poor capture of the transcript, with both of the clusters consisting primarily of CD4+ T cells. Cluster 13 had higher 112 expression of the two and was promoted by VAX1 and OTUD7B sgRNAs. VAV1 sgRNAs were strongly enriched in both IFNG- and 1L²-expressing clusters, suggesting that VAV1-mediated potentiation of T cell stimulation may drive differentiation towards multiple distinct cytokine-producing populations.

We also identified two distinct clusters of cells expressing JFNG (clusters I and 12) containing both CD4- and CD8′⁺ T cells. Cluster 1 was marked by high expression of CCL3 and CCL4 and was enriched for sgRNAs with strong activation score potentiation, such as VAV1, CD28, and FOXQ1. By contrast, Cluster 12 was enriched for sgRNAs known to activate the NF xB pathway, such as IL1RI, TRAF3IP2, TNFRSFIA, and TNIFRSFIB. These observations suggest that potentiated stimulation/costimulation may drive T cells to an activated IFNG expressing state distinct from more specific signaling through the NF-κB pathway. Activation of a subset of TNFRSF receptor genes (TNFRSFIA, TNFRSFlB, LTBR, and CD27), also promoted cell states (clusters 5 and 6) marked by the high expression of cell-cycle genes. LTBR and CD27 sgRNAs were almost exclusively found in cells of this cluster, whereas TNFRSFIA/B sgRNAs appeared to push cells to both proliferative and FNG-expressing states. Thus, CRISPRa Perturb seq reveals how regulators of cytokine production both tune T cell activation and program cells into different stimulation-responsive states,

### Discussion

Paired CRISPRa and CRISPRi screens complement one another to decode the genetic programs regulating stimulation-responsive cytokine production in primary human T cells. CRISPRi identified required cytokine regulators, whereas CRISPRa uncovered key signaling bottlenecks in pathway function as well as regulators that are not necessarily active in ex vivo-cultured T cells. Future screens performed in various other experimental conditions have potential to identify additional regulators of T cell states and functions.

The technologies developed in this study enable screening approaches in primary human T cells and other primary cell types, such as screens for functional noncoding regions of the human genome (18, 38, 39). Furthermore, this screening framework is adaptable to other non-heritable editing applications of the CRISPR toolkit (40). continuing to expand opportunities to interrogate complex biological questions in primary cells, especially when CRISPR perturbations are coupled with single-cell analyses.

### BIBLIOGRAPHY

1. A. K. Abbas, E. Trotta, D. R Simeonov, A. Marson, J. A. Bluestone, Revisiting IL-2: Biology and therapeutic prospects. Sci Inmunol. 3 (2018), doi:10.1126/sciimmunol.aat1482.
2. L. Ni, J. Lu, Interferon gamrna in cancer immunotherapy. Cancer Med. 7, 4509-4516 (2018).
3. F. Castro, A. P. Cardoso, R. M. Giongalves, K. Serre, M. J. Oliveira, Interferon-Gamma at the Crossroads of Tumor Immune Surveillance or Evasion. Front. Immunol. 9, 847 (2018).
4. L. B. Ivashkiv, IFNy: signalling, epigenetics and roles in immunity, metabolism, disease and cancer immunotherapy. Nat. Rev. Immunol. 18, 545-558 (2018).
5. T. R. Malek, The biology of interleukin-2. Annu. Rev. Immunol. 26, 453-479 (2008).
6. D. A. Boardman, M. K. Levings, Cancer immunotherapies repurposed for use in autoimmunity. Nat Biorned Eng. 3, 259-263 (2019).
7. J. Gao, L. Z. Shi, H. Zhao, J. Chen, L. Xiong, Q. He, T. Chen, J. Roszik, C. Bernatchez, S. E. Woodman, P.-L. Chen, P. Hw u, J, P. Allison, A. Futreal, J. A. Wargo, P. Sharma, Loss of IFN-,y Pathway Genes in Tumor Cells as a Mechanism of Resistance to Anti-CTLA-4 Therapy. Cell. 167, 397-404.e9 (2016).
8. J. M. Zaretsky, A. Garcia-Diaz, D. S. Shin, H. Escuin-Ordinas, W. Hugo, S. Hu-Lieskovan, D. Y. Torrejon, G. Abril-Rodriguez, S. Sandoval, L. Barthly, J. Saco, B. Hornet Moreno, R. Mezzadra, B. Chmielowski, K. Ruchalski, 1 P. Shintakur, P. J, Sanchez, C. Puig-Saus, C. Cherry, E. Seja, X Kong, J. Pang, B. Berent-Maoz, B. Comin-Anduix, T. G. Graeber, P. C. Tumeh, T. N. M. Schumacher, R. S. Lo, A. Ribas, Mutations Associated with Acquired Resistance to PD-1 Blockade in Melanoma. N. Engl. J. Med. 375, 819-829 (2016).
9. M. Ayers, J. Lunceford, M. Nebozhyn, E. Murphy, A. Loboda, D. R. Kaufman, A. Albright, J. D. Cheng, S. P. Kang, V. Shankaran, S. A. Piha-Paul, J. Yearley, T. Y. Seiwert, A. Ribas, T. K. McClanahan, IFN-γ-related mRNA profile predicts clinical response to PD-1 blockade. J. Clin. Invest. 127, 2930-2940 (201 7).
10. R. R. Bartelt, N. Cruz-Orcutt, M. Collins, J. C. D. Houtman, Comparison of T cell receptor induced proximal signaling and downstream functions in immortalized and primary T cells. PLoS One. 4, e5430 (2009).
11. H. Colin-York, S. Kumari, L. Barbieri, L. Cords, M. Fritzsche, Distinct actin cytoskeleton behaviour in primary and imrnortalised T-cells. J. Cell Sci. 133 (2019), doi:10.1242/jcs.232322.
12. F. Astoul, C. Edmunds, D. A. Cantrell, S. G. Ward, PI 3-K and T-cell activation: limitations of T-leukemic cell lines as signaling models. Trends Immunol. 22, 490-496 (2001).
13. &. Parnas, M. Jovanovic, T. M. Fisenhaure, R. H. Herbst, A. Dixit, C. J. Ye, D. Przybylski, R. J. Platt, 1. Tirosh, N. F. Sanjana, 0. Shalem, R. Satija, R. Raychowdhury, P. Mertins, S. A. Carr, F. Zhang, N. Hacohen, A. Regev, A Genome-wide CRISPR Screen in Primary Immune Cells to Dissect Regulatory Networks. Cell. 162, 675-686 (2015).
14. M. B. Dong, G. Wang, R. D. Chow, L. Ye, L. Zhu, X. Dai, J. J. Park, H. R. Kim, Y. Errami, C. D. Guzmnan, X. Zhou, K. Y. Chen, P. A. Renauer, Y. Du, J. Shen, S, Z. Lam, J. J. Zhou, D. R. L.annin, R, S. Herbst, S. Chen, Systematic immunotherapy Target Discovery Using Genome Scale In Vivo CRJSPR Screens in CD8 T Cells. Cell, 178, 1189-1204.e23 (2019).
15. J. Henriksson, X. Chen, T. Comes, U. Ullah, K. B. Meyer, R. hiragaia, G. Duddy, J. Pramanik, K. Yusa., R. L.ahesmaa, S. A. Teichmann, Genome-wide CRISPR_Screens in T Helper Cells Reveal Pervasive Crosstalk between Activation and Differentiation. Cell. 176, 882-896.ei 8 (2019).
16. E. Shifrut, J. Carnevale, V. Tobin, T. L. Roth, J. M. Woo, C. T. Biui, P. J. Li, M. E. Diolaiti, A. Ashworth, A. Marson, Genome-wide CRISPR Screens in Primary Human T Cells Reveal Key Regulators ofnImmune Function. Cell. 175, 1958-1971iel5 (2018).
17. P. Y. Ting, A. E. Parker, J. S. Lee, C. Trussell, 0. Sharif, F. Luna, G. Federe, S. W. Barnes, J. R. Walker, J. Vance, M.-Y. Gao, H. E. Klock, S. Clarkson, C. Russ, L J. Miraglia, M. P. Cooke, A. E. Boitano, P. McNamara, J. Lamb, C. Schmedt, J. L. Snead, Guide Swap enables genome-scale pooled CRISPR-Cas9 screening in human primary cells. Nat. Methods. 15, 941-946 (2018).
18. D. R. Sirneonov, B. G. Gowen, M. Boontanrart, T. L. Roth, J. D. Gagnon, M. R. Mumbach, A. T. Satpathy, Y. Lee, N. L. Bray, A. Y. Chan, D. S. Lituiev, M. L. Nguyen, R. E. Gate, M. Subramianiam, Z. Li, J. M. Woo, T. Mitros, (G J. Ray, G. L. Curie, N. Naddaf, J. S. Chu, H. Ma, E. Boyer, F. Van Gool, H. Huang, R. Liu, V. R. Tobin, K. Schumann, M. J. Daly, K. K. Farh, K. M. Ansel, C. J. Ye, W. J. Greenleaf, M. S. Anderson, J. A. Bluestone, H. Y. Chang, J. E. Corn, A. Marson, Discovery of stimulation-responsive inmune enhancers with CRISPR activation. Nature. 549, 111-115 (2017).
19. Y. Liu, C. Yu, T. P. Daley, F. Wang, W. S. Cao, S. Bhate, X. Lin, C. Still 2nd, H. Liu, D. Zhao, H. Wang, X. S. Xie, S. Ding, W. H. Wong, M. Wernig, L. S. Qi, CRISPR Activation Screens Systematically Identify Factors that Drive Neuronal Fate and Reprogramming. Cell Stem Cell. 23, 758-771.e8 (2018).
20. X. Chen, L. Kozhaya, C. Tastan, L. Placek, M. Dogan, M. Horne, R. Abblett, E. Karhan, M. Vaeth, S. Feske, D. Unutmaz, Functional Interrogation of Primary Human T Cells via CRISPR Genetic Editing. J. Immunol. 201, 1586-1598 (2018).
21. R. Nasrallah, C. I. Imnianowski, L. Bossini-Castillo, F. M. Grant, M. Dogan, L. Placek, L. Kozhaya, P. Kuo, F. Sadiyah, S. K. Whiteside, M R, Mumbach, D. Glinos, P. Vardaka, C. E Whyte, T. Lozano, T. Fujita, H, Fujii, A, Liston, S, Andrews, A. Cozzani, I. Yang, S. Mitra, E. Lugli, H. Y. Chang, D. Unutmaz, G. Trynka, R. Roychoudhuri, A distal enhancer at risk locus I1 g3.5 promotes suppression of colitis by Treg cells. Nature. 583, 447-452 (2020).
22. K. R. Sanson, R. E. Hanna, M. Hegde, K. F. Donovan, C. Strand, M. E. Sullender, E. W. Vaimberg, A. Goodale, D. E. Root, F. Piccioni, J. G. Doench, Optimized libraries for CRISPR Cas9 genetic screens with multiple modalities. Nat. Commun. 9, 1-15 (2018).
23. S. Konermann, MI. D. Brigham, A. E. Trevino, J. Joung, 0. 0. Abudayyeh, C. Barcena, P. D. Hsu, N. Habib, J. S. Gootenberg, H. Nishimasu, 0. Nureki, F. Zhang, Genome-scale transcriptional activation by an engineered CRISPR-Cas9 complex. Nature. 517,583-588 (2014).
24S. M Kaech, W, Cui, Transcriptional control of effector and memory CD8+ T cell differentiation. Nat. Rev. Immunol. 12, 749-761 (2012).
25. J. Zhu, H. Yamane, W. E Paul, Differentiation of effector CD4 T cell populations (*). Annu. Rev. Immunol. 28, 445-489 (2010).
26. V. Lazarevic, L. H. Glimcher, G. M. Lord, T-bet: a bridge between innate and adaptive immunity. Nat. Rev. Immunol. 13, 777-789 (2013).
27. C. M. Evans, R. G. Jenner, Transcription factor interplay in T helper cell differentiation. Brief. Funct. Gaenomics. 12, 499-511 (2013).
28. A. H. Courtney, W.-L. Lo, A. Weiss, TCR Signaling: Mechanisms of Initiation and Propagation. Trends Biochem. Sci. 43, 108-123 (2018).
29. G. Gaud, R. Lesourne, P. E. Love, Regulatory mechanisms in T cell receptor signalling. Nat. Rev. Immunol. 18, 485-497 (2018).
30. S. J. Holland, X. C. Liao, M. K. Mendenhall, X. Zhou, J. Pardo, P. Chu, C. Spencer, A. Fu, N. Sheng, P. Yu, E. Pali, A. Nagin, M. Shen, S. Yu, E. Chan, X. Wu, C. Li, M Woisetschlager, G. Aversa, F. Kolbinger, M. K. Bennett, S. Molineaux, Y. Luo, D. G.

Payan, H. S. Mancebo, J. Wi, Functional cloning of Src-like adapter protein-2 (SLAP-2), a novel inhibitor of antigen receptor signaling. J. Exp. Med. 194, 1263-12⁷6 (2001).
31. J.-W. Shui, J. S. Boomer, J. Han, J. Xu, G. A. Dement, G. Zhou, T.-H. Tan, Hematopoietic progenitor kinase I negatively regulates T cell receptor signaling and T cell-mediated immiune responses. Nat. Immunol. 8, 84-91 (2006).
32. T. Hart, A, H. Y. Tong, K-. Chan, J. Van Leeuwen, A. Seetharaman. M. Aregger, M, Chandrashekhar, N. Hustedt, S. Seth, A, Noonan. A. Habsid, 0. Sizova, L. Nedyalkova, R-Climie, L. Tworzyanski, K. Lawson, M. A. Sartori, S. Alibeh, D. Tieu, S. Masud, P.

### Mero, A. Weiss, K. R-Brown, M. UJsaj, M. Billmann, M. Rahman, M. Constanzo, C. L.

Myers, B. I. Andrews, C. Boone, D. Durocher, J. Moffat, Evaluation and Design of Genome-Wide CRISPR/SpCas9 Knockout Screens. G3. 7, 2719 2727 (2017).
33. S. R. Ferdosi, R. Ewaisha, F. Moghadam, S. Krishna, J. (G. Park, M. R. Ebrahimkhani, S. Kiani, K. S. Anderson, Multifunctional CRISPR-Cas9 with engineered immunosilenced human T cell epitopes. Nat. Commurn. 10, 1842 (201 9).
34. H. Hu, H. Wang, Y. Xiao, J. Jin, J.-H. Chang, Q. Zou, X. Xie, X. Cheng, S.-C. Sun, Otud7b facilitates T cell activation and inflammatory responses by regulating Zap70 ubiquitination. J. Exp. Med. 213, 399-414 (2016).
35. E. P. Mirnitou, A. Cheng, A. Montalbano, S. Hao, M, Stoeckius, M. Legut, T. Roush, A. Herrera, E. Papalexi, Z. Ouyang, R. Satija, N. E. Sanjana, S. B. Koralov, P. Smibert, Multiplexed detection of proteins, transcriptomes, clonotypes and CRISPR perturbations in single cells. Nat. Methods. 16, 409-412 (2019).
36. C. Alda-Catalinas, D. Bredikhin, 1. Hernando-Herraez, F. Santos, 0. Kubinyecz, M. A. Eckersley-Maslin, 0. Stegle, W. Reik, A Single-Cell Transcriptonics CRISPR-Activation Screen Identifies Epigenetic Regulators of the Zygotic Genome Activation Program. Cell Syst. 11, 25-41.e9 (2020).
37. J. M. Replogle, T. M. Norman, A. Xu, J. A. Hussmann, J. Chen, J. Z. Cogan, E. J. Meer, J. M. Terry, D. P. Riordan, N. Srinivas, I. T. Fiddes, J. G. Arthur, L. J. Alvarado, K. A. Pfeiffer, T. S. Mikkelsen, J. S. Weissman, B. Adamson, Combinatorial single-cell CRISPR screens by direct guide RNA capture and targeted sequencing. Nat. Biotechnol. 38, 954-961 (2020).
38. P, I Thakore, A. M, D'Ippolito, L. Song, A. Safi, N. K. Shivakurnar, A, M. Kabadi, T. E. Reddy, G. E. Crawford, C. A. Gersbach, Highly specific epigenome editing by CRISPR-Cas9 repressors for silencing of distal regulatory elements. Nat. Methods. 12, i143-1149 (2015).
39. C. P, Fulco, M. Munschauer, R. Anyoha, G. Munson, S. R. Grossman, E. M. Perez, M. Kane, B. Cleary, E. S. Lander, J. M. Engreitz, Systematic mapping of functional enhancer-promoter connections with CRISPR interference. Science. 354, 769-773 (2016).
40. X. Xu, L. S. Qi, A CRISPR.-dCas Toolbox for Genetic Engineering and Synthetic Bi1ology. J. Mol. Biol. 431, 34-47 (2019).
41. 1. Chun, K. H. Kim, Y H. Chiang, W. Xie, Y. C. G. Lee, R. Pajarillo, A. Rotolo, 0. Shestova, S. J. [long, M. Abdel-Mohsen, M. Wysocka, H. J. Ballard, D. M. Barrett, A. D. Posey, D. Powell Jr, S. .i Gill, S. J. Schuster, S. K. Barta, A. H. Rook, C. H. June, M. Ruella, CRISPR-Cas9 knock out of CD5 enhances the anti-tumor activity of chimeric Antigen Receptor T cells. Blood. 136, 51-52 (2020).
42 R. C. Lynn, E. W. Weber, E. Sotillo, D. Gennert, P. Xu, Z. Good, H. Anbunathan, J. Lattin, R. Jones, V. Tieu, S. Nagaraja, J. Granja, C. F. A. de Bourcy, R. Majzner, A. T. Satpathy, S. R. Quake, M]Vf Monje, H. Y. Chang, C. L. Mackall, c-Jun overexpression in CAR T cells induces exhaustion resistance. Nature. 576, 293-300 (2019).
43. P. Datlinger, A. F. Rendeiro, C. Schmidl, T. Krausgruber, P. Traxler, J. Klugharnmer, L. C. Schuster, A. Kuchler, D. Alpar, C. Bock, Pooled CRISPR screening with single-cell transcriptome readout. Nat. Methods. 14, 297-301 (2017).
44. J. W. Freimer, 0. Shaked, S. Naqvi, N. Sinnott-Armstrong, A. Kathiria, A. F. Chen, 0.1. T. Cortez, W. J. Greenleaf, J. K. Pritehard, A. Marson, Systematic discovery and perturbation of regulatory genes in human T cells reveals the architecture of immune networks. bioRxiv (2021), p. 2021.04.18.440363.
45. W. Li, H. Xu, T. Xiao, L. Cong, M. I. Love, F. Zhang, R. A. Irizarry, J. S. Liu, M. Brow,vn, X. S. Liu, MAGeCK enables robust identification of essential genes from genome-scale CRISPR/Cas9 knockout screens. Genome Biol. 15, 554 (2014).
46. G. Korotkevich, V. Sukhov, N. Budin, B. Shpak, M. N. Artyomov, A. Sergushichev, Fast gene set enrichment analysis. bioRxiv (2016), , doi:10.i101/060012.
47. H. K. Finucane, B. Bulik-Sullivan, A. Gusev, Ci. Trynka, Y. Reshef, P.-R. Loh, V. Anttila, H. Xu, C. Zang, K. Fart, S. Ripke, F. R. Day, ReproGen Consortium, Schizophrenia Working Group of the Psychiatric Genomics Consortium, RACI Consortium, S. Purcell, E. Stahl, S. Lindstrom, J. R. B. Perry, Y. Okada, S. Raycha.udhuri, M. J. Daly, N. Patterson, B. M. Neale, A. L. Price, Partitioning heritability by functional annotation using genome-wide association summary statistics. Nat. Genet. 47, 1228-1235 (2015).
48. M. A. Horlbeck, L. A. Gilhert, J. E. Villalta, B. Adamson, R. A. Pak, Y. CThen, A. P. Fields, C. Y. Park, J. E. Corn, M. Kampmann, J. S. Weissman, Compact and highly active next-generation libraries for CRISPR-mediated gene repression and activation. Elife. 5 (2016), doi:10,7554/eLife.1 9760.
49. M. Martin, Cutadapt removes adapter sequences from high-throughput sequencing reads. EMBnet.journal. 17, 10-12 (2011).
50. A. Dobin, C. A. Davis, F. Schlesinger, J. Drenkow, C. Zaleski, S. Jha, P. Batut, M. Chaisson, T. R. Gingeras, STAR: ultrafast universal RNA-seq aligner. Bioinformatics. 29, 15-21 (2013).
51. Y. Lia.o, G. K. Srnyth, W. Shi, featureCounts: an efficient general-purpose program for assigning sequence reads to genonic features. Bioinformatics. 30, 923-930 (2014).
52. M. E. Ritchie, B. Phipson, D. Wu, Y. Hu, C. W. Law, W. Shi, G. K. Smyth, limma powers differential expression analyses for RNA-sequencing and microarray studies. Nucleic Acids Res. 43, e47 (2015).
53. H. Heaton, A. M. Talman, A. Knights, M. Imaz, D. J. Gaffney, R. Durbin, M. Henberg, M. K. N. Lawniczak, Souporcell: robust clustering of single-cell RNA-seq data by genotype without reference genotypes. Nat. Methods. 17, 615-620 (2020).
54. R. Satija, J. A. Farrell, D. Gennert, A. F. Schier, A. Regev, Spatial reconstruction of single-cell gene expression data. Nat. Biotechnol. 33, 495-502 (2015).
55. C. Hafemeister, R. Satija, Normalization and variance stabilization of single-cell RNA-seq data using regularized negative binomial regression. Cenome Biol. 20, 296 (2019).
56. Z. Steinhart, Code repository for CRISPR activation and interference screens decode stimulation responses in primary human T cells, Zenodo (2022); 10.5281/zenodo.578465
57. B. J. Schmiedel, D. Singh, A. Madrigal, A. G. Valdovino-Gonzalez, B. M. White, J. Zapardiel Gonzalo, B. Ha, G. Altay, J. A. Greenbaum, CG. McVicker, G. Seumois, A. Rao, M. Kronenberg, B. Peters, P. Vijayanand, Impact of Genetic Polymorphisms on Human Immune Cell Gene Expression, Cell. 175, 1701-1715.e16 (2018).
58. Immunological Genome Project, ImmGen at 15. Nat. Immunol. 21, 700-703 (2020),
59. B. P. Nicolet, A. Guislain, F. P. J. van Alphen, R. Gomez-Eerland, T. N. M. Schumacher, M. van den Biggelaar, M. C. Wolkers, CD29 identifies IFN-γ-producing human CD8+ T cells with an increased cytotoxic potential. Proc. Natl. Acad. Sci. U.S.A. 117, 6686-6696 (2020).

### Example 3

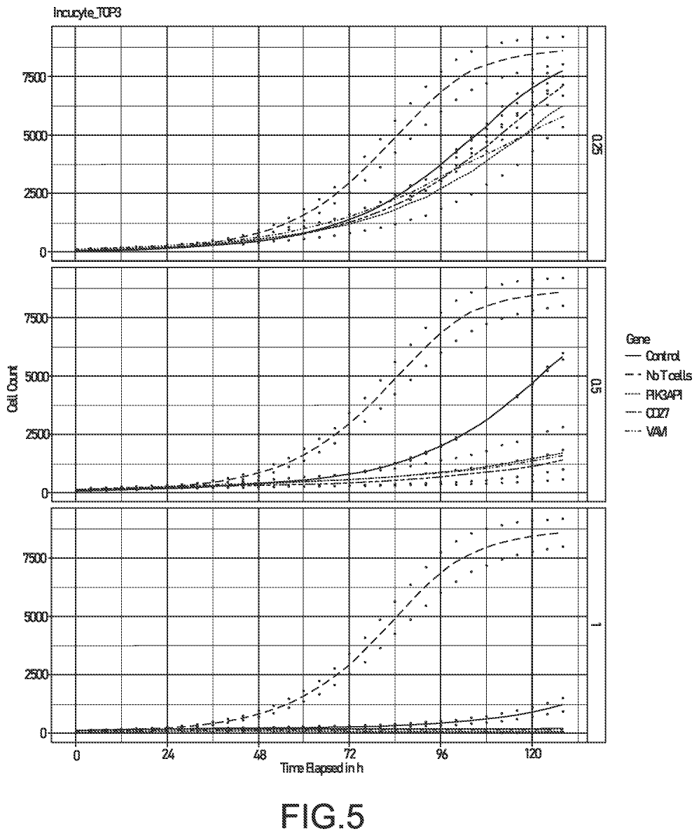

In vitro data using the identified hits for T cell cancer therapies. For this assay, T cells from two human blood donors were virally transduced with the 1G4 anti-cancer T cell receptor as well as the respective gene from the CRISPRa screens (or “empty” virus as control) and cocultured with NYESO expressing A375 melanoma cells. A live imaging system recorded the cancer cell counts every 4h. T cells transduced with the target genes VAV1, PIK3API and CD27 showed enhanced cancer killing (FIG. 5).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements:

1. A method comprising contacting one or more test agents with one or more T cells to form an assay mixture, and detecting or quantifying interferon-γ production, interleukin-2 production, cellular proliferation, or a combination thereof in the assay mixture or within one or more T cells, to generate a detected or quantified level of interferon-γ production, interleukin-2 production, cellular proliferation, or a combination thereof.
2. The method of statement 1, further comprising comparing the detected or quantified level of interferon-f production, the detected or quantified level of interleukin-2 production, the detected or quantified level of cellular proliferation, or a combination thereof with a control.
3. The method of statement 1 or 2, further comprising measuring the quantity of one or more of the regulators listed in Tables 1-7 or FIGS. 1-4 in the assay mixture or in one or more of the T cells.
4. The method of statement 1, 2, or 3, wherein one or more of the T cells initially contacted with the test agent naturally expresses any of the regulators listed in Tables 1-7 or FIGS. 1-4.
5. The method of statement 1-3 or 4, wherein one or more of the T cells initially contacted with the test agent do not express one or more of the regulators listed in any of Tables 1-⁷ or FIGS. 1-4.
6. The method of statement 1-4 or 5, wherein one or more of T cells initially contacted with the test agent have the potential to express one or more of the regulators but when initially mixed with a test agent the cells do not express detectable amounts of one or more of the regulators.
7. The method of statement 1-5 or 6, wherein at least one of the T cells is a mutant T cell comprising a knock-down or knockout mutation that reduces expression or activity of one or more of the regulators listed in any of Tables 1-7 or FIGS. 1-4.
8. The method of statement 7, further comprising: modifying the one or more mutant T cells to express or over-express the one or more regulators listed in Tables 1-7 or FIGS. 1-4; and detecting or quantifying interferon-γ production, interleukin-2 production, cellular proliferation, or a combination thereof in a second assay mixture or within the one or more of the mutant T cells.
9. The method of statement 1-7 or 8, wherein the one or more of the T cells is in a population of T cells.
10. The method of statement 1-8 or 9, wherein one or more of T cells comprises one or more cytotoxic T cells, chimeric antigen receptor T cells, helper T cells, regulatory T cells, naive T cells, activated T cells, CD4 T cells, CD8 T cells, gamma delta T cells, natural killer (NK) cells, induced pluripotent stem cell-derived immune (e.g., lyrnphoid and/or myeloid) cells, or a combination thereof,
11. The method of statement 1-9 or 10, further comprising adding at least one second cell type to the assay mixture before detecting or quantifying the interferon-v production, interleukin-2 production, cellular proliferation, or a combination thereof.
12. The method of statement 11, wherein the second cell type is one or more types of cancer cells, one or more types of immune cells, or a combination thereof.
13. The method of statement 12, wherein one or more of the cancer cells comprise leukemia cells, lymphoma cells, Hodgkin's disease cells, sarcomas of the soft tissue and bone, lung cancer cells, mesothelioma, esophagus cancer cells, stomach cancer cells, pancreatic cancer cells, hepatobiliary cancer cells, small intestinal cancer cells, colon cancer cells, colorectal cancer cells, rectum cancer cells, kidney cancer cells, urethral cancer cells, bladder cancer cells, prostate cancer cells, testis cancer cells, cervical cancer cells, ovarian cancer cells, breast cancer cells, endocrine system cancer cells, skin cancer cells, central nervous system cancer cells, melanoma cells of cutaneous and/or intraocular origin, cancer cells associated with AIDS, or a combination thereof.
14. The method of statement 12 or 13, wherein one or more of cancer cells comprise metastatic cancer cells.
15. The method of statement 12, 13 or 14, wherein one or more of cancer cells comprise micrometastatic tumor cells, megametastatic tumor cells, recurrent cancer cells, or a combination thereof.
16. The method of statement 12-14 or 15, wherein one or more of the immune cells comprises macrophages, natural killer cells, dendritic cells, B cells, chimeric antigen receptor cells, cytotoxic T cells, helper T cells, regulatory T cells, naive T cells, activated T cells, CD4 T cells, CD8 T cells, gamma delta T cells, natural killer (NK) cells, induced pluripotent stem cell-derived immune (e.g., lymphoid and/or myeloid) cells, or a combination thereof.
17. The method of statement 12-15 or 16, further comprising measuring the cellular proliferation of at least one of the second cell types.
18. The method of statement 1-16 or 17, further comprising identifying one or more of the test agents that modulates the level of interferon-γ production, the level of interleukin-2 production, the level of cellular proliferation, or a combination thereof of one or more of the T cells, to thereby identify one or more useful test agents.
19. The method of statement 1-17 or 18, further comprising identifying one or more of the test agents that modulates expression or activity of one or more of the regulators listed in any of Tables 1-7 or FIGS. 1-4, to thereby identify one or more useful test agents.
20. The method of statement 1 9, further comprising measuring binding of one or more useful test agents to a protein encoded by, or nucleic acid comprising, one or more the regulators listed in any of Tables 1-7 or FIGS. 1-4.
21. The method of statement 19 or 20, further comprising administering one or more useful test agents to one or more experimental animals.
22. The method of statement 21, wherein one or more of the experimental animals has a disease, infection, or medical condition.
23. The method of statement 22, wherein the disease or condition is cancer, an immune disorder, or an immune condition.
24. The method of statement 23, wherein the immune disorder or immune condition is an autoimmune disorder, Graves disease, arthritis, psoriasis, Celiac disease, vitiligo, rheumatoid arthritis, lupus, Crohn's disease, multiple sclerosis, type 1 diabetes, alopecia, inflammatory bowel disease (IBD), Guillain-Barre syndrome, chronic inflammatory demyelinating polyneuropathy, or a combination thereof.
25. The method of statement 21-23 or 24, further comprising monitoring the one or more experimental animals for symptoms of the disease or condition, for toxic side effects of the useful test agents, or a combination thereof.
26. The method of statement 21-24 or 25, further comprising monitoring immune cell numbers and/or types in the one or more experimental animals.
27. The method of statement 22-25 or 26, further comprising identifying one or more useful test agents as a therapeutic agent useful for treatment of the disease or condition.
28. A composition comprising a useful test agent or a therapeutic agent identified by the method of any of claims 1-27.
29. A method comprising ex vivo modification of any of the genes listed in Tables 1-7 or FIGS. 1-4 within at least one lymphoid or myeloid cell, or a combination thereof to generate at least one modified lymphoid cell, at least one modified myeloid cell, or a mixture of modified lymphoid and modified myeloid cells.
30. The method of statement 29, wherein the modification is one or more deletion, substitution or insertion into one or more genomic sites of any of the genes listed in Tables 1-7 or FIGS. 1-4.
31. The method of statement 29 or 30, wherein the modification is one or more CRISPR-mediated modifications or activations of any of the genes listed in Tables 1-7 or FIGS. 1-4.
32. The method of statement 29, 30 or 31, further comprising administering at least one modified lymphoid cell, at least one modified myeloid cell, or a mixture of modified lymphoid and modified myeloid cells to a subject.
33. The method of statement 29, 30 or 31 further comprising incubating the at least one modified lymphoid cell, at least one modified myeloid cell, or a mixture of modified lymphoid and modified myeloid cells to form a population of modified cells.
34. The method of statement 33, further comprising administering the population of modified cells to a subject.
35. The method of statement 32 or 34, wherein the subject has a disease or condition.
36. The method of statement 35, wherein the disease or condition is an immune condition or cancer.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modified cations and variations are considered to be within the scope of this invention as defined by the appended claims and statements of the invention.

The invention has been described broadly and generically herein, Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method comprising ex vivo modification of any of the genes listed in Tables 1-7 or FIGS. 1-4 within at least one lymphoid or myeloid cell, or a combination thereof to generate at least one modified lymphoid cell, at least one modified myeloid cell, or a mixture of modified lymphoid and modified myeloid cells.

2. The method of claim 1, wherein the modification is one or more deletion, substitution or insertion into one or more endogenous genomic sites of any of the genes listed in Tables 1-7 or FIGS. 1-4.

3. The method of claim 1, wherein the modification is reduction of expression or translation of any of the genes listed in Tables 1-7 or FIGS. 1-4.

4. The method of claim 3, wherein the reduction of expression or translation is by an inhibitory nucleic acid.

5. The method of claim 1, wherein the modification is increased expression of any of the genes listed in Tables 1-7 or FIGS. 1-4.

6. The method of claim 5, wherein the increased expression is by modification of one or more promoters of any of the genes listed in Tables 1-7 or FIGS. 1-4.

7. The method of claim 1, wherein the modification is one or more CRISPR-mediated modifications or activations of any of the genes listed in Tables 1-7 or FIGS. 1-4.

8. The method of claim 1, wherein the modification is transformation of at least one lymphoid or myeloid cell, or a combination thereof, with one or more expression cassettes comprising a promoter operably linked to a nucleic acid segment comprising a coding region of any of the genes listed in Tables 1-7 or FIGS. 1-4.

9. The method of claim 1, further comprising administering at least one modified lymphoid cell, at least one modified myeloid cell, or a mixture of modified lymphoid and modified myeloid cells to a subject.

10. The method of claim 1, further comprising incubating the at least one modified lymphoid cell, at least one modified myeloid cell, or a mixture of modified lymphoid and modified myeloid cells to form a population of modified cells.

11. The method of claim 10, further comprising administering the population of modified cells to a subject.

12. The method of claim 9, wherein the subject has a disease or condition.

13. The method of claim 12, wherein the disease or condition is an immune condition or cancer.

14. A method comprising contacting at least one test agent with test cells to provide a test assay mixture, and measuring:
a. cellular proliferation of the test cells, cytokine release by the test cells, or a combination thereof;
b. activation of the test cells;
c. expression or activity of any of the regulators listed in Tables 1-7 or FIGS. 1-4 in the cells; or
d. a combination thereof.

15. The method of claim 14, further comprising comparing the measured results to control results.

16. The method of claim 15, wherein control results are results of the test cells measured without any of the test agents.

17. The method of claim 14, wherein the test cells comprise lymphoid and/or myeloid cells.

18. The method of claim 14, wherein the test cells comprise cytotoxic T cells, helper T cells, regulatory T cells, naive T cells, activated T cells, CD4 T cells, CD8 T cells, gamma delta T cells, chimeric antigen receptor (CAR) cells, natural killer (NK) cells, induced pluripotent stem cell-derived immune cells, or a combination thereof.

19. The method of claim 13, wherein the immune condition is an autoimmune disorder, Graves disease, arthritis, psoriasis, Celiac disease, vitiligo, rheumatoid arthritis, lupus, Crohn's disease, multiple sclerosis, type 1 diabetes, alopecia, inflammatory bowel disease (IBD), Guillain-Barre syndrome, chronic inflammatory demyelinating polyneuropathy, or a combination thereof.

20. The method of claim 13, wherein the cancer is leukemia, lymphoma, Hodgkin's disease, sarcomas of the soft tissue and bone, lung cancer, mesothelioma, esophagus cancer, stomach cancer, pancreatic cancer, hepatobiliary cancer, small intestinal cancer, colon cancer, colorectal cancer, rectum cancer, kidney cancer, urethral cancer, bladder cancer, prostate cancer, testis cancer, cervical cancer, ovarian cancer, breast cancer, endocrine system cancer, skin cancer, central nervous system cancer, melanoma, cancer associated with AIDS, or a combination thereof.


SEQUENCE LISTING (165 sequences)

SEQ ID NO: 1 - protein, 508 aa, Homo sapiens
MSSPDGPSFPSGPLSGGASPSGDEGFFPFVLERRDSFLGGGPGPEEPEDLALQLQQKEKDLLLAAELGKMLLERNEELRRQLETLSAQHLEREERLQQENHELRRGLAARGAEWEARAVELEGDVEALRAQLGEQRSEQQDSGRERARALSELSEQNLRLSQQLAQASQTEQELQRELDALRGQCQAQALAGAELRTRLESLQGENQMLQSRRQDLEAQIRGLREEVEKGEGRLQTTHEELLLLRRERREHSLELERARSEAGEALSALRRLQRRVSELEEESRLQDADVSAASLQSELAHSLDDGDQGQGADAPGDTPTTRSPKTRKASSPQPSPPEEILEPPKKRTSLSPAEILEEKEVEVAKLQDEISLQQAELQSLREELQRQKELRAQEDPGEALHSALSDRDEAVNKALELSLQLNRVSLERDSLSRELLRAIRQKVALTQELEAWQDDMQVVIGQQLRSQRQKELSASASSSTPRRAAPRFSLRLGPGPAGGFLSNLFRRT

SEQ ID NO: 2 - protein, 598 aa, Homo sapiens
MRGGGGCVLALGGQRGFQKERRRMASGNGLPSSSALVAKGPCALGPFPRYIWIHQDTPQDSLDKTCHEIWKRVQGLPEASQPWTSMEQLSVPVVGTLRGNELSFQEEALELSSGKDEISLLVEQEFLSLTKEHSILVEESSGELEVPGSSPEGTRELAPCILAPPLVAGSNERPRASIIVGDKLLKQKVAMPVISSRQDCDSATSTVTDILCAAEVKSSKGTEDRGRILGDSNLQVSKLLSQFPLKSTETSKVPDNKNVLDKTRVTKDFLQDNLFSGPGPKEPTGLSPFLLLPPRPPPARPDKLPELPAQKRQLPVFAKICSKPKADPAVERHHLMEWSPGTKEPKKGQGSLFLSQWPQSQKDACGEEGCCDAVGTASLTLPPKKPTCPAEKNLLYEFLGATKNPSGQPRLRNKVEVDGPELKFNAPVTVADKNNPKYTGNVFTPHFPTAMTSATLNQPLWLNLNYPPPPVFTNHSTFLQYQGLYPQQAARMPYQQALHPQLGCYSQQVMPYNPQQMGQQIFRSSYTPLLSYIPFVQPNYPYPQRTPPKMSANPRDPPLMAGDGPQYLFPQGYGFGSTSGGPLMHSPYFSSSGNGINF

SEQ ID NO: 3 - protein, 1251 aa, Homo sapiens
MLGWVQRVLPQPPGTPRKTKMQEEEEVEPEPEMEAEVEPEPNPEEAETESESMPPEESFKEEEVAVADPSPQETKEAALTSTISLRAQGAEISEMNSPSRRVLTWLMKGVEKVIPQPVHSITEDPAQILGHGSTGDTGCTDEPNEALEAQDTRPGLRLLLWLEQNLERVLPQPPKSSEVWRDEPAVATGAASDPAPPGRPQEMGPKLQARETPSLPTPIPLQPKEEPKEAPAPEPQPGSQAQTSSLPPTRDPARLVAWVLHRLEMALPQPVLHGKIGEQEPDSPGICDVQTISILPGGQVEPDLVLEEVEPPWEDAHQDVSTSPQGTEVVPAYEEENKAVEKMPRELSRIEEEKEDEEEEEEEEEEEEEEEVTEVLLDSCVVSQVGVGQSEEDGTRPQSTSDQKLWEEVGEEAKKEAEEKAKEEAEEVAEEEAEKEPQDWAETKEEPEAEAEAASSGVPATKQHPEVQVEDTDADSCPLMAEENPPSTVLPPPSPAKSDTLIVPSSASGTHRKKLPSEDDEAEELKALSPAESPVVAWSDPTTPKDTDGQDRAASTASTNSAIINDRLQELVKLFKERTEKVKEKLIDPDVTSDEESPKPSPAKKAPEPAPDTKPAEAEPVEEEHYCDMLCCKFKHRPWKKYQFPQSIDPLTNLMYVLWLFFVVMAWNWNCWLIPVRWAFPYQTPDNIHHWLLMDYLCDLIYFLDITVFQTRLQFVRGGDIITDKKDMRNNYLKSRRFKMDLLSLLPLDFLYLKVGVNPLLRLPRCLKYMAFFEFNSRLESILSKAYVYRVIRTTAYLLYSLHLNSCLYYWASAYQGLGSTHWVYDGVGNSYIRCYYFAVKTLITIGGLPDPKTLFEIVFQLLNYFTGVFAFSVMIGQMRDVVGAATAGQTYYRSCMDSTVKYMNFYKIPKSVQNRVKTWYEYTWHSQGMLDESELMVQLPDKMRLDLAIDVNYNIVSKVALFQGCDRQMIFDMLKRLRSVVYLPNDYVCKKGEIGREMYIIQAGQVQVLGGPDGKSVLVTLKAGSVFGEISLLAVGGGNRRTANVVAHGFTNLFILDKKDLNEILVHYPESQKLLRKKARRMLRSNNKPKEEKSVLILPPRAGTPKLFNAALAMTGKMGGKGAKGGKLAHLRARLKELAALEAAAKQQELVEQAKSSQDVKGEEGSAAPDQHTHPKEAATDPPAPRTPPEPPGSPPSSPPPASLGRPEGEEEGPAEPEEHSVRICMSPGPEPGEQILSVKMPEEREEKAE

SEQ ID NO: 4 - protein, 511 aa, Homo sapiens
MATVQEKAAALNLSALHSPAHRPPGFSVAQKPFGATYVWSSIINTLQTQVEVKKRRHRLKRHNDCFVGSEAVDVIFSHLIQNKYFGDVDIPRAKVVRVCQALMDYKVFEAVPTKVFGKDKKPTFEDSSCSLYRFTTIPNQDSQLGKENKLYSPARYADALFKSSDIRSASLEDLWENLSLKPANSPHVNISATLSPQVINEVWQEETIGRLLQLVDLPLLDSLLKQQEAVPKIPQPKRQSTMVNSSNYLDRGILKAYSDSQEDEWLSAAIDCLEYLPDQMVVEISRSFPEQPDRTDLVKELLFDAIGRYYSSREPLLNHLSDVHNGIAELLVNGKTEIALEATQLLLKLLDFQNREEFRRLLYFMAVAANPSEFKLQKESDNRMVVKRIFSKAIVDNKNLSKGKTDLLVLFLMDHQKDVFKIPGTLHKIVSVKLMAIQNGRDPNRDAGYIYCQRIDQRDYSNNTEKTTKDELLNLLKTLDEDSKLSAKEKKKLLGQFYKCHPDIFIEHFGD

SEQ ID NO: 5 - protein, 279 aa, Homo sapiens
MGLSPGAEGEYALRLPRIPPPLPKPASRTASTGPKDQPPALRRSAVPHSGLNSISPLELEESVGFAALVQLPAKQPPPGTLEQGRSIQQGEKAVVSLETTPSQKADWSSIPKPENEGKLIKQAAEGKPRPRPGDLIEIFRIGYEHWAIYVEDDCVVHLAPPSEEFEVGSITSIFSNRAVVKYSRLEDVLHGCSWKVNNKLDGTYLPLPVDKIIQRTKKMVNKIVQYSLIEGNCEHFVNGLRYGVPRSQQVEHALMEGAKAAGAVISAVVDSIKPKPITA

SEQ ID NO: 6 - protein, 382 aa, Homo sapiens
MLRWTVHLEGGPRRVNHAAVAVGHRVYSFGGYCSGEDYETLRQIDVHIFNAVSLRWTKLPPVKSAIRGQAPVVPYMRYGHSTVLIDDTVLLWGGRNDTEGACNVLYAFDVNTHKWFTPRVSGTVPGARDGHSACVLGKIMYIFGGYEQQADCFSNDIHKLDTSTMTWTLICTKGSPARWRDFHSATMLGSHMYVFGGRADRFGPFHSNNEIYCNRIRVFDTRTEAWLDCPPTPVLPEGRRSHSAFGYNGELYIFGGYNARLNRHFHDLWKFNPVSFTWKKIEPKGKGPCPRRRQCCCIVGDKIVLFGGTSPSPEEGLGDEFDLIDHSDLHILDFSPSLKTLCKLAVIQYNLDQSCLPHDIRWELNAMTTNSNISRPIVSSHG

SEQ ID NO: 7 - protein, 638 aa, Homo sapiens
MVVSADPLSSERAEMNILEINQELRSQLAESNQQFRDLKEKFLITQATAYSLANQLKKYKCEEYKDIIDSVLRDELQSMEKLAEKLRQAEELRQYKALVHSQAKELTQLREKLREGRDASRWLNKHLKTLLTPDDPDKSQGQDLREQLAEGHRLAEHLVHKLSPENDEDEDEDEDDKDEEVEKVQESPAPREVQKTEEKEVPQDSLEECAVTCSNSHNPSNSNQPHRSTKITFKEHEVDSALVVESEHPHDEEEEALNIPPENQNDHEEEEGKAPVPPRHHDKSNSYRHREVSFLALDEQKVCSAQDVARDYSNPKWDETSLGFLEKQSDLEEVKGQETVAPRLSRGPLRVDKHEIPQESLDGCCLTPSILPDLTPSYHPYWSTLYSFEDKQVSLALVDKIKKDQEEIEDQSPPCPRLSQELPEVKEQEVPEDSVNEVYLTPSVHHDVSDCHQPYSSTLSSLEDQLACSALDVASPTEAACPQGTWSGDLSHHRSEVQISQAQLEPSTLVPSCLRLQLDQGFHCGNGLAQRGLSSTTCSFSANADSGNQWPFQELVLEPSLGMKNPPQLEDDALEGSASNTQGRQVTGRIRASLVLILKTIRRRLPFSKWRLAFRFAGPHAESAEIPNTAERMQRMIG

SEQ ID NO: 8 - protein, 638 aa, Homo sapiens; identical to SEQ ID NO: 7

SEQ ID NO: 9 - protein, 300 aa, Homo sapiens
MEEEASSPGLGCSKPHLEKLTLGITRILESSPGVTEVTIIEKPPAERHMISSWEQKNNCVMPEDVKNFYLMTNGFHMTWSVKLDEHIIPLGSMAINSISKLTQLTQSSMYSLPNAPTLADLEDDTHEASDDQPEKPHFDSRSVIFELDSCNGSGKVCLVYKSGKPALAEDTEIWFLDRALYWHFLTDTFTAYYRLLITHLGLPQWQYAFTSYGISPQAKQWFSMYKPITYNTNLLTEETDSFVNKLDPSKVFKSKNKIVIPKKKGPVQPAGGQKGPSGPSGPSTSSTSKSSSGSGNPTRK

SEQ ID NO: 10 - protein, 657 aa, Homo sapiens
MIESQEPVTFEDVAVDFTQEEWQQLNPAQKTLHRDVMLETYNHLVSVGCSGIKPDVIFKLEHGKDPWIIESELSRWIYPDRVKGLESSQQIISGELLFQREILERAPKDNSLYSVLKIWHIDNQMDRYQGNQDRVLRQVTVISRETLTDEMGSKYSAFGKMFNRCTDLAPLSQKFHKFDSCENSLKSNSDLLNYNRSYARKNPTKRFRCGRPPKYNASCSVPEKEGFIHTGMEPYGDSQCEKVLSHKQAHVQYKKFQAREKPNVCSMCGKAFIKKSQLIIHQRIHTGEKPYVCGDCRKAFSEKSHLIVHQRIHTGEKPYECTKYGRAFSRKSPFTVHQRVHTGEKPYECFECPKAFSQKSHLIIHQRVHTREKPFECSECRKAFCEMSHLFIHQITHTGKKPYECTECGKTFPRKTQLIIHQRTHTGEKPYKCGECGKTFCQQSHLIGHQRIHTGEKPYVCTDCGKAFSQKSHLTGHQRLHTGEKPYMCTECGKSFSQKSPLIIHQRIHTGEKPYQCGECGKTFSQKSLLIIHLRVHTGEKPYECTECGRAFSLKSHLILHQRGHTGEKPYECSECGKAFCGKSPLIIHQKTHPREKTPECAESGMTFFWKSQMITYQRRHTGEKPSRCSDCGKAFCQHVYFTGHQNPYRKDTLYIC

SEQ ID NO: 11 - protein, 904 aa, Homo sapiens
MLETYNSLVSLQELVSFEEVAVHFTWEEWQDLDDAQRTLYRDVMLETYSSLVSLGHCITKPEMIFKLEQGAEPWIVEETPNLRLSAVQIIDDLIERSHESHDRFFWQIVITNSNTSTQERVELGKTFNLNSNHVLNLIINNGNSSGMKPGQFNDCQNMLFPIKPGETQSGEKPHVCDITRRSHRHHEHLTQHHKIQTLLQTFQCNEQGKTFNTEAMFFIHKRVHIVQTFGKYNEYEKACNNSAVIVQVITQVGQPTCCRKSDFTKHQQTHTGEKPYECVECEKPSISKSDLMLQCKMPTEEKPYACNWCEKLFSYKSSLIIHQRIHTGEKPYGCNECGKTFRRKSFLTLHERTHTGDKPYKCIECGKTFHCKSLLTLHHRTHSGEKPYQCSECGKTFSQKSYLTIHHRTHTGEKPYACDHCEEAFSHKSRLTVHQRTHTGEKPYECNECGKPFINKSNLRLHQRTHTGEKPYECNECGKTFHRKSFLTIHQWTHTGEKPYECNECGKTFRCKSFLTVHQRTHAGEKPYACNECGKTYSHKSYLTVHHRTHTGEKPYECNECGKSFHCKSFLTIHQRTHAGKKPYECNECEKTFINKLNLGIHKITHTGERPYECNECGKTFRQKSNLSTHQGTHTGEKPYVCGKTFHRKSFLTIHQRTHTGKNRMDVMNVEKLFVRNHTLLYIRELTPGKSPMNVMNVENPFIRRQIFRSIKVFTRGRNPMNVANVEKPCQKSVLTVHHRTHTGEKPYECNECGKTFCHKSNLSTHQGTHSGEKPYECDECRKTFYDKTVLTIHQRTHTGEKPFECKECRKTFSQKSKLFVHHRTHTGEKPFRCNECRKTFSQKSGLSIHQRTHTGEKPYECKECGKTFCQKSHLSRHQQTHIGEKSDVAEAGYVFPQNHSFFP

SEQ ID NO: 12 - protein, 930 aa, Homo sapiens
MLLGWASLLLCAFRLPLAAVGPAATPAQDKAGQPPTAAAAAQPRRRQGEEVQERAEPPGHPHPLAQRRRSKGLVQNIDQLYSGGGKVGYLVYAGGRRFLLDLERDGSVGIAGFVPAGGGTSAPWRHRSHCFYRGTVDGSPRSLAVFDLCGGLDGFFAVKHARYTLKPLLRGPWAEEEKGRVYGDGSARILHVYTREGFSFEALPPRASCETPASTPEAHEHAPAHSNPSGRAALASQLLDQSALSPAGGSGPQTWWRRRRRSISRARQVELLLVADASMARLYGRGLQHYLLTLASIANRLYSHASIENHIRLAVVKVVVLGDKDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEETFGSTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRICLQGKCVDKTKKKYYSTSSHGNWGSWGSWGQCSRSCGGGVQFAYRHCNNPAPRNNGRYCTGKRAIYRSCSLMPCPPNGKSFRHEQCEAKNGYQSDAKGVKTFVEWVPKYAGVLPADVCKLTCRAKGTGYYVVFSPKVTDGTECRLYSNSVCVRGKCVRTGCDGIIGSKLQYDKCGVCGGDNSSCTKIVGTFNKKSKGYTDVVRIPEGATHIKVRQFKAKDQTRFTAYLALKKKNGEYLINGKYMISTSETIIDINGTVMNYSGWSHRDDFLHGMGYSATKEILIVQILATDPTKPLDVRYSFFVPKKSTPKVNSVTSHGSNKVGSHTSQPQWVTGPWLACSRTCDTGWHTRTVQCQDGNRKLAKGCPLSQRPSAFKQCLLKKC

SEQ ID NO: 13 - DNA, 2025 bp, Homo sapiens
agagcgagaagatgatgcatgtgagccctgcccttgggaagcttccaggttgggaaatgaggaatgagcctgacacccagggcccagagagacccaggacagaggcaggtcaggaggcagacacgcgctgctgggtaatgacgacagcaccagtaatcacggctactccttgttaagtacttactaagtgcaagactccaagctaagcaattaatagaccttttcttgtttaatccttaccacaattccatagggttgagtaggaagtccttcttgaagtctaatctcaagaattcatgccatggattgggccaatgttccacctttattggagtctggggacttaggtggaaaaggcagaactggctggttggaggagccacctccctgcaaggggccaggagggagattacggaggcgccaagcccagagctccaacatgcacctgccacagctccaggggagatcgggggcctgccaatttaccccgccccatgatctcatggctgtgtgcgccaggcactggctcagggagcagcatctcacagagaagatacttggatgggccacaggccaaaactgggccaaaaccctggagagggggacctggctcaaggccacaccacatttccatgtcattttccagtggcaccaactgagctggacagatgttctcacagagctacacatgccacagtcatcactagttgcgagagtctccagtgttcattaagcctattttgctggaagtttagtccctggaggattagtccctgcccttaaggagtgccaaagaagactttggaatctaagtcacatctcccctgctgcaattttttcctcttgataatcaaggaatctcactgataaaactctcaatgaggagcagctctccccatgagcaagttcttgccttcattgccagggaggccccagctaaggttatactgggaaaggaatctctggggagtacctggaagaggtcagcttctcctcaaaggaagcctctccagctggtgtactgcaaagccttccaagaccaagttccctcttcctggcctctggaaccagcctgtgtgccagcgctgtgtccccagtaacacacatgagtctctccctcagaagccacagaaggatgcagagagagaccaacccaggattgattctcagcacttactaattgtgtggacataactgctctggacctcagctttgctatctataaaatgagcaccctttttatagatatgaacactttaagagagtcataaagattgaagttgatttgtgctgcgcctggcacagaggtctccctcagtaaatggcagccactgctattggggtgccccaacaccccttgcccctgcccaccattagctttcacttaggcccacactgagggtgtggctgttgtgttgggggaaggaaaaaaaccatgcctgggttgcagggcccccaccaccaactggtctgcttttgtgggaagcattttctgatgccctcgccctacccgtgggctggttgactaagtgtccagcatcatgaggaaaagagcagggttcagggacagcttggctcagccctgaaactcatctgggcctaggtccagatgagaggcaagcctgggaggccttccgttgtacccttgcctatcctgcagcaccctctagcctgcaggccgctcctgggtggcatagcaccttggatgtcaggtgtgggccttcccaggcacatggcatgagggggcacttctgtgggttgttgggggcaggaagggatgtgtctccagctggacttgggctctcgcattttggggcccagccatgccaggacagcacacatgggcgcttagtgcgaatctcatgatggagcagaggaggagcaaagcaaaaccagggagtccccaggcccctgcttctgccccgcccagagacagtggagcaggtgtctccagcttcttaacctcaacgcacagtaagaaatacattttacagcaaccaatagacacatacatttacaaaacga

SEQ ID NO: 14 - protein, 534 aa, Homo sapiens
MRRHSETDVEEQTQELKTITQLQEQCRALQIQGVKENMDQNKATLALLRSNIRRGAQDWALAKKYDQWTISKACGKNLPLRLAHCRSTMEVVREKLRKYVFDRVNMHNLLIHLVRRRGQKLESMQLELDSLRSQPDASKEELRLLQIIRQLENNIEKTMIKIITSQNIHLLYLDLLDYLKTVLAGYPIELDKLQNLVVNYCSELSDMKIMSQDAMMITDEVKRNMRQREASFIEERRARENRLNQQKKLIDKIHTKETSEKYRRGQMDLDFPSNLMSTETLKLRRKETSTAEMEYQSGVTAVVEKVKSAVRCSHVWDITSRFLAQRNTEENLELQMEDCEEWRVQLKALVKQLELEEAVLKFRQKPSSISFKSVEKKMTDMLKEEEERLQLAHSNMTKGQELLLTIQMGIDNLYVRLMGINLPATQREVVLSNTLDLNSKLAYCEGKLTYLADRVQMVSRTEEGDTKVRDTLESSTLMEKYNTRISFENREEDMIDTFQFPDMDHSYVPSRAEIKRQAQRLIEGKLKAAKKKKK

SEQ ID NO: 15 - protein, 399 aa, Homo sapiens
MERKNPSRESPRRLSAKVGKGTEMKKVARQLGMAAAESDKDSGFSDGSSECLSSAEQMESEDMLSALGWSREDRPRQNSKTAKNAFPTLSPMVVMKNVLVKQGSSSSQLQSWTVQPSFEVISAQPQLLFLHPPVPSPVSPCHTGEKKSDSRNYLPILNSYTKIAPHPGKRGLSLGPEEKGTSGVQKKICTERLGPSLSSSEPTKAGAVPSSPSTPAPPSAKLAEDSALQGVPSLVAGGSPQTLQPVSSSHVAKAPSLTFASPASPVCASDSTLHGLESNSPLSPLSANYSSPLWAAEHLCRSPDIFSEQRQSKHRRFQNTLVVLHKSGLLEITLKTKELIRQNQATQVELDQLKEQTQLFIEATKSRAPQAWAKLQASLTPGSSNTGSDLEAFSDHPAI

SEQ ID NO: 16 - protein, 768 aa, Homo sapiens
MSNLSKGTGSRKDTKMRIRAFPMTMDEKYVNSIWDLLKNAIQEIQRKNNSGLSFEELYRNAYTMVLHKHGEKLYTGLREVVTEHLINKVREDVLNSLNNNFLQTLNQAWNDHQTAMVMIRDILMYMDRVYVQQNNVENVYNLGLIIFRDQVVRYGCIRDHLRQTLLDMIARERKGEVVDRGAIRNACQMLMILGLEGRSVYEEDFEAPFLEMSAEFFQMESQKFLAENSASVYIKKVEARINEEIERVMHCLDKSTEEPIVKVVERELISKHMKTIVEMENSGLVHMLKNGKTEDLGCMYKLFSRVPNGLKTMCECMSSYLREQGKALVSEEGEGKNPVDYIQGLLDLKSRFDRFLLESFNNDRLFKQTIAGDFEYFLNLNSRSPEYLSLFIDDKLKKGVKGLTEQEVETILDKAMVLFRFMQEKDVFERYYKQHLARRLLTNKSVSDDSEKNMISKLKTECGCQFTSKLEGMFRDMSISNTTMDEFRQHLQATGVSLGGVDLTVRVLTTGYWPTQSATPKCNIPPAPRHAFEIFRRFYLAKHSGRQLTLQHHMGSADLNATFYGPVKKEDGSEVGVGGAQVTGSNTRKHILQVSTFQMTILMLFNNREKYTFEEIQQETDIPERELVRALQSLACGKPTQRVLTKEPKSKEIENGHIFTVNDQFTSKLHRVKIQTVAAKQGESDPERKETRQKVDDDRKHEIEAAIVRIMKSRKKMQHNVLVAEVTQQLKARFLPSPVVIKKRIEGLIEREYLARTPEDRKVYTYVA

SEQ ID NO: 17 - protein, 327 aa, Homo sapiens
MRALPGLLEARARTPRLLLLQCLLAAARPSSADGSAPDSPFTSPPLREEIMANNFSLESHNISLTEHSSMPVEKNITLERPSNVNLTCQFTTSGDLNAVNVTWKKDGEQLENNYLVSATGSTLYTQYRFTIINSKQMGSYSCFFREEKEQRGTFNFKVPELHGKNKPLISYVGDSTVLTCKCQNCFPLNWTWYSSNGSVKVPVGVQMNKYVINGTYANETKLKITQLLEEDGESYWCRALFQLGESEEHIELVVLSYLVPLKPFLVIVAEVILLVATILLCEKYTQKKKKHSDEGKEFEQIEQLKSDDSNGIENNVPRHRKNESLGQ

SEQ ID NO: 18 - protein, 681 aa, Homo sapiens
MTASPDYLVVLFGITAGATGAKLGSDEKELILLFWKVVDLANKKVGQLHEVLVRPDQLELTEDCKEETKIDVESLSSASQLDQALRQFNQSVSNELNIGVGTSFCLCTDGQLHVRQILHPEASKKNVLLPECFYSFFDLRKEFKKCCPGSPDIDKLDVATMTEYLNFEKSSSVSRYGASQVEDMGNIILAMISEPYNHRFSDPERVNYKFESGTCSKMELIDDNTVVRARGLPWQSSDQDIARFFKGLNIAKGGAALCLNAQGRRNGEALVRFVSEEHRDLALQRHKHHMGTRYIEVYKATGEDFLKIAGGTSNEVAQFLSKENQVIVRMRGLPFTATAEEVVAFFGQHCPITGGKEGILFVTYPDGRPTGDAFVLFACEEYAQNALRKHKDLLGKRYIELFRSTAAEVQQVLNRFSSAPLIPLPTPPIIPVLPQQFVPPTNVRDCIRLRGLPYAATIEDILDFLGEFATDIRTHGVHMVLNHQGRPSGDAFIQMKSADRAFMAAQKCHKKNMKDRYVEVFQCSAEEMNFVLMGGTLNRNGLSPPPCKLPCLSPPSYTFPAPAAVIPTEAAIYQPSVILNPRALQPSTAYYPAGTQLFMNYTAYYPSPPGSPNSLGYFPTAANLSGVPPQPGTVVRMQGLAYNTGVKEILNFFQGYQYATEDGLIHTNDQARTLPKEWVCI

SEQ ID NO: 19 - protein, 735 aa, Homo sapiens
MTPELMIKACSFYTGHLVKTHFCTWRDIARTNENVVLAEKMNRAVTCYNFRLQKSVFHHWHSYMEDQKEKLKNILLRIQQIIYCHKLTIILTKWRNTARHKSKKKEDELILKHELQLKKWKNRLILKRAAAEESNFPERSSSEVFLVDETLKCDISLLPERAILQIFFYLSLKDVIICGQVNHAWMLMTQLNSLWNAIDFSSVKNVIPDKYIVSTLQRWRLNVLRLNFRGCLLRPKTFRSVSHCRNLQELNVSDCPTFTDESMRHISEGCPGVLCLNLSNTTITNRTMRLLPRHFHNLQNLSLAYCRRFTDKGLQYLNLGNGCHKLIYLDLSGCTQISVQGFRYIANSCTGIMHLTINDMPTLTDNCVKALVEKCSRITSLVFTGAPHISDCTFRALSACKLRKIRFEGNKRVTDASFKFIDKNYPNLSHIYMADCKGITDSSLRSLSPLKQLTVLNLANCVRIGDMGLKQFLDGPASMRIRELNLSNCVRLSDASVMKLSERCPNLNYLSLRNCEHLTAQGIGYIVNIFSLVSIDLSGTDISNEGLNVLSRHKKLKELSVSECYRITDDGIQAFCKSSLILEHLDVSYCSQLSDMIIKALAIYCINLTSLSIAGCPKITDSAMEMLSAKCHYLHILDISGCVLLTDQILEDLQIGCKQLRILKMQYCTNISKKAAQRMSSKVQQQEYNTNDPPRWFGYDREGNPVTELDNITSSKGALELTVKKSTYSSEDQAA

SEQ ID NO: 20 - protein, 875 aa, Homo sapiens
MASLDLPYRCPRCGEHKRFRSLSSLRAHLEYSHTYETLYILSKTNSICDGAAAAAAAAAAASGFPLAPEPAALLAVPGARREVFESTSFQGKEQAAGPSPAAPHLLHHHHHHAPLAHFPGDLVPASLPCEELAEPGLVPAAAARYALREIEIPLGELFARKSVASSACSTPPPGPGPGPCPGPASASPASPSPADVAYEEGLARLKIRALEKLEVDRRLERLSEEVEQKIAGQVGRLQAELERKAAELETARQESARLGREKEELEERASELSRQVDVSVELLASLKQDLVHKEQELSRKQQEVVQIDQFLKETAAREASAKLRLQQFIEELLERADRAERQLQVISSSCGSTPSASLGRGGGGGGAGPNARGPGRMREHHVGPAVPNTYAVSRHGSSPSTGASSRVPAASQSSGCYDSDSLELPRPEEGAPEDSGPGGLGTRAQAANGGSERSQPPRSSGLRRQAIQNWQRRPRRHSTEGEEGDVSDVGSRTTESEAEGPLDAPRPGPAMAGPLSSCRLSARPEGGSGRGRRAERVSPSRSNEVISPEILKMRAALFCIFTYLDTRTLLHAAEVCRDWRFVARHPAVWTRVLLENARVCSKFLAMLAQWCTQAHSLTLQNLKPRQRGKKESKEEYARSTRGCLEAGLESLLKAAGGNLLILRISHCPNILTDRSLWLASCYCRALQAVTYRSATDPVGHEVIWALGAGCREIVSLQVAPLHPCQQPTRFSNRCLQMIGRCWPHLRALGVGGAGCGVQGLASLARNCMRLQVLELDHVSEITQEVAAEVCREGLKGLEMLVLTATPVTPKALLHFNSICRNLKSIVVQIGIADYFKEPSSPEAQKLFEDMVTKLQALRRRPGFSKILHIKVEGGC

SEQ ID NO: 21 - protein, 271 aa, Homo sapiens
MFRDFGEPGPSSGNGGGYGGPAQPPAAAQAAQQKFHLVPSINTMSGSQELQWMVQPHFLGPSSYPRPLTYPQYSPPQPRPGVIRALGPPPGVRRRPCEQISPEEEERRRVRRERNKLAAAKCRNRRKELTDFLQAETDKLEDEKSGLQREIEELQKQKERLELVLEAHRPICKIPEGAKEGDTGSTSGTSSPPAPCRPVPCISLSPGPVLEPEALHTPTLMTTPSLTPFTPSLVFTYPSTPEPCASAHRKSSSSSGDPSSDPLGSPTLLAL

SEQ ID NO: 22 - protein, 505 aa, Homo sapiens
MDPGNENSATEAAAIIDLDPDFEPQSRPRSCTWPLPRPEIANQPSEPPEVEPDLGEKVHTEGRSEPILLPSRLPEPAGGPQPGILGAVTGPRKGGSRRNAWGNQSYAELISQAIESAPEKRLTLAQIYEWMVRTVPYFKDKGDSNSSAGWKNSIRHNLSLHSKFIKVHNEATGKSSWWMLNPEGGKSGKAPRRRAASMDSSSKLLRGRSKAPKKKPSVLPAPPEGATPTSPVGHFAKWSGSPCSRNREEADMWTTFRPRSSSNASSVSTRLSPLRPESEVLAEEIPASVSSYAGGVPPTLNEGLELLDGLNLTSSHSLLSRSGLSGFSLQHPGVTGPLHTYSSSLFSPAEGPLSAGEGCFSSSQALEALLTSDTPPPPADVLMTQVDPILSQAPTLLLLGGLPSSSKLATGVGLCPKPLEAPGPSSLVPTLSMIAPPPVMASAPIPKALGTPVLTPPTEAASQDRMPQDLDLDMYMENLECDMDNIISDLMDEGEGLDFNFEPDP

SEQ ID NO: 23 - protein, 418 aa, Homo sapiens
MGEEGTGGTVHLLCLAASSGVPLFCRSSRGGAPARQQLPFSVIGSLNGVHMFGQNLEVQLSSARTENTTVVWKSFHDSITLIVLSSEVGISELRLERLLQMVFGAMVLLVGLEELTNIRNVERLKKDLRASYCLIDSFLGDSELIGDLTQCVDCVIPPEGSLLQEALSGFAEAAGTTFVSLVVSGRVVAATEGWWRLGTPEAVLLPWLVGSLPPQTARDYPVYLPHGSPTVPHRLLTLTLLPSLELCLLCGPSPPLSQLYPQLLERWWQPLLDPLRACLPLGPRALPSGFPLHTDILGLLLLHLELKRCLFTVEPLGDKEPSPEQRRRLLRNFYTLVTSTHFPPEPGPPEKTEDEVYQAQLPRACYLVLGTEEPGTGVRLVALQLGLRRLLLLLSPQSPTHGLRSLATHTLHALTPLL

SEQ ID NO: 24 - protein, 464 aa, Homo sapiens
MVRLAAELLLLLGLLLLTLHITVLRGSGAADGPDAAAGNASQAQLQNNLNVGSDTTSETSFSLSKEAPREHLDHQAAHQPFPRPRFRQETGHPSLQRDFPRSFLLDLPNFPDLSKADINGQNPNIQVTIEVVDGPDSEADKDQHPENKPSWSVPSPDWRAWWQRSLSLARANSGDQDYKYDSTSDDSNFLNPPRGWDHTAPGHRTFETKDQPEYDSTDGEGDWSLWSVCSVTCGNGNQKRTRSCGYACTATESRTCDRPNCPGIEDTFRTAATEVSLLAGSEEFNATKLFEVDTDSCERWMSCKSEFLKKYMHKVMNDLPSCPCSYPTEVAYSTADIFDRIKRKDFRWKDASGPKEKLEIYKPTARYCIRSMLSLESTTLAAQHCCYGDNMQLITRGKGAGTPNLISTEFSAELHYKVDVLPWIICKGDWSRYNEARPPNNGQKCTESPSDEDYIKQFQEAREY

SEQ ID NO: 25 - protein, 709 aa, Homo sapiens
MWWGGRGQSFNIAPQKEEPEMGSVQENRMPEPRSRQPSSCLASRCLPGEQILAWAPGVRKGLEPELSGTLICTNFRVTFQPCGWQWNQDTPLNSEYDFALVNIGRLEAVSGLSRVQLLRPGSLHKFIPEEILIHGRDFRLLRVGFEAGGLEPQAFQVTMAIVQARAQSNQAQQYSGITLSKAGQGSGSRKPPIPLMETAEDWETERKKQAARGWRVSTVNERFDVATSLPRYFWVPNRILDSEVRRAFGHFHQGRGPRLSWHHPGGSDLLRCGGFYTASDPNKEDIRAVELMLQAGHSDVVLVDTMDELPSLADVQLAHLRLRALCLPDSSVAEDKWLSALEGTRWLDYVRACLRKASDISVLVTSRVRSVILQERGDRDLNGLLSSLVQLLSAPEARTLFGFQSLVQREWVAAGHPFLTRLGGTGASEEAPVFLLFLDCVWQLLQQFPADFEFSEFFLLALHDSVRVPDTLTFLRNTPWERGKQSGQLNSYTQVYTPGYSQPPAGNSFNLQLSVWDWDLRYSNAQILQFQNPGYDPEHCPDSWLPRPQPSFMVPGPPSSVWLFSRGALTPLNQLCPWRDSPSLLAVSSRWLPRPAISSESLADQEWGLPSHWGACPLPPGLLLPGYLGPQIRLWRRCYLRGRPEVQMGLSAPTISGLQDELSHLQELLRKWTPRISPEDHSKKRDPHTILNPTEIAGILKGRAEGDLG

SEQ ID NO: 26 - protein, 375 aa, Homo sapiens
MDELQDVQLTEIKPLLNDKNGTRNFQDFDCQEHDIETTHGVVHVTIRGLPKGNRPVILTYHDIGLNHKSCFNAFFNFEDMQEITQHFAVCHVDAPGQQEGAPSFPTGYQYPTMDELAEMLPPVLTHLSLKSIIGIGVGAGAYILSRFALNHPELVEGLVLINVDPCAKGWIDWAASKLSGLTTNVVDIILAHHFGQEELQANLDLIQTYRMHIAQDINQDNLQLFLNSYNGRRDLEIERPILGQNDNKSKTLKCSTLLVVGDNSPAVEAVVECNSRLNPINTTLLKMADCGGLPQVVQPGKLTEAFKYFLQGMGYIPSASMTRLARSRTHSTSSSLGSGESPFSRSVTSNQSDGTQESCESPDVLDRHQTMEVSC

SEQ ID NO: 27 - protein, 608 aa, Homo sapiens
MAESIIIRVQSPDGVKRITATKRETAATFLKKVAKEFGFQNNGFSVYINRNKTGEITASSNKSLNLLKIKHGDLLFLFPSSLAGPSSEMETSVPPGFKVFGAPNVVEDEIDQYLSKQDGKIYRSRDPQLCRHGPLGKCVHCVPLEPFDEDYLNHLEPPVKHMSFHAYIRKLTGGADKGKFVALENISCKIKSGCEGHLPWPNGICTKCQPSAITLNRQKYRHVDNIMFENHTVADRFLDFWRKTGNQHFGYLYGRYTEHKDIPLGIRAEVAAIYEPPQIGTQNSLELLEDPKAEVVDEIAAKLGLRKVGWIFTDLVSEDTRKGTVRYSRNKDTYFLSSEECITAGDFQNKHPNMCRLSPDGHFGSKFVTAVATGGPDNQVHFEGYQVSNQCMALVRDECLLPCKDAPELGYAKESSSEQYVPDVFYKDVDKFGNEITQLARPLPVEYLIIDITTTFPKDPVYTFSISQNPFPIENRDVLGETQDFHSLATYLSQNTSSVFLDTISDFHLLLFLVTNEVMPLQDSISLLLEAVRTRNEELAQTWKRSEQWATIEQLCSTVGGQLPGLHEYGAVGGSTHTATAAMWACQHCTFMNQPGTGHCEMCSLPRT

SEQ ID NO: 28 - protein, 596 aa, Homo sapiens
MGRGARAAAAQSRWGRASRASVSPGRTIRSAPAVGEAQETEAAPEKENRVDVGAEERAAATRPRQKSWLVRHFSLLLRRDRQAQKAGQLFSGLLALNVVFLGGAFICSMIFNKVAVTLGDVWILLATLKVLSLLWLLYYVASTTRRPHAVLYQDPHAGPLWVRGSLVLFGSCTFCLNIFRVGYDVSHIRCKSQLDLVFSVIEMVFIGVQTWVLWKHCKDCVRVQTNFTRCGLMLTLATNLLLWVLAVTNDSMHREIEAELGILMEKSTGNETNTCLCLNATACEAFRRGFLMLYPFSTEYCLICCAVLFVMWKNVGRHVAPHMGAHPATAPFHLHGAIFGPLLGLLVLLAGVCVFVLFQIEASGPAIACQYFTLYYAFYVAVLPTMSLACLAGTAIHGLEERELDTVKNPTRSLDVVLLMGAALGQMGIAYFSIVAIVAKRPHELLNRLILAYSLLLILQHIAQNLFIIEGLHRRPLWETVPEGLAGKQEAEPPRRGSLLELGQGLQRASLAYIHSYSHLNWKRRALKEISLFLILCNITLWMMPAFGIHPEFENGLEKDFYGYQIWFAIVNFGLPLGVFYRMHSVGGLVEVYLGA

SEQ ID NO: 29 - protein, 926 aa, Homo sapiens
MVSSVLPNPTSAECWAALLHDPMTLDMDAVLSDFVRSTGAEPGLARDLLEGKNWDLTAALSDYEQLRQVHTANLPHVFNEGRGPKQPEREPQPGHKVERPCLQRQDDIAQEKRLSRGISHASSAIVSLARSHVASECNNEQFPLEMPIYTFQLPDLSVYSEDFRSFIERDLIEQATMVALEQAGRLNWWSTVCTSCKRLLPLATTGDGNCLLHAASLGMWGFHDRDLVLRKALYTMMRTGAEREALKRRWRWQQTQQNKEEEWEREWTELLKLASSEPRTHFSKNGGTGGGVDNSEDPVYESLEEFHVFVLAHILRRPIVVVADTMLRDSGGEAFAPIPFGGIYLPLEVPPNRCHCSPLVLAYDQAHFSALVSMEQRDQQREQAVIPLTDSEHKLLPLHFAVDPGKDWEWGKDDNDNARLAHLILSLEAKLNLLHSYMNVTWIRIPSETRAPLAQPESPTASAGEDVQSLADSLDSDRDSVCSNSNSNNGKNGKDKEKEKQRKEKDKTRADSVANKLGSFSKTLGIKLKKNMGGLGGLVHGKMGRANSANGKNGDSAERGKEKKAKSRKGSKEESGASASTSPSEKTTPSPTDKAAGASPAEKGGGPRGDAWKYSTDVKLSLNILRAAMQGERKFIFAGLLLTSHRHQFHEEMIGYYLTSAQERFSAEQEQRRRDAATAAAAAAAAAAATAKRPPRRPETEGVPVPERASPGPPTQLVLKLKERPSPGPAAGRAARAAAGGTASPGGGARRASASGPVPGRSPPAPARQSVIHVQASGARDEACAPAVGALRPCATYPQQNRSLSSQSYSPARAAALRTVNTVESLARAVPGALPGAAGTAGAAEHKSQTYTNGFGALRDGLEFADADAPTARSNGECGRGGPGPVQRRCQRENCAFYGRAETEHYCSYCYREELRRRREARGARP

SEQ ID NO: 30 - protein, 1141 aa, Homo sapiens
MAVPGDAARVRDKPVHSGVSQAPTAGRDCHHRADPASPRDSGCRGCWGDLVLQPLRSSRKLSSALCAGSLSFLLALLVRLVRGEVGCDLEQCKEAAAAEEEEAAPGAEGGVFPGPRGGAPGGGARLSPWLQPSALLFSLLCAFFWMGLYLLRAGVRLPLAVALLAACCGGEALVQIGLGVGEDHLLSLPAAGVVLSCLAAATWLVLRLRLGVLMIALTSAVRTVSLISLERFKVAWRPYLAYLAGVLGILLARYVEQILPQSAEAAPREHLGSQLIAGTKEDIPVFKRRRRSSSVVSAEMSGCSSKSHRRTSLPCIPREQLMGHSEWDHKRGPRGSQSSGTSITVDIAVMGEAHGLITDLLADPSLPPNVCTSLRAVSNLLSTQLTFQAIHKPRVNPVTSLSENYTCSDSEESSEKDKLAIPKRLRRSLPPGLLRRVSSTWTTTTSATGLPTLEPAPVRRDRSTSIKLQEAPSSSPDSWNNPVMMTLTKSRSFTSSYAISAANHVKAKKQSRPGALAKISPLSSPCSSPLQGTPASSLVSKISAVQFPESADTTAKQSLGSHRALTYTQSAPDLSPQILTPPVICSSCGRPYSQGNPADEPLERSGVATRTPSRTDDTAQVTSDYETNNNSDSSDIVQNEDETECLREPLRKASACSTYAPETMMFLDKPILAPEPLVMDNLDSIMEQLNTWNFPIFDLVENIGRKCGRILSQVSYRLFEDMGLFEAFKIPIREFMNYFHALEIGYRDIPYHNRIHATDVLHAVWYLTTQPIPGLSTVINDHGSTSDSDSDSGFTHGHMGYVFSKTYNVTDDKYGCLSGNIPALELMALYVAAAMHDYDHPGRTNAFLVATSAPQAVLYNDRSVLENHHAAAAWNLFMSRPEYNFLINLDHVEFKHFRFLVIEAILATDLKKHFDFVAKFNGKVNDDVGIDWTNENDRLLVCQMCIKLADINGPAKCKELHLQWTDGIVNEFYEQGDEEASLGLPISPFMDRSAPQLANLQESFISHIVGPLCNSYDSAGLMPGKWVEDSDESGDTDDPEEEEEEAPAPNEEETCENNESPKKKTFKRRKIYCQITQHLLQNHKMWKKVIEEEQRLAGIENQSLDQTPQSHSSEQIQAIKEEEEEKGKPRGEEIPTQKPDQ

SEQ ID NO: 31 - protein, 870 aa, Homo sapiens
MDSTKEKCDSYKDDLLLRMGLNDNKAGMEGLDKEKINKIIMEATKGSRFYGNELKKEKQVNQRIENMMQQKAQITSQQLRKAQLQVDRFAMELEQSRNLSNTIVHIDMDAFYAAVEMRDNPELKDKPIAVGSMSMLSTSNYHARRFGVRAAMPGFIAKRLCPQLIIVPPNFDKYRAVSKEVKEILADYDPNFMAMSLDEAYLNITKHLEERQNWPEDKRRYFIKMGSSVENDNPGKEVNKLSEHERSISPLLFEESPSDVQPPGDPFQVNFEEQNNPQILQNSVVFGTSAQEVVKEIRFRIEQKTTLTASAGIAPNTMLAKVCSDKNKPNGQYQILPNRQAVMDFIKDLPIRKVSGIGKVTEKMLKALGIITCTELYQQRALLSLLFSETSWHYFLHISLGLGSTHLTRDGERKSMSVERTFSEINKAEEQYSLCQELCSELAQDLQKERLKGRTVTIKLKNVNFEVKTRASTVSSVVSTAEEIFAIAKELLKTEIDADFPHPLRLRLMGVRISSFPNEEDRKHQQRSIIGFLQAGNQALSATECTLEKTDKDKFVKPLEMSHKKSFFDKKRSERKWSHQDTFKCEAVNKQSFQTSQPFQVLKKKMNENLEISENSDDCQILTCPVCFRAQGCISLEALNKHVDECLDGPSISENFKMFSCSHVSATKVNKKENVPASSLCEKQDYEAHPKIKEISSVDCIALVDTIDNSSKAESIDALSNKHSKEECSSLPSKSFNIEHCHQNSSSTVSLENEDVGSFRQEYRQPYLCEVKTGQALVCPVCNVEQKTSDLTLFNVHVDVCLNKSFIQELRKDKFNPVNQPKESSRSTGSSSGVQKAVTRTKRPGLMTKYSTSKKIKPNNPKHTLDIFFK

SEQ ID NO: 32 - protein, 57 aa, Homo sapiens
MLCAHFSDQGPAHLTTSKSAFLSNKKTSTLKHLLGETRSDGSACNSGISGGRGRKIP

SEQ ID NO: 33 - protein, 418 aa, Homo sapiens
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 34 - protein, 353 aa, Homo sapiens
MPSPVGLLRALPLPWPQFLACTLRRLAGPRESTGPSQKPPPLCSVPCRVPAMTEEVAREALLSFVDSKCCYSSTVAGDLVIQELKRQTLCRYRLETFSESRISEWTFQPFTNHSVDGPQRGASPRLWDIKVQGPPMFQEDTRKFQVPHSSLVKECHKCHGRGRYKCSGCHGAGTVRCPSCCGAKRKAKQSRRCQLCAGSGRRRCSTCSGRGNKTCATCKGEKKLLHFIQLVIMWKNSLFEFVSEHRLNCPRELLAKAKGENLFKDENSVVYPIVDFPLRDISLASQRGIAEHSAALASRARVLQQRQTIELIPLTEVHYWYQGKTYVYYIYGTDHQVYAVDYPERYCCGCTIV

SEQ ID NO: 35 - protein, 202 aa, Homo sapiens
MCTGGCARCLGGTLIPLAFFGFLANILLFFPGGKVIDDNDHLSQEIWFFGGILGSGVLMIFPALVFLGLKNNDCCGCCGNEGCGKRFAMFTSTIFAVVGFLGAGYSFIISAISINKGPKCLMANSTWGYPFHDGDYLNDEALWNKCREPLNVVPWNLTLFSILLVVGGIQMVLCAIQVVNGLLGTLCGDCQCCGCCGGDGPV

SEQ ID NO: 36 - protein, 1499 aa, Homo sapiens
MEREPAGTEEPGPPGRRRRREGRTRTVRSNLLPPPGAEDPAAGAAKGERRRRRGCAQHLADNRLKTTKYTLLSFLPKNLFEQFHRPANVYFVFIALLNFVPAVNAFQPGLALAPVLFILAITAFRDLWEDYSRHRSDHKINHLGCLVFSREEKKYVNRFWKEIHVGDFVRLRCNEIFPADILLLSSSDPDGLCHIETANLDGETNLKRRQVVRGFSELVSEFNPLTFTSVIECEKPNNDLSRFRGCIIHDNGKKAGLYKENLLLRGCTLRNTDAVVGIVIYAGHETKALLNNSGPRYKRSKLERQMNCDVLWCVLLLVCMSLFSAVGHGLWIWRYQEKKSLFYVPKSDGSSLSPVTAAVYSFLTMIIVLQVLIPISLYVSIEIVKACQVYFINQDMQLYDEETDSQLQCRALNITEDLGQIQYIFSDKTGTLTENKMVFRRCTVSGVEYSHDANAQRLARYQEADSEEEEVVPRGGSVSQRGSIGSHQSVRVVHRTQSTKSHRRTGSRAEAKRASMLSKHTAFSSPMEKDITPDPKLLEKVSECDKSLAVARHQEHLLAHLSPELSDVFDFFIALTICNTVVVTSPDQPRTKVRVRFELKSPVKTIEDFLRRFTPSCLTSGCSSIGSLAANKSSHKLGSSFPSTPSSDGMLLRLEERLGQPTSAIASNGYSSQADNWASELAQEQESERELRYEAESPDEAALVYAARAYNCVLVERLHDQVSVELPHLGRLTFELLHTLGFDSVRKRMSVVIRHPLTDEINVYTKGADSVVMDLLQPCSSVDARGRHQKKIRSKTQNYLNVYAAEGLRTLCIAKRVLSKEEYACWLQSHLEAESSLENSEELLFQSAIRLETNLHLLGATGIEDRLQDGVPETISKLRQAGLQIWVLTGDKQETAVNIAYACKLLDHDEEVITLNATSQEACAALLDQCLCYVQSRGLQRAPEKTKGKVSMRFSSLCPPSTSTASGRRPSLVIDGRSLAYALEKNLEDKFLFLAKQCRSVLCCRSTPLQKSMVVKLVRSKLKAMTLAIGDGANDVSMIQVADVGVGISGQEGMQAVMASDFAVPKFRYLERLLILHGHWCYSRLANMVLYFFYKNTMFVGLLFWFQFFCGFSASTMIDQWYLIFFNLLFSSLPPLVTGVLDRDVPANVLLTNPQLYKSGQNMEEYRPRTFWFNMADAAFQSLVCFSIPYLAYYDSNVDLFTWGTPIVTIALLTFLLHLGIETKTWTWLNWITCGFSVLLFFTVALIYNASCATCYPPSNPYWTMQALLGDPVFYLTCLMTPVAALLPRLFFRSLQGRVFPTQLQLARQLTRKSPRRCSAPKETFAQGRLPKDSGTEHSSGRTVKTSVPLSQPSWHTQQPVCSLEASGEPSTVDMSMPVREHTLLEGLSAPAPMSSAPGEAVLRSPGGCPEESKVRAASTGRVTPLSSLFSLPTFSLLNWISSWSLVSRLGSVLQFSRTEQLADGQAGRGLPVQPHSGRSGLQGPDHRLLIGASSRRSQ

SEQ ID NO: 37 - protein, 80 aa, Homo sapiens
MSQQKQQSWKPPNVPKCSPPQRSNPCLAPYSTPCGAPHSEGCHSSSQRPEVQKPRRARQKLRCLSRGTTYHCKEEECEGD

SEQ ID NO: 38 - protein, 227 aa, Homo sapiens
MAPYHIRKYQESDRKSVVGLLSGGMAEHAPATFRRLLKLPRTLILLLGGALALLLVSGSWILALVFSLSLLPALWFLAKKPWTRYVDIALRTDMSDITKSYLSECGSCFWVAESEEKVVGTVGALPVDDPTLREKRLQLFHLSVDNEHRGQGIAKALVRTVLQFARDQGYSEVVLDTSNIQLSAMGLYQSLGFKKTGQSFFHVWARLVDLHTVHFIYHLPSAQAGRL

SEQ ID NO: 39 - protein, 150 aa, Homo sapiens
MSGELSNRFQGGKAFGLLKARQERRLAEINREFLCDQKYSDEENLPEKLTAFKEKYMEFDLNNEGEIDLMSLKRMMEKLGVPKTHLEMKKMISEVTGGVSDTISYRDFVNMMLGKRSAVLKLVMMFEGKANESSPKPVGPPPERDIASLP

SEQ ID NO: 40 - protein, 201 aa, Homo sapiens
MTEKAPEPHVEEDDDDELDSKLNYKPPPQKSLKELQEMDKDDESLIKYKKTLLGDGPVVTDPKAPNVVVTRLTLVCESAPGPITMDLTGDLEALKKETIVLKEGSEYRVKIHFKVNRDIVSGLKYVQHTYRTGVKVDKATFMVGSYGPRPEEYEFLTPVEEAPKGMLARGTYHNKSFFTDDDKQDHLSWEWNLSIKKEWTE

SEQ ID NO: 41 - protein, 1417 aa, Homo sapiens
MAAVPQNNLQEQLERHSARTLNNKLSLSKPKFSGFTFKKKTSSDNNVSVTNVSVAKTPVLRNKDVNVTEDFSFSEPLPNTTNQQRVKDFFKNAPAGQETQRGGSKSLLPDFLQTPKEVVCTTQNTPTVKKSRDTALKKLEFSSSPDSLSTINDWDDMDDFDTSETSKSFVTPPQSHFVRVSTAQKSKKGKRNFFKAQLYTTNTVKTDLPPPSSESEQIDLTEEQKDDSEWLSSDVICIDDGPIAEVHINEDAQESDSLKTHLEDERDNSEKKKNLEEAELHSTEKVPCIEFDDDDYDTDFVPPSPEEIISASSSSSKCLSTLKDLDTSDRKEDVLSTSKDLLSKPEKMSMQELNPETSTDCDARQISLQQQLIHVMEHICKLIDTIPDDKLKLLDCGNELLQQRNIRRKLLTEVDFNKSDASLLGSLWRYRPDSLDGPMEGDSCPTGNSMKELNFSHLPSNSVSPGDCLLTTTLGKTGFSATRKNLFERPLFNTHLQKSFVSSNWAETPRLGKKNESSYFPGNVLTSTAVKDQNKHTASINDLERETQPSYDIDNFDIDDFDDDDDWEDIMHNLAASKSSTAAYQPIKEGRPIKSVSERLSSAKTDCLPVSSTAQNINFSESIQNYTDKSAQNLASRNLKHERFQSLSFPHTKEMMKIFHKKFGLHNFRTNQLEAINAALLGEDCFILMPTGGGKSLCYQLPACVSPGVTVVISPLRSLIVDQVQKLTSLDIPATYLTGDKTDSEATNIYLQLSKKDPIIKLLYVTPEKICASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQRDGLAALAYHAGLSDSARDEVQQKWINQDGCQVICATIAFGMGIDKPDVRFVIHASLPKSVEGYYQESGRAGRDGEISHCLLFYTYHDVTRLKRLIMMEKDGNHHTRETHFNNLYSMVHYCENITECRRIQLLAYFGENGFNPDFCKKHPDVSCDNCCKTKDYKTRDVTDDVKSIVRFVQEHSSSQGMRNIKHVGPSGRFTMNMLVDIFLGSKSAKIQSGIFGKGSAYSRHNAERLFKKLILDKILDEDLYINANDQAIAYVMLGNKAQTVLNGNLKVDFMETENSSSVKKQKALVAKVSQREEMVKKCLGELTEVCKSLGKVFGVHYFNIFNTVTLKKLAESLSSDPEVLLQIDGVTEDKLEKYGAEVISVLQKYSEWTSPAEDSSPGISLSSSRGPGRSAAEELDEEIPVSSHYFASKTRNERKRKKMPASQRSKRRKTASSGSKAKGGSATCRKISSKTKSSSIIGSSSASHTSQATSGANSKLGIMAPPKPINRPFLKPSYAFS

SEQ ID NO: 42 - protein, 648 aa, Homo sapiens
MEAKFLASALSFLSIFLAIYAQSLANDLSKESTEFEESPTIYYGDPVVNLGQPFSITCIIPITDQIHWLKNGEPITRHNLRHGRDDHAYVLSESAIEGEKHKIEAHLSVRHALKVHEGRYQCNRRRGSYILHVRDPKGVGAGAGEPTESGYQTIDELTPNSADDFFTRAWLEQQQQQQQLPHQSHKLHKSHLGYGNASLSGSQPWHPSAGGGGIHRVYSATPPDFPPPRLNLLEQTVAPPEPPTILYNPNPTHPTASATATETSVLLTTAHHHAHHQQQLQQQSQHTLNAFQLPLPPRPNPGQNERYQTYAPHYVPPVVVSGAGAGAGADPGAGASGEQTTISAATSTRAMMGGGGGVAGAGFSAGASGPMLGAGGHMLMGGQGHQVHLQHQTLLPVKMDKLVPNYDNAEHQMKFYDIRSPLVLSCNVKDGTPGGVLIWKKNGTAVTDVPSLRGRFKLIADENKFIIDKTDTNDDGKYSCEFDGVSKEIEVIARVVVRVPSNTAVVEGEKMSVTCSVVGTKPELTWTFANVTLTNATDRFILKPDDNGVPNAILTLDNVTLDDRGEYKCIGRNAANVYGGNTTTPASDVTTVRVKGKFAALWPFLGICAEVLILCIIILIYEKRRNKSELEESDTDPQEQKKKRRNYD

SEQ ID NO: 43 - protein, 525 aa, Homo sapiens
MAAGGSGGRASCPPGVGVGPGTGGSPGPSANAAATPAPGNAAAAAAAAAAAAAAPGPTPPAPPGPGTDAQAAGAERAEEAAGPGAAALQREAAYNWQASKPTVQERFAFLFNNEVLCDVHFLVGKGLSSQRIPAHRFVLAVGSAVFDAMFNGGMATTSTEIELPDVEPAAFLALLKFLYSDEVQIGPETVMTTLYTAKKYAVPALEAHCVEFLKKNLRADNAFMLLTQARLFDEPQLASLCLENIDKNTADAITAEGFTDIDLDTLVAVLERDTLGIREVRLFNAVVRWSEAECQRQQLQVTPENRRKVLGKALGLIRFPLMTIEEFAAGPAQSGILVDREVVSLFLHFTVNPKPRVEFIDRPRCCLRGKECSINRFQQVESRWGYSGTSDRIRFSVNKRIFVVGFGLYGSIHGPTDYQVNIQIIHTDSNTVLGQNDTGFSCDGSASTFRVMFKEPVEVLPNVNYTACATLKGPDSHYGTKGLRKVTHESPTTGAKTCFTFCYAAGNNNGTSVEDGQIPEVIFYT

SEQ ID NO: 44 - protein, 329 aa, Homo sapiens
MELHILEHRLQVASVAKESIPLFTYGLIKLAFLSSKTRCKFFSLTETPEDYTIIVDEEGFLELPSSEHLSVADATWLALNVVSGGGSFSSSQPIGVTKIAKSVIAPLADQNISVFMLSTYQTDFILVRERDLPFVTHTLSSEFTILRVVNGETVAAENLGITNGFVKPKLVQRPVIHPLSSPSNRFCVTSLDPDTLPAVATLLMDVMFYSNGVKDPMATGDDCGHIRFFSFSLIEGYISLVMDVQTQQRFPSNLLFTSASGELWKMVRIGGQPLGFDECGIVAQISEPLAAADIPAYYISTFKFDHALVPEENINGVISALKVSQAEKH

SEQ ID NO: 45 - protein, 900 aa, Homo sapiens
MGDSGSRRSTLVSRLPIFRRSINRRHDSLPSSPSSSNTVGVHSSSPSSTNSSSGSTGKRRSIFRTPSISFHHKKGSEPKQEPTNQNLSISNGAQPGHSNMQKLSLEEHIKTRGRHSVGFSSSRNKKITRSLTEDFEREKEHSTNKNVFINCLSSGKSEGDDSGFTEDQTRRSVKQSTRKLLPKSFSSHYKFSKPVLQSQSISLVQQSEFSLEVTQYQEREPVLVRASPSCSVDVTERAGSSLQSPLLSADLTTAQTPSEFLALTEDSVSEMDAFSKSGSMASHCDNFGHNDSTSQMSLNSAAVTKTTTELTGTVPCAIMSPGKYRLEGQCSTESNSLPETSAANQKEVLLQIAELPATSVSHSESNLPADSEREENIGLQNGETMLGTNSPRKLGFYEQHKAIAEHVKGIHPISDSKIIPTSGDHHIFNKTSHGYEANPAKVLASSLSPFREGRFIERRLRSSSEGTAGSSRMILKPKDGNIEEVNSLRKQRAGSSSSKMNSLDVLNNLGSCELDEDDLMLDLEFLEEQSLHPSVCREDSYHSVVSCAAVVLTPMEPMIEMKKREEPEFPEPSKQNLSLKLTKDVDQEARCSHISRMPNSPSADWPLQGVEENGGIDSLPFRLMLQDCTAVKTLLLKMKRVLQESADMSPASSTTSLPVSPLTEEPVPFKDIMKDECSMLKLQLKEKDELISQLQEELGKVRHLQKAFASRVDKSTQTELLCYDGLNLKRLETVQGGREATYRNRIVSQNLSTRDRKAIHTPTEDRFRYSAADQTSPYKNKTCQLPSLCLSNFLKDKELAEVIKHSRGTYETLTSDVTQNLRATVGQSSLKPTAKTEGLSTFLEKPKDQVATARQHSTFTGRFGQPPRGPISLHMYSRKNVFLHHNLHSTELQTLGQQDG

SEQ ID NO: 46 - protein, 898 aa, Homo sapiens
MAAAAAEEGMEPRALQYEQTLMYGRYTQDLGAFAKEEAARIRLGGPEPWKGPPSSRAAPELLEYGRSRCARCRVCSVRCHKFLVSRVGEDWIFLVLLGLLMALVSWVMDYAIAACLQAQQWMSRGLNTSILLQYLAWVTYPVVLITFSAGFTQILAPQAVGSGIPEMKTILRGVVLKEYLTLKTFIAKVIGLTCALGSGMPLGKEGPFVHIASMCAALLSKFLSLFGGIYENESRNTEMLAAACAVGVGCCFAAPIGGVLFSIEVTSTFFAVRNYWRGFFAATFSAFIFRVLAVWNRDEETITALFKTRFRLDFPFDLQELPAFAVIGIASGFGGALFVYLNRKIVQVMRKQKTINRFLMRKRLLFPALVTLLISTLTFPPGFGQFMAGQLSQKETLVTLFDNRTWVRQGLVEELEPPSTSQAWNPPRANVFLTLVIFILMKFWMSALATTIPVPCGAFMPVFVIGAAFGRLVGESMAAWFPDGIHTDSSTYRIVPGGYAVVGAAALAGAVTHTVSTAVIVFELTGQIAHILPVMIAVILANAVAQSLQPSLYDSIIRIKKLPYLPELGWGRHQQYRVRVEDIMVRDVPHVALSCTFRDLRLALHRTKGRMLALVESPESMILLGSIERSQVVALLGAQLSPARRRQHMQERRATQTSPLSDQEGPPTPEASVCFQVNTEDSAFPAARGETHKPLKPALKRGPSVTRNLGESPTGSAESAGIALRSLFCGSPPPEAASEKLESCEKRKLKRVRISLASDADLEGEMSPEEILEWEEQQLDEPVNFSDCKIDPAPFQLVERTSLHKTHTIFSLLGVDHAYVTSIGRLIGIVTLKELRKAIEGSVTAQGVKVRPPLASFRDSATSSSDTETTEVHALWGPHSRHGLPREGSPSDSDDKCQ

SEQ ID NO: 47 - protein, 575 aa, Homo sapiens
MFGIQDTLGRGPTLKEKSLGAEMDSVRSWVRNVGVVDANVAAQSGVALSRAHFEKQPPSNLRKSNFFHFVLALYDRQGQPVEIERTAFVDFVENDKEQGNEKTNNGTHYKLQLLYSNGVRTEQDLYVRLIDSVTKQPIAYEGQNKNPEMCRVLLTHEVMCSRCCEKKSCGNRNETPSDPVIIDRFFLKFFLKCNQNCLKTAGNPRDMRRFQVVLSTTVNVDGHVLAVSDNMFVHNNSKHGRRARRLDPSEATPCIKAISPSEGWTTGGAMVIIIGDNFFDGLQVVFGTMLVWSELITPHAIRVQTPPRHIPGVVEVTLSYKSKQFCKGAPGRFIYTALNEPTIDYGFQRLQKVIPRHPGDPERLAKEMLLKRAADLVEALYGTPHNNQDIILKRAADIAEALYSVPRNPSQLPALSSSPAHSGMMGINSYGSQLGVSISESTQGNNQGYIRNTSSISPRGYSSSSTPQQSNYSTSSNSMNGYSNVPMANLGVPGSPGFLNGSPTGSPYGIMSSSPTVGSSSTSSILPFSSSVFPAVKQKSAFAPVIRPQGSPSPACSSGNGNGFRAMTGLVVPPM

SEQ ID NO: 48 - protein, 434 aa, Homo sapiens
MSRSRHLGKIRKRLEDVKSQWVRPARADFSDNESARLATDALLDGGSEAYWRVLSQEGEVDFLSSVEAQYIQAQAREPPCPPDTLGGAEAGPKGLDSSSLQSGTYFPVASEGSEPALLHSWASAEKPYLKEKSSATVYFQTVKHNNIRDLVRRCITRTSQVLVILMDVFTDVEIFCDILEAANKRGVFVCVLLDQGGVKLFQEMCDKVQISDSHLKNISIRSVEGEIYCAKSGRKFAGQIREKFIISDWRFVLSGSYSFTWLCGHVHRNILSKFTGQAVELFDEEFRHLYASSKPVMGLKSPRLVAPVPPGAAPANGRLSSSSGSASDRTSSNPFSGRSAGSHPGTRSVSASSGPCSPAAPHPPPPPRFQPHQGPWGAPSPQAHLSPRPHDGPPAAVYSNLGAYRPTRLQLEQLGLVPRLTPTWRPFLQASPHF

SEQ ID NO: 49 - protein, 379 aa, Homo sapiens
MSSAPEKQQPPHGGGGGGGGGGGAAMDPASSGPSKAKKTNAGIRRPEKPPYSYIALIVMAIQSSPTKRLTLSEIYQFLQSRFPFFRGSYQGWKNSVRHNLSLNECFIKLPKGLGRPGKGHYWTIDPASEFMFEEGSFRRRPRGFRRKCQALKPMYSMMNGLGFNHLPDTYGFQGSAGGLSCPPNSLALEGGLGMMNGHLPGNVDGMALPSHSVPHLPSNGGHSYMGGCGGAAAGEYPHHDSSVPASPLLPTGAGGVMEPHAVYSGSAAAWPPSASAALNSGASYIKQQPLSPCNPAANPLSGSLSTHSLEQPYLHQNSHNAPAELQGIPRYHSQSPSMCDRKEFVFSFNAMASSSMHSAGGGSYYHQQVTYQDIKPCVM

SEQ ID NO: 50 - protein, 420 aa, Homo sapiens
MALYCGDNFGVYSQPGLPPPAATAAAPGAPPAARAPYGLADYAAPPAAAANPYLWLNGPGVGGPPSAAAAAAAAYLGAPPPPPPPGAAAGPFLQPPPAAGTFGCSQRPFAQPAPAAPASPAAPAGPGELGWLSMASREDLMKMVRPPYSYSALIAMAIQSAPERKLTLSHIYQFVADSFPFYQRSKAGWQNSIRHNLSLNDCFKKVPRDEDDPGKGNYWTLDPNCEKMFDNGNFRRKRKRRSEASNGSTVAAGTSKSEEGLSSGLGSGVGGKPEEESPSTLLRPSHSPEPPEGTKSTASSPGGPMLTSTPCLNTFFSSLSSLSVSSSVSTQRALPGSRHLGIQGAQLPSSGVFSPTSISEASADTLQLSNSTSNSTGQRSSYYSPFPASTSGGQSSPFSSPFHNFSMVNSLIYPREGSEV

SEQ ID NO: 51 - protein, 175 aa, Homo sapiens
MTRTPVGSARTRPKPRKLGPQRGKALQASSRLSESPALVKKRMPDACTLGRAGIGLPKMCLHMAVRHSKAQKTGPGILQQRQKPPAPRASGGPALLGKRRGCSEAGSASLEPLSSSRAAAGCLNQVPLSPFLAGPRNTRRLPAPERERIELAATLCLEGWPLRCLASKGKLHCVY

SEQ ID NO: 52 - protein, 299 aa, Homo sapiens
MEELLPDGQIWANMDPEERMLAAATAFTHICAGQGEGDVRREAQSIQYDPYSKASVAPGKRPALPVQLQYPHVESNVPSETVSEASQRLRKPVMKRKVLRRKPDGEVLVTDESIISESESGTENDQDLWDLRQRLMNVQFQEDKESSFDVSQKFNLPHEYQGISQDQLICSLQREGMGSPAYEQDLIVASRPKSFILPKLDQLSRNRGKTDRVARYFEYKRDWDSIRLPGEDHRKELRWGVREQMLCRAEPQSKPQHIYVPNNYLVPTEKKRSALRWGVRCDLANGVIPRKLPFPLSPS

SEQ ID NO: 53 - protein, 1786 aa, Homo sapiens
MGLLQLLAFSFLALCRARVRAQEPEFSYGCAEGSCYPATGDLLIGRAQKLSVTSTCGLHKPEPYCIVSHLQEDKKCFICNSQDPYHETLNPDSHLIENVVTTFAPNRLKIWWQSENGVENVTIQLDLEAEFHFTHLIMTFKTFRPAAMLIERSSDFGKTWGVYRYFAYDCEASFPGISTGPMKKVDDIICDSRYSDIEPSTEGEVIFRALDPAFKIEDPYSPRIQNLLKITNLRIKFVKLHTLGDNLLDSRMEIREKYYYAVYDMVVRGNCFCYGHASECAPVDGFNEEVEGMVHGHCMCRHNTKGLNCELCMDFYHDLPWRPAEGRNSNACKKCNCNEHSISCHFDMAVYLATGNVSGGVCDDCQHNTMGRNCEQCKPFYYQHPERDIRDPNFCERCTCDPAGSQNEGICDSYTDFSTGLIAGQCRCKLNVEGEHCDVCKEGFYDLSSEDPFGCKSCACNPLGTIPGGNPCDSETGHCYCKRLVTGQHCDQCLPEHWGLSNDLDGCRPCDCDLGGALNNSCFAESGQCSCRPHMIGRQCNEVEPGYYFATLDHYLYEAEEANLGPGVSIVERQYIQDRIPSWTGAGFVRVPEGAYLEFFIDNIPYSMEYDILIRYEPQLPDHWEKAVITVQRPGRIPTSSRCGNTIPDDDNQVVSLSPGSRYVVLPRPVCFEKGTNYTVRLELPQYTSSDSDVESPYTLIDSLVLMPYCKSLDIFTVGGSGDGVVTNSAWETFQRYRCLENSRSVVKTPMTDVCRNIIFSISALLHQTGLACECDPQGSLSSVCDPNGGQCQCRPNVVGRTCNRCAPGTFGFGPSGCKPCECHLQGSVNAFCNPVTGQCHCFQGVYARQCDRCLPGHWGFPSCQPCQCNGHADDCDPVTGECLNCQDYTMGHNCERCLAGYYGDPIIGSGDHCRPCPCPDGPDSGRQFARSCYQDPVTLQLACVCDPGYIGSRCDDCASGYFGNPSEVGGSCQPCQCHNNIDTTDPEACDKETGRCLKCLYHTEGEHCQFCRFGYYGDALQQDCRKCVCNYLGTVQEHCNGSDCQCDKATGQCLCLPNVIGQNCDRCAPNTWQLASGTGCDPCNCNAAHSFGPSCNEFTGQCQCMPGFGGRTCSECQELFWGDPDVECRACDCDPRGIETPQCDQSTGQCVCVEGVEGPRCDKCTRGYSGVFPDCTPCHQCFALWDVIIAELTNRTHRFLEKAKALKISGVIGPYRETVDSVERKVSEIKDILAQSPAAEPLKNIGNLFEEAEKLIKDVTEMMAQVEVKLSDTTSQSNSTAKELDSLQTEAESLDNTVKELAEQLEFIKNSDIRGALDSITKYFQMSLEAEERVNASTTEPNSTVEQSALMRDRVEDVMMERESQFKEKQEEQARLLDELAGKLQSLDLSAAAEMTCGTPPGASCSETECGGPNCRTDEGERKCGGPGCGGLVTVAHNAWQKAMDLDQDVLSALAEVEQLSKMVSEAKLRADEAKQSAEDILLKTNATKEKMDKSNEELRNLIKQIRNFLTQDSADLDSIEAVANEVLKMEMPSTPQQLQNLTEDIRERVESLSQVEVILQHSAADIARAEMLLEEAKRASKSATDVKVTADMVKEALEEAEKAQVAAEKAIKQADEDIQGTQNLLTSIESETAASEETLFNASQRISELERNVEELKRKAAQNSGEAEYIEKVVYTVKQSAEDVKKTLDGELDEKYKKVENLIAKKTEESADARRKAEMLQNEAKTLLAQANSKLQLLKDLERKYEDNQRYLEDKAQELARLEGEVRSLLKDISQKVAVYSTCL

SEQ ID NO: 54 - protein, 61 aa, Homo sapiens
MQPRTQPLAQTLPFFLGGAPRDTGLRVPVIKMGTGWEGFQRTLKEVAYILLCCWCIKELLD

SEQ ID NO: 55 - protein, 713 aa, Homo sapiens
MARARGSPCPPLPPGRMSWPHGALLFLWLFSPPLGAGGGGVAVTSAAGGGSPPATSCPVACSCSNQASRVICTRRDLAEVPASIPVNTRYLNLQENGIQVIRTDTFKHLRHLEILQLSKNLVRKIEVGAFNGLPSLNTLELFDNRLTTVPTQAFEYLSKLRELWLRNNPIESIPSYAFNRVPSLRRLDLGELKRLEYISEAAFEGLVNLRYLNLGMCNLKDIPNLTALVRLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYAPVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTKIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 56 - protein, 359 aa, Homo sapiens
MIAAQAKLVYQLNKYYTERCQARKAAIAKTIREVCKVVSDVLKEVEVQEPRFISSLSEIDARYEGLEVISPTEFEVVLYLNQMGVFNFVDDGSLPGCAVLKLSDGRKRSMSLWVEFITASGYLSARKIRSRFQTLVAQAVDKCSYRDVVKMIADTSEVKLRIRERYVVQITPAFKCTGIWPRSAAQWPMPHIPWPGPNRVAEVKAEGFNLLSKECYSLTGKQSSAESDAWVLQFGEAENRLLMGGCRNKCLSVLKTLRDRHLELPGQPLNNYHMKTLLLYECEKHPRETDWDESCLGDRLNGILLQLISCLQCRRCPHYFLPNLDLFQGKPHSALESAAKQTWRLAREILTNPKSLDKL

SEQ ID NO: 57 - protein, 497 aa, Homo sapiens
MASPAPPEHAEEGCPAPAAEEQAPPSPPPPQASPAERQQQEEEAQEAGAAEGAGLQVEEAAGRAAAAVTWLLGEPVLWLGCRADELLSWKRPLRSLLGFVAANLLFWFLALTPWRVYHLISVMILGRVIMQIIKDMVLSRTRGAQLWRSLSESWEVINSKPDERPRLSHCIAESWMNFSIFLQEMSLFKQQSPGKFCLLVCSVCTFFTILGSYIPGVILSYLLLLCAFLCPLFKCNDIGQKIYSKIKSVLLKLDFGIGEYINQKKRERSEADKEKSHKDDSELDFSALCPKISLTVAAKELSVSDTDVSEVSWTDNGTFNLSEGYTPQTDTSDDLDRPSEEVFSRDLSDFPSLENGMGTNDEDELSLGLPTELKRKKEQLDSGHRPSKETQSAAGLTLPLNSDQTFHLMSNLAGDVITAAVTAAIKDQLEGVQQALSQAAPIPEEDTDTEEGDDFELLDQSELDQIESELGLTQDQEAEAQQNKKSSGFLSNLLGGH

SEQ ID NO: 58 - protein, 467 aa, Homo sapiens
MHSTTPISSLFSFTSPAVKRLLGWKQGDEEEKWAEKAVDSLVKKLKKKKGAMDELERALSCPGQPSKCVTIPRSLDGRLQVSHRKGLPHVIYCRVWRWPDLQSHHELKPLECCEFPFGSKQKEVCINPYHYRRVETPVLPPVLVPRHSEYNPQLSLLAKFRSASLHSEPLMPHNATYPDSFQQPPCSALPPSPSHAFSQSPCTASYPHSPGSPSEPESPYQHSVDTPPLPYHATEASETQSGQPVDATADRHVVLSIPNGDFRPVCYEEPQHWCSVAYYELNNRVGETFQASSRSVLIDGFTDPSNNRNRFCLGLLSNVNRNSTIENTRRHIGKGVHLYYVGGEVYAECVSDSSIFVQSRNCNYQHGFHPATVCKIPSGCSLKVFNNQLFAQLLAQSVHHGFEVVYELTKMCTIRMSFVKGWGAEYHRQDVTSTPCWIEIHLHGPLQWLDKVLTQMGSPHNPISSVS

SEQ ID NO: 59 - protein, 1347 aa, Homo sapiens
MENLPFPLKLLSASSLNAPSSTPWVLDIFLTLVFALGFFFLLLPYLSYFRCDDPPSPSPGKRKCPVGRRRRPRGRMKNHSLRAGRECPRGLQETSDLLSQLQSLLGPHLDKGDFGQLSGPDPPGEVGERAPDGASQSSHEPMEDAAPILSPLASPDPQAKHPQDLASTPSPGPMTTSVSSLSASQPPEPSLPLEHPSPEPPALFPHPPHTPDPLACSPPPPKGFTAPPLRDSTLITPSHCDSVALPLGTVPQSLSPHEDLVASVPAISGLGGSNSHVSASSRWQETARTSCAFNSSVQQDHLSRHPPETYQMEAGSLFLLSSDGQNAVGIQVTETAKVNIWEEKENVGSFTDRMTPEKHLNSLRNLAKSLDAEQDTTNPKPFWNMGENSKQLPGPQKLSDPRLWQESFWKNYSQLFWGLPSLHSESLVANAWVTDRSYTLQSPPFLFNEMSNVCPIQRETTMSPLLFQAQPPSHLGPECQPFISSTPQFRPTPMAQAEAQAHLQSSFPVLSPAFPSLIKNTGVACPASQNKVQALSLPETQHPEWPLLRRQLEGRLALPSRVQKSQDVFSVSTPNLPQESLTSILPENFPVSPELRRQLEQHIKKWIIQHWGNLGRIQESLDLMQLRDESPGTSQAKGKPSPWQSSMSTGESSKEAQKVKFQLERDPCPHLGQILGETPQNLSRDMKSFPRKVLGVTSEESERNLRKPLRSDSGSDLLRCTERTHIENILKAHMGRNLGQTNEGLIPVRVRRSWLAVNQALPVSNTHVKTSNLAAPKSGKACVNTAQVLSFLEPCTQQGLGAHIVRFWAKHRWGLPLRVLKPIQCFKLEKVSSLSLTQLAGPSSATCESGAGSEVEVDMFLRKPPMASLRKQVLTKASDHMPESLLASSPAWKQFQRAPRGIPSWNDHGPLKPPPAGQEGRWPSKPLTYSLTGSTQQSRSLGAQSSKAGETREAVPQCRVPLETCMLANLQATSEDMHGFEAPGTSKSSLHPRVSVSQDPRKLCLMEEVVNEFEPGMATKSETQPQVCAAVVLLPDGQASVVPHASENLVSQVPQGHLQSMPAGNMRASQELHDLMAARRSKLVHEEPRNPNCQGSCKNQRPMFPPIHKSEKSRKPNLEKHEERLEGLRTPQLTPVRKTEDTHQDEGVQLLPSKKQPPSVSHFGGNIKQFFQWIFSKKKSKPAPVTAESQKTVKNRSCVYSSSAEAQGLMTAVGQMLDEKMSLCHARHASKVNQHKQKFQAPVCGFPCNHRHLFYSEHGRILSYAASSQQATLKSQGCPNRDRQIRNQQPLKSVRCNNEQWGLRHPQILHPKKAVSPVSPLQHWPKTSGASSHHHHCPRHCLLWEGI

SEQ ID NO: 60 - protein, 1031 aa, Homo sapiens
MPPGRWHAAYPAQAQSSRERGRLQTVKKEEEDESYTPVQAARPQTLNRPGQELFRQLFRQLRYHESSGPLETLSRLRELCRWWLRPDVLSKAQILELLVLEQFLSILPGELRVWVQLHNPESGEEAVALLEELQRDLDGTSWRDPGPAQSPDVHWMGTGALRSAQIWSLASPLRSSSALGDHLEPPYEIEARDFLAGQSDTPAAQMPALFPREGCPGDQVTPTRSLTAQLQETMTFKDVEVTFSQDEWGWLDSAQRNLYRDVMLENYRNMASLVGPFTKPALISWLEAREPWGLNMQAAQPKGNPVAAPTGDDLQSKTNKFILNQEPLEEAETLAVSSGCPATSVSEGIGLRESFQQKSRQKDQCENPIQVRVKKEETNFSHRTGKDSEVSGSNSLDLKHVTYLRVSGRKESLKHGCGKHFRMSSHHYDYKKYGKGLRHMIGGFSLHQRIHSGLKGNKKDVCGKDFSLSSHHQRGQSLHTVGVSFKCSDCGRTFSHSSHLAYHQRLHTQEKAFKCRVCGKAFRWSSNCARHEKIHTGVKPYKCDLCEKAFRRLSAYRLHRETHAKKKFLELNQYRAALTYSSGFDHHLGDQSGEKLFDCSQCRKSFHCKSYVLEHQRIHTQEKPYKCTKCRKTFRWRSNFTRHMRLHEEEKFYKQDECREGFRQSPDCSQPQGAPAVEKTFLCQQCGKTFTRKKTLVDHQRIHTGEKPYQCSDCGKDFAYRSAFIVHKKKHAMKRKPEGGPSFSQDTVFQVPQSSHSKEEPYKCSQCGKAFRNHSFLLIHQRVHTGEKPYKCRECGKAFRWSSNLYRHQRIHSLQKQYDCHESEKTPNVEPKILTGEKRFWCQECGKTFTRKRTLLDHKGIHSGEKRYKCNLCGKSYDRNYRLVNHQRIHSTERPFKCQWCGKEFIGRHTLSSHQRKHTRAAQAERSPPARSSSQDTKLRLQKLKPSEEMPLEDCKEACSQSSRLTGLQDISIGKKCHKCSICGKTFNKSSQLISHKRFHTRERPFKCSKCGKTFRWSSNLARHMKNHIRD

SEQ ID NO: 61 - protein, 1075 aa, Homo sapiens
MRGGKCNMLSSLGCLLLCGSITLALGNAQKLPKGKRPNLKVHINTTSDSILLKFLRPSPNVKLEGLLLGYGSNVSPNQYFPLPAEGKFTEAIVDAEPKYLIVVRPAPPPSQKKSCSGKTRSRKPLQLVVGTLTPSSVFLSWGFLINPHHDWTLPSHCPNDRFYTIRYREKDKEKKWIFQICPATETIVENLKPNTVYEFGVKDNVEGGIWSKIFNHKTVVGSKKVNGKIQSTYDQDHTVPAYVPRKLIPITIIKQVIQNVTHKDSAKSPEKAPLGGVILVHLIIPGLNETTVKLPASLMFEISDALKTQLAKNETLALPAESKTPEVEKISARPTTVTPETVPRSTKPTTSSALDVSETTLASSEKPWIVPTAKISEDSKVLQPQTATYDVFSSPTTSDEPEISDSYTATSDRILDSIPPKTSRTLEQPRATLAPSETPFVPQKLEIFTSPEMQPTTPAPQQTTSIPSTPKRRPRPKPPRTKPERTTSAGTITPKISKSPEPTWTTPAPGKTQFISLKPKIPLSPEVTHTKPAPKQTPRAPPKPKTSPRPRIPQTQPVPKVPQRVTAKPKTSPSPEVSYTTPAPKDVLLPHKPYPEVSQSEPAPLETRGIPFIPMISPSPSQEELQTTLEETDQSTQEPFTTKIPRTTELAKTTQAPHRFYTTVRPRTSDKPHIRPGVKQAPRPSGADRNVSVDSTHPTKKPGTRRPPLPPRPTHPRRKPLPPNNVTGKPGSAGIISSGPITTPPLRSTPRPTGTPLERIETDIKQPTVPASGEELENITDFSSSPTRETDPLGKPRFKGPHVRYIQKPDNSPCSITDSVKRFPKEEATEGNATSPPQNPPTNLTVVTVEGCPSFVILDWEKPLNDTVTEYEVISRENGSFSGKNKSIQMTNQTFSTVENLKPNTSYEFQVKPKNPLGEGPVSNTVAFSTESADPRVSEPVSAGRDAIWTERPFNSDSYSECKGKQYVKRTWYKKFVGVQLCNSLRYKIYLSDSLTGKFYNIGDQRGHGEDHCQFVDSFLDGRTGQQLTSDQLPIKEGYFRAVRQEPVQFGEIGGHTQINYVQWYECGTTIPGKW

SEQ ID NO: 62 - protein, 272 aa, Homo sapiens
MGNTTSDRVSGERHGAKAARSEGAGGHAPGKEHKIMVGSTDDPSVFSLPDSKLPGDKEFVSWQQDLEDSVKPTQQARPTVIRWSEGGKEVFISGSFNNWSTKIPLIKSHNDFVAILDLPEGEHQYKFFVDGQWVHDPSEPVVTSQLGTINNLIHVKKSDFEVFDALKLDSMESSETSCRDLSSSPPGPYGQEMYAFRSEERFKSPPILPPHLLQVILNKDTNISCDPALLPEPNHVMLNHLYALSIKDSVMVLSATHRYKKKYVTTLLYKPI

SEQ ID NO: 63 - protein, 295 aa, Homo sapiens
MELIQDTSRPPLEYVKGVPLIKYFAEALGPLQSFQARPDDLLINTYPKSGTTWVSQILDMIYQGGDLEKCNRAPIYVRVPFLEVNDPGEPSGLETLKDTPPPRLIKSHLPLALLPQTLLDQKVKVVYVARNPKDVAVSYYHFHRMEKAHPEPGTWDSFLEKFMAGEVSYGSWYQHVQEWWELSRTHPVLYLFYEDMKENPKREIQKILEFVGRSLPEETMDFMVQHTSFKEMKKNPMTNYTTVPQELMDHSISPFMRKGMAGDWKTTFTVAQNERFDADYAEKMAGCSLSFRSEL

SEQ ID NO: 64 - protein, 491 aa, Homo sapiens
MAGQGLPLHVATLLTGLLECLGFAGVLFGWPSLVFVFKNEDYFKDLCGPDAGPIGNATGQADCKAQDERFSLIFTLGSFMNNFMTFPTGYIFDRFKTTVARLIAIFFYTTATLIIAFTSAGSAVLLFLAMPMLTIGGILFLITNLQIGNLFGQHRSTIITLYNGAFDSSSAVFLIIKLLYEKGISLRASFIFISVCSTWHVARTFLLMPRGHIPYPLPPNYSYGLCPGNGTTKEEKETAEHENRELQSKEFLSAKEETPGAGQKQELRSFWSYAFSRRFAWHLVWLSVIQLWHYLFIGTLNSLLTNMAGGDMARVSTYTNAFAFTQFGVLCAPWNGLLMDRLKQKYQKEARKTGSSTLAVALCSTVPSLALTSLLCLGFALCASVPILPLQYLTFILQVISRSFLYGSNAAFLTLAFPSEHFGKLFGLVMALSAVVSLLQFPIFTLIKGSLQNDPFYVNVMFMLAILLTFFHPFLVYRECRTWKESPSAIA

SEQ ID NO: 65 - protein, 519 aa, Homo sapiens
MSYPQFGYPYSSAPQFLMATNSLSTCCESGGRTLADSGPAASAQAPVYCPVYESRLLATARHELNSAAALGVYGGPYGGSQGYGNYVTYGSEASAFYSLNSFDSKDGSGSAHGGLAPAAAAYYPYEPALGQYPYDRYGTMDSGTRRKNATRETTSTLKAWLQEHRKNPYPTKGEKIMLAIITKMTLTQVSTWFANARRRLKKENKMTWPPRNKCADEKRPYAEGEEEEGGEEEAREEPLKSSKNAEPVGKEEKELELSDLDDFDPLEAEPPACELKPPFHSLDGGLERVPAAPDGPVKEASGALRMSLAAGGGAALDEDLERARSCLRSAAAGPEPLPGAEGGPQVCEAKLGFVPAGASAGLEAKPRIWSLAHTATAAAAAATSLSQTEFPSCMLKRQGPAAPAAVSSAPATSPSVALPHSGALDRHQDSPVTSLRNWVDGVFHDPILRHSTLNQAWATAKGALLDPGPLGRSLGAGANVLTAPLARAFPPAVPQDAPAAGAARELLALPKAGGKPFCA

SEQ ID NO: 66 - RNA, 9 nt, Artificial Sequence
  A synthetic sequence
uucaagaga

SEQ ID NO: 67 - DNA, 20 bp, Artificial Sequence
  A synthetic sequence
ccaccgctgtccaccgcctc

SEQ ID NO: 68 - DNA, 20 bp, Artificial Sequence
  A synthetic sequence
gacccaaggccagcggccgc

SEQ ID NO: 69 - DNA, 20 bp, Artificial Sequence
  A synthetic sequence
ggagggaggagaaccagcag

SEQ ID NO: 70 - DNA, 20 bp, Artificial Sequence
  A synthetic sequence
aaattgcagccggagcgcgg

SEQ ID NO: 71 - DNA, 20 bp, Artificial Sequence
  A synthetic sequence
tccggataaaccgaggcacg

SEQ ID NO: 72 - DNA, 20 bp, Artificial Sequence
  A synthetic sequence
gcgaagcatcttcaccaccc

SEQ ID NO: 73 - DNA, 20 bp, Artificial Sequence
  A synthetic sequence
gacccagcactgcagcctgg

SEQ ID NO: 74 - DNA, 20 bp, Artificial Sequence
  A synthetic sequence
aaacttatgcggcgtttcct

SEQ ID NO: 75 - DNA, 20 bp, Artificial Sequence
  A synthetic sequence
atcactttaaaaccacctct

SEQ ID NO: 76 - DNA, 20 bp, Artificial Sequence
  A synthetic sequence
ctgaaaaaggaaggagttga

SEQ ID NO: 77 - DNA, 20 bp, Artificial Sequence
  A synthetic sequence
cgcgagcacagctaaggcca

SEQ ID NO: 78 - DNA, 20 bp, Artificial Sequence
  A synthetic sequence
gagtagcgcgagcacagcta

SEQ ID NO: 79 - DNA, 20 bp, Artificial Sequence
  A synthetic sequence
ggccgagatgtctcgctccg

SEQ ID NO: 80 - DNA, 20 bp, Artificial Sequence
  A synthetic sequence
aacaaagcaccctccccact

SEQ ID NO: 81 - DNA, 20 bp, Artificial Sequence
  A synthetic sequence
caaacaggcgtatctgtgtg

SEQ ID NO: 82 - DNA, 20 bp, Artificial Sequence
  A synthetic sequence
ctctgcaaccaggagcccag

SEQ ID NO: 83 - DNA, 20 bp, Artificial Sequence
  A synthetic sequence
cactgttgtcttatcagacg

SEQ ID NO: 84 - DNA, 20 bp, Artificial Sequence
  A synthetic sequence
ctcgtctgataagacaacag

SEQ ID NO: 85 - DNA, 20 bp, Artificial Sequence
  A synthetic sequence
gtttgttcttagggtaacag

SEQ ID NO: 86 - DNA, 20 bp, Artificial Sequence
  A synthetic sequence
actcagccattttattagaa

SEQ ID NO: 87 - DNA, 20 bp, Artificial Sequence
  A synthetic sequence
gagcagcctgtctttatcgg

SEQ ID NO: 88 - DNA, 20 bp, Artificial Sequence
  A synthetic sequence
gttctccgggcccctcctac

SEQ ID NO: 89 - DNA, 20 bp, Artificial Sequence
  A synthetic sequence
cgcctggtgcgcgcccgttg

SEQ ID NO: 90 - DNA, 20 bp, Artificial Sequence
  A synthetic sequence
gaggccacactgcagcgcgg

SEQ ID NO: 91 - DNA, 20 bp, Artificial Sequence
  A synthetic sequence
tgggtctgtctcatcgtcaa

SEQ ID NO: 92 - DNA, 20 bp, Artificial Sequence
  A synthetic sequence
gtggcacaggtgggcataat

SEQ ID NO: 93 - DNA, 20 bp, Artificial Sequence
  A synthetic sequence
gggtggagagttgggacacc

SEQ ID NO: 94 - DNA, 20 bp, Artificial Sequence
  A synthetic sequence
gctcggctgggccagtccgc

SEQ ID NO: 95 - DNA, 20 bp, Artificial Sequence
  A synthetic sequence
tccattcagtcagtctttgg

SEQ ID NO: 96 - DNA, 20 bp, Artificial Sequence
  A synthetic sequence
gagagctatcacctaagtgt

SEQ ID NO: 97 - DNA, 20 bp, Artificial Sequence
  A synthetic sequence
tatctggccctgggtgcttg

SEQ ID NO: 98 - DNA, 20 bp, Artificial Sequence
  A synthetic sequence
ggtgccgcccccagcgtagg

SEQ ID NO: 99 - DNA, 20 bp, Artificial Sequence
  A synthetic sequence
gacaggctcagctatgaaga

SEQ ID NO: 100 - DNA, 20 bp, Artificial Sequence
  A synthetic sequence
gcggcagtgcggcggatgta

SEQ ID NO: 101 - DNA, 20 bp, Artificial Sequence
  A synthetic sequence
tccccctccagctgcaacgg

SEQ ID NO: 102 - DNA, 20 bp, Artificial Sequence
  A synthetic sequence
ggaggactaccaagaggggg

SEQ ID NO: 103 - DNA, 20 bp, Artificial Sequence
  A synthetic sequence
acagtcgtgcagtgcagctg

SEQ ID NO: 104 - DNA, 20 bp, Artificial Sequence
  A synthetic sequence
ggggctctgagagcctctga

SEQ ID NO: 105 - DNA, 20 bp, Artificial Sequence
  A synthetic sequence
gagtcaacggggaataccat

SEQ ID NO: 106 - DNA, 20 bp, Artificial Sequence
  A synthetic sequence
gaaccattagatcaatgcga

SEQ ID NO: 107 - DNA, 20 bp, Artificial Sequence
  A synthetic sequence
ggggagcggcgctaaaggcg

SEQ ID NO: 108 - DNA, 20 bp, Artificial Sequence
  A synthetic sequence
gaaaacacggggtcacgcgc

SEQ ID NO: 109 - DNA, 20 bp, Artificial Sequence
  A synthetic sequence
acctgcacccgcggccgttg

SEQ ID NO: 110 - DNA, 20 bp, Artificial Sequence
  A synthetic sequence
gccgagtcccgcaggcgggg

SEQ ID NO: 111 - DNA, 20 bp, Artificial Sequence
  A synthetic sequence
ttgggagtggtcggattggt

SEQ ID NO: 112 - DNA, 20 bp, Artificial Sequence
  A synthetic sequence
ggcacaaggcagccagatct

SEQ ID NO: 113 - DNA, 20 bp, Artificial Sequence
  A synthetic sequence
aaagggtgtgtggagaaatg

SEQ ID NO: 114 - DNA, 20 bp, Artificial Sequence
  A synthetic sequence
gagcgcgcaaccaggaccac

SEQ ID NO: 115 - DNA, 20 bp, Artificial Sequence
  A synthetic sequence
ccaggcctgtgtcgagtggg

SEQ ID NO: 116 - DNA, 20 bp, Artificial Sequence
  A synthetic sequence
gaggaggagccatggggcgg

SEQ ID NO: 117 - DNA, 21 bp, Artificial Sequence
  A synthetic sequence
ggaagtagaatgtgcctggat

SEQ ID NO: 118 - DNA, 22 bp, Artificial Sequence
  A synthetic sequence
gagaccacagttagagaaccac

SEQ ID NO: 119 - DNA, 20 bp, Artificial Sequence
  A synthetic sequence
cgacagttcagccatcactt

SEQ ID NO: 120 - DNA, 21 bp, Artificial Sequence
  A synthetic sequence
cactgtttttccaagacctca

SEQ ID NO: 121 - DNA, 22 bp, Artificial Sequence
  A synthetic sequence
ctccagaggtttgagttcttct

SEQ ID NO: 122 - DNA, 19 bp, Artificial Sequence
  A synthetic sequence
gcgaagagagccacttctg

SEQ ID NO: 123 - DNA, 20 bp, Artificial Sequence
  A synthetic sequence
ctggtgttgcctcttgtgat

SEQ ID NO: 124 - DNA, 20 bp, Artificial Sequence
  A synthetic sequence
cagcttggacactggatctc

SEQ ID NO: 125 - DNA, 19 bp, Artificial Sequence
  A synthetic sequence
aagcagacggaaagtgagg

SEQ ID NO: 126 - DNA, 22 bp, Artificial Sequence
  A synthetic sequence
gatcctcaagtactttcagcca

SEQ ID NO: 127 - DNA, 20 bp, Artificial Sequence
  A synthetic sequence
tgatctccaagtctgtctgc

SEQ ID NO: 128 - DNA, 18 bp, Artificial Sequence
  A synthetic sequence
acaacttcgtgcactcca

SEQ ID NO: 129 - DNA, 23 bp, Artificial Sequence
  A synthetic sequence
tctgcgtgaatcctagatttctg

SEQ ID NO: 130 - DNA, 18 bp, Artificial Sequence
  A synthetic sequence
gccgaacttctcacagca

SEQ ID NO: 131 - DNA, 22 bp, Artificial Sequence
  A synthetic sequence
gtttttgatccagacccagatg

SEQ ID NO: 132 - DNA, 21 bp, Artificial Sequence
  A synthetic sequence
caagactgagatgcacaagtg

SEQ ID NO: 133 - DNA, 20 bp, Artificial Sequence
  A synthetic sequence
gtaagcaggaagagaagcca

SEQ ID NO: 134 - DNA, 21 bp, Artificial Sequence
  A synthetic sequence
tcttgctattgaccgatgctt

SEQ ID NO: 135 - DNA, 23 bp, Artificial Sequence
  A synthetic sequence
gcaacaaaaagaaacgagatgac

SEQ ID NO: 136 - DNA, 22 bp, Artificial Sequence
  A synthetic sequence
ttcctgctatgattttctccca

SEQ ID NO: 137 - DNA, 20 bp, Artificial Sequence
  A synthetic sequence
aaactcaccaggatgctcac

SEQ ID NO: 138 - DNA, 21 bp, Artificial Sequence
  A synthetic sequence
gtgtacttgctgatcaactgc

SEQ ID NO: 139 - DNA, 18 bp, Artificial Sequence
  A synthetic sequence
agtgtcagtggtgttggc

SEQ ID NO: 140 - DNA, 19 bp, Artificial Sequence
  A synthetic sequence
cctgcacggctacattgag

SEQ ID NO: 141 - DNA, 19 bp, Artificial Sequence
  A synthetic sequence
gtggctatggttggaggtc

SEQ ID NO: 142 - DNA, 20 bp, Artificial Sequence
  A synthetic sequence
cactgccgaggaatgaagag

SEQ ID NO: 143 - DNA, 20 bp, Artificial Sequence
  A synthetic sequence
gagtcctttcgtttccagca

SEQ ID NO: 144 - DNA, 18 bp, Artificial Sequence
  A synthetic sequence
cagcctctgcctcaatgg

SEQ ID NO: 145 - DNA, 21 bp, Artificial Sequence
  A synthetic sequence
gctgagaagttggaagtggaa

SEQ ID NO: 146 - DNA, 21 bp, Artificial Sequence
  A synthetic sequence
cttaacaacctgctaccccat

SEQ ID NO: 147 - DNA, 21 bp, Artificial Sequence
  A synthetic sequence
gcccattattcagagcgagta

SEQ ID NO: 148 - DNA, 21 bp, Artificial Sequence
  A synthetic sequence
gtggcggatttgatcatttgg

SEQ ID NO: 149 - DNA, 26 bp, Artificial Sequence
  A synthetic sequence
ccacagatcagaaacccgatgaaggc

SEQ ID NO: 150 - DNA, 23 bp, Artificial Sequence
  A synthetic sequence
taaagctgtagcccgttgcctgc

SEQ ID NO: 151 - DNA, 27 bp, Artificial Sequence
  A synthetic sequence
tcggtaactgacttgaatgtccaacgc

SEQ ID NO: 152 - DNA, 37 bp, Artificial Sequence
  A synthetic sequence
tctacctctgactgtgatatttttgtgtttaaagtct

SEQ ID NO: 153 - DNA, 24 bp, Artificial Sequence
  A synthetic sequence
ttacatgcccaagaaggccacaga

SEQ ID NO: 154 - DNA, 23 bp, Artificial Sequence
  A synthetic sequence
tgtaacaccccagacccctcgaa

SEQ ID NO: 155 - DNA, 19 bp, Artificial Sequence
  A synthetic sequence
aggtgtgcgggctcaggat

SEQ ID NO: 156 - DNA, 20 bp, Artificial Sequence
  A synthetic sequence
cacttccgcatctgcccgtg

SEQ ID NO: 157 - DNA, 24 bp, Artificial Sequence
  A synthetic sequence
tgtagcagctgatccgagcctaga

SEQ ID NO: 158 - DNA, 24 bp, Artificial Sequence
  A synthetic sequence
cgagggtggaggcctgaattttga

SEQ ID NO: 159 - DNA, 25 bp, Artificial Sequence
  A synthetic sequence
tgcttctctctctgtcttcgggtga

SEQ ID NO: 160 - DNA, 23 bp, Artificial Sequence
  A synthetic sequence
acggtgttctgtttctcctggca

SEQ ID NO: 161 - DNA, 27 bp, Artificial Sequence
  A synthetic sequence
agagagcagactggaagaaaacagtgg

SEQ ID NO: 162 - DNA, 24 bp, Artificial Sequence
  A synthetic sequence
cagatgtcccagttcctgtgcctt

SEQ ID NO: 163 - DNA, 23 bp, Artificial Sequence
  A synthetic sequence
tgcagggtttcaccaggatccac

SEQ ID NO: 164 - DNA, 26 bp, Artificial Sequence
  A synthetic sequence
aattcacgcagaaggaaccagacagt

SEQ ID NO: 165 - DNA, 14 bp, Artificial Sequence
  A synthetic sequence
gtttaagagctatg